(19) United States
(12) Patent Application Publication
(10) Pub. No.: US 2010/0331297 A1
(43) Pub. Date: Dec. 30, 2010

(54) MODULATION OF PROTEIN TRAFFICKING

(75) Inventors: Christine Ellen Bulawa, Arlington, MA (US); Michael DeVit, Sudbury, MA (US); Daniel Elbaum, Newton, MA (US)

Correspondence Address: FISH & RICHARDSON P.C. (NY), P.O. BOX 1022, MINNEAPOLIS, MN, 55440-1022, US

(73) Assignee: FOLDRX PHARMACEUTICALS, INC., CAMBRIDGE, MA (US)

(21) Appl. No.: 12/741,992

(22) PCT Filed: Nov. 7, 2008
(86) PCT No.: PCT/US08/82909

§ 371 Date: Aug. 17, 2010

Related U.S. Application Data
(60) Provisional application No. 60/986,282, filed on Nov. 7, 2007.

(51) Int. Cl. A61K 31/52; C12P 21/06; C12N 5/02; A61K 31/519; C07D 473/00; C07D 487/02; A61P 25/16; A61P 25/28; A61P 3/10

(52) U.S. Cl. 514/210.18; 435/69.1; 435/375; 514/262.1; 514/265.1; 544/277; 544/280

(57) ABSTRACT

Compounds and compositions are provided for treatment or amelioration of one or more disorders characterized by defects in protein trafficking. A method of treating a disorder characterized by impaired protein trafficking includes administering to a subject or contacting a cell with a compound of Formula I: [formula here] or pharmaceutically acceptable salts or derivatives thereof.

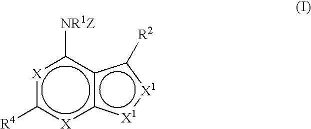

### FIELD

This invention relates to compounds and methods for modulating protein trafficking and treating or preventing disorders characterized by impaired protein trafficking.

### BACKGROUND

Disorders characterized by impaired protein trafficking are numerous and include genetic diseases such as Huntington's disease, Tay-Sachs disease, familial hypercholesterolemia, and cystic fibrosis. Mutations in genes associated with these disorders often result in proteins that improperly fold and/or are retained in the endoplasmic reticulum. As a result, these proteins are often prematurely degraded.

The failure of a cell (e.g., in a tissue) to express a sufficient amount of an essential protein, e.g., an enzyme, can result in disease states, which vary in presentation and severity among protein trafficking disorders. For example, cystic fibrosis can affect nearly the entire body, causing progressive disability and early death. Difficulty breathing is the most common symptom and results from frequent lung infections, which can be treated by antibiotics and other medications. A multitude of other symptoms, including sinus infections, poor growth, diarrhea, and infertility can result from the effects of cystic fibrosis on other parts of the body. Cystic fibrosis, like many other disorders characterized by impaired protein trafficking, can be lethal if untreated.

Other protein trafficking disorders include, for example, α-synuclein mediated disorders, or disorders in which α-synuclein fibril formation is implicated, including but not limited to, Parkinson's disease, dementia with Lewy bodies, multiple system atrophy and the Lewy body variant of Alzheimer's disease.

For example, Parkinson's disease is a neurodegenerative disorder that is pathologically characterized by the presence of intracytoplasmic Lewy bodies (Lewy in Handbuch der Neurologie, M. Lewandowski, ed., Springer, Berlin, pp. 920-933, 1912; Pollanen et al., J. Neuropath. Exp. Neurol. 52:183-191, 1993), the major components of which are filaments consisting of α-synuclein (Spillantini et al., Proc. Natl. Acad. Sci. USA 95:6469-6473, 1998; Arai et al., Neurosci. Lett. 259:83-86, 1999), an 140-amino acid protein (Ueda et al., Proc. Natl. Acad. Sci. USA 90:11282-11286, 1993). Two dominant mutations in α-synuclein causing familial early onset Parkinson's disease have been described suggesting that Lewy bodies contribute mechanistically to the degeneration of neurons in Parkinson's disease and related disorders (Polymeropoulos et al., Science 276:2045-2047, 1997; Kruger et al., Nature Genet. 18:106-108, 1998; Zarranz et al., Ann. Neurol. 55:164-173, 2004). Triplication and duplication mutation of the α-synuclein gene have been linked to early-onset of Parkinson's disease (Singleton et al., Science 302:841, 2003; Chartier-Harlin at al. Lancet 364:1167-1169, 2004; Ibanez et al., Lancet 364:1169-1171, 2004). In vitro studies have demonstrated that recombinant α-synuclein can indeed form Lewy body-like fibrils (Conway et al., Nature Med. 4:1318-1320, 1998; Hashimoto et al., Brain Res. 799:301-306, 1998; Nahri et al., J. Biol. Chem. 274:9843-9846, 1999). Both Parkinson's disease-linked α-synuclein mutations accelerate this aggregation process, demonstrating that such in vitro studies may have relevance for Parkinson's disease pathogenesis. α-synuclein aggregation and fibril formation fulfills of the criteria of a nucleation-dependent polymerization process (Wood et al., J. Biol. Chem. 274:19509-19512, 1999). In this regard α-synuclein fibril formation resembles that of Alzheimer's β-amyloid protein (Aβ) fibrils. α-synuclein recombinant protein, and non-Aβ component (known as NAC), which is a 35-amino acid peptide fragment of α-synuclein, both have the ability to form fibrils when incubated at 37° C., and are positive with amyloid stains such as Congo red (demonstrating a red/green birefringence when viewed under polarized light) and Thioflavin S (demonstrating positive fluorescence) (Hashimoto et al., Brain Res. 799:301-306, 1998; Ueda et al., Proc. Natl. Acad. Sci. USA 90:11282-11286, 1993).

Synucleins are a family of small, presynaptic neuronal proteins composed of α-, β-, and γ-synucleins, of which only α-synuclein aggregates have been associated with several neurological diseases (Ian et al., Clinical Neurosc. Res. 1:445-455, 2001; Trojanowski and Lee, Neurotoxicology 23:457-460, 2002). The role of synucleins (and in particular, α-synuclein) in the etiology of a number of neurodegenerative and/or amyloid diseases has developed from several observations. Pathologically, α-synuclein was identified as a major component of Lewy bodies, the hallmark inclusions of Parkinson's disease, and a fragment thereof was isolated from amyloid plaques of a different neurological disease, Alzheimer's disease. Biochemically, recombinant α-synuclein was shown to form amyloid-like fibrils that recapitulated the ultrastructural features of α-synuclein isolated from patients with dementia with Lewy bodies, Parkinson's disease and multiple system atrophy. Additionally, the identification of mutations within the α-synuclein gene, albeit in rare cases of familial Parkinson's disease, demonstrated an unequivocal link between synuclein pathology and neurodegenerative diseases. The common involvement of α-synuclein in a spectrum of diseases such as Parkinson's disease, dementia with Lewy bodies, multiple system atrophy and the Lewy body variant of Alzheimer's disease has led to the classification of these diseases under the umbrella term of “synucleinopathies.”

Fibrillization and aggregation of α-synuclein is thought to play major role in neuronal dysfunction and death of dopaminergic neurons in PD. Mutations in α-synuclein or genomic triplication of wild type α-synuclein (leading to its overexpression) cause certain rare familial forms of Parkinson's disease. In vitro and in vivo models suggest that over-expression of wild-type α-synuclein induces neuronal cell death. See, e.g., Polymeropoulos, et al. (1997) Science 276(5321):2045-7, Kruger, et al. (1998) Nat. Genet. 18(2):106-8, Singleton, et al. (2003) Science 302(5646):841, Miller, et al. (2004) Neurology 62(10):1835-8, Hashimoto, et al. (2003) Ann N Y Acad. Sci. 991:171-88, Lo Bianco, et al. (2002) Proc Natl Acad Sci USA. 99(16):10813-8, Lee, et al. (2002) Proc Natl Acad Sci USA. 99 (13):8968-73, Masliah, et al. (2000) Science 287(5456):1265-9, Auluck, et al. (2002) Science 295(5556):865-8, Oluwatosin-Chigbu et al. (2003) Biochem Biophys Res Commun 309(3): 679-84, Klucken et al. (2004) J Biol. Chem. 279(24):25497-502. Protecting neurons from the toxic effects of α-synuclein is a promising strategy for treating Parkinson's disease and other synucleinopathies such as Lewy body dementia.

Thus, there is a need for compounds and compositions that rescue protein trafficking in order to treat diseases and disorders mediated by protein trafficking, such as cystic fibrosis and Parkinson's disease.

### SUMMARY

Provided herein are compounds, compositions containing the compounds, and methods of use of the compounds to rescue impaired protein trafficking. Also provided are methods of treatment or amelioration of one or more symptoms of disorders associated with impaired protein trafficking. Such disorders include, for example, cystic fibrosis.

Use of any of the described compounds for the treatment or amelioration of one or more symptoms of disorders associated with impaired protein trafficking is also contemplated. Furthermore, use of any of the described compounds for the manufacture of a medicament for the treatment of disorders associated with impaired protein trafficking is also contemplated. A method of treating a subject for a disorder characterized by impaired protein trafficking includes administering to the subject an effective amount of a compound represented by the following structural formula:

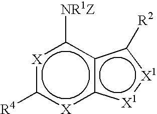

or pharmaceutically acceptable salts thereof, wherein the disorder is not a synucleinopathy.

A method of increasing protein trafficking in a cell includes contacting the cell with an effective amount of a compound represented by the above structural formula, or pharmaceutically acceptable salts thereof, wherein the cell is not characterized by impaired synuclein trafficking.

A method of treating a disorder characterized by impaired protein trafficking, includes administering a compound to a subject or contacting a cell with the compound, wherein the compound is represented by the above structural formula, or pharmaceutically acceptable salts thereof, wherein the disorder is not a synucleinopathy.

In various embodiments of above methods, in the compound represented by the above structural formula:

m is 1 or 2;

each X is independently N, CH, or C(C₁-C₄ alkyl);

each X¹ is independently N, NR³, CH, or C(C₁-C₄ alkyl);

R¹ and Z are each independently R⁵, C(O)R⁵, COOR⁵, C(O)NR⁵R⁵, or S(O)ₘR⁵; or, NR¹Z, taken together, is N═CH—NR⁵R⁵

R² and R³ are each independently H, halo, pseudohalo, CN, SR⁵, R⁵, OR⁵, OC(O)R⁵, NR⁵R⁵, NR⁵R⁶, COOR⁵, NO₂, C(O)R⁵, C(O)C(O)R⁵, C(O)NR⁵R⁵, S(O)ₘR⁵, S(O)ₘNR⁵R⁵, NR⁵C(O)NR⁵R⁵, NR⁵C(O)C(O)R⁵, NR⁵C(O)R⁵, NR⁵(COOR⁵), NR⁵C(O)R⁸, NR⁵S(O)ₘNR⁵R⁵, NR⁵S(O)ₘR⁵, NR⁵S(O)ₘR⁸, NR⁵C(O)C(O)NR⁵R⁵, NR⁵C(O)C(O)NR⁵R⁶, P(O)R⁵R⁵, P(O)(NR⁵R⁵)₂, P(O)(NR⁵R⁶)₂, P(O)(NR⁶R⁶)₂, or P(O)(OR⁵)₂;

R⁴ is independently H, halo, pseudohalo, CN, SR⁵, OR⁵, OC(O)R⁵, NR⁵R⁵, NR⁵R⁶, COOR⁵, NO₂, C(O)R⁵, C(O)C(O)R⁵, C(O)NR⁵R⁵, S(O)ₘR⁵, S(O)ₘNR⁵R⁵, NR⁵C(O)NR⁵R⁵, NR⁵C(O)C(O)R⁵, NR⁵C(O)R⁵, NR⁵(COOR⁵), NR⁵C(O)R⁸, NR⁵S(O)ₘNR⁵R⁵, NR⁵S(O)ₘR⁵, NR⁵S(O)ₘR⁸, NR⁵C(O)C(O)NR⁵R⁵, or NR⁵C(O)C(O)NR⁵R⁶; or optionally substituted alkyl, aryl, aralkyl, heteroaryl, or heteroaralkyl; and

each R⁵, R⁶, and R⁸ is independently H or optionally substituted alkyl, alkenyl, alkynyl, cycloalkyl, cycloalkenyl, aryl, heteroaryl, or heterocyclyl.

In some embodiments of above methods, in the compound represented by the above structural formula:

m is 1 or 2;

each X is independently N or CH;

each X¹ is independently N, NR³ or CH;

R¹ and Z are each independently R⁵, C(O)R⁵, COOR⁵, C(O)NR⁵R⁵, or S(O)ₘR⁵;

R² and R³ are each independently H, halo, pseudohalo, CN, SR⁵, R⁵, OR⁵, OC(O)R⁵, NR⁵R⁵, NR⁵R⁶, COOR⁵, NO₂, C(O)R⁵, C(O)C(O)R⁵, C(O)NR⁵R⁵, S(O)ₘR⁵, S(O)ₘNR⁵R⁵, NR⁵C(O)NR⁵R⁵, NR⁵C(O)C(O)R⁵, NR⁵C(O)R⁵, NR⁵(COOR⁵), NR⁵C(O)R⁸, NR⁵S(O)ₘNR⁵R⁵, NR⁵S(O)ₘR⁵, NR⁵S(O)ₘR⁸, NR⁵C(O)C(O)NR⁵R⁵, NR⁵C(O)C(O)NR⁵R⁶, P(O)R⁵R⁵, P(O)(NR⁵R⁵)₂, P(O)(NR⁵R⁶)₂, P(O)(NR⁶R⁶)₂, or P(O)(OR⁵)₂;

R⁴ is independently H, halo, pseudohalo, CN, SR⁵, OR⁵, OC(O)R⁵, NR⁵R⁵, NR⁵R⁶, COOR⁵, NO₂, C(O)R⁵, C(O)C(O)R⁵, C(O)NR⁵R⁵, S(O)ₘR⁵, S(O)ₘNR⁵R⁵, NR⁵C(O)NR⁵R⁵, NR⁵C(O)C(O)R⁵, NR⁵C(O)R⁵, NR⁵(COOR⁵), NR⁵C(O)R⁸, NR⁵S(O)ₘNR⁵R⁵, NR⁵S(O)ₘR⁵, NR⁵S(O)ₘR⁸, NR⁵C(O)C(O)NR⁵R⁵, or NR⁵C(O)C(O)NR⁵R⁶; or optionally substituted alkyl, aryl, aralkyl, heteroaryl, or heteroaralkyl; and

each R⁵, R⁶, and R⁸ is independently H or optionally substituted alkyl, alkenyl, alkynyl, cycloalkyl, cycloalkenyl, aryl, heteroaryl, or heterocyclyl.

In some embodiments, R² is independently H, halo, pseudohalo, (CH₂)ₙ—Y, or (CH═CH)ₙ—Y, where Y is unsubstituted or substituted aryl, heteroaryl, alkyl, or cycloalkyl. In various embodiments, substituents for Y are independently selected from the group consisting of halo, pseudohalo, alkyl, cycloalkyl, aryl, aralkyl, NO₂, alkoxy, aryloxy, arylalkyoxy, CF₃, OCF₃, CN, NR⁵R⁶, NR⁵COR⁶, (CH₂)ₙOR⁶, SR⁶, CO₂H, CO₂R⁶, CONR⁶R⁵, COR⁶, and SO₂NR⁵R⁶. In some embodiments, R³ is independently substituted or unsubstituted alkyl, alkenyl, alkynyl, aryl, aralkyl, cycloalkyl, (CH₂)ₙ-cycloalkyl, or adamantly. In some embodiments, R⁴ is independently H, NH₂, NR⁵R⁶, NR⁵COR⁶, or unsubstituted or substituted alkyl or aryl. R¹, Z, R⁵, and R⁶ are independently selected from H, unsubstituted or substituted alkyl, aralkyl, aryl, alkaryl, or cycloalkyl, COR^{o7}, where R^{o7} is unsubstituted or substituted alkyl or aryl, SO₂R^{o8}, where R^{o8} is aryl or substituted aryl, and (CH₂)ₙ-cycloalkyl, where the cycloalkyl may be substituted. In some embodiments, X is independently CH or N.

In some embodiments, the compound is represented by the following structural formula, wherein R³ is independently optionally substituted alkyl, cycloalkyl, alkoxy, aryl, aralkyl, heteroaryl, or heteroaralkyl:

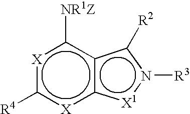

In some embodiments, the compound is represented by one of the following structural formulae:

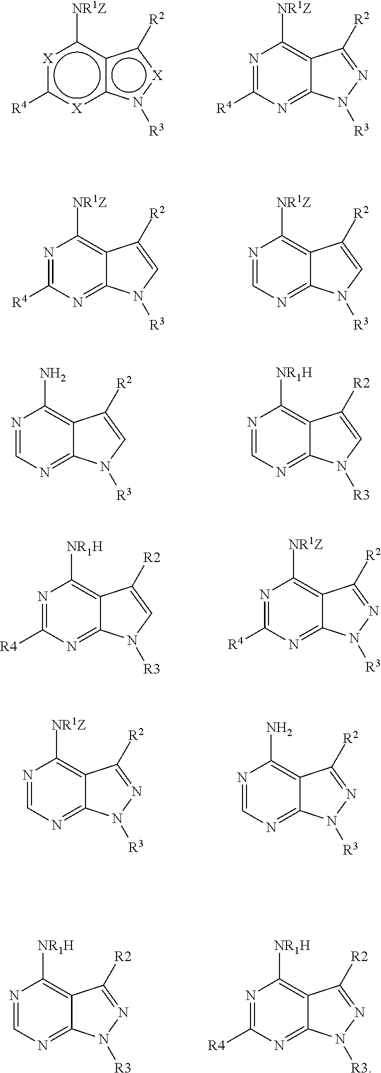

In various embodiments, R¹ and Z are each independently selected from the group consisting of hydrogen, or substituted or unsubstituted alkyl, alkylcarbonyl, arylcarbonyl, aralkylcarbonyl, haloarylcarbonyl, arylsulfonyl, aralkylsulfonyl, and haloarylsulfonyl. In some embodiments, R¹ is independently H and Z is H. In some embodiments, R¹ is independently methyl and Z is H. In certain embodiments, R¹ is H.

In various embodiments, R² is independently hydrogen, halo, or optionally substituted aryl, heteroaryl, aralkyl, or aralkenyl. In some embodiments, R² is independently H, halo, CN, NO₂, NH₂, or C₁-C₁₀ alkyl optionally substituted with 1-3 independent halo, SR⁵, OR⁵, OC(O)R⁵, NR⁵R⁵; COOR⁵, NO₂, CN, C(O)R⁵, OC(O)NR⁵R⁵, or C(O)NR⁵R⁵. In certain embodiments, R² is independently H, F, Cl, Br, CF₃, CCl₃, CN, NO₂, NH₂, or C₁-C₆ alkyl. In various embodiments, R² is independently aryl, heteroaryl, aralkyl, or heteroaralkyl, each independently substituted with: H, halo, SR⁵, OR⁵, OC(O)R⁵, NR⁵R⁵, COOR⁵, NO₂, CN, C(O)R⁵, OC(O) NR⁵R⁵, or C(O)NR⁵R⁵; or aryl, C₁-C₁₀ alkyl, or C₂-C₁₀ alkenyl each optionally substituted with 1-3 independent aryl, halo, SR⁵, OR⁵, OC(O)R⁵, NR⁵R⁵, COOR⁵, NO₂, CN, C(O)R⁵, OC(O) NR⁵R⁵, or C(O)NR⁵R⁵. The optionally substituted aryl, heteroaryl, aralkyl, or heteroaralkyl groups, e.g., in R² can be independently selected from phenyl, napthyl, benzyl, phenylethylene, napthylmethylene, phenoxymethylene, napthyloxymethylene, pyridylmethylene, benzofurylmethylene, dihydrobenzofurylmethylene, benzodioxolmethylene, indanylmethylene, furyl, thienyl, pyridyl, benzothienyl, and benzofuryl. The optional substituents for the aryl, heteroaryl, aralkyl, or heteroaralkyl groups in R² can independently be: H, F, Cl, Br, OH, C₁-C₆ alkoxy, amino, C₁-C₆ alkylamino, COOH, COO—C₁-C₆ alkyl, NO₂, CN, or C(O)—C₁-C₆ alkyl; or C₁-C₆ alkyl, C₂-C₆ alkenyl, or aryl optionally substituted with phenyl, F, Cl, Br, C₁-C₆ alkoxy, COOH, COO—C₁-C₆ alkyl, NO₂, or CN.

In some embodiments, R³ is independently selected from the group consisting of substituted or unsubstituted alkyl, cycloalkyl, aryl, and aralkyl. In various embodiments, R³ is independently H, C₃-C₁₀ cycloalkyl, or C₂-C₁₀ alkynyl; or C₁-C₁₀ alkyl or C₂-C₁₀ alkenyl each optionally substituted with 1-3 halo, CF₃, SR⁵, OR⁵, OC(O)R⁵, NR⁵R⁵, COOR⁵, NO₂, CN, C(O)R⁵, OC(O)NR⁵R⁵, or C(O)NR⁵R⁵. In some embodiments, R³ is independently H, C₁-C₈ alkyl optionally substituted with 1-3 halo, OR⁵, NR⁵R⁵, COOR⁵, C(O)R⁵, C(O)NR⁵R⁵, C₂-C₆ alkenyl, or C₂-C₆ alkynyl; or cyclopropyl, cyclopropylmethyl, cyclobutyl, cyclobutylmethyl, cyclopentyl, cyclopentylmethyl, cyclohexyl, or cyclohexylmethyl. In various embodiments, R³ is independently aryl, heteroaryl, aralkyl, heteroaralkyl, heterocyclyl, or heterocyclyalkyl, each substituted with: H, alkyl, halo, OR⁵, OC(O)R⁵, NR⁵R⁵, COOR⁵, NO₂, CN, C(O)R⁵, OC(O)NR⁵R⁵, or C(O)NR⁵R⁵; or optionally substituted aryl, heteroaryl, or heterocyclyl. The aryl, heteroaryl, aralkyl, heteroaralkyl, heterocyclyl, or heterocyclyalkyl groups, e.g., represented by R³, can be independently selected from benzyl, pyridyl, pyridylmethylene, furyl, thienyl, tetrahydrofuryl, or tetrahydrothienyl. In certain embodiments, substituents for the aryl, heteroaryl, aralkyl, heteroaralkyl, heterocyclyl, or heterocyclyalkyl groups represented by R³ can independently be: H, F, Cl, Br, SR⁵, OR⁵, NR⁵R⁵, COOR⁵, NO₂, CN, C(O)R⁵; or C₁-C₆ alkyl, C₂-C₆ alkenyl, or aryl optionally substituted with phenyl, F, Cl, Br, SR⁵, OR⁵, COOR⁵, NO₂, or CN.

In some embodiments, R⁴ is independently H, alkyl, cycloalkyl, or alkylcycloalkyl. In various embodiments, R⁴ is independently aryl; heteroaryl; C₁-C₁₀ alkyl or C₂-C₁₀ alkenyl, each optionally substituted with 1-3 independent aryl, or heteroaryl; C₂-C₁₀ alkynyl; halo; haloalkyl; CF₃; SR⁵; OR⁵; OC(O)R⁵; NR⁵R⁵; NR⁵R⁶; COOR⁵; NO₂; CN; C(O)R⁵; C(O)C(O)R⁵; C(O)NR⁵R⁵; S(O)ₘR⁵; S(O)ₘNR⁵R⁵; NR⁵C(O)NR⁵R⁵; NR⁵C(O)C(O)R⁵; NR⁵C(O)R⁵; NR⁵(COOR⁵); NR⁵C(O)R⁸; NR⁵S(O)ₘNR⁵R⁵; NR⁵S(O)ₘR⁵; NR⁵S(O)ₘR⁸; NR⁵C(O)C(O)NR⁵R⁵; or NR⁵C(O)C(O)NR⁵R⁶. In some embodiments, R⁴ is independently H, OR⁵, OC(O)R⁵, NR⁵R⁵, COOR⁵, NO₂, CN, C(O)R⁵, C(O)C(O)R⁵, or C(O)NR⁵R⁵; or C₁-C₁₀ alkyl optionally substituted with 1-3 halo, OR⁵, OC(O)R⁵, NR⁵R⁵; COOR⁵, NO₂, CN, C(O)R⁵, OC(O)NR⁵R⁵, or C(O)NR⁵R⁵. In certain embodiments, R⁴ is independently H, CF₃, CCl₃, amino, C₁-C₆ alkoxy, COOH, COO—C₁-C₆ alkyl, OC(O)—C₁-C₆ alkyl, phenoxy, or alkylphenoxy; or C₁-C₆ alkyl optionally substituted with amino, COOH, COO—C₁-C₆ alkyl or OC(O)—C₁-C₆ alkyl, or 1 or 2 C₁-C₆ alkoxy. In some embodiments, R⁴ is independently an optionally substituted aryl, aralkyl, heteroaryl, or heteroaralkyl, wherein the optional substituents can include halo, CF₃, SR⁵, OR⁵, OC(O)R⁵, NR⁵R⁵, COOR⁵, NO₂, CN, C(O)R⁵, OC(O)NR⁵R⁵, C(O) NR⁵R⁵, N(R⁵)C(O)R⁵, N(R⁵)(COOR⁵), or S(O)ₘNR⁵R⁵. The aryl, aralkyl, heteroaryl, and heteroaralkyl groups, e.g., as represented by R⁴, can be independently selected from phenyl, benzyl, pyridyl, pyridylmethylene, furyl, furylmethylene, thienyl, thienylmethylene, pyrazolyl, and pyrazolylmethylene. In various embodiments, the optional substituents for the aryl, aralkyl, heteroaryl, or heteroaralkyl groups represented by R⁴ are independently F, Cl, OH, amino, NO₂, C₁-C₆ alkoxy, C₁-C₆ alkyl, phenoxy, or alkylphenoxy; or phenyl, imidazolyl, or morpholino optionally substituted with F, Cl, amino, NO₂, C₁-C₆ alkoxy, or C₁-C₆ alkyl.

In certain embodiments, the compound is selected from the compounds set forth in FIG. 1A, 1B, 1C, 1D, 1E, 1F, 2, 3A, 3B, 4A, 4B, 5A, 5B, 6, 7, 8A, 8B, 8C, 9A, 9B, 9C, or 9D. In some embodiments, the compound is selected from the compounds set forth in Table I.

In various embodiments, a compound is represented by the following structural formula:

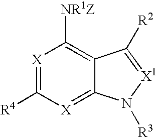

or pharmaceutically acceptable salts thereof.

In various embodiments, the compound is represented by one of the following structural formulas:

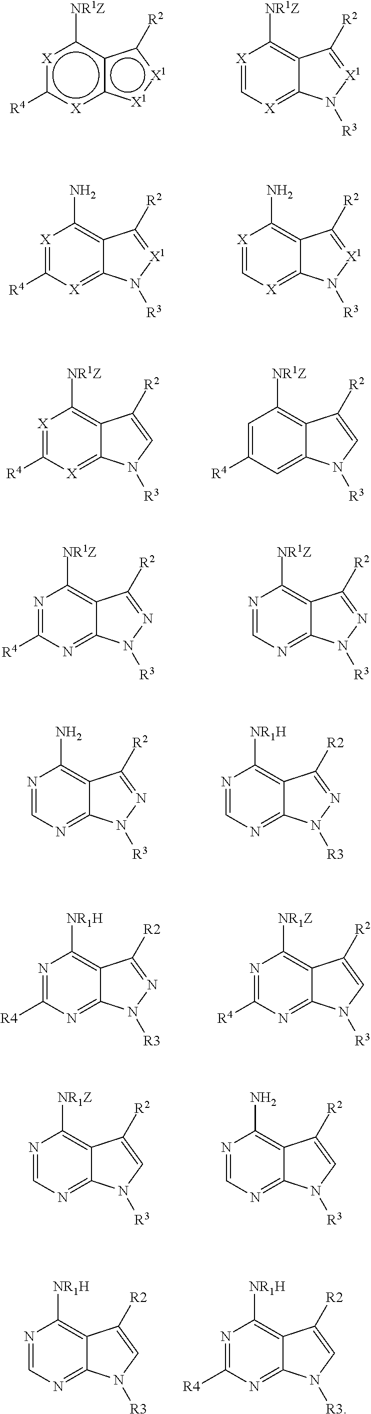

In various embodiments, in the above structural formulae:

m is 1 or 2;

each X and X¹ is independently N, CH, or C(C₁-C₄ alkyl);

R¹ and Z are each independently H, R⁵, C(O)R⁶, COOR⁵, C(O)NR⁶R⁶, or S(O)ₘR⁵; or, NR¹Z, taken together, is N═CH—NR⁵R⁵

R² is SR⁹, OR⁵, OC(O)R⁵, NR⁵R⁵, NR⁵R⁶, COOR⁵, C(O)R⁵, C(O)H, C(O)C(O)R⁵, C(O)NR⁵R⁵, C(O)NR⁵R⁶, C(O)NR⁶R⁶, S(O)ₘR⁹, S(O)ₘNR⁵R⁵, S(O)ₘNR⁵R⁶, NR⁵C(O)NR⁵R⁵, NR⁶C(O)NR⁶R⁶, NR⁵C(O)C(O)R⁵, NR⁵C(O)C(O)R⁵, NR⁵C(O)R⁵, NR⁶C(O)R⁵, NR⁵(COOR⁵), NR⁶(COOR⁵), NR⁵C(O)R⁸, NR⁶C(O)R⁸, NR⁵S(O)ₘNR⁵R⁵, NR⁶S(O)ₘNR⁶R⁶, NR⁵S(O)ₘR⁵, NR⁶S(O)ₘR⁵, NR⁵S(O)ₘR⁸, NR⁶S(O)ₘR⁸, NR⁵C(O)C(O)NR⁵R⁵, NR⁶C(O)C(O)NR⁵R⁶, or NR⁵C(O)C(O)NR⁵R⁶;

R³ is R¹⁰, COOR⁵, C(O)R⁵, C(O)C(O)R⁵, C(O)NR⁵R⁵, C(O)NR⁵R⁶, C(O)NR⁶R⁶, S(O)ₘR⁵, S(O)ₘNR⁵R⁵, S(O)ₘNR⁵R⁶, P(O)R⁵R⁵, P(O)(NR⁵R⁵)₂, P(O)(NR⁵R⁶)₂, P(O)(NR⁶R⁶)₂, or P(O)(OR⁵)₂;

R⁴ is H, halo, pseudohalo, CN, SR⁵, OR⁵, OC(O)R⁵, NR⁵R⁵, NR⁵R⁶, COOR⁵, NO₂, C(O)R⁵, C(O)C(O)R⁵, C(O)NR⁵R⁵, C(O)NR⁵R⁶, C(O)NR⁶R⁶, S(O)ₘR⁵, S(O)ₘNR⁵R⁵, S(O)ₘNR⁵R⁶, NR⁵C(O)NR⁵R⁵, NR⁶C(O)NR⁶R⁶, NR⁵C(O)C(O)R⁵, NR⁵C(O)C(O)R⁵, NR⁵C(O)R⁵, NR⁶C(O)R⁵, NR⁵(COOR⁵), NR⁶(COOR⁵), NR⁵C(O)R⁸, NR⁶C(O)R⁸, NR⁵S(O)ₘNR⁵R⁵, NR⁶S(O)ₘNR⁶R⁶, NR⁵S(O)ₘR⁵, NR⁶S(O)ₘR⁵, NR⁵S(O)ₘR⁸, NR⁶S(O)ₘR⁸, NR⁵C(O)C(O)NR⁵R⁵, NR⁶C(O)C(O)NR⁵R⁶, or NR⁵C(O)C(O)NR⁵R⁶, NR⁶C(O)C(O)NR⁵R⁶, or NR⁵C(O)C(O)NR⁵R⁶, or optionally substituted alkyl, aryl, araalkyl, heteroaryl, or heteroaralkyl; and

each R⁵ is independently optionally substituted alkyl, alkenyl, alkynyl, cycloalkyl, cycloalkenyl, aryl, heteroaryl, or heterocyclyl,

each R⁶ and R⁸ is independently H or optionally substituted alkyl, alkenyl, alkynyl, cycloalkyl, cycloalkenyl, aryl, heteroaryl, or heterocyclyl,

each R⁹ is independently optionally substituted alkyl containing 2 or more carbons, alkenyl, alkynyl, cycloalkyl, cycloalkenyl, aryl, heteroaryl, or heterocyclyl, and

each R¹⁰ is independently optionally substituted alkyl, alkenyl, alkynyl, cycloalkyl, cycloalkenyl, aryl, heteroaryl, or heterocyclyl, excluding optionally substituted dihydrofur-2-yl and tetrahydrofur-2-yl.

In various embodiments, in the above structural formulae:

m is 1 or 2;

each X is independently N, CH, or C(C₁-C₄ alkyl);

each X¹ is independently N, NR³, CH, or C(C₁-C₄ alkyl);

R¹ and Z are each independently R⁵, C(O)R⁵, COOR⁵, C(O)NR⁵R⁵, or S(O)ₘR⁵; or, NR¹Z, taken together, is N═CH—NR⁵R⁵

R² is N₃-substituted alkyl, alkenyl, alkynyl, cycloalkyl, cycloalkenyl, aryl, heteroaryl, or heterocyclyl, which may be further optionally substituted;

R³ is independently H, halo, pseudohalo, CN, SR⁵, R⁵, OR⁵, OC(O)R⁵, NR⁵R⁵, NR⁵R⁶, COOR⁵, NO₂, C(O)R⁵, C(O)C(O)R⁵, C(O)NR⁵R⁵, S(O)ₘR⁵, S(O)ₘNR⁵R⁵, NR⁵C(O)NR⁵R⁵, NR⁵C(O)C(O)R⁵, NR⁵C(O)R⁵, NR⁵(COOR⁵), NR⁵C(O)R⁸, NR⁵S(O)ₘNR⁵R⁵, NR⁵S(O)ₘR⁵, NR⁵S(O)ₘR⁸, NR⁵C(O)C(O)NR⁵R⁵, NR⁵C(O)C(O)NR⁵R⁶, P(O)R⁵R⁵, P(O)(NR⁵R⁵)₂, P(O)(NR⁵R⁶)₂, P(O)(NR⁶R⁶)₂, or P(O)(OR⁵)₂;

R⁴ is independently H, halo, pseudohalo, CN, SR⁵, OR⁵, OC(O)R⁵, NR⁵R⁵, NR⁵R⁶, COOR⁵, NO₂, C(O)R⁵, C(O)C(O)R⁵, C(O)NR⁵R⁵, S(O)ₘR⁵, S(O)ₘNR⁵R⁵, NR⁵C(O)NR⁵R⁵, NR⁵C(O)C(O)R⁵, NR⁵C(O)R⁵, NR⁵(COOR⁵), NR⁵C(O)R⁸, NR⁵S(O)ₘNR⁵R⁵, NR⁵S(O)ₘR⁵, NR⁵S(O)ₘR⁸, NR⁵C(O)C(O)NR⁵R⁵, or NR⁵C(O)C(O)NR⁵R⁶; or optionally substituted alkyl, aryl, aralkyl, heteroaryl, or heteroaralkyl; and

each R⁵, R⁶, and R⁸ is independently H or optionally substituted alkyl, alkenyl, alkynyl, cycloalkyl, cycloalkenyl, aryl, heteroaryl, or heterocyclyl.

In various embodiments, the compounds above are provided that:

when R² is C(O)R⁵ then R³ is not methyl, 2-propyl, cyclopentyl, or 4-piperidyl;

when each X and X¹ is N and R³ is CH₃, R⁴ is not N(CH₃)₂ or S-alkyl;

when Z, R¹ and R⁴ are H; each X and X¹ is N; R² is CO substituted with methyl, phenyl, 4-bromophenyl, 4-chlorophenyl, 4-chlorophenyl, naphth-2-yl, (3-methyl-5-phenyl)thiazol-2-yl, 4-(piperidin-1-ylsulfonyl)phenyl, thien-2-yl, or benzothiazol-2-yl, then R³ is not phenyl, 4-chlorophenyl, or 4-methylphenyl;

when Z, R¹ and R⁴ are H; each X and X¹ is N; R² is CONH₂, then R³ is not methyl, phenyl, or CH₂OCH₂CH₂OH;

when Z, R¹ and R⁴ are H; each X and X¹ is N; R² is alkoxy, then R³ is not tert-butyl;

when Z, R¹ and R⁴ are H; each X is N; X¹ is CH; R² is benzoyl substituted at the meta position with: NH₂, NHSO₂-(chloro-substituted phenyl), NHSO₂-thien-2-yl, NHCONH-(halo or methyl substituted phenyl), NHCONH-(methybenzyl), NHCONH-cyclohexyl, or NHCO-(chloro phenyl); then R³ is not CH₂-cyclopropyl;

when Z, R¹ and R⁴ are H; each X is N; X¹ is CH; R³ is CH₂O-benzyl, CH₂O-alkyl, alkyl or alkenyl optionally substituted with hydroxyl, alkoxy, hydroxyalkyl or hydroxyalkyloxy; or optionally substituted aralkyl; then R² is not CONH₂;

when Z, R¹ and R⁴ are H; each X is N; X¹ is CH; R² is S-phenyl substituted with NH₂, NC(O)O-t-butyl, NC(O)NH-(2-fluorophenyl), NS(O)₂-(mono or di-fluorophenyl) then R³ is not cyclopentyl;

when Z, R¹ and R⁴ are H; each X and X¹ is CH; R³ is 2-(morpholin-1-yl)ethylene; then R² is not CO-tetramethylcyclopropane;

when Z, R¹ and R⁴ are H; each X and X¹ is CH; R³ is methyl, then R² is not COH or carboxyl

when Z, R¹ and R⁴ are H; each X is N, X¹ is N or CH, and R³ is 4-(4-methyl-piperizin-1-yl)cyclohexyl, 4-(N-morpholinyl)cyclohexyl or phenyl, R² is not CONH-(optionally substituted phenyl) or N (optionally substituted phenyl) C(O)(phenyl or alkylphenyl); and

when each X and X¹ is N, R⁴ is H or phenyl, Z is H or optionally substituted phenyl, R¹ is H, and R² is NH-(pyridyl or optionally substituted phenyl), R³ is not methyl, hydroxyalkyl, benzyl or 6-p-tolylpyridazin-3-yl.

In some embodiments, the compound above is provided subject to one or more of the following:
when R² is C(O)R⁵ then R³ is not methyl, 2-propyl, cyclopentyl, or 4-piperidyl; or, in some embodiments, R³ is not alkyl or piperidyl;
when each X and X¹ is N and R³ is alkyl, R⁴ is not N(alkyl)₂ or S-alkyl;
when Z, R¹ and R⁴ are H; each X and X¹ is N; R² is COR⁵ or CONH₂, then R³ is not methyl, CH₂OCH₂CH₂OH, or optionally alkylated or halogenated phenyl; or in some embodiments, R³ is not alkyl, hydroxyalkoxyalkyl, or optionally alkylated or halogenated phenyl;
when Z, R¹ and R⁴ are H; each X and X¹ is N; R² is alkoxy, then R³ is not tert-butyl;
when Z, R¹ and R⁴ are H; each X is N; X¹ is CH; R² is substituted benzoyl; then R³ is not alkyl-cycloalkyl;
when Z, R¹ and R⁴ are H; each X is N; X¹ is CH; R³ is optionally substituted alkyl or alkenyl; then R² is not CONH₂;
when Z, R¹ and R⁴ are H; each X is N; X¹ is CH; R² is S-(substituted phenyl), then R³ is not cycloalkyl;
when Z, R¹ and R⁴ are H; each X and X¹ is CH;
R³ is alkyl or morpholinylethylene; then R² is not CO-cycloalkyl, COH or carboxyl;
when Z, R¹ and R⁴ are H; each X is N, X¹ is N or CH, and R³ is phenyl or cycloalkyl, R² is not CONHR⁵ or NR⁵C(O)R⁵;
when each X and X¹ is N, R⁴ is H or phenyl, Z is H or optionally substituted phenyl, R¹ is H and R² is NH-(optionally substituted phenyl or pyridyl), R³ is not methyl, hydroxyalkyl, benzyl or 6-p-tolylpyridazin-3-yl.

In various embodiments, the compound of the invention excludes one or more compounds selected from FIG. 1A, 1B, 1C, 1D, 1E, 1F, 3B, 4B, 8A, 8B, 8C, 9A, 9B, 9C, or 9D.

In various embodiments, the compound is set forth in FIG. 2, 3A, 4A, 5A, 5B, 6, or 7. In certain embodiments, one or more of the compounds set forth in FIG. 2, 3A, 4A, 5A, 5B, 6, or 7 may be excluded. In some embodiments, the compound is selected from Table I.

In various embodiments, R² is independently SR⁹, OR⁵, OC(O)R⁵, NR⁵R⁵, NR⁵R⁶, COOR⁵, C(O)H, C(O)C(O)R⁵, C(O)NR⁵R⁵, C(O)NR⁵R⁶, S(O)ₘR⁹, S(O)ₘNR⁵R⁵, S(O)ₘNR⁵R⁶, NR⁵C(O)NR⁵R⁵, NR⁶C(O)NR⁶R⁶, NR⁵C(O)C(O)R⁵, NR⁵C(O)C(O)R⁵, NR⁵C(O)R⁵, NR⁶C(O)R⁵, NR⁵(COOR⁵), NR⁶(COOR⁵), NR⁵C(O)R⁸, NR⁶C(O)R⁸, NR⁵S(O)ₘNR⁵R⁵, NR⁶S(O)ₘNR⁶R⁶, NR⁵S(O)ₘR⁵, NR⁶S(O)ₘR⁵, NR⁵S(O)ₘR⁸, NR⁶S(O)ₘR⁸, NR⁵C(O)C(O)NR⁵R⁵, NR⁶C(O)C(O)NR⁵R⁶, or NR⁵C(O)C(O)NR⁵R⁶. In certain embodiments, R² is independently SR⁹, OR⁵, OC(O)R⁵, NR⁵R⁵, NR⁵R⁶, COOR⁵, C(O)H, C(O)C(O)R⁵, S(O)ₘR⁹, S(O)ₘNR⁵R⁵, S(O)ₘNR⁵R⁶, NR⁵C(O)NR⁵R⁵, NR⁶C(O)NR⁶R⁶, NR⁵C(O)C(O)R⁵, NR⁵C(O)C(O)R⁵, NR⁵C(O)R⁵, NR⁶C(O)R⁵, NR⁵(COOR⁵), NR⁶(COOR⁵), NR⁵C(O)R⁸, NR⁶C(O)R⁸, NR⁵S(O)ₘNR⁵R⁵, NR⁶S(O)ₘNR⁶R⁶, NR⁵S(O)ₘR⁵, NR⁶S(O)ₘR⁵, NR⁵S(O)ₘR⁸, NR⁶S(O)ₘR⁸, NR⁵C(O)C(O)NR⁵R⁵, NR⁶C(O)C(O)NR⁵R⁶, or NR⁵C(O)C(O)NR⁵R⁶. In some embodiments, R² is independently NR⁵R⁵, NR⁵R⁶, NR⁵C(O)NR⁵R⁵, NR⁶C(O)NR⁶R⁶, NR⁵C(O)C(O)R⁵, NR⁵C(O)C(O)R⁵, NR⁵C(O)R⁵, NR⁶C(O)R⁵, NR⁵(COOR⁵), NR⁶(COOR⁵), NR⁵C(O)R⁸, NR⁶C(O)R⁸, NR⁵S(O)ₘNR⁵R⁵, NR⁶S(O)ₘNR⁶R⁶, NR⁵S(O)ₘR⁵, NR⁶S(O)ₘR⁵, NR⁵S(O)ₘR⁸, NR⁶S(O)ₘR⁸, NR⁵C(O)C(O)NR⁵R⁵, NR⁶C(O)C(O)NR⁵R⁶, or NR⁵C(O)C(O)NR⁵R⁶.

In various embodiments, R² is independently OR⁵. In some embodiments, R² is independently SR⁹. In certain embodiments, R² is independently NR⁵R⁵ or NR⁵R⁶. In particular embodiments, R² is independently S(O)ₘR⁹, S(O)ₘNR⁵R⁵, or S(O)ₘNR⁵R⁶. In various embodiments, R⁵ is independently optionally substituted aryl or heteroaryl, or in some embodiments, optionally substituted alkyl, cycloalkyl or heteroalkyl. In various embodiments, R⁹ can independently include optionally substituted aryl or heteroaryl, or in some embodiments, optionally substituted cycloalkyl, heteroalkyl, or alkyl with 2 or more carbons.

A method of treating a disorder characterized by impaired protein trafficking includes administering to a subject or contacting a cell with a compound of any of the preceding embodiments.

In various embodiments, the disorder is a lysosomal storage disorder. In some embodiments, the lysosomal storage disorder is Fabry disease, Farber disease, Gaucher disease, GM₁-gangliosidosis, Tay-Sachs disease, Sandhoff disease, GM₂ activator disease, Krabbe disease, metachromatic leukodystrophy, Niemann-Pick disease (types A, B, and C), Hurler disease, Scheie disease, Hunter disease, Sanfilippo disease, Morquio disease, Maroteaux-Lamy disease, hyaluronidase deficiency, aspartylglucosaminuria, fucosidosis, mannosidosis, Schindler disease, sialidosis type 1, Pompe disease, Pycnodysostosis, ceroid lipofuscinosis, cholesterol ester storage disease, Wolman disease, Multiple sulfatase, galactosialidosis, mucolipidosis (types II, III, and IV), cystinosis, sialic acid storage disorder, chylomicron retention disease with Marinesco-Sjögren syndrome, Hermansky-Pudlak syndrome, Chediak-Higashi syndrome, Danon disease, or Geleophysic dysplasia. In some embodiments, the disorder is characterized by an impaired delivery of cargo to a cellular compartment. Lysosomal storage disorders are reviewed in, e.g., Wilcox (2004) J. Pediatr. 144:S3-S14.

In some embodiments, the disorder characterized by impaired protein trafficking is cystic fibrosis. The cystic fibrosis can be characterized by impaired protein trafficking, by impaired cystic fibrosis transmembrane conductance regulator (CFTR) activity, or by both impaired protein trafficking and impaired CFTR activity.

In some embodiments, the disorder characterized by impaired protein trafficking is diabetes, e.g., diabetes mellitus. In some embodiments, the disorder is not diabetes, e.g., diabetes mellitus.

In some embodiments, the disorder characterized by impaired protein trafficking is characterized by an impaired delivery of cargo to a cellular compartment.

In some embodiments, the disorder characterized by impaired protein trafficking is characterized by a Rab27a mutation or a deficiency of Rab27a. The disorder can be, e.g., Griscelli syndrome.

In various embodiments, the disorder is a synucleinopathy. The synucleinopathy can be Parkinson's disease, familial Parkinson's disease, Lewy body disease, the Lewy body variant of Alzheimer's disease, dementia with Lewy bodies, multiple system atrophy, or the Parkinsonism-dementia complex of Guam.

Synucleins are a family of small, presynaptic neuronal proteins composed of alpha-, beta-, and gamma-synucleins, of which only alpha-synuclein aggregates have been associated with several neurological diseases (Ian et al., Clinical Neurosc. Res. 1:445-455, 2001; Trojanowski and Lee, Neurotoxicology 23:457-460, 2002). The role of synucleins (and in particular, alpha-synuclein) in the etiology of a number of neurodegenerative and/or amyloid diseases has developed from several observations. Pathologically, alpha-synuclein was identified as a major component of Lewy bodies, the hallmark inclusions of Parkinson's disease, and a fragment thereof was isolated from amyloid plaques of a different neurological disease, Alzheimer's disease. Biochemically, recombinant alpha-synuclein was shown to form amyloid-like fibrils that recapitulated the ultrastructural features of alpha-synuclein isolated from patients with dementia with Lewy bodies, Parkinson's disease and multiple system atrophy. Additionally, the identification of mutations within the alpha-synuclein gene, albeit in rare cases of familial Parkinson's disease, demonstrated an unequivocal link between synuclein pathology and neurodegenerative diseases. The common involvement of alpha-synuclein in a spectrum of diseases such as Parkinson's disease, dementia with Lewy bodies, multiple system atrophy and the Lewy body variant of Alzheimer's disease has led to the classification of these diseases under the umbrella term of “synucleinopathies.” In some embodiments, the disorder characterized by impaired protein trafficking is not a synucleinopathy.

In various embodiments, the disorder characterized by impaired protein trafficking is hereditary emphysema, α-1-antitrypsin deficiency, hereditary hemochromatosis, oculocutaneous albinism, protein C deficiency, type I hereditary angioedema, congenital sucrase-isomaltase deficiency, Crigler-Najjar type II, Laron syndrome, hereditary Myeloperoxidase, primary hypothyroidism, congenital long QT syndrome, thyroxine binding globulin deficiency, familial hypercholesterolemia, familial chylomicronemia, abeta-lipoproteinema, low plasma lipoprotein a levels, hereditary emphysema with liver injury, congenital hypothyroidism, osteogenesis imperfecta, hereditary hypofibrinogenemia, α-1-antichymotrypsin deficiency, nephrogenic diabetes insipidus, neurohypophyseal diabetes, insipidus, Charcot-Marie-Tooth syndrome, Pelizaeus Merzbacher disease, von Willebrand disease type IIA, combined factors V and VIII deficiency, spondylo-epiphyseal dysplasia tarda, choroideremia, I cell disease, Batten disease, ataxia telangiectasias, acute lymphoblastic leukemia, acute myeloid leukemia, myeloid leukemia, ADPKD-autosomal dominant polycystic kidney disease, microvillus inclusion disease, tuberous sclerosis, oculocerebro-renal syndrome of Lowe, amyotrophic lateral sclerosis, myelodysplastic syndrome, Bare lymphocyte syndrome, Tangier disease, familial intrahepatic cholestasis, X-linked adreno-leukodystrophy, Scott syndrome, Hermansky-Pudlak syndrome types 1 and 2, Zellweger syndrome, rhizomelic chondrodysplasia puncta, autosomal recessive primary hyperoxaluria, Mohr Tranebjaerg syndrome, spinal and bullar muscular atrophy, primary ciliary diskenesia (Kartagener's syndrome), Miller Dieker syndrome, lissencephaly, motor neuron disease, Usher's syndrome, Wiskott-Aldrich syndrome, Optiz syndrome, Huntington's disease, hereditary pancreatitis, anti-phospholipid syndrome, overlap connective tissue disease, Sjögren's syndrome, stiff-man syndrome, Brugada syndrome, Finnish congenital nephritic syndrome, Dubin-Johnson syndrome, X-linked hypophosphosphatemia, Pendred syndrome, persistent hyperinsulinemic hypoglycemia of infancy, hereditary spherocytosis, aceruloplasminemia, infantile neuronal ceroid lipofuscinosis, pseudoachondroplasia and multiple epiphyseal, Stargardt-like macular dystrophy, X-linked Charcot-Marie-Tooth disease, autosomal dominant retinitis pigmentosa, Wolcott-Rallison syndrome, Cushing's disease, limb-girdle muscular dystrophy, mucoploy-saccharidosis type IV, Finnish hereditary familial amyloidosis, Glycogen storage disease type IV (Andersen's disease), sarcoma, chronic myelomonocytic leukemia, cardiomyopathy, faciogenital dysplasia, Torsion disease, Huntington and spinocerebellar ataxias, hereditary hyperhomosyteinemia, polyneuropathy, lower motor neuron disease, pigmented retinitis, seronegative polyarthritis, interstitial pulmonary fibrosis, Raynaud's phenomenon, Wegner's granulomatosis, preoteinuria, CDG-Ia, CDG-Ib, CDG-Ic, CDG-Id, CDG-Ie, CDG-If, CDG-IIa, CDG-IIb, CDG-IIc, CDG-IId, Ehlers-Danlos syndrome, multiple exostoses, Griscelli syndrome (type 1 or type 2), or X-linked non-specific mental retardation. Disorders characterized by impaired protein trafficking are reviewed in Aridor et al. (2000) Traffic 1:836-51 and Aridor et al. (2002) Traffic 3:781-90.

A method of treating a disorder characterized by impaired protein trafficking, includes administering to a subject or contacting a cell with a compound represented in any of FIG. 3B, 4B, 8A, 8B, 8C, 9A, 9B, 9C, or 9D or pharmaceutically acceptable salts or derivatives thereof.

The compounds also include the neutral or non-salt form of the compounds, for example, neutral or non-salt forms of the claimed compounds and the specific compounds disclosed in the Figures, in Table I, or in the Examples.

Also provided are pharmaceutically-acceptable derivatives, including salts, esters, enol ethers, enol esters, solvates, hydrates and prodrugs of the compounds described herein. Pharmaceutically-acceptable salts, include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc, aluminum, and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates.

Further provided are pharmaceutical compositions containing any of the compounds described herein and a pharmaceutically acceptable carrier. In one embodiment, the pharmaceutical compositions are formulated for single dosage administration.

The subject treated according to the methods described herein can be a human or another mammal such as a mouse, rat, cow, pig, dog, cat, or monkey.

Also disclosed is a method of producing a protein, which method includes the steps of: culturing a cell in the presence of a compound described herein (e.g., a compound depicted in Table I); and purifying a protein produced by the cell, wherein the culturing of the cell in the presence of the compound results in enhanced production of the purified protein as compared to culture of the cell in the absence of the compound. The protein can be a recombinant protein encoded by a heterologous nucleic acid. In some embodiments, the protein is a secreted protein and/or a glycosylated protein. For example, the protein can be a cytokine, a lymphokine, a growth factor, or an antibody. The cell used in the protein production methods can be, e.g., an insect cell, a mammalian cell (e.g., a Chinese Hamster Ovary cell), a fungal cell, or a bacterial cell.

In practicing the methods, effective amounts of the compounds or compositions containing therapeutically effective concentrations of the compounds are administered.

Articles of manufacture are provided containing packaging material, a compound or composition provided herein which is useful for treating or ameliorating one or more symptoms of protein trafficking disorders, and a label that indicates that the compound or composition is useful for treating or ameliorating one or more symptoms of protein trafficking disorders.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1a and 1b set forth the structures for certain compounds, e.g., according to Formula I, as described herein.

FIGS. 1c and 1d set forth the structures for certain compounds.

FIG. 1e sets forth the structures for certain free base compounds.

FIG. 1f sets forth the structures for certain compounds as hydrochloride salts

FIG. 2 sets forth the structures for certain compounds.

FIGS. 3A and 3B sets forth the structures for certain compounds.

FIGS. 4A and 4B sets forth the structures for certain compounds.

FIGS. 5A and 5B sets forth the structures for certain compounds.

FIG. 6 sets forth the structures for certain compounds.

FIG. 7 sets forth the structures for certain compounds.

FIGS. 8A-8C sets forth the structures for certain compounds.

FIGS. 9A-9D sets forth the structures for certain compounds.

FIGS. 10A and 11A show Ypt1-ts Western blot data versus concentration for compounds 25 and 5 (see compound structures in Table I), respectively at various concentrations from 0-10 μM.

FIGS. 10B and 11B are plots of densitometry data from FIGS. 10A and 11A.

### DETAILED DESCRIPTION

### A. Definitions

Unless defined otherwise, all technical and scientific terms used herein have the same meaning as is commonly understood by one of ordinary skill in the art to which this document pertains. Although methods and materials similar or equivalent to those described herein can be used in the practice or testing of the present invention, the preferred methods and materials are described below. All patents, applications, published applications and other publications are incorporated by reference in their entirety. In the event that there are a plurality of definitions for a term herein, those in this section prevail unless stated otherwise. In addition, the materials, methods, and examples are illustrative only and not intended to be limiting.

Also provided are methods of treating or ameliorating one or more symptoms of protein trafficking disorders. Such disorders include, for example, cystic fibrosis.

In some embodiments, the disorder characterized by impaired protein trafficking is diabetes (e.g., diabetes mellitus).

In some embodiments, the disorder characterized by impaired protein trafficking is a synucleinopathy. Examples of synucleinopathies include Parkinson's disease, Lewy body disease, the Lewy body variant of Alzheimer's disease, dementia with Lewy bodies, multiple system atrophy, or the Parkinsonism-dementia complex of Guam.

As used herein, α-synuclein refers to one in a family of structurally related proteins that are prominently expressed in the central nervous system. Aggregated α-synuclein proteins form brain lesions that are hallmarks of some neurodegenerative diseases (synucleinopathies). The gene for α-synuclein, which is called SNCA, is on chromosome 4q21. One form of hereditary Parkinson disease is due to mutations in SNCA. Another form of hereditary Parkinson disease is due to a triplication of SNCA. Synucleins are a family of small, presynaptic neuronal proteins composed of α-, β-, and γ-synucleins, of which only α-synuclein aggregates have been associated with several neurological diseases (Ian et al., Clinical Neurosc. Res. 1:445-455, 2001; Trojanowski and Lee, Neurotoxicology 23:457-460, 2002). The role of synucleins (and in particular, α-synuclein) in the etiology of a number of neurodegenerative and/or amyloid diseases has developed from several observations. Pathologically, α-synuclein was identified as a major component of Lewy bodies, the hallmark inclusions of Parkinson's disease, and a fragment thereof was isolated from amyloid plaques of a different neurological disease, Alzheimer's disease. Biochemically, recombinant α-synuclein was shown to form amyloid-like fibrils that recapitulated the ultrastructural features of α-synuclein isolated from patients with dementia with Lewy bodies, Parkinson's disease and multiple system atrophy. Additionally, the identification of mutations within the α-synuclein gene, albeit in rare cases of familial Parkinson's disease, demonstrated an unequivocal link between synuclein pathology and neurodegenerative diseases. The common involvement of α-synuclein in a spectrum of diseases such as Parkinson's disease, dementia with Lewy bodies, multiple system atrophy and the Lewy body variant of Alzheimer's disease has led to the classification of these diseases under the umbrella term of “synucleinopathies.”

In some embodiments, the disorder characterized by impaired protein trafficking is not a synucleinopathy.

In some embodiments, the disorder characterized by impaired protein trafficking is a lysosomal storage disorder such as Fabry disease, Farber disease, Gaucher disease, GM₁-gangliosidosis, Tay-Sachs disease, Sandhoff disease, GM₂ activator disease, Krabbe disease, metachromatic leukodystrophy, Niemann-Pick disease (types A, B, and C), Hurler disease, Scheie disease, Hunter disease, Sanfilippo disease, Morquio disease, Maroteaux-Lamy disease, hyaluronidase deficiency, aspartylglucosaminuria, fucosidosis, mannosidosis, Schindler disease, sialidosis type 1, Pompe disease, Pycnodysostosis, ceroid lipofuscinosis, cholesterol ester storage disease, Wolman disease, Multiple sulfatase, galactosialidosis, mucolipidosis (types II, III, and IV), cystinosis, sialic acid storage disorder, chylomicron retention disease with Marinesco-Sjögren syndrome, Hermansky-Pudlak syndrome, Chediak-Higashi syndrome, Danon disease, or Geleophysic dysplasia. Lysosomal storage disorders are reviewed in, e.g., Wilcox (2004) J. Pediatr. 144:S3-S14.

In some embodiments, the disorder characterized by impaired protein trafficking is characterized by an impaired delivery of cargo to a cellular compartment.

In some embodiments, the disorder characterized by impaired protein trafficking is characterized by a Rab27a mutation or a deficiency of Rab27a. The disorder can be, e.g., Griscelli syndrome.

In some embodiments, the disorder characterized by impaired protein trafficking is hereditary emphysema, hereditary hemochromatosis, oculocutaneous albinism, protein C deficiency, type I hereditary angioedema, congenital sucrase-isomaltase deficiency, Crigler-Najjar type II, Laron syndrome, hereditary Myeloperoxidase, primary hypothyroidism, congenital long QT syndrome, thyroxine binding globulin deficiency, familial hypercholesterolemia, familial chylomicronemia, abeta-lipoproteinema, low plasma lipoprotein a levels, hereditary emphysema with liver injury, congenital hypothyroidism, osteogenesis imperfecta, hereditary hypofibrinogenemia, α-1-antichymotrypsin deficiency, nephrogenic diabetes insipidus, neurohypophyseal diabetes, insipidus, Charcot-Marie-Tooth syndrome, Pelizaeus Merzbacher disease, von Willebrand disease type IIA, combined factors V and VIII deficiency, spondylo-epiphyseal dysplasia tarda, choroideremia, I cell disease, Batten disease, ataxia telangiectasias, acute lymphoblastic leukemia, acute myeloid leukemia, myeloid leukemia, ADPKD-autosomal dominant polycystic kidney disease, microvillus inclusion disease, tuberous sclerosis, oculocerebro-renal syndrome of Lowe, amyotrophic lateral sclerosis, myelodysplastic syndrome, Bare lymphocyte syndrome, Tangier disease, familial intrahepatic cholestasis, X-linked adreno-leukodystrophy, Scott syndrome, Hermansky-Pudlak syndrome types 1 and 2, Zellweger syndrome, rhizomelic chondrodysplasia puncta, autosomal recessive primary hyperoxaluria, Mohr Tranebjaerg syndrome, spinal and bullar muscular atrophy, primary ciliary diskenesia (Kartagener's syndrome), Miller Dieker syndrome, lissencephaly, motor neuron disease, Usher's syndrome, Wiskott-Aldrich syndrome, Optiz syndrome, Huntington's disease, hereditary pancreatitis, anti-phospholipid syndrome, overlap connective tissue disease, Sjögren's syndrome, stiff-man syndrome, Brugada syndrome, congenital nephritic syndrome of the Finnish type, Dubin-Johnson syndrome, X-linked hypophosphosphatemia, Pendred syndrome, persistent hyperinsulinemic hypoglycemia of infancy, hereditary spherocytosis, aceruloplasminemia, infantile neuronal ceroid lipofuscinosis, pseudoachondroplasia and multiple epiphyseal, Stargardt-like macular dystrophy, X-linked Charcot-Marie-Tooth disease, autosomal dominant retinitis pigmentosa, Wolcott-Rallison syndrome, Cushing's disease, limb-girdle muscular dystrophy, mucoploy-saccharidosis type IV, hereditary familial amyloidosis of Finish, Glycogen storage disease type IV (Andersen's disease), sarcoma, chronic myelomonocytic leukemia, cardiomyopathy, faciogenital dysplasia, Torsion disease, Huntington and spinocerebellar ataxias, hereditary hyperhomosyteinemia, polyneuropathy, lower motor neuron disease, pigmented retinitis, seronegative polyarthritis, interstitial pulmonary fibrosis, Raynaud's phenomenon, Wegner's granulomatosis, preoteinuria, CDG-Ia, CDG-Ib, CDG-Ic, CDG-Id, CDG-Ie, CDG-If, CDG-IIa, CDG-IIb, CDG-IIc, CDG-IId, Ehlers-Danlos syndrome, multiple exostoses, Griscelli syndrome (type 1 or type 2), or X-linked non-specific mental retardation. Disorders characterized by impaired protein trafficking are reviewed in Aridor et al. (2000) Traffic 1:836-51 and Aridor et al. (2002) Traffic 3:781-90.

As used herein, pharmaceutically acceptable derivatives of a compound include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs.

Pharmaceutically acceptable esters include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids.

Pharmaceutically acceptable enol ethers include, but are not limited to, derivatives of formula C═C(OR) where R is hydrogen, alkyl, alkenyl, alkynyl, aryl, heteroaryl, aralkyl, heteroaralkyl, cycloalkyl or heterocyclyl.

Pharmaceutically acceptable enol esters include, but are not limited to, derivatives of formula C═C(OC(O)R) where R is hydrogen, alkyl, alkenyl, alkynyl, aryl, heteroaryl, aralkyl, heteroaralkyl, cycloalkyl or heterocyclyl. Pharmaceutically acceptable solvates and hydrates are complexes of a compound with one or more solvent or water molecules, or 1 to about 100, or 1 to about 10, or one to about 2, 3 or 4, solvent or water molecules.

Also included in the present invention are pharmaceutically acceptable salts of the disclosed compounds. These disclosed compounds can have one or more sufficiently acidic protons that can react with a suitable organic or inorganic base to form a base addition salt. When it is stated that a compound has a hydrogen atom bonded to an oxygen, nitrogen, or sulfur atom, it is contemplated that the compound also includes salts thereof where such a hydrogen atom has been reacted with a suitable organic or inorganic base to form a base addition salt. Base addition salts include those derived from inorganic bases, such as ammonium or alkali or alkaline earth metal hydroxides, carbonates, bicarbonates, and the like, and organic bases such as alkoxides, alkyl amides, alkyl and aryl amines, and the like. Such bases useful in preparing the salts of this invention thus include sodium hydroxide, potassium hydroxide, ammonium hydroxide, potassium carbonate, and the like.

For example, pharmaceutically acceptable salts of the disclosed compounds can include those formed by the reaction of the disclosed compounds with one equivalent of a suitable base to form a monovalent salt (i.e., the compound has single negative charge that is balanced by a pharmaceutically acceptable counter cation, e.g., a monovalent cation) or with two equivalents of a suitable base to form a divalent salt (e.g., the compound has a two-electron negative charge that is balanced by two pharmaceutically acceptable counter cations, e.g., two pharmaceutically acceptable monovalent cations or a single pharmaceutically acceptable divalent cation). “Pharmaceutically acceptable” means that the cation is suitable for administration to a subject. Examples include alkali metal cations, such as but not limited Li⁺, Na⁺, K⁺; alkali earth metal cations, such as but not limited to Ba²⁺, Mg²⁺, Ca²⁺; transition metal cations, such as but not limited to Zn²⁺ and other metal salts; and NR₄⁺, wherein each R is independently hydrogen, an optionally substituted aliphatic group (e.g., a hydroxyalkyl group, aminoalkyl group or ammoniumalkyl group) or optionally substituted aryl group, or two R groups, taken together, form an optionally substituted non-aromatic heterocyclic ring optionally fused to an aromatic ring. For example, salts can be formed with amines including, but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine and tris(hydroxymethyl)aminomethane. In some embodiments, the pharmaceutically acceptable cation is Li⁺, Na⁺, K⁺, NH₃(C₂H₅OH)⁺ or N(CH₃)₃(C₂H₅OH)⁺.

Pharmaceutically acceptable salts of the disclosed compounds with a sufficiently basic group, such as an amine, can be formed by reaction of the disclosed compounds with an organic or inorganic acid to form an acid addition salt. Acids commonly employed to form acid addition salts from compounds with basic groups can include inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, and the like. Examples of such salts include nitrates, borates, trifluoroacetates, sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, butyrates, valerates, isobutyrates, caproates, heptanoates, propiolates, oxalates, malonates, succinates, suberates, sebacates, fumarates, maleates, butyne-1,4-dioates, hexyne-1,6-dioates, ascorbates, salicylates, benzoates, chlorobenzoates, methylbenzoates, dinitrobenzoates, hydroxybenzoates, methoxybenzoates, phthalates, sulfonates, benzenesulfonates, toluenesulfonates, xylenesulfonates, phenylacetates, phenylpropionates, phenylbutyrates, citrates, lactates, gamma-hydroxybutyrates, glycolates, tartrates, methanesulfonates, propanesulfonates, naphthalene-1-sulfonates, naphthalene-2-sulfonates, mandelates, and the like. In certain embodiments, the disclosed compound forms a pharmaceutically acceptable salt with HCl, HF, HBr, HI, trifluoracetic acid, or sulfuric acid. In particular embodiments, the disclosed compound forms a pharmaceutically acceptable salt with sulfuric acid.

Various embodiments are directed to pharmaceutically acceptable salts of the compounds described herein, in contrast to the free base of the respective compounds. In some embodiments, the pharmaceutically acceptable salt is the hydrochloride.

Also included are pharmaceutically acceptable solvates. As used herein, the term “solvate” means a compound of the present invention or a salt thereof, that further includes a stoichiometric or non-stoichiometric amount of solvent, e.g., water or organic solvent, bound by non-covalent intermolecular forces.

As used herein, treatment means any manner in which one or more of the symptoms of a disorder are ameliorated or otherwise beneficially altered. Treatment also encompasses any pharmaceutical use of the compounds and compositions herein, such as use for treating disorders in which protein trafficking defects are implicated. Treatment includes therapeutic administration to a subject having such a protein trafficking disorder, wherein the treatment can ward off, hinder, slow, stop, decrease, or interrupt the course, incidence, or occurrence of the protein trafficking disorder. Treatment also includes prophylactic administration to a subject at risk of a protein trafficking disorder, or at risk of worsening of a protein trafficking disorder or symptoms thereof. The prophylactic administration of the compounds tends to lower the risk of having a protein trafficking disorder, or the risk of worsening of a protein trafficking disorder, wherein the prophylactic administration tends to ward off, hinder, slow, stop, decrease, or interrupt the course, incidence, or occurrence such risks. As used herein, amelioration of the symptoms of a particular disorder by administration of a particular compound or pharmaceutical composition refers to any lessening, whether permanent or temporary, lasting or transient that can be attributed to or associated with administration of the composition.

As used herein, IC₅₀ refers to an amount, concentration or dosage of a particular test compound that achieves a 50% inhibition of a maximal response.

As used herein, EC₅₀ refers to a dosage, concentration or amount of a particular test compound that elicits a dose-dependent response at 50% of maximal expression of a particular response that is induced, provoked or potentiated by the particular test compound, such as modulation of CFTR (cystic fibrosis transmembrane conductance regulator) activity, in an assay that measures such response.

As used herein, MRC (Minimum Rescue Concentration) is the minimum concentration of a compound at which cell growth or restoration of cell viability above background is observed as a particular response that is induced, provoked or potentiated by the particular test compound. For example, cell viability or growth can be rescued in a cytotoxic environment, e.g, in the presence of α-synuclein-induced cytotoxicity. Furthermore, for example, cell viability or growth can be measured in the presence of a temperature sensitive mutant at the restrictive temperature.

As used herein, a prodrug is a compound that, upon in vivo administration, is metabolized by one or more steps or processes or otherwise converted to the biologically, pharmaceutically or therapeutically active form of the compound. To produce a prodrug, the pharmaceutically active compound is modified such that the active compound will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

It is to be understood that the compounds provided herein may contain chiral centers. Such chiral centers may be of either the (R) or (S) configuration, or may be a mixture thereof. Thus, the compounds provided herein may be enantiomerically pure, or be stereoisomeric or diastereomeric mixtures. In the case of amino acid residues, such residues may be of either the L- or D-form. The configuration for naturally occurring amino acid residues is generally L. When not specified the residue is the L form. As used herein, the term “amino acid” refers to α-amino acids which are racemic, or of either the D- or L-configuration. The designation “d” preceding an amino acid designation (e.g., dAla, dSer, dVal, etc.) refers to the D-isomer of the amino acid. The designation “dl” preceding an amino acid designation (e.g., dlPip) refers to a mixture of the L- and D-isomers of the amino acid. It is to be understood that the chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

As used herein, substantially pure means sufficiently homogeneous to appear free of readily detectable impurities as determined by standard methods of analysis, such as thin layer chromatography (TLC), gel electrophoresis, high performance liquid chromatography (HPLC) and mass spectrometry (MS), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound may, however, be a mixture of stereoisomers. In such instances, further purification might increase the specific activity of the compound.

As used herein, “alkyl,” “alkenyl” and “alkynyl” carbon chains, if not specified, contain from 1 to 20 carbons, or 1 or 2 to 16 carbons, and in various embodiments are straight, branched, or cyclic, or in some embodiments, are straight or branched. Alkenyl carbon chains of from 2 to 20 carbons, in certain embodiments, contain 1 to 8 double bonds and alkenyl carbon chains of 2 to 16 carbons, in certain embodiments, contain 1 to 5 double bonds. Alkynyl carbon chains of from 2 to 20 carbons, in certain embodiments, contain 1 to 8 triple bonds, and the alkynyl carbon chains of 2 to 16 carbons, in certain embodiments, contain 1 to 5 triple bonds. Exemplary alkyl, alkenyl and alkynyl groups herein include, but are not limited to, methyl, ethyl, propyl, isopropyl, isobutyl, n-butyl, sec-butyl, tert-butyl, isopentyl, neopentyl, tert-pentyl, isohexyl, allyl (propenyl) and propargyl (propynyl). As used herein, lower alkyl, lower alkenyl, and lower alkynyl refer to carbon chains having from about 1 or about 2 carbons up to about 6 carbons. As used herein, “alk(en)(yn)yl” refers to an alkyl group containing at least one double bond and at least one triple bond.

As used herein, “cycloalkyl” refers to a saturated mono- or multi-cyclic ring system, in certain embodiments of 3 to 10 carbon atoms, in other embodiments of 3 to 6 carbon atoms; cycloalkenyl and cycloalkynyl refer to mono- or multicyclic ring systems that respectively include at least one double bond and at least one triple bond. Cycloalkenyl and cycloalkynyl groups may, in certain embodiments, contain 3 to 10 carbon atoms, with cycloalkenyl groups, in further embodiments, containing 4 to 7 carbon atoms and cycloalkynyl groups, in further embodiments, containing 8 to 10 carbon atoms. The ring systems of the cycloalkyl, cycloalkenyl and cycloalkynyl groups may be composed of one ring or two or more rings which may be joined together in a fused, bridged or spiro-connected fashion. “Cycloalk(en)(yn)yl” refers to a cycloalkyl group containing at least one double bond and at least one triple bond.

As used herein, “aryl” refers to optionally substituted aromatic monocyclic or multicyclic groups containing from 6 to 19 carbon atoms. Examples of “aryl” groups include phenyl, biphenyl, and the like. Aryl groups also include fused polycyclic aromatic ring systems such as naphthyl, tetrahydronapthyl, pyrenyl, anthracyl, 9,10-dihydroanthracyl, fluorenyl, indenyl, indanyl, and the like, in which a carbocyclic aromatic ring is fused to one or more other aryl, cycloalkyl, or cycloaliphatic rings.

As used herein, “heteroaryl” refers to an optionally substituted monocyclic or multicyclic aromatic ring system, in certain embodiments, of about 5 to about 15 members where one or more, in various embodiments 1 to 4, or in some embodiments 1 to 3, of the atoms in the ring system is a heteroatom, including but not limited to, nitrogen, oxygen or sulfur. The heteroaryl group may be optionally fused to a benzene ring. Examples of heteroaryl groups include optionally substituted pyridyl, pyrimidyl, pyrazinyl, triazinyl, pyranyl, pyrrolyl, imidazolyl, pyrazolyl, 1,2,3-trizaolyl, 1,2,4-triazolyl, tetrazolyl, thienyl, thiazoyl, isothiazolyl, furanyl, oxazolyl, isooxazolyl, and the like. Heteroaryl groups also include fused polycyclic aromatic ring systems in which a heteroaryl ring is fused to one or more other heteroaryl, aryl, heterocyclyl, cycloalkyl, or cycloaliphatic rings, for example, optionally substituted quinolinyl, isoquinolinyl, quinazolinyl, napthyridyl, pyridopyrimidyl, benzothienyl, benzothiazolyl, benzoisothiazolyl, thienopyridyl, thiazolopyridyl, isothiazolopyridyl, benzofuranyl, benzooxazolyl, benzoisooxazolyl, furanopyridyl, oxazolopyridyl, isooxazolopyridyl, indolyl, isoindolyl, benzimidazolyl, benzopyrazolyl, pyrrolopyridyl, isopyrrolopyridyl, imidazopyridyl, pyrazolopyridyl, and the like.

Any ring recited as a substituent herein can be bonded via any substitutable atom in the ring.

As used herein, a “heteroarylium” group is a heteroaryl group that is positively charged on one or more of the heteroatoms.

As used herein, “heterocyclyl” refers to an optionally substituted monocyclic or multicyclic non-aromatic ring system, in various embodiments of 3 to 10 members, in another embodiment of 4 to 7 members, in a further embodiment of 5 to 6 members, where one or more, in some embodiments, 1 to 4, in certain embodiments, 1 to 3, of the atoms in the ring system is a heteroatom, including but not limited to, nitrogen, oxygen or sulfur. Examples of heterocyclyl groups include oxazolinyl, thiazolinyl, oxazolidinyl, thiazolidinyl, tetrahydrofuranyl, tetrahyrothiophenyl, morpholino, thiomorpholino, pyrrolidinyl, piperazinyl, piperidinyl, thiazolidinyl, and the like. In embodiments where the heteroatom(s) is(are) nitrogen, the nitrogen is optionally substituted with alkyl, alkenyl, alkynyl, aryl, heteroaryl, aralkyl, heteroaralkyl, cycloalkyl, heterocyclyl, cycloalkylalkyl, heterocyclylalkyl, acyl, guanidino, or the nitrogen may be quaternized to form an ammonium group where the substituents are selected as above.

As used herein. “lone pair,” when referring to a substitution variable on a nitrogen atom, means that the substitution variable represents the Lewis structure electron pair for the corresponding nitrogen, and no substituting functional group is bound to the indicated position.

As used herein, “aralkyl” refers to an alkyl group in which one of the hydrogen atoms of the alkyl is replaced by an aryl group.

As used herein, “heteroaralkyl” refers to an alkyl group in which one of the hydrogen atoms of the alkyl is replaced by a heteroaryl group.

As used herein, “halo”, “halogen” or “halide” refers to F, Cl, Br or I.

As used herein, pseudohalides or pseudohalo groups are groups that can be bioisosteric for halides or otherwise tend to behave substantially similar to halides. Such compounds can be used in the same manner and treated in the same manner as halides. Pseudohalides include, but are not limited to, cyanide, cyanate, thiocyanate, selenocyanate, trifluoromethoxy, and azide.

As used herein, “haloalkyl” refers to an alkyl group in which one or more of the hydrogen atoms are replaced by halogen. Such groups include, but are not limited to, chloromethyl, trifluoromethyl and 1-chloro-2-fluoroethyl.

As used herein, “haloalkoxy” refers to RO— in which R is a haloalkyl group.

As used herein, “sulfinyl” or “thionyl” refers to —S(O)—. As used herein, “sulfonyl” or “sulfuryl” refers to —S(O)₂—. As used herein, “sulfo” refers to —S(O)₂O—.

As used herein, “carboxy” refers to a divalent radical, —C(O)O—.

As used herein, “aminocarbonyl” refers to —C(O)NH₂.

As used herein, “alkylaminocarbonyl” refers to —C(O)NHR in which R is alkyl, including lower alkyl. As used herein, “dialkylaminocarbonyl” refers to —C(O)NR′R in which R′ and R are independently alkyl, including lower alkyl; “carboxamide” refers to groups of formula —NR′COR in which R′ and R are independently alkyl, including lower alkyl.

As used herein, “diarylaminocarbonyl” refers to —C(O)NRR′ in which R and R′ are independently selected from aryl, including lower aryl, such as phenyl.

As used herein, “arylalkylaminocarbonyl” refers to —C(O)NRR′ in which one of R and R′ is aryl, including lower aryl, such as phenyl, and the other of R and R′ is alkyl, including lower alkyl.

As used herein, “arylaminocarbonyl” refers to —C(O)NHR in which R is aryl, including lower aryl, such as phenyl.

As used herein, “hydroxycarbonyl” refers to —COOH.

As used herein, “alkoxycarbonyl” refers to —C(O)OR in which R is alkyl, including lower alkyl.

As used herein, “aryloxycarbonyl” refers to —C(O)OR in which R is aryl, including lower aryl, such as phenyl.

As used herein, “heteroaryloxycarbonyl” refers to —C(O)OR in which R is heteroaryl, including lower heteroaryl, such as pyridyl.

As used herein, “alkoxy” and “alkylthio” refer to RO— and RS—, in which R is alkyl, including lower alkyl.

As used herein, “aryloxy” and “arylthio” refer to RO— and RS—, in which R is aryl, including lower aryl, such as phenyl.

As used herein, “alkylene” refers to a straight, branched or cyclic, in certain embodiments straight or branched, divalent aliphatic hydrocarbon group, in various embodiments having from 1 to about 20 carbon atoms, in another embodiment having from 1 to 12 carbons. In a further embodiment alkylene includes lower alkylene. There may be optionally inserted along the alkylene group one or more oxygen, sulfur, including S(═O) and S(═O)₂ groups, or substituted or unsubstituted nitrogen atoms, including —NR— and —N⁺RR— groups, where the nitrogen substituent(s) is(are) alkyl, aryl, aralkyl, heteroaryl, heteroaralkyl or COR′, where R′ is alkyl, aryl, aralkyl, heteroaryl, heteroaralkyl, —OY or —NYY, where Y is hydrogen, alkyl, aryl, heteroaryl, cycloalkyl or heterocyclyl. Alkylene groups include, but are not limited to, methylene (—CH₂—), ethylene (—CH₂CH₂—), propylene (—(CH₂)₃—), methylenedioxy (—O—CH₂—O—) and ethylenedioxy (—O—(CH₂)₂—O—). The term “lower alkylene” refers to alkylene groups having 1 to 6 carbons. In certain embodiments, alkylene groups are lower alkylene, including alkylene of 1 to 3 carbon atoms.

As used herein, “azaalkylene” refers to —(CRR)ₙ—NR—(CRR)ₘ—, where n and m are each independently an integer from 0 to 4. As used herein, “oxaalkylene” refers to —(CRR)ₙ—O—(CRR)ₘ—, where n and m are each independently an integer from 0 to 4. As used herein, “thiaalkylene” refers to —(CRR)ₙ—S—(CRR)ₘ—, —(CRR)ₙ—S(═O)—(CRR)ₘ—, and —(CRR)ₙ—S(═O)₂—(CRR)ₘ— where n and m are each independently an integer from 0 to 4.

As used herein, “alkenylene” refers to a straight, branched or cyclic, in various embodiments straight or branched, divalent aliphatic hydrocarbon group, in certain embodiments having from 2 to about 20 carbon atoms and at least one double bond, in other embodiments 1 to 12 carbons. In further embodiments, alkenylene groups include lower alkenylene. There may be optionally inserted along the alkenylene group one or more oxygen, sulfur or substituted or unsubstituted nitrogen atoms, where the nitrogen substituent is alkyl. Alkenylene groups include, but are not limited to, —CH═CH—CH═CH— and —CH═CH—CH₂—. The term “lower alkenylene” refers to alkenylene groups having 2 to 6 carbons. In certain embodiments, alkenylene groups are lower alkenylene, including alkenylene of 3 to 4 carbon atoms.

As used herein, “alkynylene” refers to a straight, branched or cyclic, in certain embodiments straight or branched, divalent aliphatic hydrocarbon group, in various embodiments having from 2 to about 20 carbon atoms and at least one triple bond, in another embodiment 1 to 12 carbons. In a further embodiment, alkynylene includes lower alkynylene. There may be optionally inserted along the alkynylene group one or more oxygen, sulfur or substituted or unsubstituted nitrogen atoms, where the nitrogen substituent is alkyl. Alkynylene groups include, but are not limited to, —C≡C—C≡C—, —C≡C— and —C≡C—CH₂—. The term “lower alkynylene” refers to alkynylene groups having 2 to 6 carbons. In certain embodiments, alkynylene groups are lower alkynylene, including alkynylene of 3 to 4 carbon atoms.

As used herein, “alk(en)(yn)ylene” refers to a straight, branched or cyclic, in certain embodiments straight or branched, divalent aliphatic hydrocarbon group, in various embodiments having from 2 to about 20 carbon atoms and at least one triple bond, and at least one double bond; in another embodiment 1 to 12 carbons. In further embodiments, alk(en)(yn)ylene includes lower alk(en)(yn)ylene. There may be optionally inserted along the alkynylene group one or more oxygen, sulfur or substituted or unsubstituted nitrogen atoms, where the nitrogen substituent is alkyl. Alk(en)(yn)ylene groups include, but are not limited to, —C═C—(CH₂)ₙ—C≡C—, where n is 1 or 2. The term “lower alk(en)(yn)ylene” refers to alk(en)(yn)ylene groups having up to 6 carbons. In certain embodiments, alk(en)(yn)ylene groups have about 4 carbon atoms.

As used herein, “cycloalkylene” refers to a divalent saturated mono- or multicyclic ring system, in certain embodiments of 3 to 10 carbon atoms, in other embodiments 3 to 6 carbon atoms; cycloalkenylene and cycloalkynylene refer to divalent mono- or multicyclic ring systems that respectively include at least one double bond and at least one triple bond. Cycloalkenylene and cycloalkynylene groups may, in certain embodiments, contain 3 to 10 carbon atoms, with cycloalkenylene groups in certain embodiments containing 4 to 7 carbon atoms and cycloalkynylene groups in certain embodiments containing 8 to 10 carbon atoms. The ring systems of the cycloalkylene, cycloalkenylene and cycloalkynylene groups may be composed of one ring or two or more rings which may be joined together in a fused, bridged or spiro-connected fashion. “Cycloalk(en)(yn)ylene” refers to a cycloalkylene group containing at least one double bond and at least one triple bond.

As used herein, “arylene” refers to a monocyclic or polycyclic, in certain embodiments monocyclic, divalent aromatic group, in various embodiments having from 5 to about 20 carbon atoms and at least one aromatic ring, in another embodiment 5 to 12 carbons. In further embodiments, arylene includes lower arylene. Arylene groups include, but are not limited to, 1,2-, 1,3- and 1,4-phenylene. The term “lower arylene” refers to arylene groups having 6 carbons.

As used herein, “heteroarylene” refers to a divalent monocyclic or multicyclic aromatic ring system, in various embodiments of about 5 to about 15 atoms in the ring(s), where one or more, in certain embodiments 1 to 3, of the atoms in the ring system is a heteroatom, that is, an element other than carbon, including but not limited to, nitrogen, oxygen or sulfur. The term “lower heteroarylene” refers to heteroarylene groups having 5 or 6 atoms in the ring. As used herein, “heterocyclylene” refers to a divalent monocyclic or multicyclic non-aromatic ring system, in certain embodiments of 3 to 10 members, in various embodiments 4 to 7 members, in another embodiment 5 to 6 members, where one or more, including 1 to 3, of the atoms in the ring system is a heteroatom, that is, an element other than carbon, including but not limited to, nitrogen, oxygen or sulfur.

As used herein, “alkylidene” refers to a divalent group, such as ═CR′R″, which is attached to one atom of another group, forming a double bond. Alkylidene groups include, but are not limited to, methylidene (═CH₂) and ethylidene (═CHCH₃). As used herein, “arylalkylidene” refers to an alkylidene group in which either R′ or R″ is an aryl group. “Cycloalkylidene” groups are those where R′ and R″ are linked to form a carbocyclic ring. “Heterocyclylid-ene” groups are those where at least one of R′ and R″ contain a heteroatom in the chain, and R′ and R″ are linked to form a heterocyclic ring.

As used herein, “amido” refers to the divalent group —C(O)NH—. “Thioamido” refers to the divalent group —C(S)NH—. “Oxyamido” refers to the divalent group —OC(O)NH—. “Thiaamido” refers to the divalent group —SC(O)NH—. “Dithiaamido” refers to the divalent group —SC(S)NH—. “Ureido” refers to the divalent group —HNC(O)NH—. “Thioureido” refers to the divalent group —HNC(S)NH—.

As used herein, “semicarbazide” refers to —NHC(O)NHNH—. “Carbazate” refers to the divalent group —OC(O)NHNH—. “Isothiocarbazate” refers to the divalent group —SC(O)NHNH—. “Thiocarbazate” refers to the divalent group —OC(S)NHNH—. “Sulfonylhydrazide” refers to the divalent group —SO₂NHNH—. “Hydrazide” refers to the divalent group —C(O)NHNH—. “Azo” refers to the divalent group —N═N—. “Hydrazinyl” refers to the divalent group —NH—NH—.

As used herein, “substituted alkyl,” “substituted alkenyl,” “substituted alkynyl,” “substituted cycloalkyl,” “substituted cycloalkenyl,” “substituted cycloalkynyl,” “substituted aryl,” “substituted heteroaryl,” “substituted heterocyclyl,” “substituted alkylene,” “substituted alkenylene,” “substituted alkynylene,” “substituted cycloalkylene,” “substituted cycloalkenylene,” “substituted cycloalkynylene,” “substituted arylene,” “substituted heteroarylene” and “substituted heterocyclylene” refer to alkyl, alkenyl, alkynyl, cycloalkyl, cycloalkenyl, cycloalkynyl, aryl, heteroaryl, heterocyclyl, alkylene, alkenylene, alkynylene, cycloalkylene, cycloalkenylene, cycloalkynylene, arylene, heteroarylene and heterocyclylene groups, respectively, that are substituted with one or more substituents, in certain embodiments one, two, three or four substituents, where the substituents are as defined herein. “Optionally substituted”

Suitable optional substituents for a substitutable atom any of the preceding groups, e.g., alkyl, cycloalkyl, aliphatic, cycloaliphatic, alkylene, alkenylene, alkynylene, heteroalkylene, heteroalkenylene, heteroalkynylene, heterocyclic, aryl, and heteroaryl groups, are those substituents that do not substantially interfere with the pharmaceutical activity of the disclosed compounds. A “substitutable atom” is an atom that has one or more valences or charges available to form one or more corresponding covalent or ionic bonds with a substituent. For example, a carbon atom with one valence available (e.g., —C(—H)═) can form a single bond to an alkyl group (e.g., —C(-alkyl)=), a carbon atom with two valences available (e.g., —C(H₂)—) can form one or two single bonds to one or two substituents (e.g., —C(alkyl)(H)—, —C(alkyl)(Br))—) or a double bond to one substituent (e.g., —C(═O)—), and the like. Substitutions contemplated herein include only those substitutions that form stable compounds. In some embodiments, certain suitable optional substituents can be further substituted by corresponding suitable optional substituents. In some embodiments, suitable optional substituents are not further substituted.

For example, suitable optional substituents for substitutable carbon atoms include —F, —Cl, —Br, —I, —CN, —NO₂, —N₃, —OR^{a}, —C(O)R^{a}, —OC(O)R^{a}, —C(O)OR^{a}, —SR^{a}, —C(S)R^{a}, —OC(S)R^{a}, —C(S)OR^{a}, —C(O)SR^{a}, —C(S)SR^{a}, —S(O)R^{a}, —SO₂R^{a}, —SO₃R^{a}, —OSO₂R^{a}, —OSO₃R^{a}, —PO₂R^{a}R^{b}, —OPO₂R^{a}R^{b}, —PO₃R^{a}R^{b}, —OPO₃R^{a}R^{b}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —SO₂N(R^{a}R^{b}), —NR^{c}C(O)R^{a}, —NR^{c}C(O)OR^{a}, —NR^{c}C(O)N(R^{a}R^{b}), —C(NR^{c})—N(R^{a}R^{b}), —NR^{d}—C(NR^{c})—N(R^{a}R^{b}), —NR^{a}N(R^{a}R^{b}), —CR^{c}═CR^{a}R^{b}, —C≡CR^{a}, ═O, ═S, ═CR^{a}R^{b}, ═NR^{a}, ═NOR^{a}, ═NNR^{a}, optionally substituted alkyl, optionally substituted cycloalkyl, optionally substituted aliphatic, optionally substituted cycloaliphatic, optionally substituted heterocyclic, optionally substituted benzyl, optionally substituted aryl, and optionally substituted heteroaryl, wherein R^{a}-R^{d} are each independently —H or an optionally substituted aliphatic, optionally substituted cycloaliphatic, optionally substituted heterocyclic, optionally substituted benzyl, optionally substituted aryl, or optionally substituted heteroaryl, or, —N(R^{a}R^{b}), taken together, is an optionally substituted heterocyclic group. In certain embodiments, ═O is excluded as a suitable optional substituent.

Suitable substituents for nitrogen atoms having two covalent bonds to other atoms include, for example, optionally substituted alkyl, optionally substituted cycloalkyl, optionally substituted aliphatic, optionally substituted cycloaliphatic, optionally substituted heterocyclic, optionally substituted benzyl, optionally substituted aryl, optionally substituted heteroaryl, —CN, —NO₂, —OR^{a}, —C(O)R^{a}, —OC(O)R^{a}, —C(O)OR^{a}, —SR^{a}, —S(O)R^{a}, —SO₂R^{a}, —SO₃R^{a}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —SO₂N(R^{a}R^{b}), —NR^{c}C(O)R^{a}, —NR^{c}C(O)OR^{a}, —NR^{c}C(O)N(R^{a}R^{b}), and the like.

A nitrogen-containing group, for example, a heteroaryl or non-aromatic heterocycle, can be substituted with oxygen to form an N-oxide, e.g., as in a pyridyl N-oxide, piperidyl N-oxide, and the like. For example, in various embodiments, a ring nitrogen atom in a nitrogen-containing heterocyclic or heteroaryl group can be substituted to form an N-oxide.

Suitable substituents for nitrogen atoms having three covalent bonds to other atoms include —OH, alkyl, and alkoxy (preferably C₁₋₆ alkyl and alkoxy). Substituted ring nitrogen atoms that have three covalent bonds to other ring atoms are positively charged, which is balanced by counteranions corresponding to those found in pharmaceutically acceptable salts, such as chloride, bromide, fluoride, iodide, formate, acetate and the like. Examples of other suitable counteranions are provided in the section below directed to suitable pharmacologically acceptable salts.

It will also be understood that certain disclosed compounds can be obtained as different stereoisomers (e.g., diastereomers and enantiomers) and that the invention includes all isomeric forms and racemic mixtures of the disclosed compounds and methods of treating a subject with both pure isomers and mixtures thereof, including racemic mixtures. Stereoisomers can be separated and isolated using any suitable method, such as chromatography.

It will also be understood that certain disclosed compounds can exist as or can be represented as tautomers. Tautomers are compounds that can be interconverted by migration of a hydrogen atom or proton in combination with the exchange of adjacent single bond and double bonds. In solutions where tautomerization is possible, a chemical equilibrium of the tautomers can be reached. The exact ratio of the tautomers depends on several factors, including temperature, solvent, and pH.

Where the number of any given substituent is not specified (e.g., haloalkyl), there may be one or more substituents present, up to the number of substituents chemically possible. For example, “haloalkyl” may include one or more of the same or different halogens, for example, fluoromethyl, trifluoromethyl, fluorodichloromethyl, and the like.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:942-944).

### B. Compounds

The compounds provided herein for use in the compositions and methods provided herein exhibit activity against protein trafficking mediated diseases and disorders. In various embodiments, the compounds can treat or ameliorate one or more symptoms associated with protein trafficking mediated diseases and disorders.

### C. Preparation of the Compounds

The compounds for use in the compositions and methods provided herein may be obtained from commercial sources (e.g., Aldrich Chemical Co., Milwaukee, Wis.), may be prepared by methods well known to those of skill in the art, or may be prepared by the methods shown herein, both below and in the Examples. One of skill in the art would be able to prepare all of the compounds for use herein by routine modification of these methods using the appropriate starting materials.

Certain of the compounds provided herein may be made by the synthetic routes shown below. For example, Schemes 1-28 demonstrate a number of methods to perform generic substitution of a bicyclic core such as pyrazolo-pyrimidine with various groups, e.g., R and Ar groups.

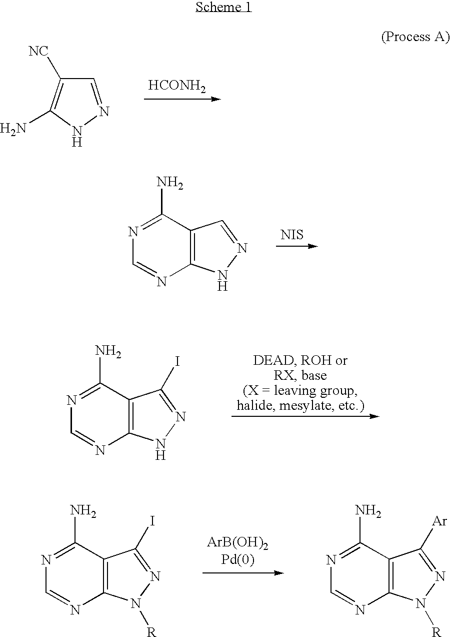

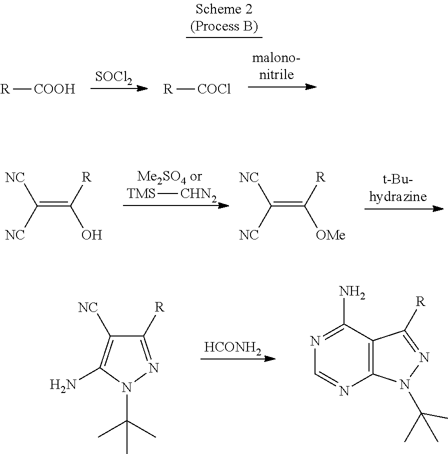

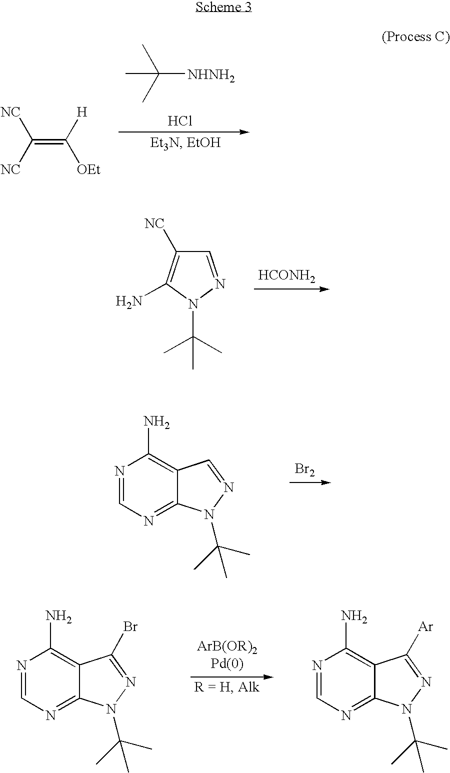

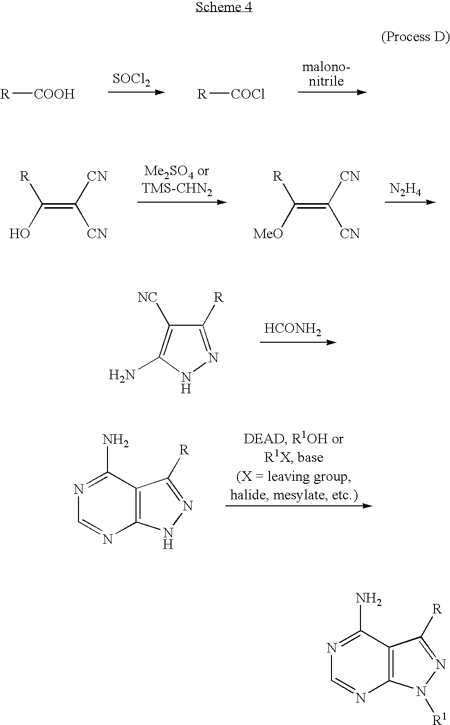

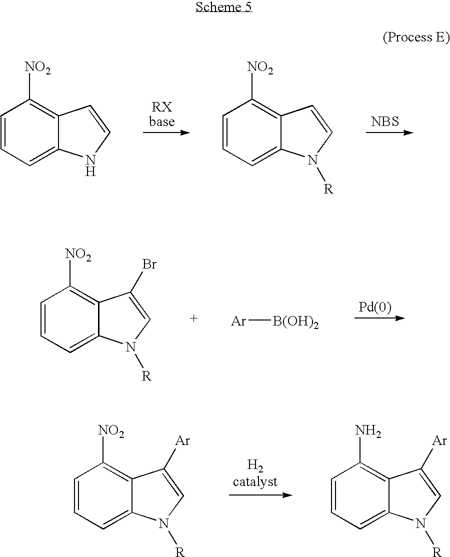

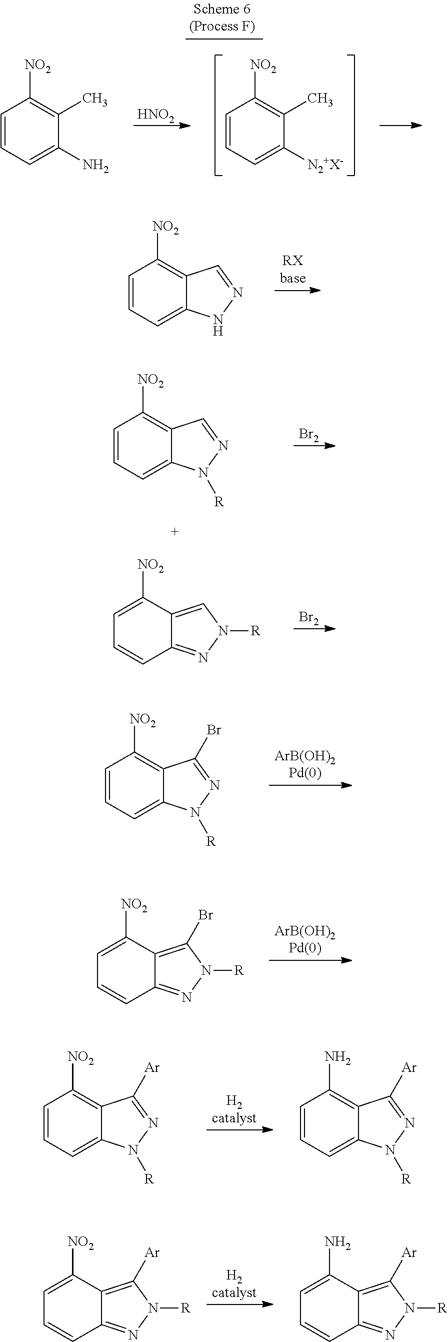

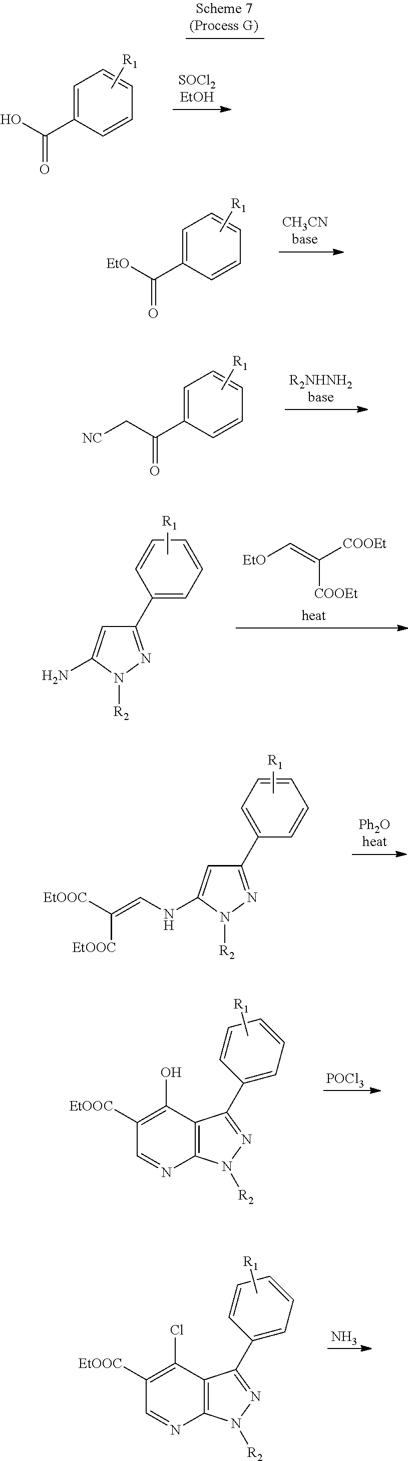

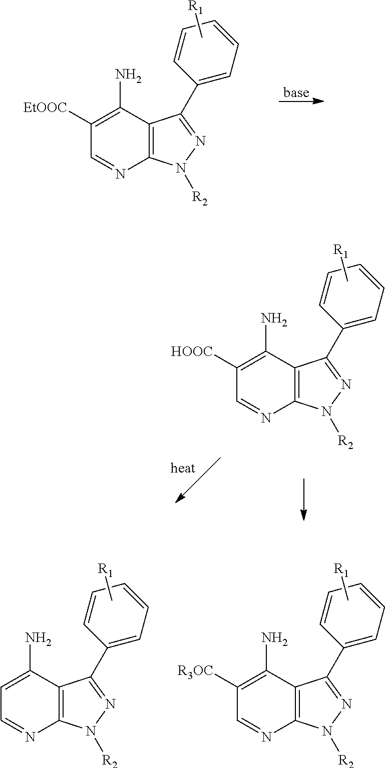

Scheme 8 depicts a synthetic method for 3-halo substituted bicyclic ring systems, e.g., the 3-iodo pyrrolopyrimidine shown. While the Mitsunobu-type reaction of Scheme 8, as depicted, proceeds without the use of protecting groups, other reactions may benefit from protection of the 4-amino group, e.g., using suitable protecting groups and strategies for protecting and deprotecting amino groups as known in the art e.g., as described in T. W. Greene and P. G. M. Wuts, Protective Groups in Organic Synthesis, 2nd Ed., John Wiley and Sons (1991), the entire teachings of which are incorporated herein by reference. For example, suitable amine protecting groups include benzyloxycarbonyl, tert butoxycarbonyl, tert butyl, benzyl and fluorenylmethyloxy carbonyl (Fmoc).

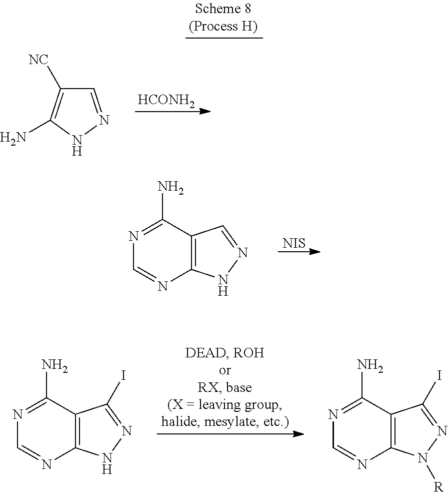

Scheme 9 depicts a synthesis of a protected-amine compound that can be employed in preparing various compounds of the instant invention. This compound may subsequently be deprotected.

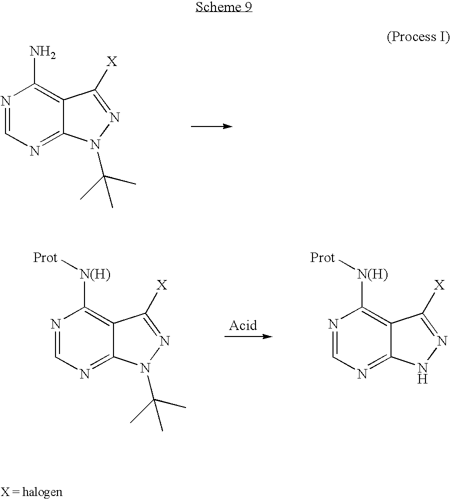

Schemes 10-15 depict various routes to incorporate substituents at the 1-nitrogen position of compounds of the invention, or intermediate useful to make compounds of the invention:

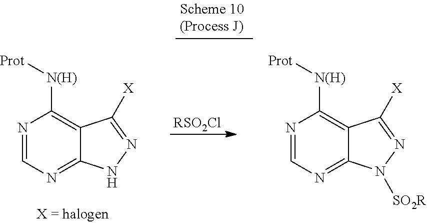

For descriptions of reactions that can be adapted to perform the transformation depicted in Scheme 10, see, for example, Bioorganic & Medicinal Chemistry Letters, 17(14), 4075-4079; 2007; Journal of Medicinal Chemistry, 50(1), 10-20; 2007; Bioorganic & Medicinal Chemistry, 14(4), 1078-1088; 2006; and PCT Int. Appl., 2007012953, 1 Feb. 2007. The entire teachings of the preceding documents is incorporated herein by reference.

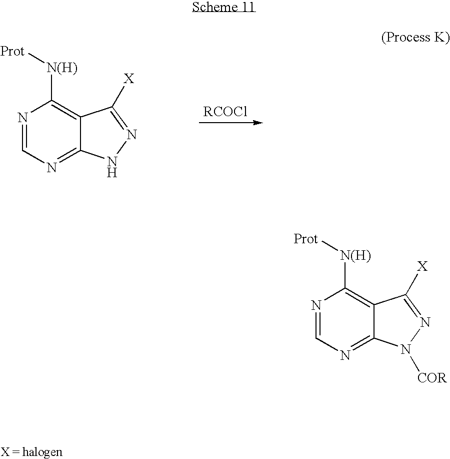

For descriptions of reactions that can be adapted to perform the transformation depicted in Scheme 11, see, for example, Tetrahedron, 31(6), 587-91; 1975, the entire teachings of which is incorporated herein by reference.

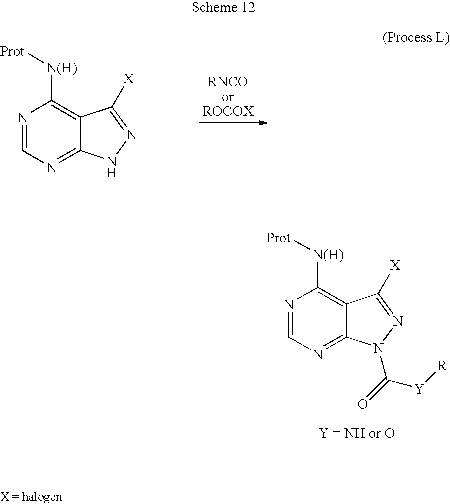

For descriptions of reactions that can be adapted to perform the transformation depicted in Scheme 12, see, for example, Journal of the Chemical Society, Perkin Transactions 1: Organic and Bio-Organic Chemistry (1972-1999), (11), 2795-802; 1979, the entire teachings of which is incorporated herein by reference.

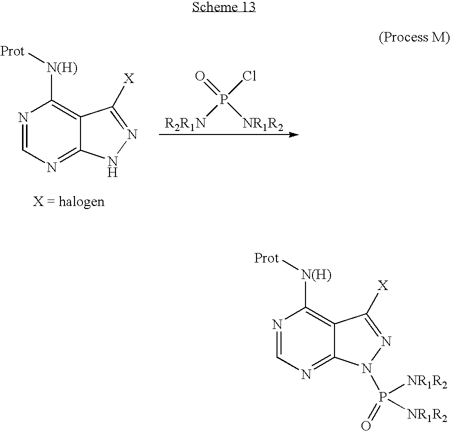

For descriptions of reactions that can be adapted to perform the transformation depicted in Scheme 13, see, for example, Journal of Organic Chemistry, 53(4), 794-9; 1988, the entire teachings of which is incorporated herein by reference.

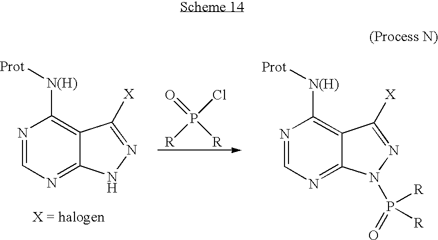

For descriptions of reactions that can be adapted to perform the transformation depicted in Scheme 14, see, for example, Organic Letters, 5(11), 1899-1902; 2003, the entire teachings of which is incorporated herein by reference.

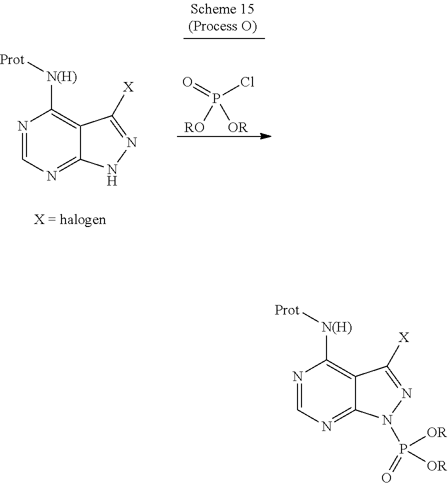

For descriptions of reactions that can be adapted to perform the transformation depicted in Scheme 15, see, for example, Journal of Organic Chemistry, 54(7), 1664-8; 1989, the entire teachings of which is incorporated herein by reference.

The methods described in Schemes 10-15 can be adapted to employ other starting compounds. See, for example, Journal of Heterocyclic Chemistry (1969), 6(2), 207-13, the entire teachings of which is incorporated herein by reference, for starting materials such as 5-halo-4-amino-pyrollo[2,3-d]pyrimidines, for example, the known 5-bromo-4-amino-pyrollo[2,3-d]pyrimidine:

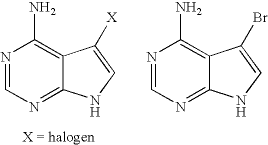

Starting materials such as 3-bromo-4-nitro-indole (Albany Molecular Research, Albany, N.Y.), and 3-bromo-4-nitro-indazole may be employed (see Journal of Heterocyclic Chemistry (1979), 16(8), 1599-603), the entire teachings of which is incorporated herein by reference.

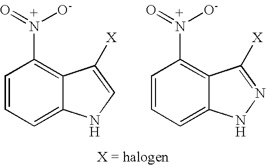

In compounds such as the preceding, the nitro groups can be reduced to amine according to methods known in the art. See also, for example, the methods depicted in Schemes 5 and 6.

Scheme 16 depicts a method for derivatizing the 3 position with sulfonamides by treating an unsubstituted, appropriately protected starting material such as that shown in Scheme 16, with chlorosulfonic acid followed by an amine.

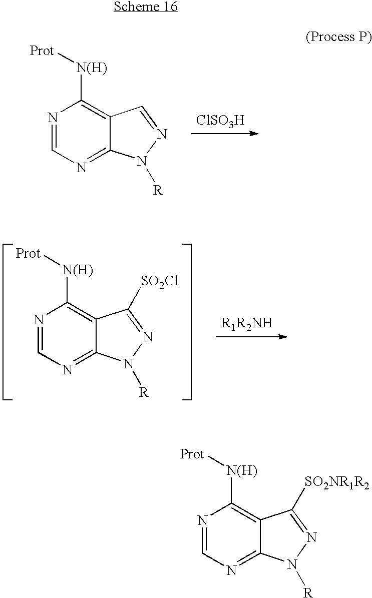

See, for example, Asian Journal of Chemistry, 17(2), 980-984; 2005, and Tetrahedron, 62(8), 1699-1707; 2006, the entire teachings of which are incorporated herein by reference.

Certain compounds of the invention can be made starting with compounds of formula 1, wherein X is either oxygen or nitrogen, as shown in Schemes 17 and 18:

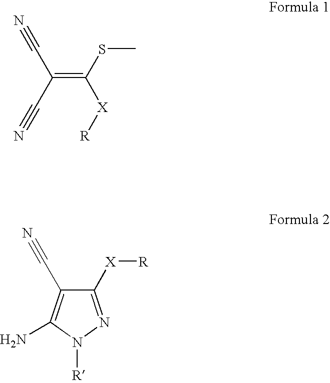

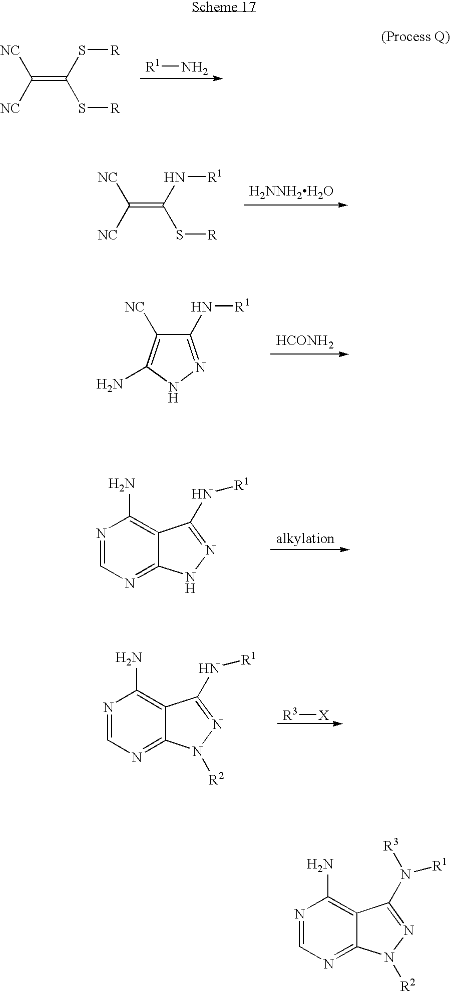

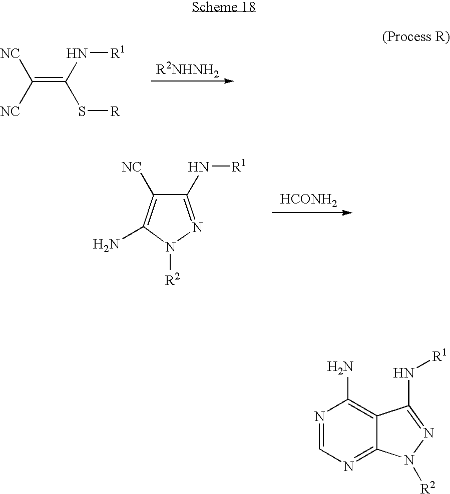

Condensation can provide the desired pyrazole, represented by structural formula 2, which can be employed as a starting material in the methods described above. See, for example, WO 1998014449, WO 1998014450, and WO 1996031510, the entire teachings of which are incorporated herein by reference.

In addition, various compounds can be synthesized using metal catalyzed coupling reactions with 3-halogeno starting materials represented by structural formula III in Scheme 19:

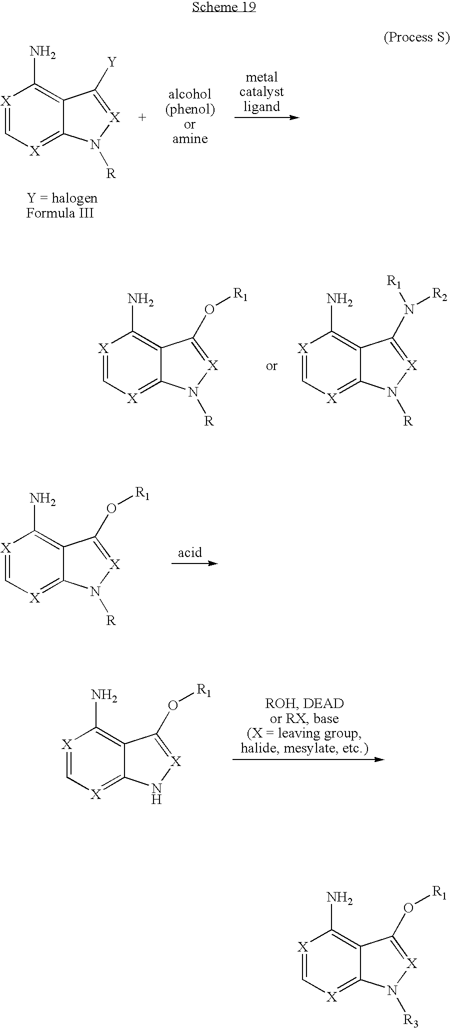

Such methods include, for example, the Ullman coupling, as well as palladium and/or copper developed independently by Buchwald and Hartwig. Methods for these couplings can be adapted from, for example, JACS 2006, 128, 8742-8743, JACS 2007, 129, 3490-3491, and Topic in Current Chemistry 2002, 219, 131-209, Angewante Chemie Int. Engl. Ed. 2006 45 4321-4326, the entire teachings of which are incorporated herein by reference. In certain applications of these methods, it may be useful to employ appropriate hydroxyl or amine protecting groups, which can be represented by R′ and R″. When R′ and R″ are suitable protecting groups, deprotection can lead to intermediates that can be further derivatized as described herein. See also the review article Angew. Chem. Int. Ed. Eng. 2003, 42, 5400-5451, the entire teachings of which are incorporated herein by reference.

Construction of thioether analogs as depicted in Scheme 20 can be accomplished by treating corresponding 3-halo (e.g., iodo) substituted compounds with a thiol, N-methylmorpholine and copper iodide in a suitable solvent at elevated temperatures. Corresponding sulfoxide and sulfone analogs can be obtained by oxidation. See, for example WO 2004 056830, the entire teachings of which is incorporated herein by reference.

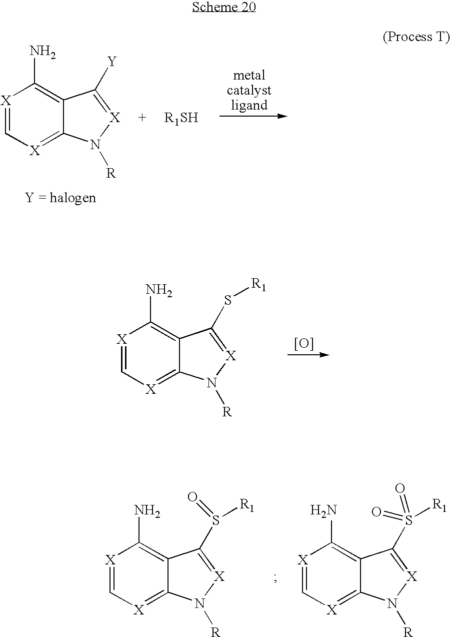

In the first step of process T, a dimer can also be isolated, for example, compound 457:

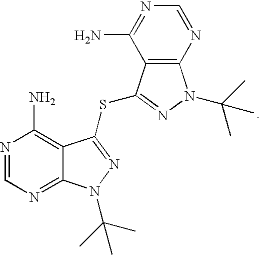

Substituents at the 6 position can be incorporated by condensation of a pyrrole or pyarazole with the appropriate nitrile or amidine (Scheme 21) as described in Heterocycles (Southwick, P., Dhawan, B., 1975, 11, 1999) and J. Chem. Soc., Perkin Trans 1 (Hanefeld, U. et al., 1996, 1545), the entire teachings of which are incorporated herein by reference.

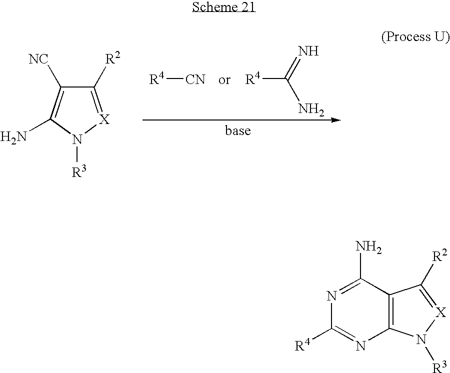

Scheme 22 delineates a procedure for the preparation of a diverse group of 3-substituted pyrazolo[3,4-d]pyrimidines via a key carbinol intermediate. The synthesis begins with the protected acid chloride and malononitrile followed by the preparation of the enol ether which is reacted with an appropriate hydrazine to furnish a substituted pyrazole as described in J. Chem. Soc., Perkin Trans 1 (Hanefeld, U. et al., 1996, 1545), the entire teachings of which are incorporated herein by reference. Cyclization to the pyrazolo[3,4-d]pyrimidine followed by the removal of the protection (for example a benzyl group) provides the carbinol. The carbinol can be oxidized to the formyl derivative which upon reaction with nucleophilic reagents such as a Grignard reagent can provide an alcohol which in turn can be reduced to the corresponding alkyl analog according to the procedures apparent to those skilled in the art. Those who are skilled in the art will appreciate that the intermediates such as the carbinol, the formyl derivative, or the alcohol may also serve as precursors for a variety of novel compounds.

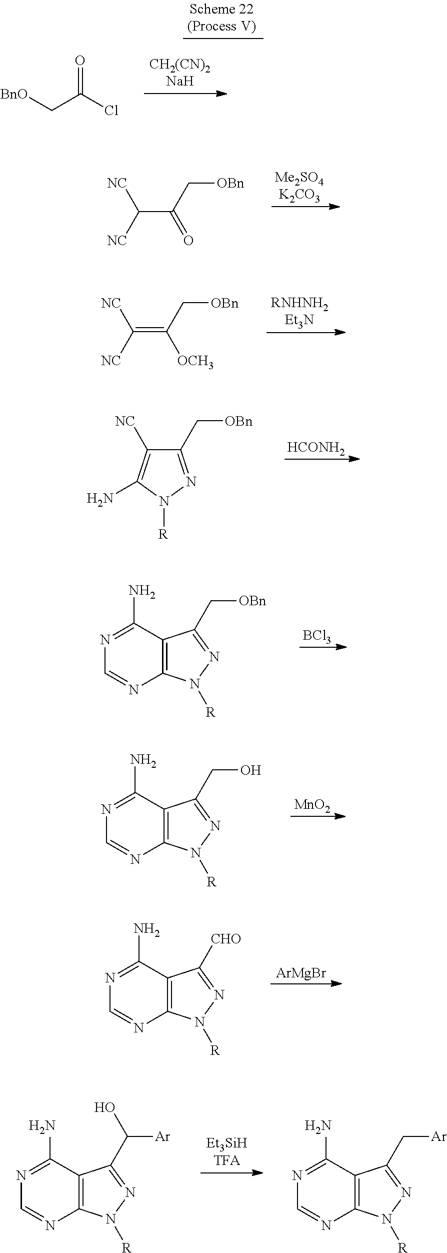

Another method for the construction of compounds with alkoxy and aryloxy substituents at the 3 position, Scheme 23, begins with the condensation of a protected alcohol with tetracyanoethylene as described in J. Amer. Chem. Soc. (Middleton, W. J., Engelhardt, V. A., 1958, 80, 2788), the entire teachings of which are incorporated herein by reference. Cyclization to the pyrazole with the appropriate hydrazine followed by formation of the pyrimidine ring provides access to the protected 3-hydroxy compound. (Those skilled in the art will appreciate that starting with a desired rather than protected alcohol provides direct access to the final compound). Deprotection followed by alkylation or arylation, provides the desired compounds.

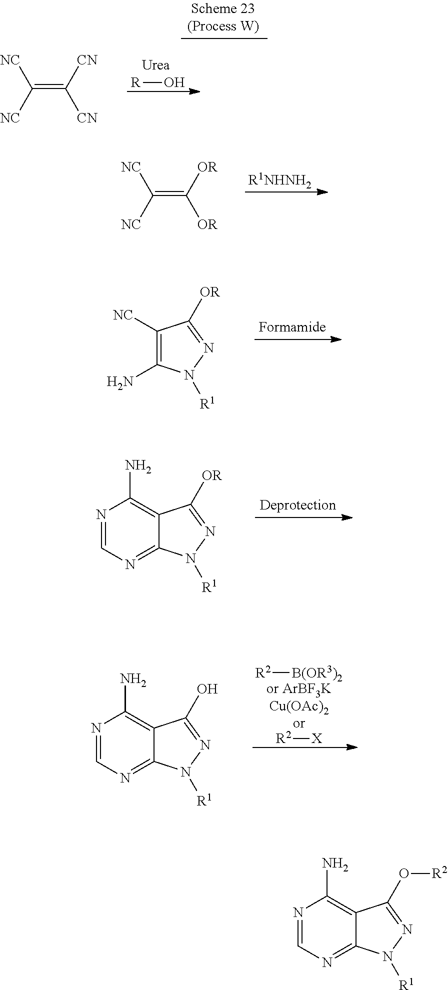

Derivatization of N-1 heterocyclic substituents can be accomplished by the method shown in Scheme 24. Deprotection of a previously installed nitrogen heterocycle followed by alkylation for example by reductive alkylation or arylation by methods described in J. Org. Chem. (Ahmed, F. A-M., et al., 1996, 61, 3849) and in Advanced Organic Chemistry (Smith, M. B., March J., Wiley, 2001, p 501-511), the entire teachings of which are incorporated herein by reference, provides the desired derivatives.

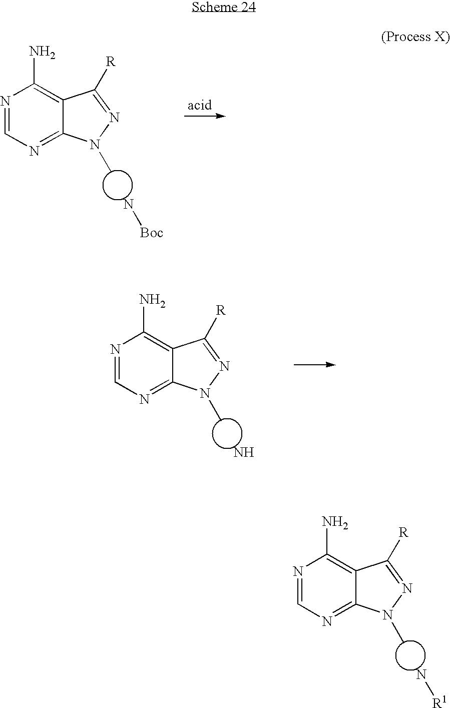

Scheme 25 outlines a procedure for the preparation of C-2 substituted pyrrolo[2,3-d]pyrimidines. When R4 represents a hydrogen, the procedure leads to the preparation of C-2 unsubstituted pyrrolo[2,3-d]pyrimidines. The procedure starts with an appropriately substituted acetophenone derived halide (X=halogen) which upon nucleophilic displacement with an amine affords an amino-ketone which can be isolated as a salt (for example hydrochloride, hydrobromide or the like) or as a free base. Reaction of the amino-ketone with malononitrile in the presence of a base (such as sodium methoxide, sodium hydride, potassium hydroxide or the like) in an aqueous or anhydrous organic solvent (such as methanol, ethanol, tetrahydrofuran, dioxane, toluene, etc.) affords a 2-amino-3-cyanopyrrole derivative as described in Bioorg. Med. Chem. Lett. (propinski, J. F., 2005, 15, 5035) and J. Chem. Soc. (Darroll, J. et al., 1960, 82, 131), the entire teachings of which are incorporated herein by reference. Cyclization with formamide leads to the substituted pyrrolo[2,3-d]pyrimidine compounds.

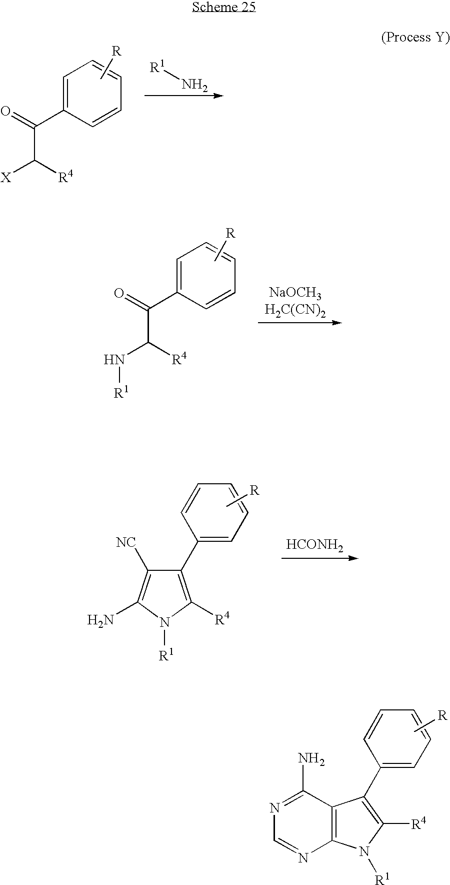

Methods to construct pyrrolo[2,3-d]pyrimidines with various substituents at N-1 (Scheme 26) begin with the condensation of hexamethylene-tetramine with a haloacetophenone as in Bioorg. Med. Chem. Lett. (Wilder, L. et al., 2001, 11, 1849; and Altmann, E. et al., 2001, 11, 853), the entire teachings of which are incorporated herein by reference. Protection with, for example, acetyl followed by two cyclization reactions provides the unsubstituted pyrrolo[2,3-d]pyrimidines. Derivatization, for example, by alkylation, acylation, sulfonylation, and arylation, can be accomplished by methods known to those skilled in the and as described in Bioorg. Med. Chem. Lett. (Altman, E. et al., 2001, 11, 853; propinski, J. F., et al., 2005, 15, 5035; and Arnold, L. D., et al., 2000, 10, 2167).

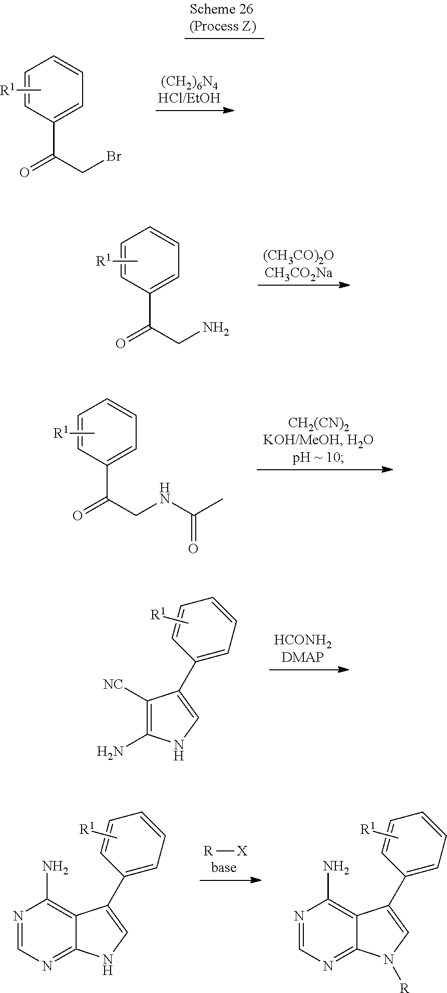

Derivatization of the C-4 amino group can be accomplished by treating the compound with a derivatizing agent, for example, an alkyl, aryl or acyl halide under the appropriate conditions as described in Advanced Organic Chemistry (Smith, M. B., March J., Wiley, 2001, p 501-511) and J. Org. Chem. (Bio, M. M. et al., 2004, 69, 6257), the entire teachings of which are incorporated herein by reference (Scheme 27). A second group, either the same or distinct from the first, can be incorporated in a second step.

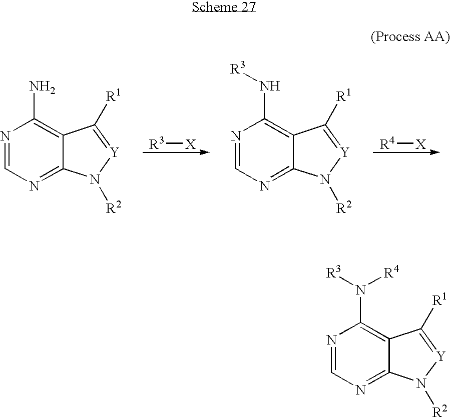

Some pyrrolo[2,3-d]pyrimidines can be conveniently constructed from C-3 halogenated precursors as shown in Scheme 28. The procedure starts with the halogenation of the unsubstituted pyrrolo[2,3-d]pyrimidine followed by derivatization of N-1 by conventional methods such as electrophilic alkylation by a halide or another compound containing a good leaving group such as a mesylate, a tosylate or the like. Subsequent transition metal catalyzed coupling of the halide with an aryl boronic acid (or an ester), an organo-zinc compound, an organo-tin compound, or a Grignard reagent leads to the formation of the 3-aryl substituted pyrrolo[2,3-d]pyrimidines. Metal catalyzed coupling of the halide with a substituted phenol, a thiophenol, or an aniline derivative leads to the compounds with a C-3 heteroatom linked aromatic derivatives of pyrrolo[2,3-d]pyrimidines (Y═O, S, NR) according to conventional methods as described in Angewand. Chem. (Burgos, C. H., et al, 2006, 45, 4321), Org. Lett. (Ma, D., Cai, Q., 2003, 5, 3799; and Buck, E., et al., 2002, 4, 1623), the entire teachings of which are incorporated herein by reference.

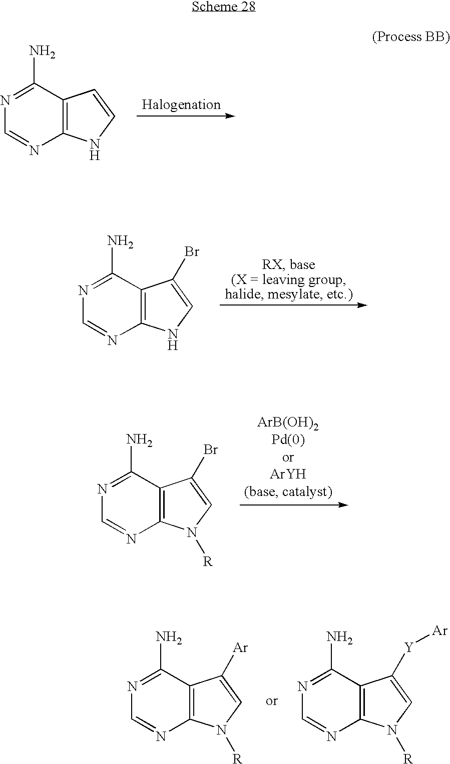

The syntheses of particular compounds prepared by the schemes shown above are also demonstrated in the Examples. Table I shows the compounds by ID, structure, measured melting point, mass spectra (API-ES), and synthetic process.

TABLE I

| | | | MS | Synthetic |
|---|---|---|---|---|
| Compound ID and structure | | M.p., ° C. | (API-ES) | Process |
| 1 | 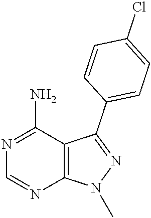 | 245-6 | 260.1 262.1 | A |
| 2 | 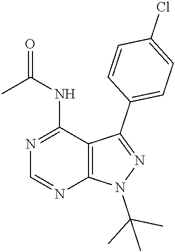 | 164-5 | 344.1 346.1 | AA |
| 3 | 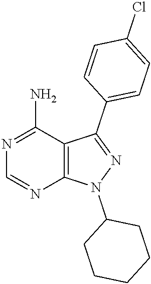 | 137-8 | 328.1 330.1 | A |
| 4 | 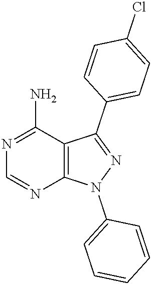 | 248-9 | 322.1 324.1 | A |
| 5 | 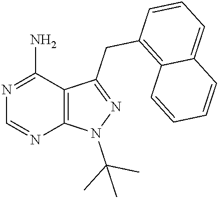 | 169-70 | 332.1 | B |
| 6 | 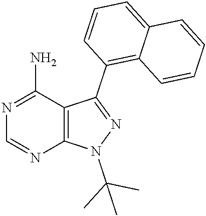 | 215-216 | 318.1 | B |
| 7 | 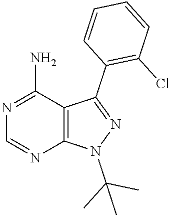 | 176-7 | 302.1 304.1 | B |
| 8 | 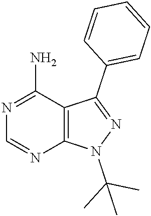 | 129-31 | 268.1 | B |
| 9 | 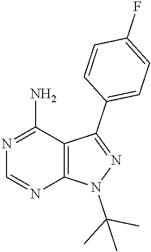 | 189-90 | 286.1 | B |
| 10 | 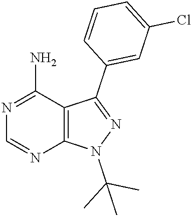 | 134-5 | 302.1 304.1 | B |
| 11 | 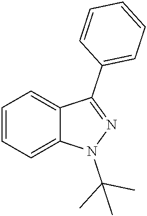 | 89-90 | 282.1 | B |
| 12 | 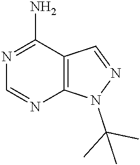 | — | 192.1 | C |
| 13 | 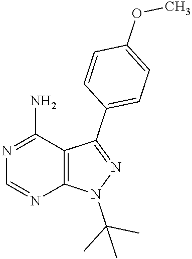 | 168-9 | 298.1 | B |
| 14 | 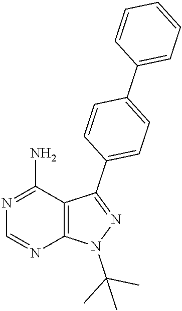 | 191-2 | 344.1 | C |
| 15 | 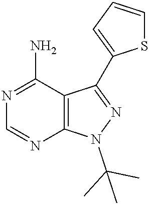 | 135-6 | 274.1 | C |
| 16 | 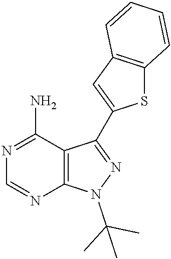 | 209-10 | 324.0 | C |
| 17 | 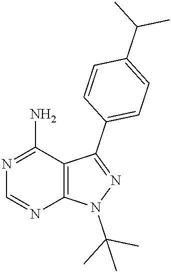 | 186-7 | 310.1 | C |
| 18 | 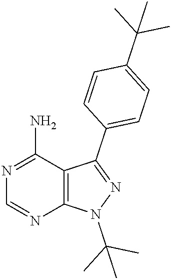 | 180-1 | 324.2 | C |
| 19 | 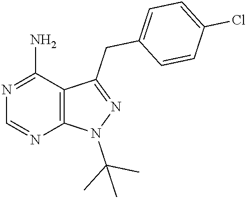 | 156-7 | 316.3 318.3 | B |
| 20 | 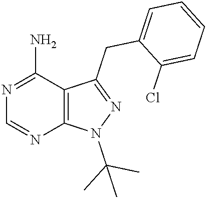 | 173-4 | 316.1 318.1 | B |
| 21 | 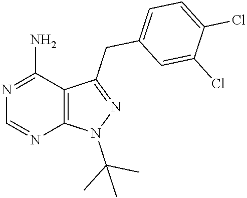 | 174-5 | 350.1 352.1 | B |
| 22 | 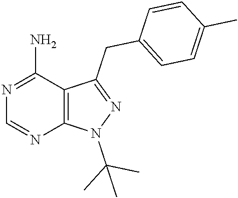 | 145-7 | 366.2 | B |
| 23 | 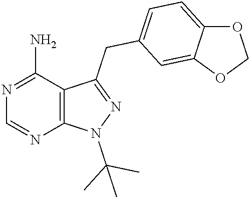 | 165-7 | 326.2 | B |
| 24 | 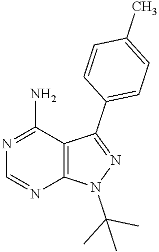 | 192-3 | 282.1 | B |
| 25 | 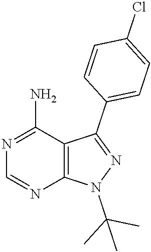 | 210-11 | 302.1 304.1 | B |
| 26 | 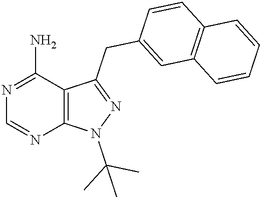 | 138-9 | 332.1 | B |
| 27 | 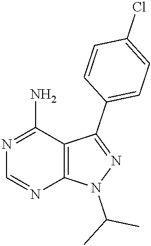 | 179-80 | 301.1 | A |
| 28 | 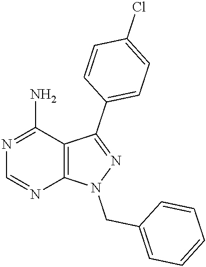 | 200-1 | 336.1 338.1 | A |
| 29 | 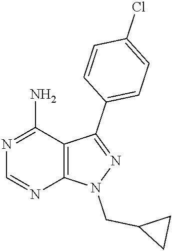 | 144-5 | 300.1 302.1 | A |
| 30 | 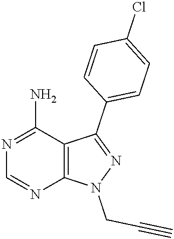 | 173-4 | 284.0 286.0 | A |
| 31 | 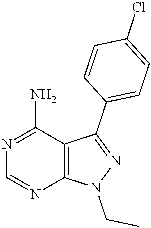 | 163-4 | 274.1 276.1 | A |
| 32 | 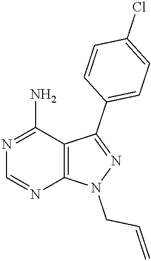 | 119-20 | 286.1 288.1 | A |
| 33 | 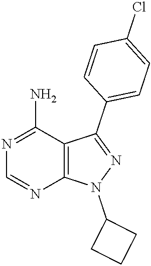 | 173-4 | 300.1 302.1 | A |
| 34 | 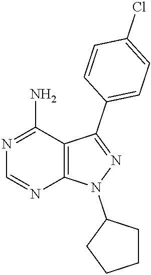 | 160-1 | 314.1 316.1 | A |
| 35 | 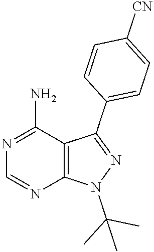 | 238-9 | 293.1 | C |
| 36 | 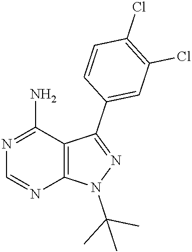 | 195-6 | 336.0 338.0 | C |
| 37 | 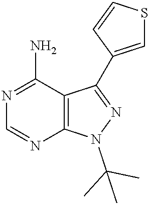 | 156-7 | 274.1 | C |
| 38 | 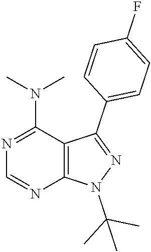 | 110-1 | 314.1 | AA |
| 39 | 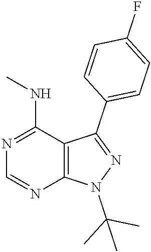 | 157-9 | 300.1 | AA |
| 40 | 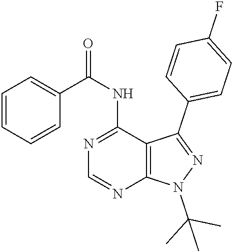 | 183-4 | 390.1 | AA |
| 41 | 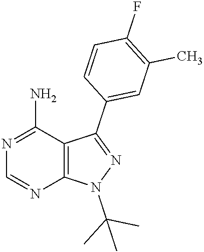 | 160-1 | 300.1 | C |
| 42 | 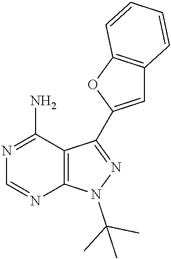 | 195-6 | 308.1 | C |
| 43 | 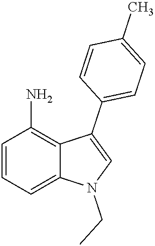 | oil | 251.2 | D |
| 44 | 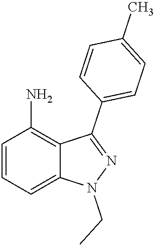 | 114-6 | 252.1 | F |
| 45 | 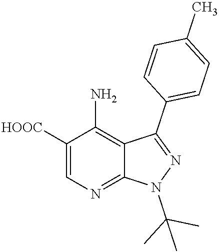 | 227-31 | 323.3 (APCI Neg) | G |
| 46 | 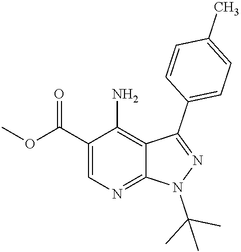 | 127-8 | 339.1 | G |
| 47 | 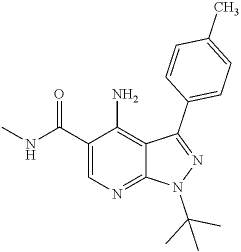 | 208-15 dec. | 336.1 | G |
| 48 | 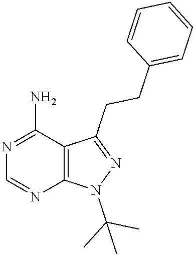 | 129-30 | 296.4 | B, reduction |
| 49 | 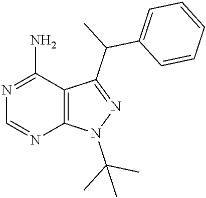 | 160-1 | 296.4 | B |
| 50 | 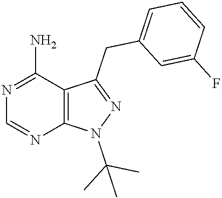 | 152-3 | 300.4 | B |
| 51 | 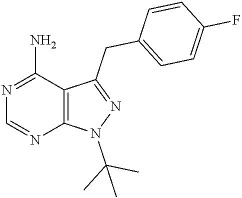 | 166-9 | 300.4 | B |
| 52 | 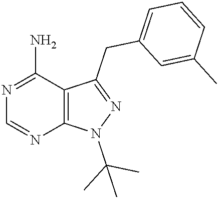 | 129-30 | 296.1 | B |
| 53 | 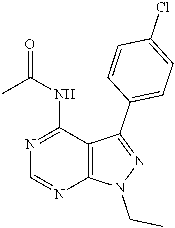 | 162-3 | 316.0 318.0 | AA |
| 54 | 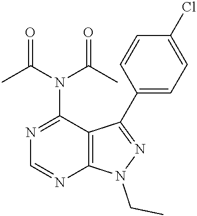 | 112-3 | 358.1 360.1 | AA |
| 55 | 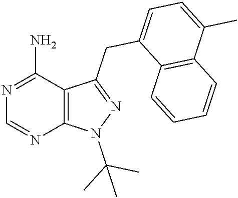 | 195-7 | 346.1 | B |
| 56 | 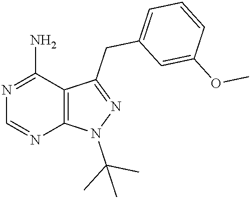 | 99-100 | 312.1 | B |
| 57 | 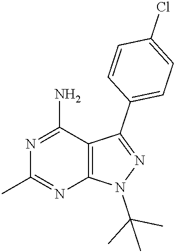 | 187-8 | 316.1 318.1 | U |
| 58 | 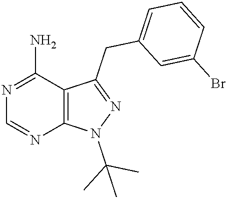 | 142-3 | 360.0 362.0 | B |
| 59 | 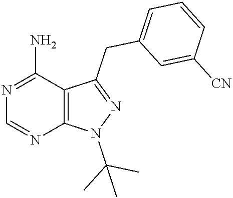 | 193-4 | 307.2 | B (No. 58), substitution |
| 60 | 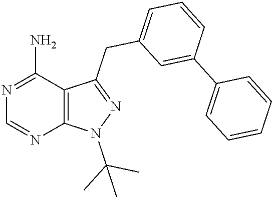 | 150-1 | 358.2 | B (No. 58), arylation |
| 61 | 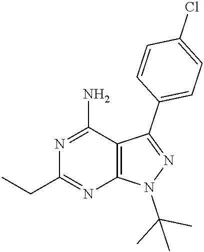 | 205-6 | 330.1 332.1 | U |
| 62 | 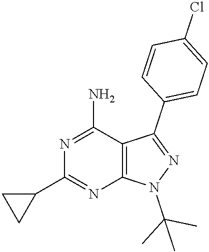 | 175-6 | 342.1 344.1 | U |
| 63 | 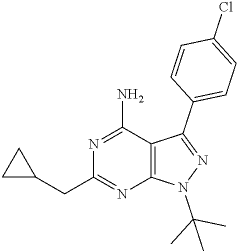 | 187-8 | 356.1 358.1 | U |
| 64 | 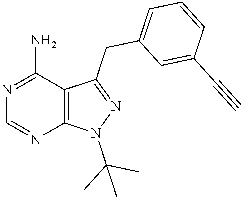 | 131-2 | 306.2 | B (No. 58), alkylation |
| 65 | 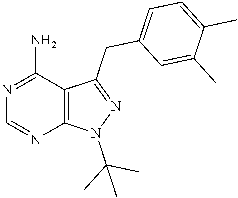 | 146-8 | 310.2 | B |
| 66 | 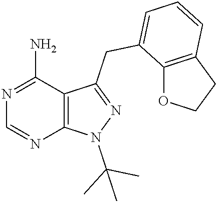 | 193-5 | 324.2 | B |
| 67 | 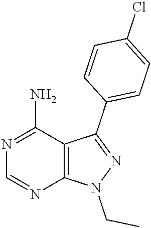 | 163-4 | 273.1 275.1 | Y |
| 68 | 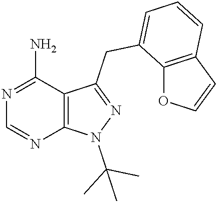 | 129-32 | 322.2 | B (No. 66), oxidation |
| 69 | 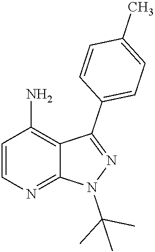 | 163-5 | 281.5 | G |
| 70 | 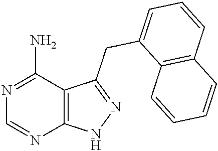 | 292-3 | 276.1 | D |
| 71 | 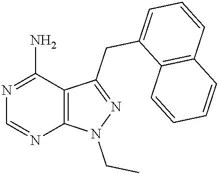 | 166-7 | 304.1 | D |
| 72 | 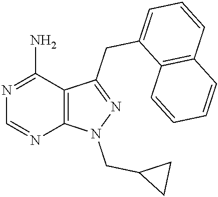 | 155-6 | 330.1 | D |
| 73 | 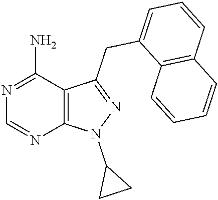 | 159-60 | 316.1 | D |
| 74 | 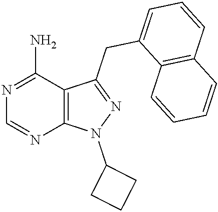 | 153-4 | 330.1 | D or B |
| 75 | 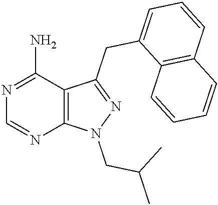 | 156-7 | 332.1 | D |
| 76 | 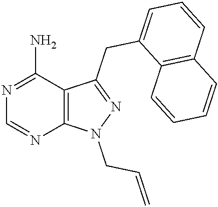 | 149-50 | 316.1 | D |
| 77 | 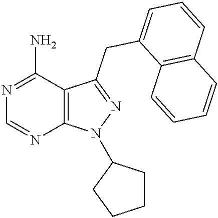 | 158-9 | 344.3 | D |
| 78 | 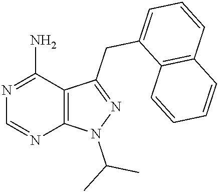 | 146-7 | 318.3 | D |
| 79 | 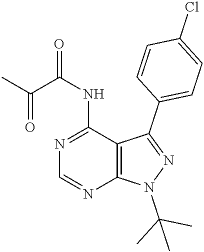 | 218-20 | 372.8 374.7 | AA |
| 80 | 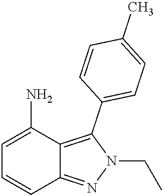 | 123-4 | 252.2 | F |
| 81 | 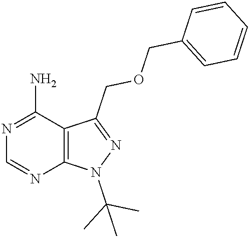 | 124-5 | 312.2 | V |
| 82 | 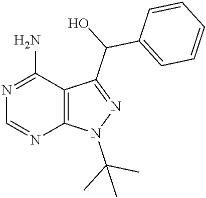 | 158-60 | 298.2 | V |
| 83 | 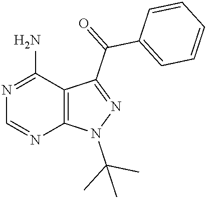 | 182-3 | 296.2 | V |
| 84 | 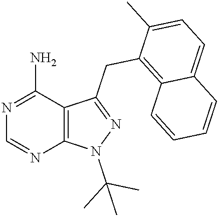 | 169-70 (TFA salt) | 346.2 | V |
| 85 | 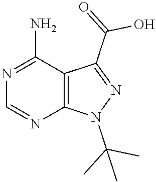 | 304-5 dec. | 233.9 (API-ES Neg) | V (No. 149), oxidation (Ex. 24) |
| 86 | 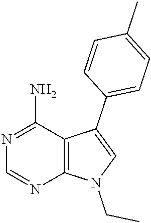 | 147-8 | 253.2 | Y |
| 87 | 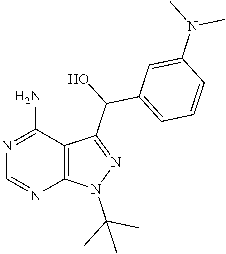 | 78-81 | 341.2 | V |
| 88 | 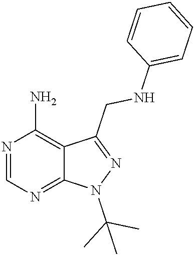 | 129-30 | 297.2 | V (No. 149), reductive amination |
| 89 | 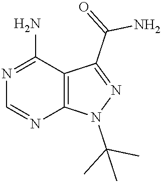 | 281-4 | 235.2 | Ex. 27 |
| 90 | 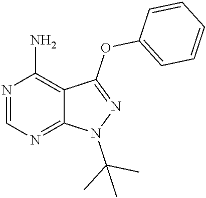 | 180-1 | 284.2 | S |
| 91 | 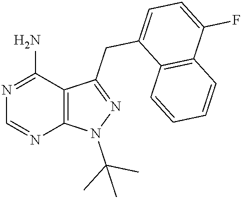 | 180-1 | 350.2 | B |
| 92 | 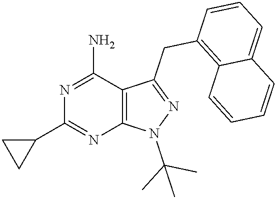 | 188-9 | 372.2 | U |
| 93 | 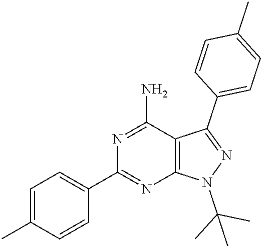 | | | Commercially availaable |
| 94 | 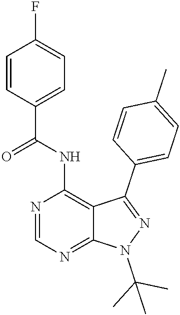 | | | Commercially available |
| 95 | 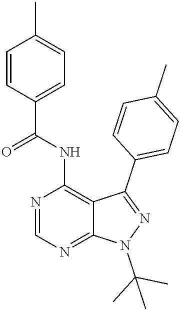 | | | Commercially available |
| 96 | 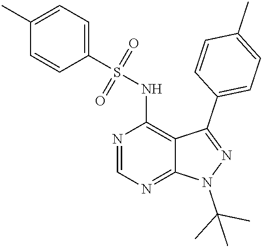 | | | Commercially available |
| 97 | 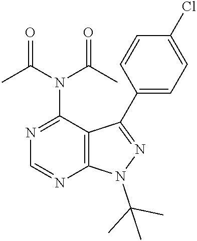 | 127-9 | 386.1 388.1 | AA |
| 98 | 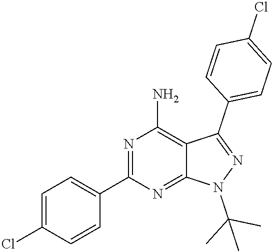 | | | Commercially available |
| 99 | 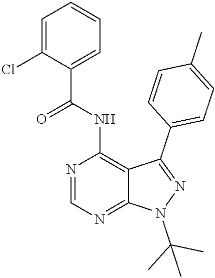 | | | Commercially available |
| 100 | 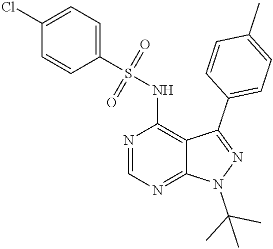 | | | Commercially available |
| 101 | 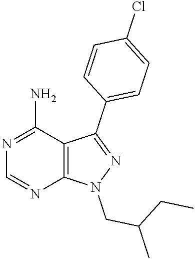 | 142-3 | 316.1 318.1 | A |
| 102 | 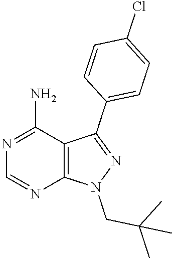 | 84-5 | 316.1 318.1 | A |
| 103 | 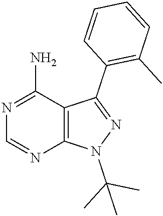 | 137-8 | 282.1 | B |
| 104 | 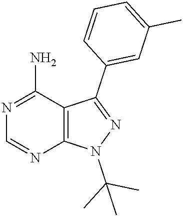 | 136-7 | 282.1 | B |
| 105 | 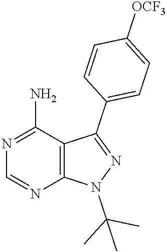 | 151-2 | 352.1 | B |
| 106 | 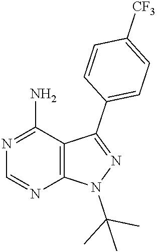 | 159-60 | 336.1 | B |
| 107 | 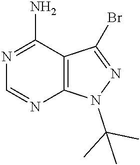 | 185-6 | 271.0 272.0 | C |
| 108 | 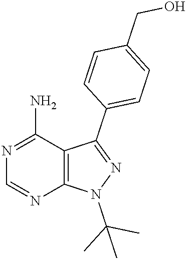 | 166-7 | 298.1 | C |
| 109 | 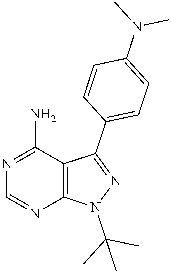 | 259-61 | 311.2 | C |
| 110 | 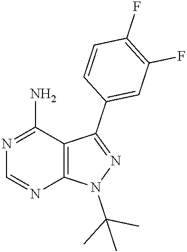 | 215-6 | 304.1 | C |
| 111 | 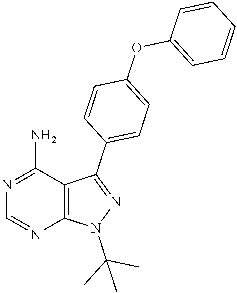 | 134-5 | 360.1 | C |
| 112 | 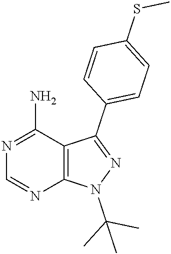 | 173-4 | 314.1 | C |
| 113 | 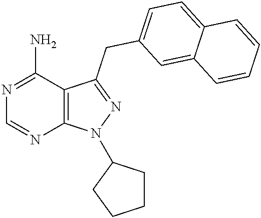 | 155-6 | 344.1 | B |
| 114 | 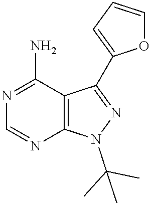 | 129-30 | 258.1 | C |
| 115 | 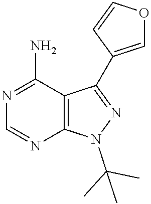 | 142-3 | 258.1 | C |
| 116 | 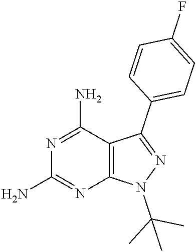 | 207-9 | 301.1 | U (Ex. 13) |
| 117 | 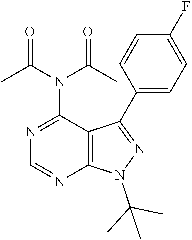 | 109-10 | 370.1 | AA (Ex. 15) |
| 118 | 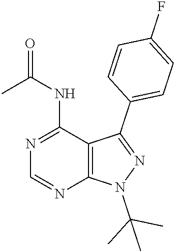 | 146-7 | 328.1 | AA (Ex. 15) |
| 119 | 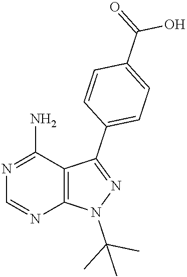 | 275-8 | 310.1 | C |
| 120 | 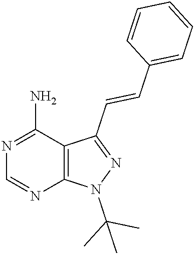 | 175-7 | 294.1 | C |
| 123 | 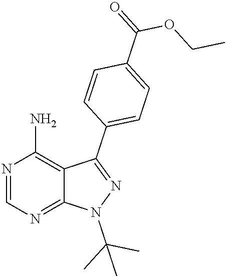 | 181-3 | 340.1 | C |
| 124 | 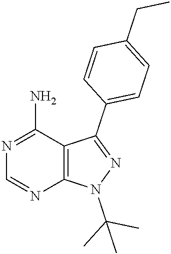 | 196-8 | 296.1 | C |
| 125 | 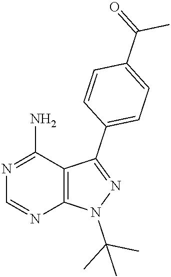 | 205-7 | 310.1 | C |
| 126 | 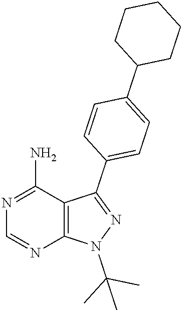 | 148-9 | 350.1 | C |
| 127 | 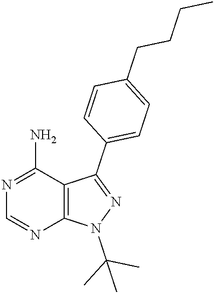 | 120-2 | 324.2 | C |
| 128 | 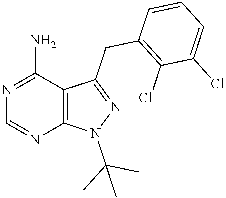 | 183-4 | 350.0 352.0 | B |
| 129 | 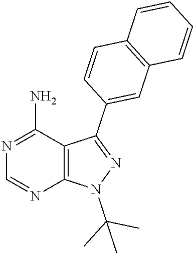 | 213-5 | 318.1 | C |
| 130 | 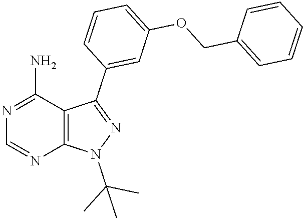 | 117-9 | 374.1 | C |
| 131 | 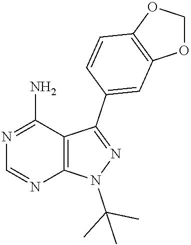 | 207-10 | 312.1 | C |
| 132 | 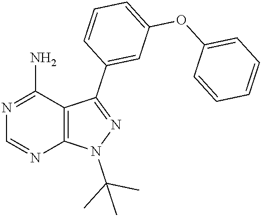 | 142-9 | 360.3 | B |
| 133 | 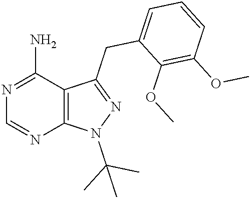 | 176-7 | 342.3 | B |
| 134 | 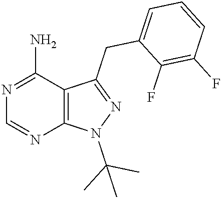 | 143-4 | 318.3 | B |
| 135 | 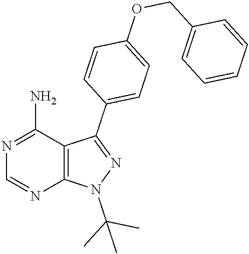 | 157-8 | 374.3 | C |
| 136 | 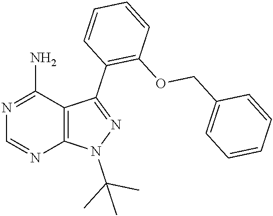 | 198-9 | 374.3 | C |
| 137 | 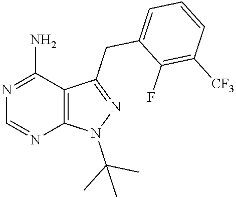 | 152-3 | 368.2 | B |
| 138 | 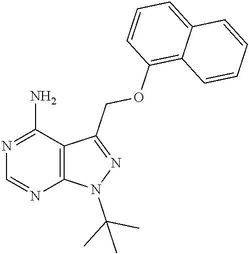 | 178-9 | 348.2 | B |
| 139 | 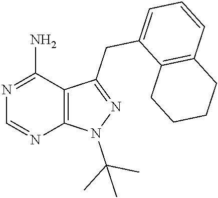 | 182-4 | 336.3 | B |
| 140 | 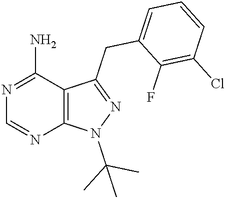 | 151-2 | 334.2 336.2 | B |
| 141 | 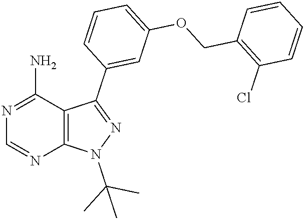 | 69-70 | 408.2 410.2 | C |
| 142 | 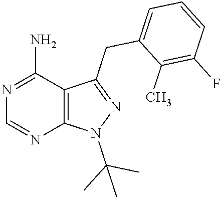 | 193-4 | 314.3 | B |
| 143 | 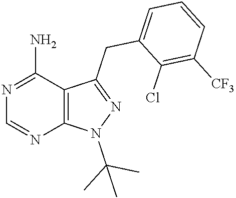 | 191-2 | 384.2 386.3 | B |
| 144 | 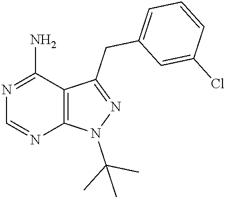 | 134-6 | 316.3 318.3 | B |
| 145 | 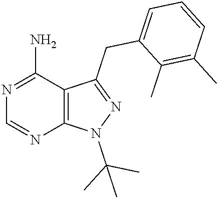 | 156-7 | 310.4 | B |
| 146 | 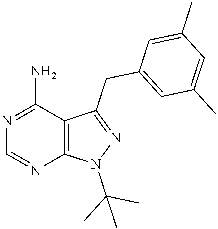 | 180-1 | 310.3 | B |
| 147 | 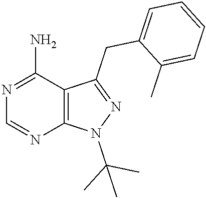 | 163-4 | 296.2 | B |
| 148 | 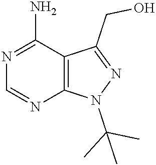 | 152-3 | 222.2 | V (Ex. 22) |
| 149 | 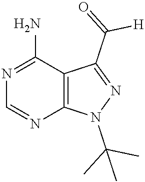 | 183-4 | 220.1 | V (Ex. 22) |
| 150 | 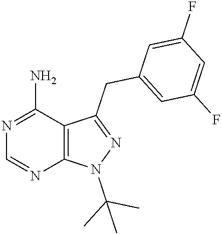 | 167-8 | 318.2 | B |
| 151 | 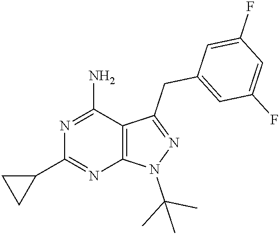 | 149-50 | 358.2 | U |
| 152 | 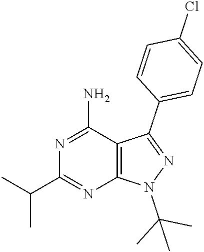 | 167-8 | 344.2 346.2 | U |
| 153 | 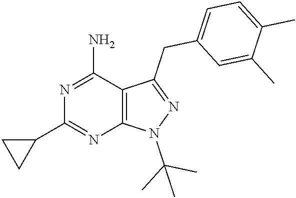 | 175-6 | 350.2 | U |
| 154 | 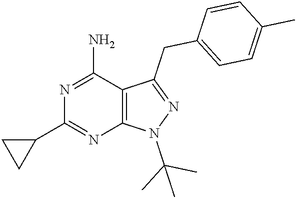 | 169-71 | 336.2 | U |
| 155 | 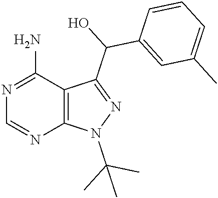 | 70-9 | 312.2 | V |
| 156 | 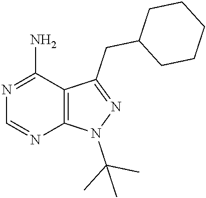 | 174-5 | 288.2 | B |
| 157 | 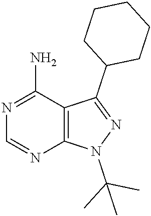 | 160-1 | 274.2 | B |
| 158 | 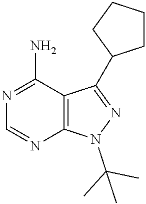 | 169-70 | 260.2 | B |
| 159 | 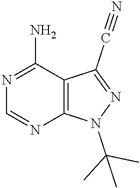 | 213-5 | 217.2 | Ex. 26 |
| 160 | 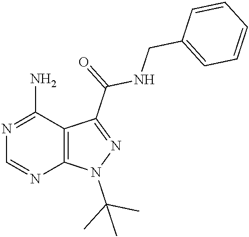 | 240-1 | 325.2 | Ex. 25 |
| 161 | 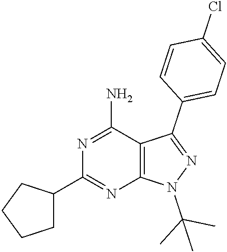 | 172-3 | 370.2 372.2 | U |
| 162 | 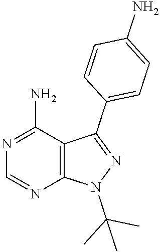 | 186-8 | 283.1 | C |
| 163 | 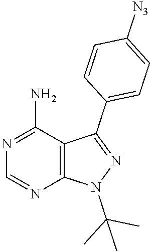 | 189-93 | 309.3 | C (No. 162), azidation |
| 164 | 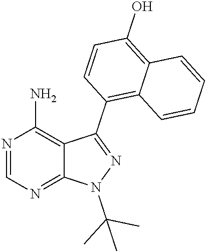 | 223-4 | 334.1 | — |
| 165 | 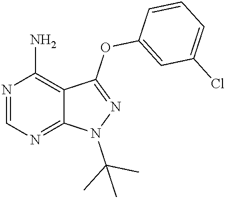 | 142-3 | 318.1 320.1 | S |
| 166 | 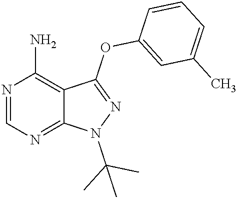 | 158-9 | 298.1 | S |
| 167 | 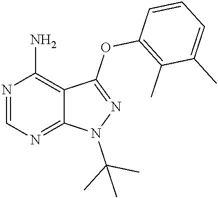 | 163-4 | 312.2 | S |
| 168 | 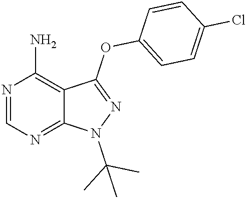 | 153-4 | 318.1 320.1 | S |
| 169 | 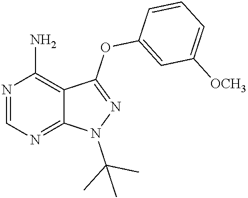 | 151-2 | 314.2 | S |
| 170 | 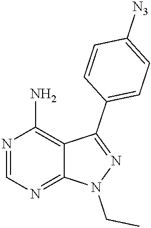 | 184-5 | 281.1 | C, azidation |
| 171 | 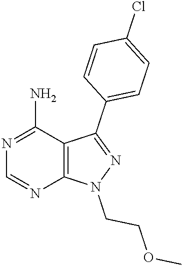 | 215-6 | 304.1 306.1 | D |
| 172 | 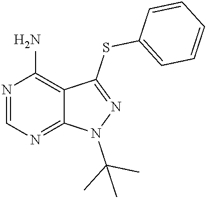 | 199-200 | 300.1 | T |
| 173 | 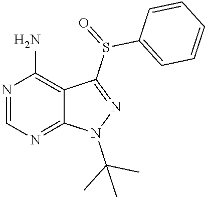 | 161-2 | 316.1 | T (Ex. 29) |
| 174 | 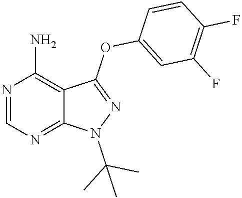 | 152-4 | 320.1 | S |
| 175 | 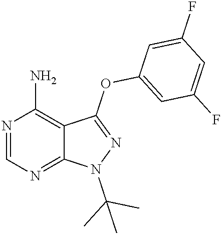 | 181-4 | 320.1 | S |
| 176 | 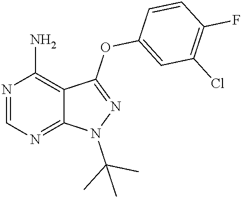 | 128-31 | 336.1 338.1 | S |
| 177 | 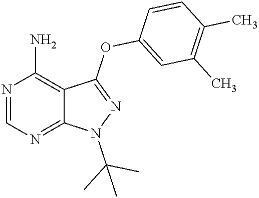 | 178-80 | 312.2 | S |
| 178 | 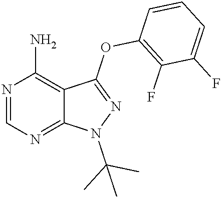 | 157-8 | 320.1 | S |
| 179 | 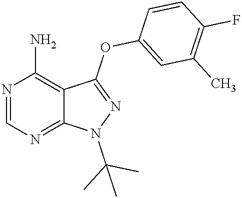 | 136-7 | 316.1 | S |
| 180 | 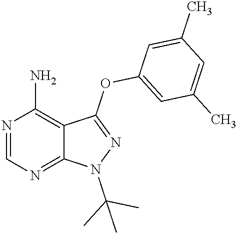 | 165-6 | 312.1 | S |
| 181 | 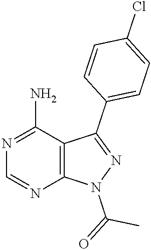 | 197-9 | 288.0 290.0 | D (Ex. 31) |
| 182 | 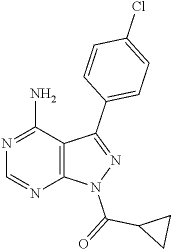 | 200-3 | 314.1 316.0 | D |
| 183 | 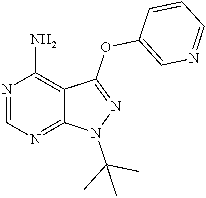 | 169-70 | 285.1 | S |
| 184 | 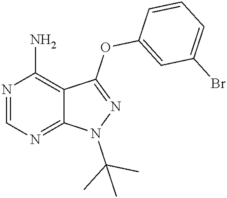 | 135-6 | 362.0 363.0 | S |
| 185 | 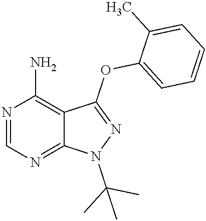 | 149-50 | 298.1 | S |
| 186 | 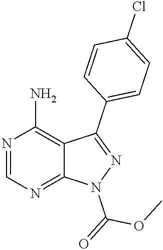 | 240 dec. | 304.1 306.0 | D (Ex. 32) |
| 187 | 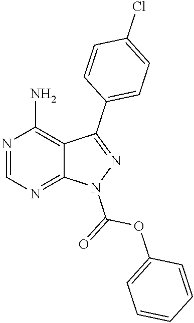 | 235 dec. | 366.1 368.0 | D |
| 188 | 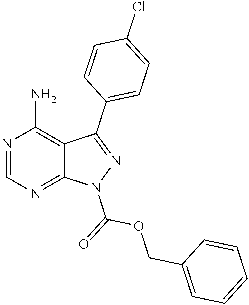 | 213 dec. | 379.8 381.7 | D |
| 189 | 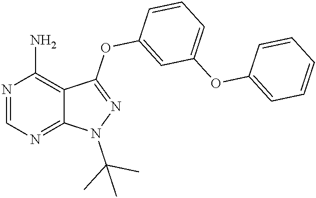 | 127-8 | 376.1 | S |
| 190 | 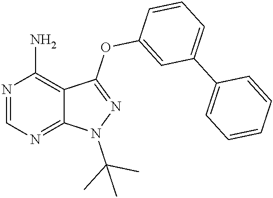 | 49-52 | 360.1 | S |
| 191 | 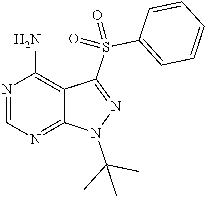 | 203-6 | 332.1 | T (Ex. 29) |
| 192 | 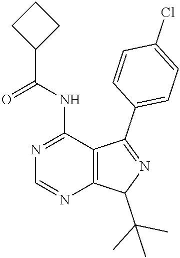 | 171-2 | 383.1 385.1 | AA |
| 193 | 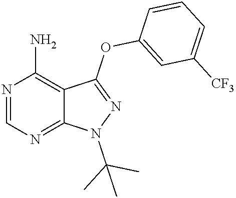 | 140-41 | 352.1 | S |
| 194 | 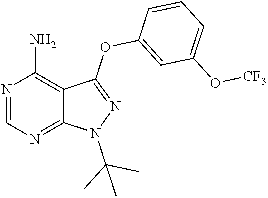 | 109-10 | 368.1 | S |
| 195 | 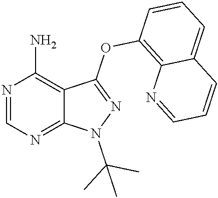 | 165-6 | 335.1 | S |
| 196 | 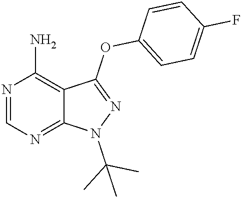 | 158-9 | 302.1 | S |
| 197 | 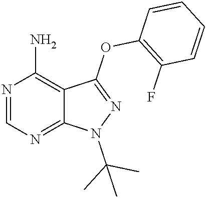 | 172-3 | 302.1 | S |
| 198 | 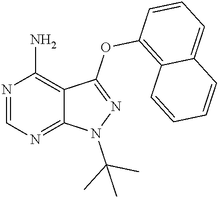 | 162-5 | 334.1 | S |
| 199 | 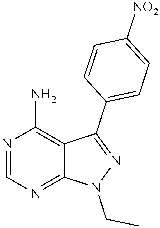 | 234-7 | 285.0 | D |
| 200 | 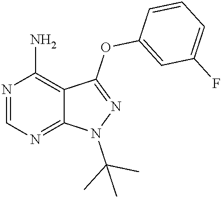 | 171-2 | 302.1 | S |
| 201 | 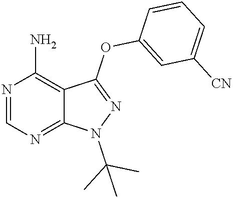 | 164-5 | 309.1 | S |
| 202 | 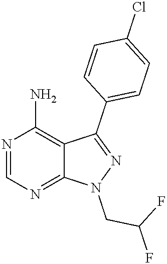 | 192-3 | 246.1 247.9 | D |
| 203 | 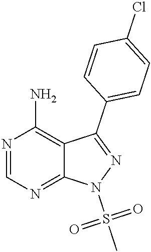 | 225-6 | 324.0 326.0 | D (Ex. 34) |
| 204 | 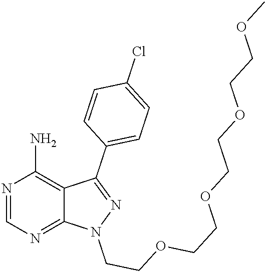 | oil | 436.1 438.1 | D |
| 205 | 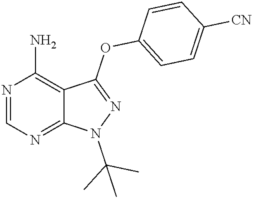 | 194-6 | 309.1 | S |
| 206 | 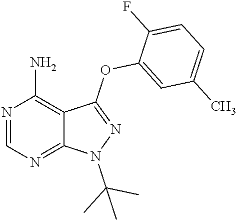 | 144-6 | 316.1 | S |
| 207 | 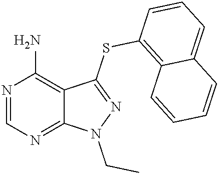 | 169-71 | 308.1 | T |
| 208 | 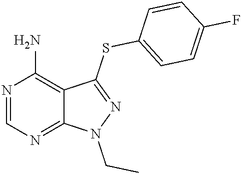 | 151-2 | 290.1 | T |
| 209 | 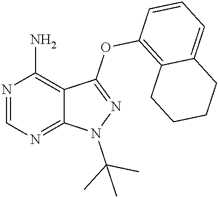 | 162-3 | 338.2 | S |
| 210 | 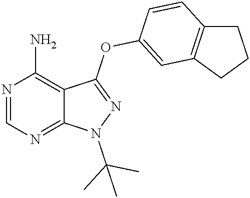 | 185-6 | 324.2 | S |
| 211 | 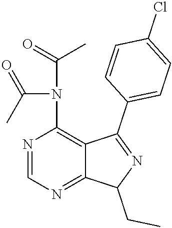 | 178-81 | 357.1 359.1 | AA (Ex. 35) |
| 212 | 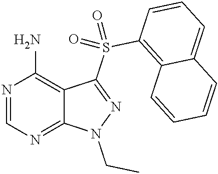 | 198-201 | 354.1 | T |
| 213 | 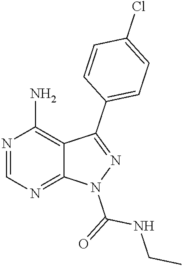 | 195 decomp. | 316.7 318.9 | L (Ex. 36) |
| 214 | 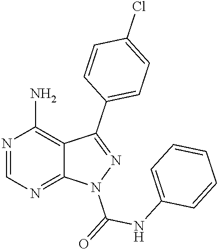 | 231 decomp. | 364.9 366.0 | L |
| 215 | 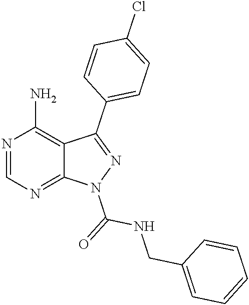 | 300 decomp. | 378.9 308.9 | L |
| 216 | 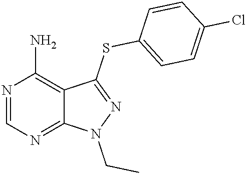 | 169-171 | 306.0 308.0 | T |
| 217 | 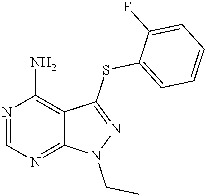 | 129-31 | 290.0 | T |
| 218 | 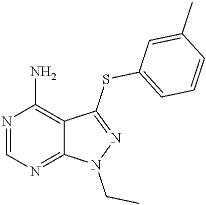 | 109-11 | 286.1 | T |
| 219 | 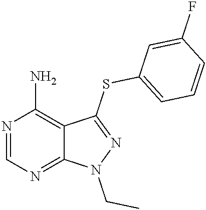 | 116-7 | 290.0 | T |
| 220 | 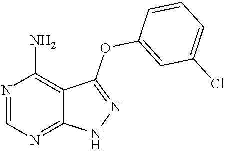 | 269-71 | 262.0 264.0 | S (Ex. 37) |
| 221 | 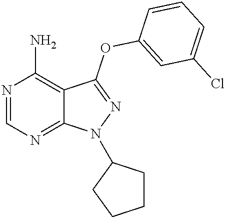 | 103-5 | 330.1 332.1 | S (Ex. 38) |
| 222 | 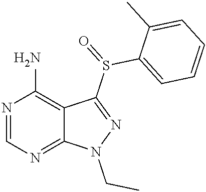 | 137-9 | 302.1 | T |
| 223 | 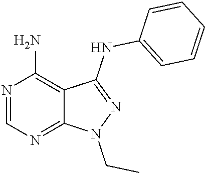 | 181-3 | 256.1 | Q (Ex. 39) |
| 224 | 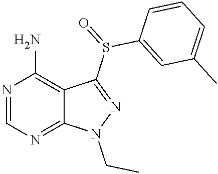 | 164-5 | 302.1 | T |
| 225 | 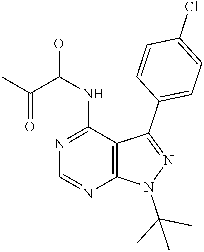 | 218-20 | 372.8 374.7 | AA |
| 226 | 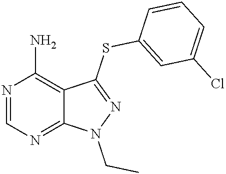 | 131-2 | 306.0 308.0 | T |
| 227 | 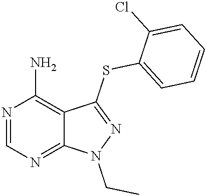 | 165-7 | 306.0 308.0 | T |
| 228 | 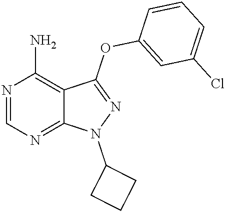 | 120-1 | 316.1 318.1 | S |
| 229 | 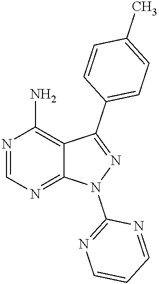 | 274-5 | 304.1 | D (Ex. 40) |
| 230 | 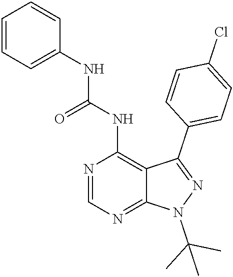 | 172-3 | 419.3 421.2 | AA (Ex. 41) |
| 231 | 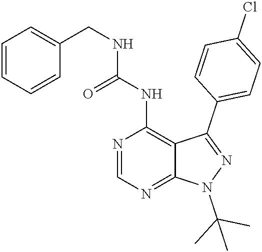 | 140-1 | 435.4 437.3 | AA |
| 232 | 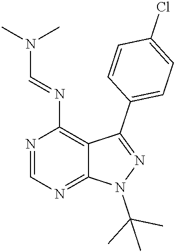 | 131-2 | 357.3 359.3 | Ex. 42 |
| 233 | 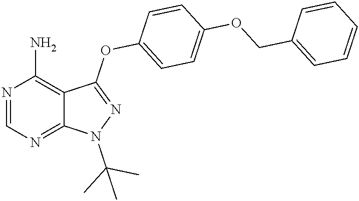 | 182-3 | 390.1 | S |
| 234 | 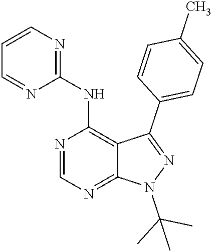 | 201-2 | 360.1 | Ex. 43 |
| 235 | 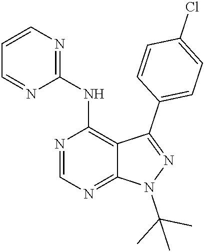 | 220-1 | 380.1 382.1 | Ex. 43 |
| 236 | 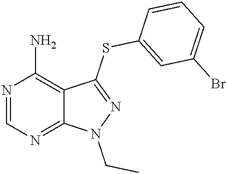 | 140-1 | 349.9 351.9 | T |
| 237 | 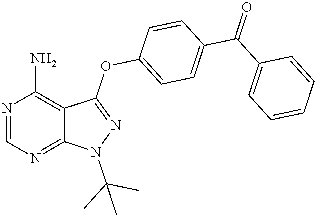 | 192-3 | 388.1 | S |
| 238 | 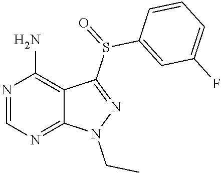 | 130-3 | 306.0 | T |
| 239 | 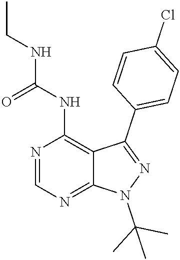 | 68-70 | 373.3 375.2 | AA |
| 240 | 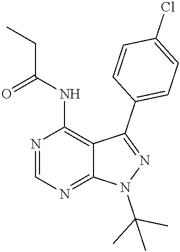 | 173-4 | 358.4 360.3 | AA |
| 241 | 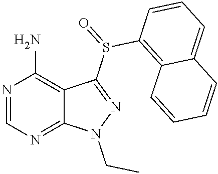 | 206-8 | 338.1 | T |
| 242 | 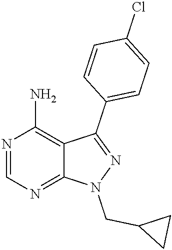 | 164-5 | 299.1 301.1 | Z (Ex. 44) |
| 243 | 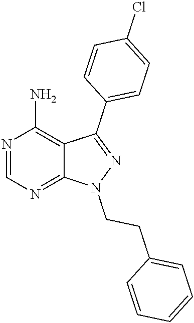 | 189-90 | 350.1 352.1 | A |
| 244 | 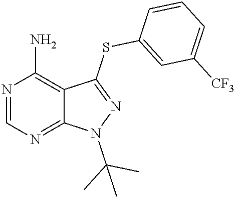 | 151-3 | 368.1 | T |
| 245 | 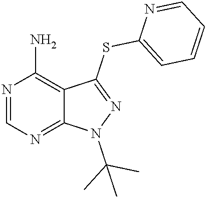 | 225-6 | 301.1 | T |
| 246 | 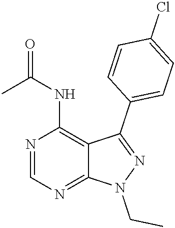 | 148-50 | 315.1 317.1 | AA |
| 247 | 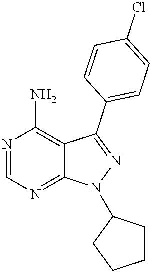 | 160-2 | 313.1 315.1 | Z |
| 248 | 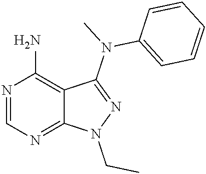 | 215-7 | 269.1 | Q (Ex. 46) |
| 249 | 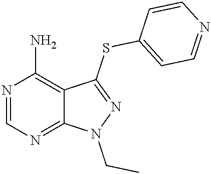 | 205-7 | 273.0 | T |
| 250 | 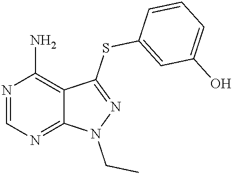 | 158-60 | 288.0 | T |
| 251 | 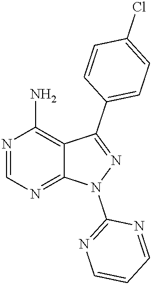 | 284-6 | 324.0 326.0 | D |
| 252 | 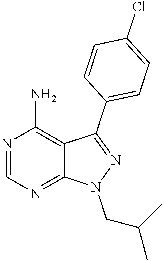 | 157-9 | 301.1 303.1 | Z |
| 253 | 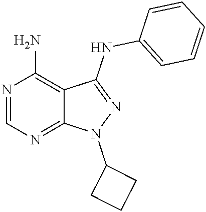 | 169-71 | 281.1 | Q (Ex. 47) |
| 254 | 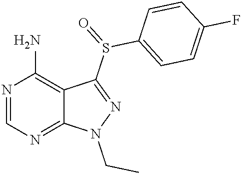 | 147-8 | 306.0 | T |
| 255 | 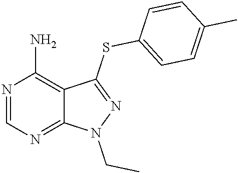 | 137-8 | 286.1 | T |
| 256 | 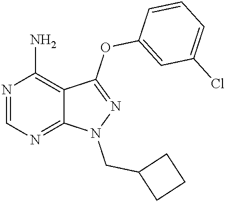 | 125-7 | 330.1 332.1 | S |
| 257 | 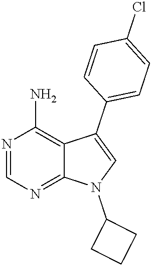 | 101-3 | 299.1 301.1 | Z |
| 258 | 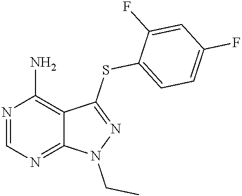 | 143-4 | 308.0 | T |
| 259 | 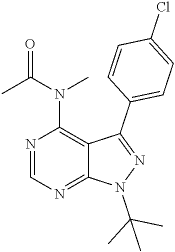 | 157-8 | 358.1 360.1 | AA (Ex. 48) |
| 260 | 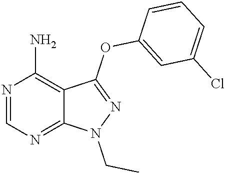 | 114-6 | 290.0 292.0 | S |
| 261 | 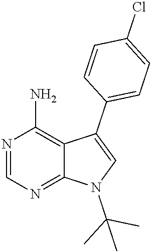 | 220-1 | 301.1 303.1 | Y |
| 262 | 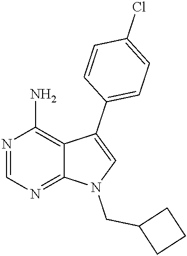 | 155-6 | 313.1 315.1 | Z |
| 263 | 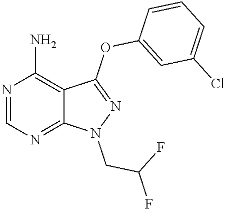 | 179-80 | 326.0 328.0 | S |
| 264 | 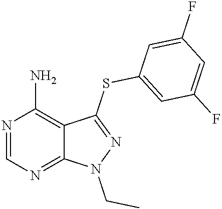 | 171-2 | 308.0 | T |
| 265 | 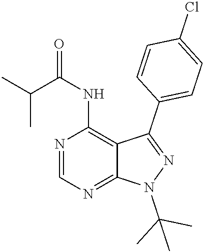 | 162-3 | 372.1 374.0 | AA |
| 266 | 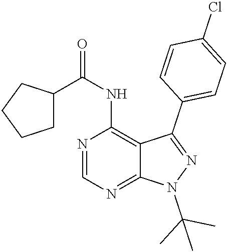 | 178-9 | 398.1 400.1 | AA |
| 267 | 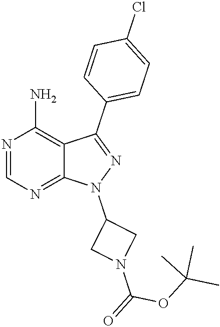 | 233-4 | 399.1 401.1 | D, X (Ex. 50) |
| 268 | 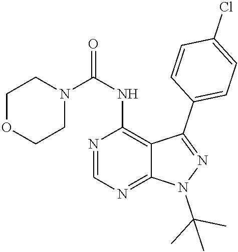 | 134-5 | 413.2 415.1 | AA |
| 269 | 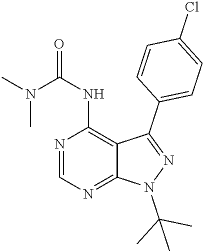 | 164-5 | 371.2 373.2 | AA |
| 270 | 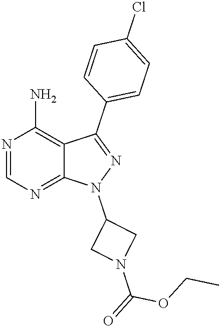 | 232-4 | 371.1 373.2 | D, X |
| 271 | 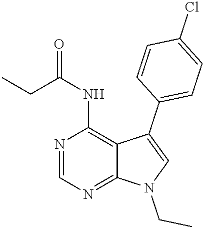 | 167-8 | 329.1 331.1 | AA |
| 272 | 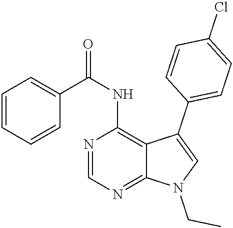 | 209-10 | 375.2 377.3 | AA |
| 273 | 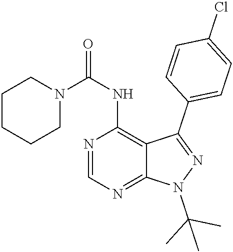 | 151-2 | 413.1 415.2 | AA |
| 274 | 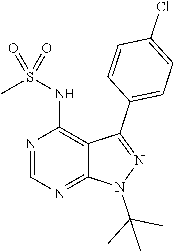 | 259-60 | 380.0 382.0 | AA (Ex. 51) |
| 275 | 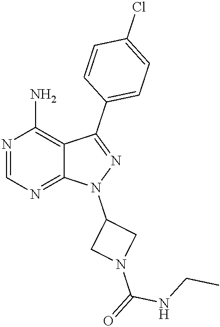 | 132-4 | 372.1 374.1 | D, X |
| 276 | 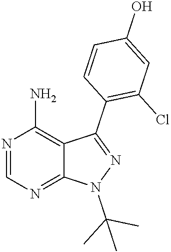 | 173-6 | 318.1 320.1 | — |
| 277 | 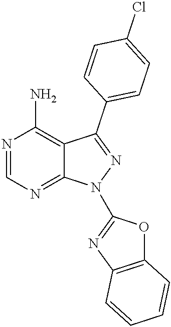 | 271-3 | 363.0 365.0 | D |
| 278 | 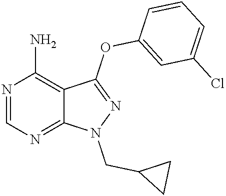 | 125-6 | 316.1 318.1 | S |
| 279 | 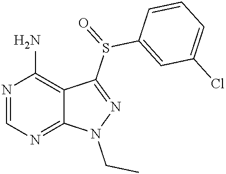 | 159-61 | 322.0 324.0 | T |
| 280 | 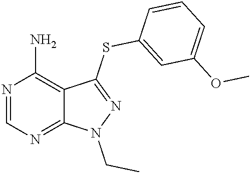 | 134-6 | 302.1 | T |
| 281 | 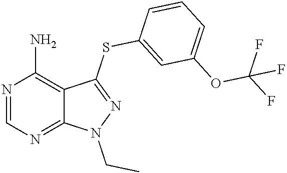 | 118-9 | 356.0 | T |
| 282 | 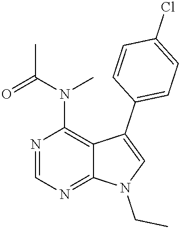 | 147-8 | 329.1 331.1 | AA |
| 283 | 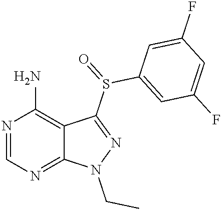 | 197-8 | 324.0 | T |
| 284 | 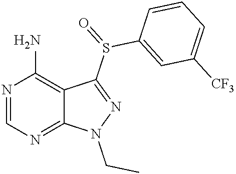 | 152-3 | 384.1 | T |
| 285 | 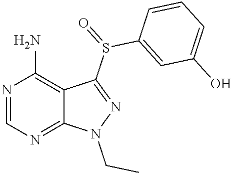 | | | T |
| 286 | 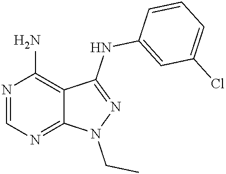 | 172-5 | 289.1 291.0 | Q |
| 287 | 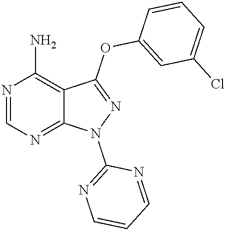 | 232-5 | 340.0 342.0 | S |
| 288 | 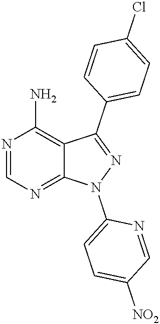 | 328-9 | 368.0 370.0 | D |
| 289 | 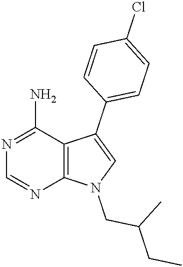 | 139-40 | 315.1 317.1 | Z |
| 290 | 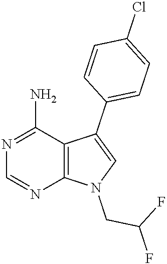 | 191-2 | 309.0 311.0 | Z |
| 291 | 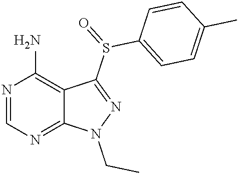 | 150-1 | 302.1 | T |
| 292 | 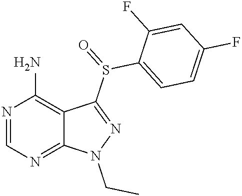 | 175-6 | 324.0 | T |
| 293 | 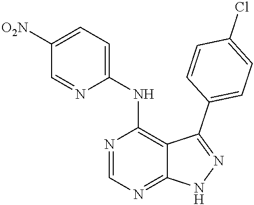 | 304-5 | 368.0 370.0 | AA |
| 294 | 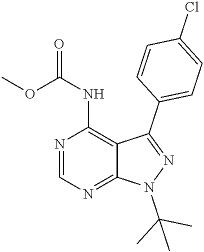 | 160-1 | 360.1 362.1 | AA |
| 295 | 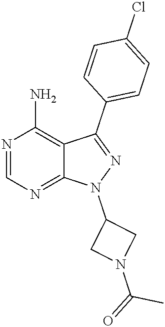 | 262-3 | 341.3 343.4 | D, X |
| 296 | 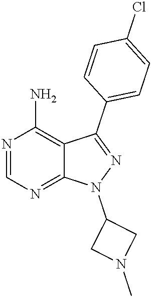 | 235-6 | 352.1 354.1 | D, X |
| 297 | 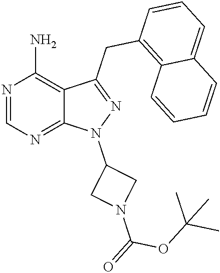 | 199-200 | 431.1 | D |
| 298 | 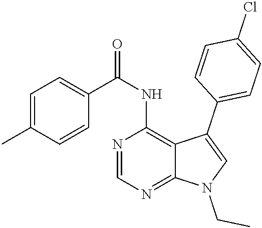 | 220-1 | 389.2 391.2 | AA |
| 299 | 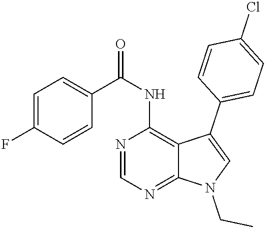 | 201-2 | 393.2 395.2 | AA |
| 300 | 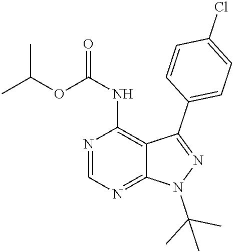 | 143-4 | 388.0 390.0 | AA |
| 301 | 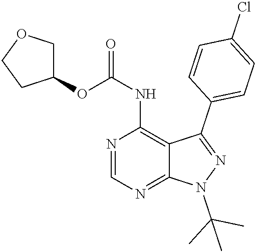 | 153-5 | 416.0 418.0 | AA |
| 302 | 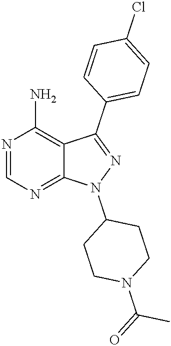 | 243-4 | 369.3 371.3 (Neg M − H) | D, X |
| 303 | 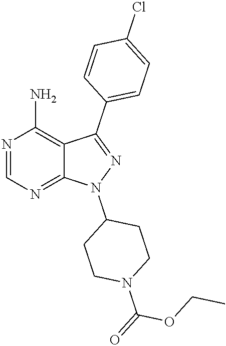 | 237-8 | 401.1 403.1 | D, X |
| 304 | 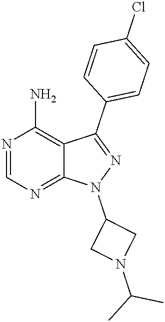 | 219-20 | 343.3 345.2 | D, X |
| 305 | 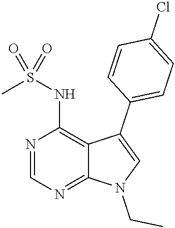 | 257-8 | 351.0 353.1 | AA |
| 306 | 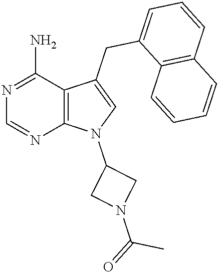 | 231-2 | 373.2 | D, X |
| 307 | 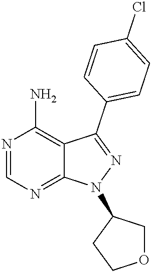 | 167-9 | 316.1 318.1 | D |
| 308 | 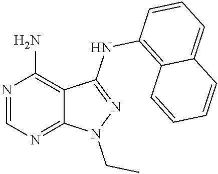 | 182-3 | 305.1 | Q |
| 309 | 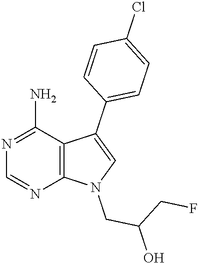 | 177-8 | 321.0 323.0 | Z |
| 310 | 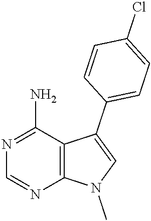 | 219-20 | 259.0 261.0 | Z |
| 311 | 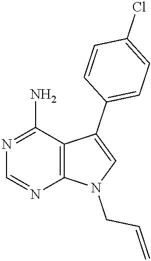 | 143-4 | 285.0 287.0 | Z |
| 312 | 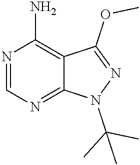 | 169-70 | 222.1 | W (Ex. 30) |
| 313 | 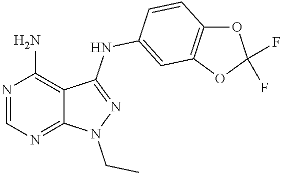 | 165-6 | 335.0 | Q |
| 314 | 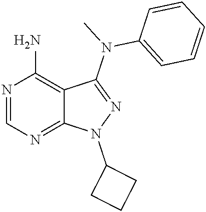 | 188-91 | 295.1 | Q |
| 315 | 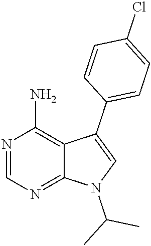 | 148-9 | 287.0 289.0 | Z |
| 316 | 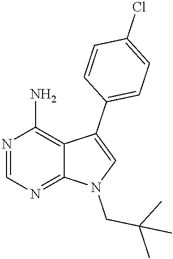 | 212-4 | 315.1 317.1 | Z |
| 317 | 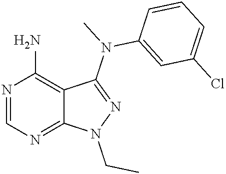 | 169-73 | 303.0 305.0 | Q |
| 318 | 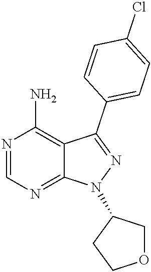 | 171-3 | 316.2 318.2 | D |
| 319 | 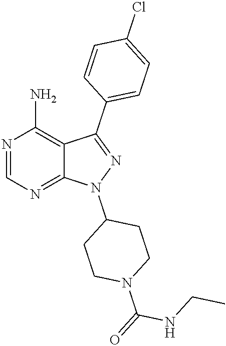 | 137-8 | 400.2 402.2 | D, X |
| 320 | 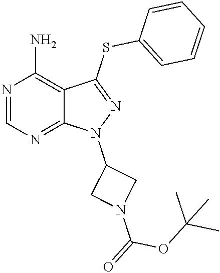 | 188-9 | 399.0 | T |
| 321 | 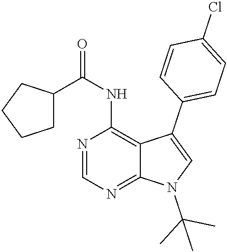 | 156-7 | 397.1 399.1 | AA |
| 322 | 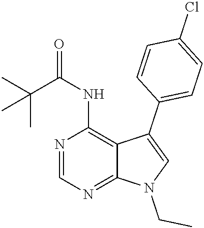 | 125-6 | 357.1 359.1 | AA |
| 323 | 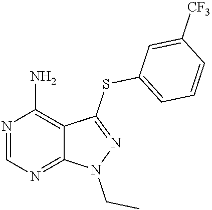 | 133-4 | 340.1 | T |
| 324 | 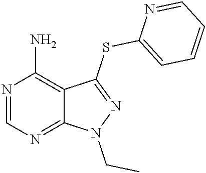 | 169-70 | 273.1 | T |
| 325 | 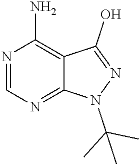 | 237-9 | 208.2 | W (Ex. 30) |
| 326 | 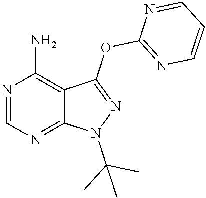 | 207-8 | 286.2 | W |
| 327 | 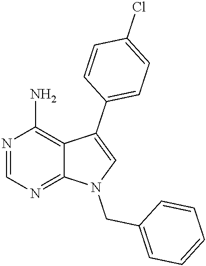 | 175-6 | 335.1 337.1 | Z |
| 328 | 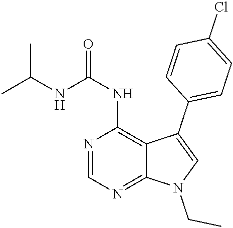 | 170-2 | 358.1 360.1 | AA |
| 329 | 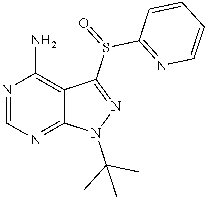 | 146-7 | 317.1 | T |
| 330 | 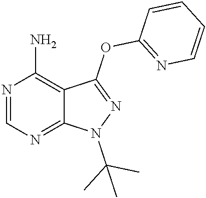 | 225-6 | 285.1 | W |
| 331 | 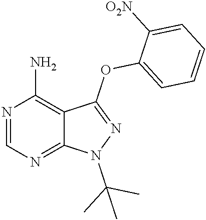 | 168-9 | 329.1 | W |
| 332 | 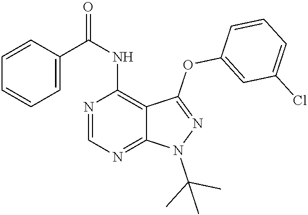 | 121-2 | 422.1 424.1 | AA (Ex. 61) |
| 333 | 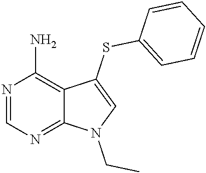 | 194-5 | 271.5 | T (Ex. 70) |
| 334 | 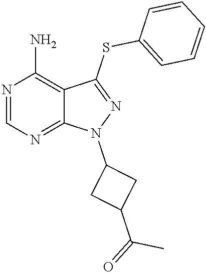 | 222-3 | 339.2 (Neg) | T, X |
| 335 | 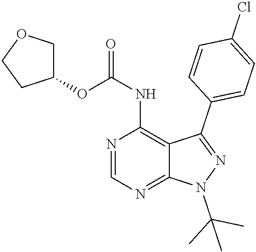 | 147-8 | 416.1 418.1 | AA |
| 336 | 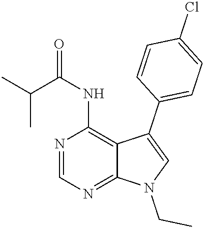 | 134-5 | 343.1 345.1 | AA |
| 337 | 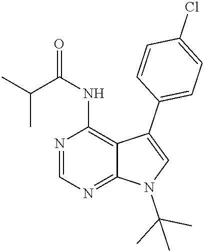 | 166-8 | 371.1 373.1 | AA |
| 338 | 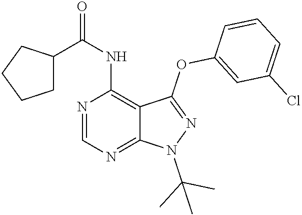 | 88-9 | 414.1 416.1 | AA |
| 339 | 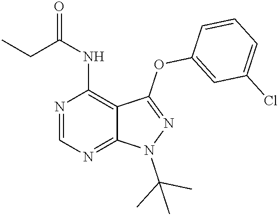 | 85-6 | 374.1 376.1 | AA |
| 340 | 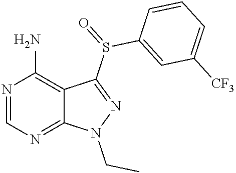 | 195-6 | 356.1 | T |
| 341 | 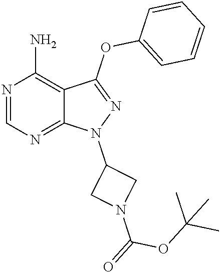 | oil | 383.1 | S |
| 342 | 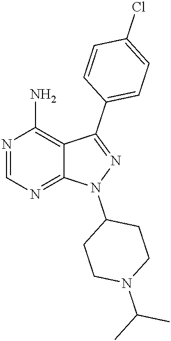 | 172-3 | 371.3 373.1 | D, X |
| 343 | 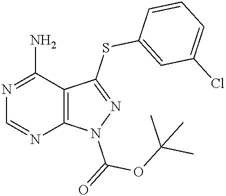 | 165-6 | 431.1 433.1 | T |
| 344 | 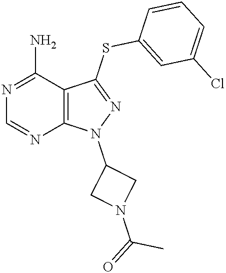 | 178-9 | 375.1 377.1 | T, X |
| 345 | 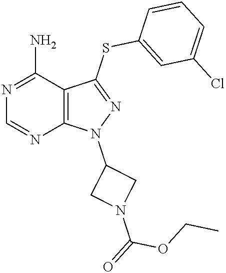 | 180-1 | 403.3 405.1 | T, X (Ex. 63) |
| 346 | 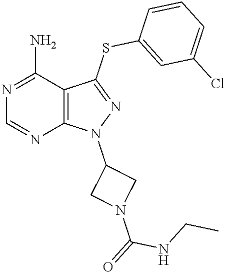 | 179 | 404.1 406.1 | T, X (Ex. 64) |
| 347 | 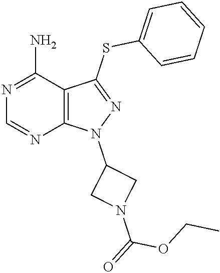 | 167-8 | 371.2 | T, X |
| 348 | 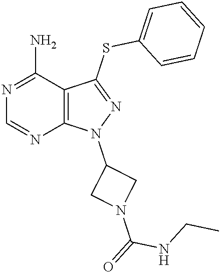 | 192-3 | 370.1 | T, X |
| 349 | 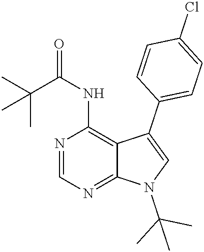 | 195-7 | 385.2 387.2 | AA |
| 350 | 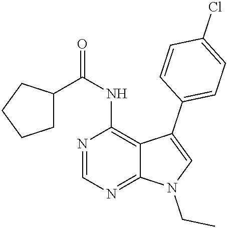 | 167-8 | 369.1 371.1 | AA |
| 351 | 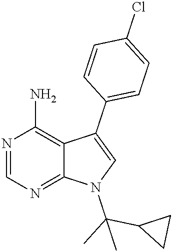 | 182-4 | 327.1 329.1 | Y |
| 352 | 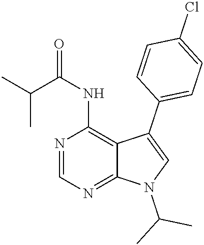 | 129-30 | 357.1 359.1 | AA |
| 353 | 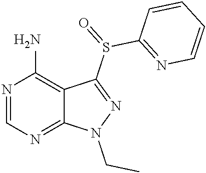 | 141-2 | 289.1 | T |
| 354 | 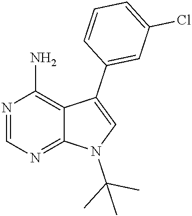 | 211-2 | 301.1 303.1 | Y |
| 355 | 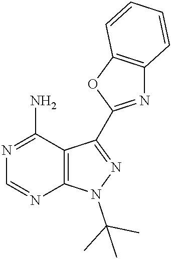 | 223-5 | 309.1 | Ex. 65 |
| 356 | 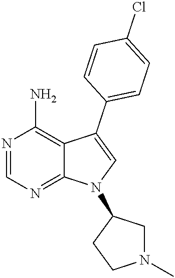 | 178-9 | 329.2 331.2 | D |
| 357 | 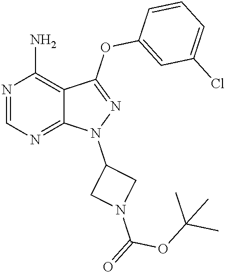 | 156-7 | 417.0 419.1 | S |
| 358 | 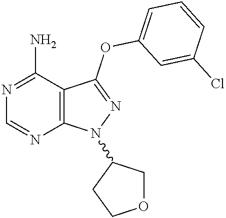 | 153-4 | 332.1 334.2 | S |
| 359 | 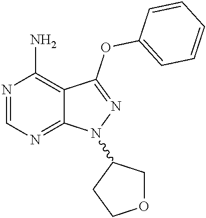 | 146-7 | 298.2 | S |
| 360 | 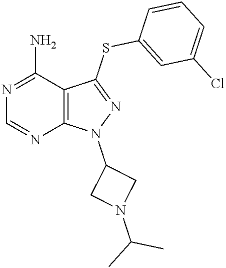 | 183-4 | 375.2 377.1 | T, X |
| 361 | 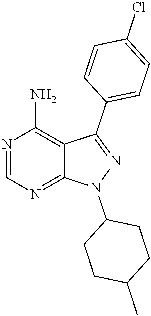 | 175-7 | 342.3 344.2 | D |
| 362 | 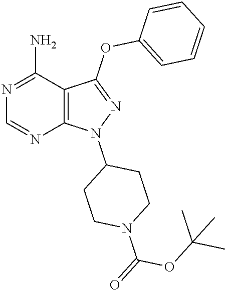 | 176-8 | 411.1 | S |
| 363 | 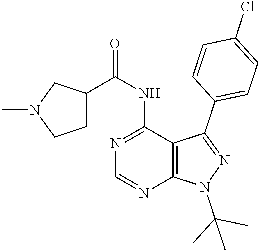 | 123-5 | 413.9 415.9 | AA |
| 364 | 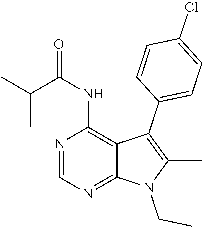 | 186-7 | 357.1 359.1 | Y, AA |
| 365 | 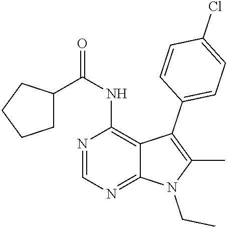 | 154-6 | 383.1 385.1 | Y, AA |
| 366 | 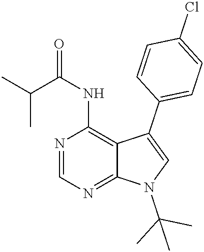 | 157-8 | 371.1 373.1 | Y, AA |
| 367 | 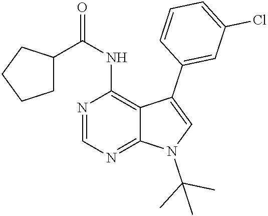 | 154-5 | 397.1 399.1 | Y, AA |
| 368 | 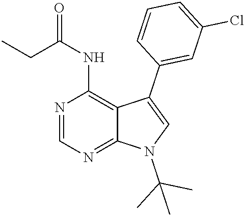 | 134-5 | 357.1 359.1 | Y, AA |
| 369 | 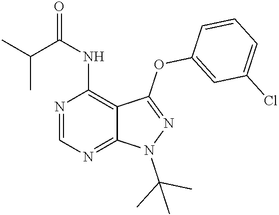 | oil | 388.1 390.1 | AA |
| 370 | 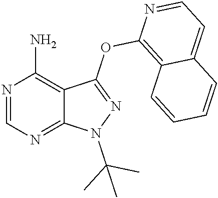 | 213-4 | 335.1 | W (Ex. 30) |
| 371 | 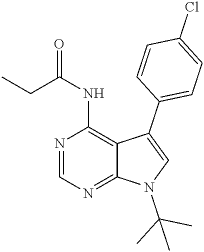 | 163-4 | 357.1 359.1 | Y, AA |
| 372 | 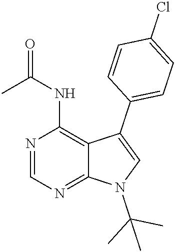 | 158-9 | 343.1 345.1 | Y, AA |
| 373 | 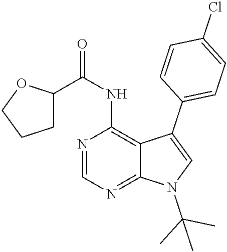 | 184-6 | 399.1 401.1 | Y, AA |
| 374 | 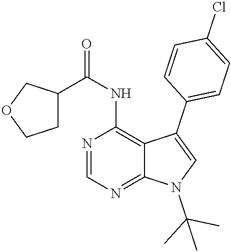 | 148-9 | 399.1 401.1 | Y, AA |
| 375 | 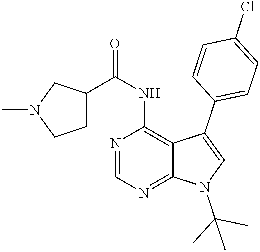 | | | Y, AA |
| 376 | 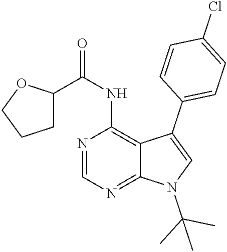 | 135-7 | 400.9 402.8 | Y, AA |
| 377 | 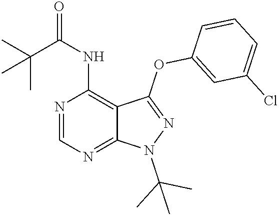 | 116-7 | 402.1 404.1 | AA |
| 378 | 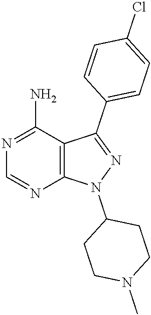 | 108-9 | 343.1 345.1 | D, X |
| 379 | 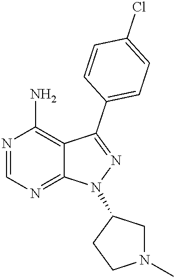 | 176-7 | 329.2 331.1 | D |
| 380 | 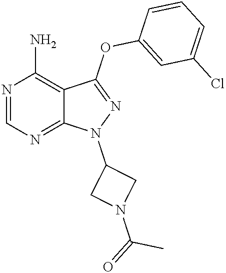 | 209-10 | 359.1 361.0 | S, X |
| 381 | 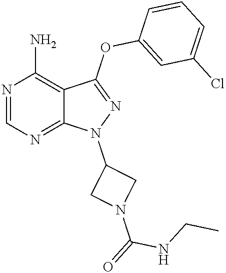 | 214-5 | 388.2 390.2 | S, X |
| 382 | 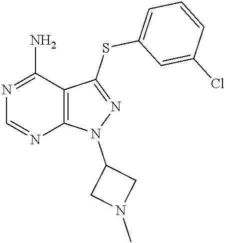 | 144-5 | 347.1 349.0 | T, X |
| 383 | 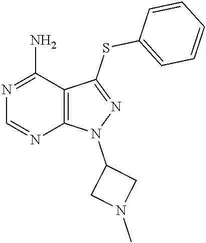 | 137-8 | 313.1 | T, X |
| 384 | 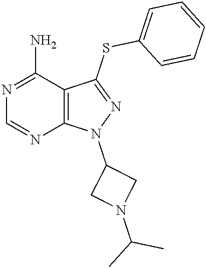 | 203-4 | 341.2 | T, X |
| 385 | 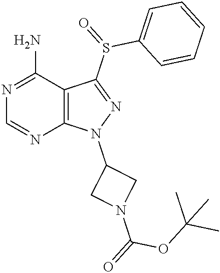 | 200-1 | 415.2 | T |
| 386 | 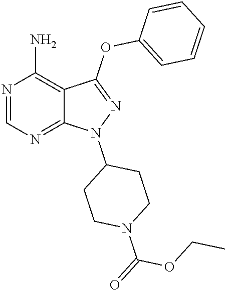 | 156-8 | 383.3 | S, X |
| 387 | 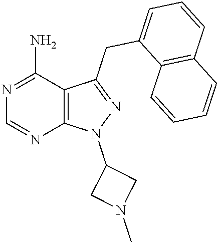 | 134-5 | 345.2 | D, x |
| 388 | 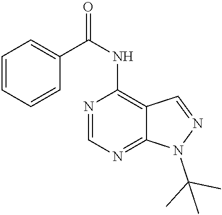 | 137-8 | 296.1 | AA |
| 389 | 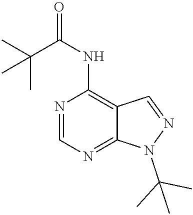 | 136-7 | 276.1 | AA |
| 390 | 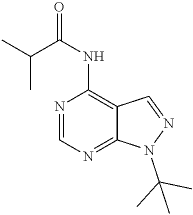 | 137-8 | 262.1 | AA |
| 391 | 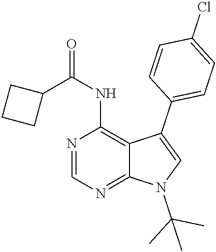 | 171-2 | 383.2 385.1 | Y, AA |
| 392 | 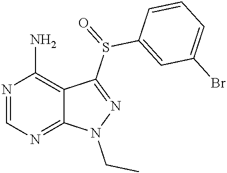 | 170-1 | 365.9 367.9 | T |
| 393 | 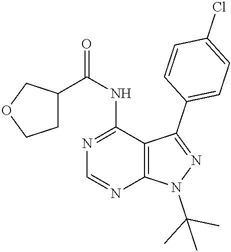 | 159-61 | 400.9 402.8 | AA |
| 394 | 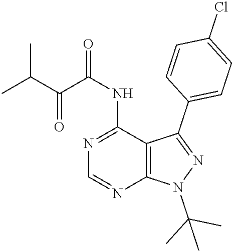 | 178-80 | 400.8 402.8 | AA |
| 395 | 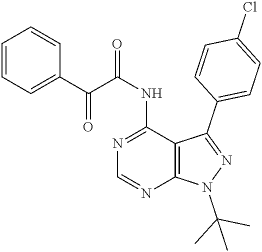 | 220-2 | 434.9 436.9 | AA |
| 396 | 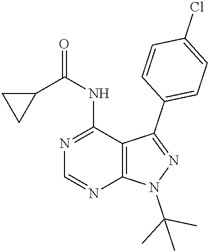 | 190-1 | 370.8 372.8 | AA |
| 397 | 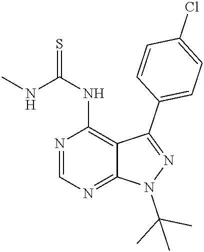 | 172-3 | 375.0 377.0 | AA (Ex. 69) |
| 398 | 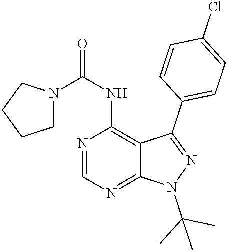 | 210-1 | 399.1 401.1 | AA |
| 399 | 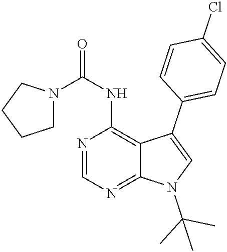 | 161-2 | 398.2 400.2 | Y, AA |
| 400 | 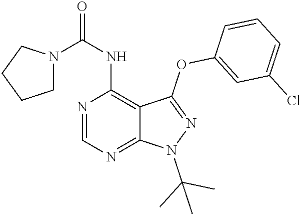 | 169-70 | 415.1 417.1 | AA |
| 401 | 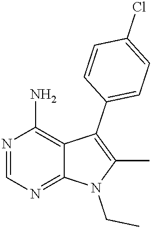 | 229-30 | 287.1 289.1 | Y |
| 402 | 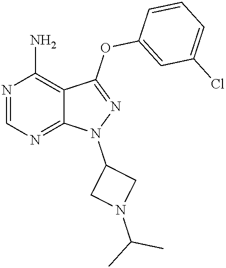 | 172-4 | 359.2 361.1 | S, X |
| 403 | 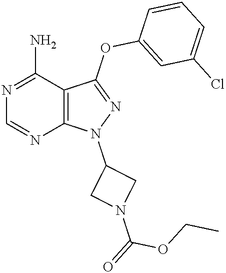 | 203-4 | 389.1 391.1 | S, X |
| 404 | 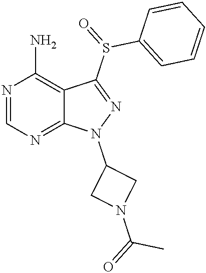 | 237-8 | 357.2 | T, X |
| 405 | 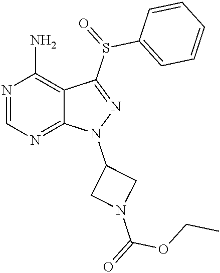 | 165-6 | 387.2 | T, X |
| 406 | 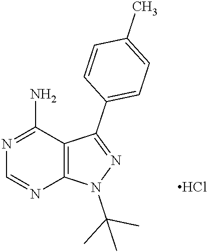 | 215-6 | 282.1 | cf. Ex. 17 |
| 407 | 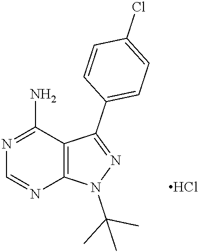 | 211-2 | 302.1 304.1 | Ex. 17 |
| 408 | 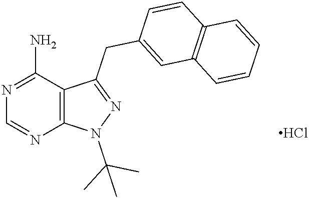 | 178-9 | 332.1 | cf. Ex. 17 |
| 409 | 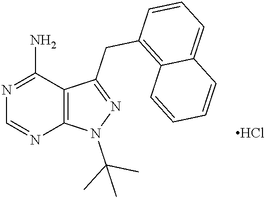 | 172-3 | 332.2 | cf. Ex. 17 |
| 410 | 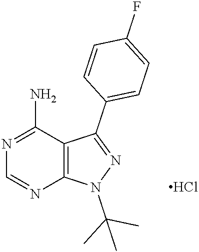 | 207-8 | 286.1 | cf. Ex. 17 |
| 411 | 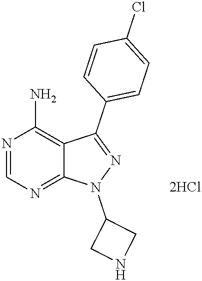 | 190-2 | 301.1 303.1 | cf. Ex. 17 |
| 412 | 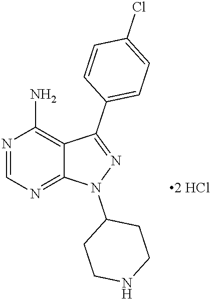 | 196-7 | 329.1 331.1 | cf. Ex. 17 |
| 413 | 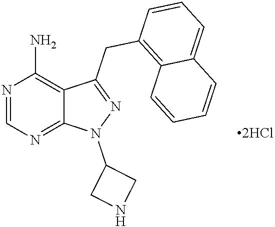 | 176-7 | 331.2 | cf. Ex. 17 |
| 414 | 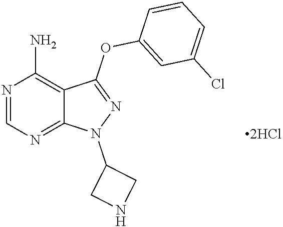 | 169-70 | 317.1 319.1 | cf. Ex. 17 |
| 415 | 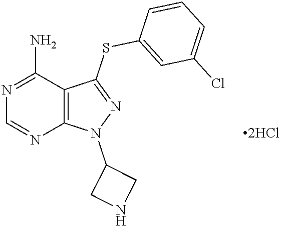 | 162 | 332.9 334.0 | cf. Ex. 17 |
| 416 | 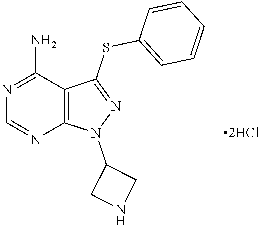 | 165-6 | 299.1 | cf. Ex. 17 |
| 417 | 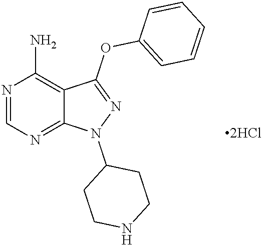 | 230-3 | 311.2 | cf. Ex. 17 |
| 418 | 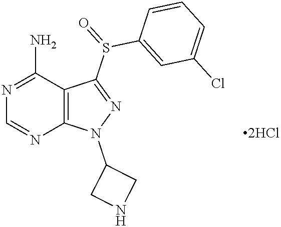 | 162 | 349.2 351.1 | cf. Ex. 17 |
| 419 | 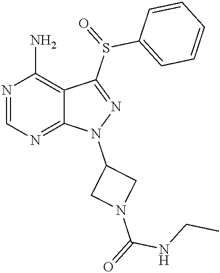 | 222-3 | 386.1 | T, X |
| 420 | 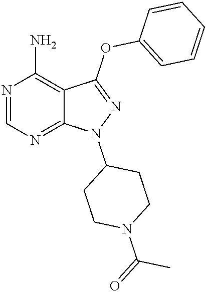 | 188-90 | 335.2 | S, X |
| 421 | 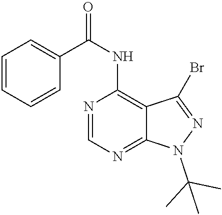 | 240-1 | 374.0 376.0 | AA |
| 422 | 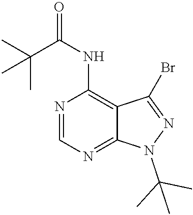 | 150-1 | 354.0 356.1 | AA |
| 423 | 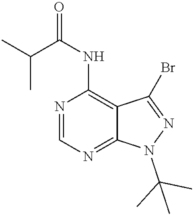 | 121-2 | 340.0 342.0 | AA |
| 424 | 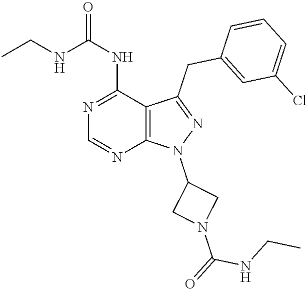 | 190-1 | 457.1 459.4 (ESI Neg) | AA |
| 425 | 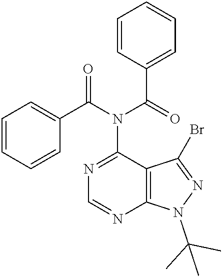 | 229-30 | 478.0 479.8 | AA |
| 426 | 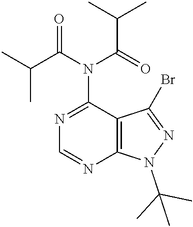 | 97-8 | 410.0 412.0 | AA |
| 427 | 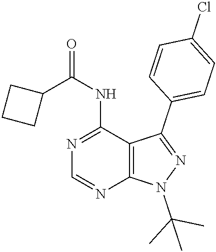 | | | AA |
| 428 | 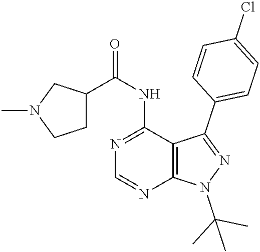 | | | AA |
| 429 | 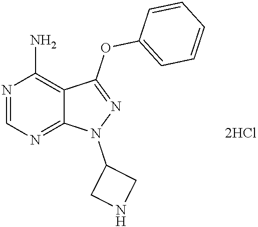 | 169-71 | 283.1 | S, X |
| 430 | 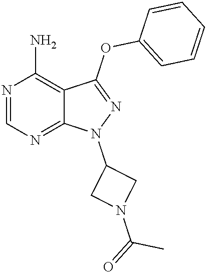 | 222-3 | 325.1 | S, X |
| 431 | 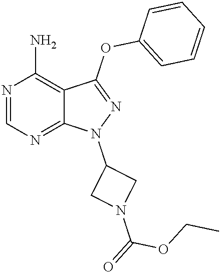 | 175-6 | 355.2 | S, X |
| 432 | 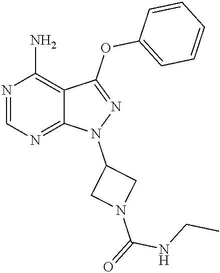 | 224-5 | 354.2 | S, X |
| 433 | 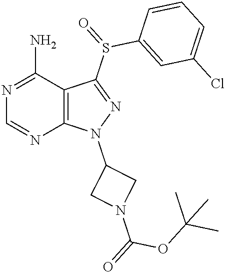 | 187-8 | 449.0 451.0 | T |
| 434 | 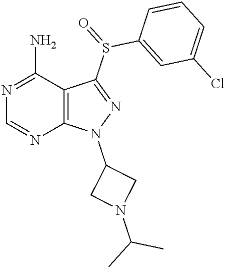 | 206-7 | 391.2 393.1 | T, X |
| 435 | 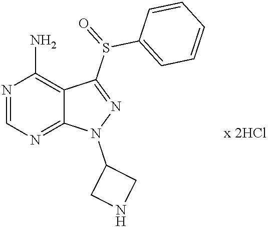 | 172 dec. | 315.0 | T, X |
| 436 | 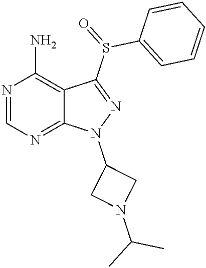 | 189-90 | 357.1 | T, X |
| 437 | 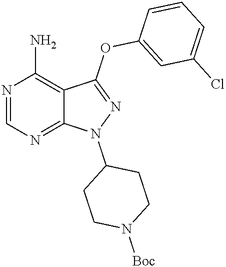 | 193-4 | 445.1 447.1 | S |
| 438 | 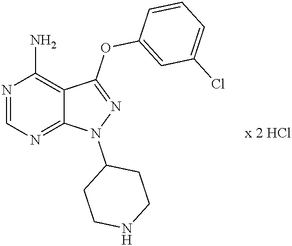 | 285-6 | 345.1 347.2 | S, X |
| 439 | 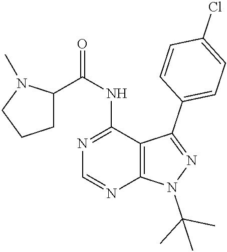 | 45-6 | 413.8 415.9 | AA |
| 440 | 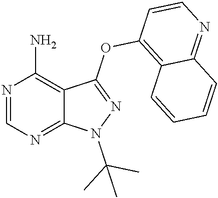 | 199-201 | 335.2 | W |
| 441 | 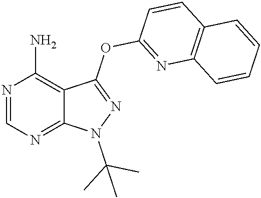 | 241-3 | 335.2 | W |
| 442 | 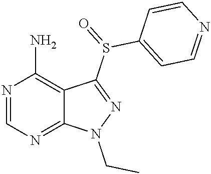 | 159-60 | 289.1 | T |
| 443 | 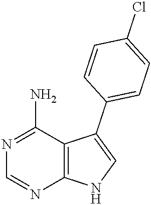 | | | Z |
| 444 | 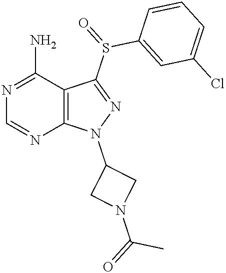 | 263-4 | 391.1 393.1 | T, X |
| 445 | 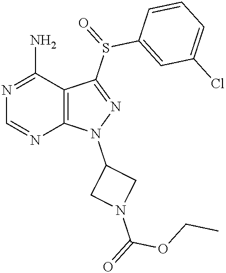 | 177-8 | 421.1 423.1 | T, X |
| 446 | 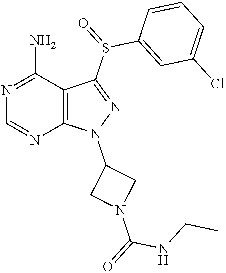 | 224-5 | 420.3 422.3 | T, X |
| 447 | 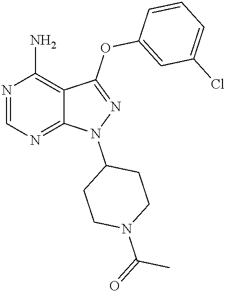 | 180-1 | 387.2 389.1 | S, X |
| 448 | 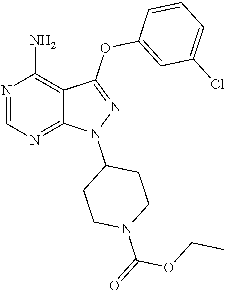 | 180-1 | 417.3 419.1 | S, X |
| 449 | 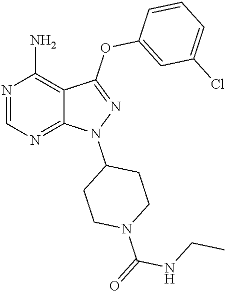 | 239-41 | 416.1 418.1 | S, X |
| 450 | 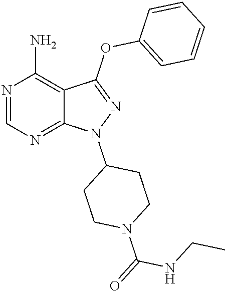 | 207-8 | 382.1 | S, X |
| 451 | 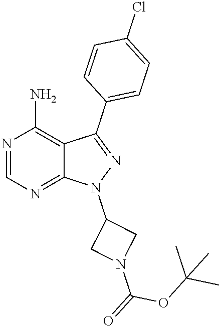 | 229-30 | 400.1 402.1 | BB |
| 452 | 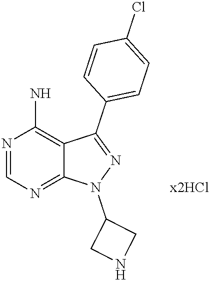 | Unknown dec. | 300.2 302.1 | BB, X |
| 453 | 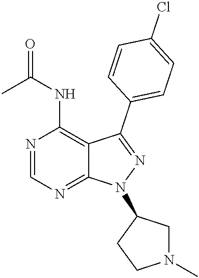 | 163-4 | 371.1 373.1 | AA |
| 454 | 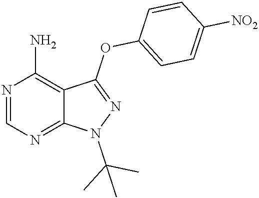 | 201-202 | 329.1, [M + H]+ | W |
| 455 | 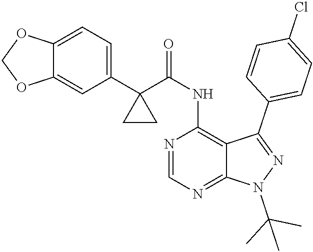 | 208-209 | 489.1, 491.1 [M + H]+ | AA |
| 456 | 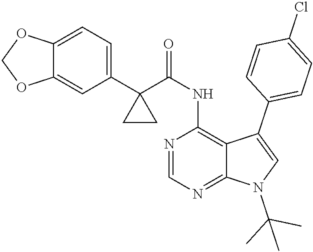 | 200-201 | 490.1, 492.1 [M + H]+ | AA |
| 457 | 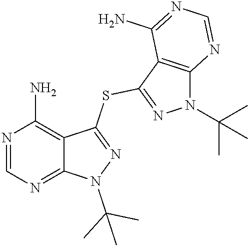 | 321-323 | 413.1 [M + H]+ | First step of process T |
| 458 | 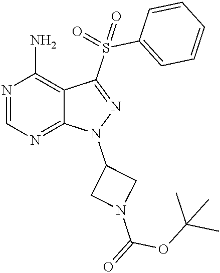 | 224-226 | | T, X |
| 459 | 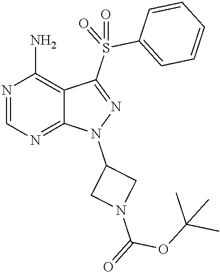 | 229-230 | | T, X |
| 460 | 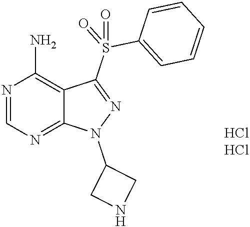 | 181-182 | | T, X |
| 461 | 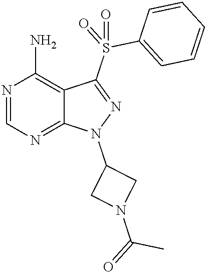 | 253 | | T, X |
| 462 | 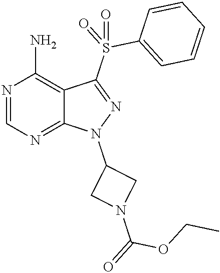 | 234-235 | | T, X |
| 463 | 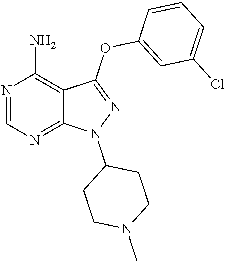 | 165-167 | | S, X |
| 464 | 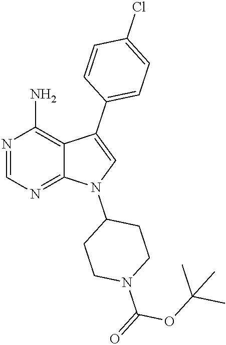 | 149-150 | | BB |
| 465 | 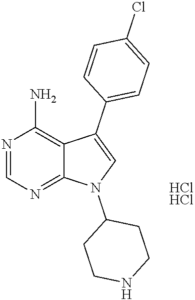 | 224-226 | | BB, X |
| 466 | 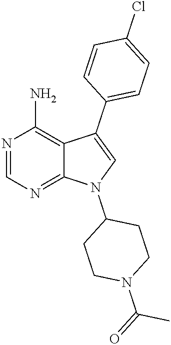 | 189-191 | | BB, X |
| 467 | 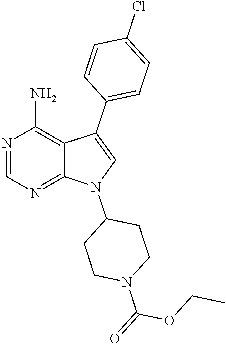 | 206-208 | | BB, X |
| 468 | 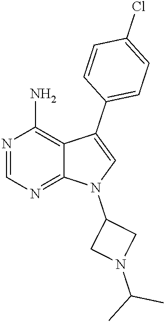 | 222-223 | | BB, X |
| 469 | 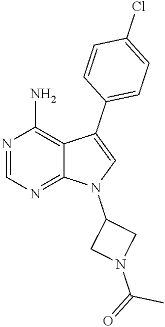 | 239-240 | | BB, X |
| 470 | 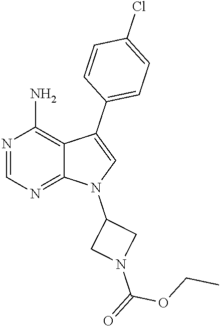 | 189-191 | | BB, X |
| 471 | 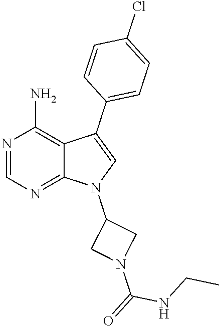 | 175-176 | | BB, X |
| 472 | 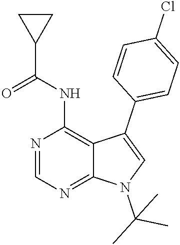 | 194-195 | | Y, AA |
| 473 | 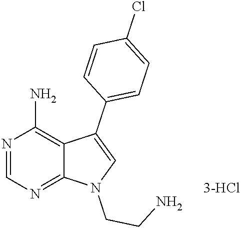 | 294-297 | | BB |
| 474 | 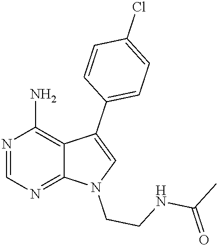 | 213-215 | | BB |
| 475 | 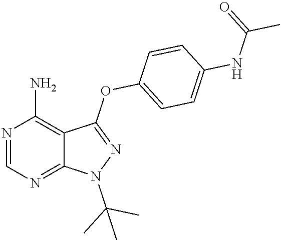 | 244-245 | | W, reduction, |

Moreover, synthetic chemistry functional group transformations useful in synthesizing the full range of the disclosed compounds are known in the art and include, for example, those described in R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995). The entire teachings of these documents are incorporated herein by reference. For example, starting with the syntheses above, one can prepare final products having a substituent such as —OH. Suitable techniques for converting the —OH group to another disclosed substituent such as a halogen are well known. For example, an —OH can be converted to —Cl, for example, using a chlorinating reagent such as thionyl chloride or N-chlorosuccinimide, optionally in combination with ultraviolet irradiation.

Suitable protecting groups and strategies for protecting and deprotecting functional groups using protecting groups useful in synthesizing the disclosed compounds are known in the art and include, for example, those described in T. W. Greene and P. G. M. Wuts, Protective Groups in Organic Synthesis, 2nd Ed., John Wiley and Sons (1991), the entire teachings of which are incorporated herein by reference. For example, suitable hydroxyl protecting groups include, but are not limited to substituted methyl ethers (e.g., methoxymethyl, benzyloxymethyl) substituted ethyl ethers (e.g., ethoxymethyl, ethoxyethyl)benzyl ethers (benzyl, nitrobenzyl, halobenzyl) silyl ethers (e.g., trimethylsilyl), esters, and the like. Examples of suitable amine protecting groups include benzyloxycarbonyl, tert-butoxycarbonyl, tert-butyl, benzyl and fluorenylmethyloxy-carbonyl (Fmoc). Examples of suitable thiol protecting groups include benzyl, tert-butyl, acetyl, methoxymethyl and the like.

The reactions described herein may be conducted in any suitable solvent for the reagents and products in a particular reaction. Suitable solvents are those that facilitate the intended reaction but do not react with the reagents or the products of the reaction. Suitable solvents can include, for example: ethereal solvents such as diethyl ether or tetrahydrofuran; ketone solvents such as acetone, methyl ethyl ketone or ethyl acetate; halogenated solvents such as dichloromethane, chloroform, carbon tetrachloride, or trichloroethane; aromatic solvents such as benzene, toluene, xylene, or pyridine; polar aprotic organic solvents such as acetonitrile, dimethyl sulfoxide, dimethyl formamide, N-methylpyrrolidone, hexamethyl phosphoramide, nitromethane, nitrobenzene, or the like; polar protic solvents such as methanol, ethanol, propanol, butanol, ethylene glycol, tetraethylene glycol, or the like; nonpolar hydrocarbons such as pentane, hexane, cyclohexane, cyclopentane, heptane, octance, or the like; basic amine solvents such as pyridine, triethyleamine, or the like; and other solvents known to the art.

Reactions or reagents which are water sensitive may be handled under anhydrous conditions. Reactions or reagents which are oxygen sensitive may be handled under an inert atmosphere, such as nitrogen, helium, neon, argon, and the like. Reactions or reagents which are light sensitive may be handled in the dark or with suitably filtered illumination.

Reactions or reagents which are temperature-sensitive, e.g., reagents that are sensitive to high temperature or reactions which are exothermic may be conducted under temperature controlled conditions. For example, reactions that are strongly exothermic may be conducted while being cooled to a reduced temperature.

Reactions that are not strongly exothermic may be conducted at higher temperatures to facilitate the intended reaction, for example, by heating to the reflux temperature of the reaction solvent. Reactions can also be conducted under microwave irradiation conditions. For example, in various embodiments of the method, the first and second reagents are reacted together under microwave irradiation.

Reactions may also be conducted at atmospheric pressure, reduced pressure compared to atmospheric, or elevated pressure compared to atmospheric pressure. For example, a reduction reaction may be conducted in the presence of an elevated pressure of hydrogen gas in combination with a hydrogenation catalyst.

Reactions may be conducted at stoichiometric ratios of reagents, or where one or more reagents are in excess. For example, in the last step of scheme 3, process C, the first reactant, organohalogen 3-bromo-1-tert-butyl-1H-pyrazolo[3,4-d]pyrimidin-4-amine, may be used in a molar ratio to the aryl boronate reactant represented by ArB(OH)₂ of about 20:1, 10:1, 5:1, 2.5:1, 2:1, 1.5:1, 1.3:1, 1.2:1, 1.1:1, 1:1, 0.91:1, 0.83:1, 0.77:1, 0.67:1, 0.5:1, 0.4:1, 0.2:1, 0.1:1 or 0.5:1. Typically, the first reactant may be used in a molar ratio to the second reactant of about 5:1, 2.5:1, 2:1, 1.5:1, 1.3:1, 1.2:1, 1.1:1, 1:1, 0.91:1, 0.83:1, 0.77:1, 0.67:1, 0.5:1, 0.4:1. In certain embodiments, the first reactant may be used in a molar ratio to the second reactant of about 1.5:1, 1.3:1, 1.2:1, 1.1:1, 1:1, 0.91:1, 0.83:1, 0.77:1, or 0.67:1. Preferably, first reactant may be used in a molar ratio to the second reactant of between about 1.1:1 and 0.9:1, typically about 1:1. The same or different ratios may be used for other reagents in this or other reactions.

### D. Formulation of Pharmaceutical Compositions

The pharmaceutical compositions provided herein contain therapeutically effective amounts of one or more of the compounds provided herein that are useful in the treatment or amelioration of one or more of the symptoms of disorders associated with protein trafficking, or in which protein trafficking is implicated, and a pharmaceutically acceptable carrier. Pharmaceutical carriers suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients.

The compositions contain one or more compounds provided herein. The compounds are, in various embodiments, formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. In various embodiments, the compounds described above are formulated into pharmaceutical compositions using techniques and procedures well known in the art (see, e.g., Ansel Introduction to Pharmaceutical Dosage Forms, Fourth Edition 1985, 126).

In the compositions, effective concentrations of one or more compounds or pharmaceutically acceptable derivatives thereof is (are) mixed with a suitable pharmaceutical carrier. The compounds may be derivatized as the corresponding salts, esters, enol ethers or esters, acetals, ketals, orthoesters, hemiacetals, hemiketals, acids, bases, solvates, hydrates or prodrugs prior to formulation, as described above. The concentrations of the compounds in the compositions are effective for delivery of an amount, upon administration, that treats or ameliorates one or more of the symptoms of disorders associated with protein trafficking or in which protein trafficking is implicated.

In various embodiments, the compositions are formulated for single dosage administration. To formulate a composition, the weight fraction of compound is dissolved, suspended, dispersed or otherwise mixed in a selected carrier at an effective concentration such that the treated condition is relieved or one or more symptoms are ameliorated.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in in vitro and in vivo systems described herein (see, e.g., EXAMPLE 1) and in U.S. patent application Ser. No. 10/826,157, filed Apr. 16, 2004, and U.S. Patent Application Publication No. 2003/0073610, and then extrapolated therefrom for dosages for humans.

The concentration of active compound in the pharmaceutical composition will depend on absorption, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of disorders associated protein trafficking or in which protein trafficking is implicated, as described herein.

In various embodiments, a therapeutically effective dosage should produce a serum concentration of active ingredient of from about 0.1 ng/ml to about 50-100 μg/ml. The pharmaceutical compositions, in another embodiment, should provide a dosage of from about 0.001 mg to about 2000 mg of compound per kilogram of body weight per day. Pharmaceutical dosage unit forms are prepared to provide from about 0.01 mg, 0.1 mg or 1 mg to about 500 mg, 1000 mg or 2000 mg, and in various embodiments from about 10 mg to about 500 mg of the active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate. Derivatives of the compounds, such as prodrugs of the compounds may also be used in formulating effective pharmaceutical compositions.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are, in various embodiments, formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, acetate, sodium citrate, cyclodextrine derivatives, sorbitan monolaurate, triethanolamine sodium acetate, triethanolamine oleate, and other such agents.

Actual methods of preparing such dosage forms are known, or will be apparent, to those skilled in this art; for example, see Remington's Pharmaceutical Sciences, Mack Publishing Company, Easton, Pa., 15th Edition, 1975.

Dosage forms or compositions containing active ingredient in the range of 0.005% to 100% with the balance made up from non-toxic carrier may be prepared. Methods for preparation of these compositions are known to those skilled in the art. The contemplated compositions may contain 0.001-%100% active ingredient, in various embodiments 0.1-95%, in another embodiment 75-85%.

1. Compositions for Oral Administration

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

a. Solid Compositions for Oral Administration

In certain embodiments, the formulations are solid dosage forms, in various embodiments, capsules or tablets. The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include microcrystalline cellulose, gum tragacanth, glucose solution, acacia mucilage, gelatin solution, molasses, polyinylpyrrolidine, povidone, crospovidones, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

The compound, or pharmaceutically acceptable derivative thereof, could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable derivative thereof as described herein. Higher concentrations, up to about 98% by weight of the active ingredient may be included.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

b. Liquid Compositions for Oral Administration

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic acid, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is in various embodiments encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. RE28,819 and 4,358,603. Briefly, such formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl)acetals of lower alkyl aldehydes such as acetaldehyde diethyl acetal.

2. Injectables, Solutions and Emulsions

Parenteral administration, in various embodiments characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Implantation of a slow-release or sustained-release system, such that a constant level of dosage is maintained (see, e.g., U.S. Pat. No. 3,710,795) is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art.

The unit-dose parenteral preparations are packaged in an ampoule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. In various embodiments, a therapeutically effective dosage is formulated to contain a concentration of at least about 0.1% w/w up to about 90% w/w or more, in certain embodiments more than 1% w/w of the active compound to the treated tissue(s).

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

3. Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in various embodiments, about neutral pH. Subsequent sterile filtration of the solution followed by lyophilization under standard conditions known to those of skill in the art provides the desired formulation. In various embodiments, the resulting solution will be apportioned into vials for lyophilization. Each vial will contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

4. Topical Administration

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable derivatives thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will, in various embodiments, have diameters of less than 50 microns, in various embodiments less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% isotonic solutions, pH about 5-7, with appropriate salts.

5. Compositions for Other Routes of Administration

Other routes of administration, such as transdermal patches, including iontophoretic and electrophoretic devices, and rectal administration, are also contemplated herein.

Transdermal patches, including iontophoretic and electrophoretic devices, are well known to those of skill in the art. For example, such patches are disclosed in U.S. Pat. Nos. 6,267,983, 6,261,595, 6,256,533, 6,167,301, 6,024,975, 6,010,715, 5,985,317, 5,983,134, 5,948,433, and 5,860,957.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The weight of a rectal suppository, in various embodiments, is about 2 to 3 gm.

Tablets and capsules for rectal administration are manufactured using the same pharmaceutically acceptable substance and by the same methods as for formulations for oral administration.

6. Targeted Formulations

The compounds provided herein, or pharmaceutically acceptable derivatives thereof, may also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In various embodiments, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

7. Articles of Manufacture

The compounds or pharmaceutically acceptable derivatives may be packaged as articles of manufacture containing packaging material, a compound or pharmaceutically acceptable derivative thereof provided herein, which is effective for modulating protein trafficking, or for treatment or amelioration of one or more symptoms of disorders in which protein trafficking is implicated, within the packaging material, and a label that indicates that the compound or composition, or pharmaceutically acceptable derivative thereof, is used for modulating a protein trafficking disorder, or for treatment or amelioration of one or more symptoms of disorders in which protein trafficking is implicated.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any disorder in which protein trafficking is implicated as a mediator or contributor to the symptoms or cause.

8. Sustained Release Formulations

Also provided are sustained release formulations to deliver the compounds to the desired target (i.e. brain or systemic organs such as lungs) at high circulating levels (between 10⁻⁹ and 10⁻⁴ M). In a certain embodiment for the treatment of cystic fibrosis, the circulating levels of the compounds can be maintained, e.g., up to 10⁻⁷ M. The levels are either circulating in the patient systemically, or in various embodiments, present in tissue of the desired target organ, or in certain embodiments, localized to particular tissues, cells, lesions, and the like, e.g., within the desired target organ.

It is understood that the compound levels are maintained over a certain period of time as is desired and can be easily determined by one skilled in the art. In various embodiments, the administration of a sustained release formulation can be effected so that a constant level of therapeutic compound is maintained between 10⁻⁸ and 10⁻⁶M between 48 to 96 hours in the sera.

Such sustained and/or timed release formulations may be made by sustained release means of delivery devices that are well known to those of ordinary skill in the art, such as those described in U.S. Pat. Nos. 3,845,770; 3,916,899; 3,536,809; 3,598,123; 4,008,719; 4,710,384; 5,674,533; 5,059,595; 5,591,767; 5,120,548; 5,073,543; 5,639,476; 5,354,556 and 5,733,566, the disclosures of which are each incorporated herein by reference. These pharmaceutical compositions can be used to provide slow or sustained release of one or more of the active compounds using, for example, hydroxypropylmethyl cellulose, other polymer matrices, gels, permeable membranes, osmotic systems, multilayer coatings, microparticles, liposomes, microspheres, or the like. Suitable sustained release formulations known to those skilled in the art, including those described herein, may be readily selected for use with the pharmaceutical compositions provided herein. Thus, single unit dosage forms suitable for oral administration, such as, but not limited to, tablets, capsules, gelcaps, caplets, powders and the like, that are adapted for sustained release are contemplated herein.

In various embodiments, the sustained release formulation contains active compound such as, but not limited to, microcrystalline cellulose, maltodextrin, ethylcellulose, and magnesium stearate. As described above, all known methods for encapsulation which are compatible with properties of the disclosed compounds are contemplated herein. The sustained release formulation is encapsulated by coating particles or granules of the pharmaceutical compositions provided herein with varying thickness of slowly soluble polymers or by microencapsulation. In various embodiments, the sustained release formulation is encapsulated with a coating material of varying thickness (e.g. about 1 micron to 200 microns) that allow the dissolution of the pharmaceutical composition about 48 hours to about 72 hours after administration to a mammal. In another embodiment, the coating material is a food-approved additive.

In another embodiment, the sustained release formulation is a matrix dissolution device that is prepared by compressing the drug with a slowly soluble polymer carrier into a tablet. In various embodiments, the coated particles have a size range between about 0.1 to about 300 microns, as disclosed in U.S. Pat. Nos. 4,710,384 and 5,354,556, which are incorporated herein by reference in their entireties. Each of the particles is in the form of a micromatrix, with the active ingredient uniformly distributed throughout the polymer.

Sustained release formulations such as those described in U.S. Pat. No. 4,710,384, which is incorporated herein by reference in its entirety, having a relatively high percentage of plasticizer in the coating in order to permit sufficient flexibility to prevent substantial breakage during compression are disclosed. The specific amount of plasticizer varies depending on the nature of the coating and the particular plasticizer used. The amount may be readily determined empirically by testing the release characteristics of the tablets formed. If the medicament is released too quickly, then more plasticizer is used. Release characteristics are also a function of the thickness of the coating. When substantial amounts of plasticizer are used, the sustained release capacity of the coating diminishes. Thus, the thickness of the coating may be increased slightly to make up for an increase in the amount of plasticizer. Generally, the plasticizer in such an embodiment will be present in an amount of about 15 to 30% of the sustained release material in the coating, in various embodiments 20 to 25%, and the amount of coating will be from 10 to 25% of the weight of the active material, and in another embodiment, 15 to 20% of the weight of active material. Any conventional pharmaceutically acceptable plasticizer may be incorporated into the coating.

The compounds provided herein can be formulated as a sustained and/or timed release formulation. All sustained release pharmaceutical products have a common goal of improving drug therapy over that achieved by their non-sustained counterparts. Ideally, the use of an optimally designed sustained release preparation in medical treatment is characterized by a minimum of drug substance being employed to cure or control the condition. Advantages of sustained release formulations may include: 1) extended activity of the composition, 2) reduced dosage frequency, and 3) increased patient compliance. In addition, sustained release formulations can be used to affect the time of onset of action or other characteristics, such as blood levels of the composition, and thus can affect the occurrence of side effects.

The sustained release formulations provided herein can be designed to initially release an amount of the therapeutic composition that promptly produces the desired therapeutic effect, and gradually and continually release of other amounts of compositions to maintain this level of therapeutic effect over an extended period of time. In order to maintain this constant level in the body, the therapeutic composition must be released from the dosage form at a rate that will replace the composition being metabolized and excreted from the body.

The sustained release of an active ingredient may be stimulated by various inducers, for example pH, temperature, enzymes, water, or other physiological conditions or compounds.

Preparations for oral administration may be suitably formulated to give controlled release of the active compound. In various embodiments, the compounds are formulated as controlled release powders of discrete microparticles that can be readily formulated in liquid form. The sustained release powder comprises particles containing an active ingredient and optionally, an excipient with at least one non-toxic polymer.

The powder can be dispersed or suspended in a liquid vehicle and will maintain its sustained release characteristics for a useful period of time. These dispersions or suspensions have both chemical stability and stability in terms of dissolution rate. The powder may contain an excipient comprising a polymer, which may be soluble, insoluble, permeable, impermeable, or biodegradable. The polymers may be polymers or copolymers. The polymer may be a natural or synthetic polymer. Natural polymers include polypeptides (e.g., zein), polysaccharides (e.g., cellulose), and alginic acid. Representative synthetic polymers include those described, but not limited to, those described in column 3, lines 33-45 of U.S. Pat. No. 5,354,556, which is incorporated by reference in its entirety. Particularly suitable polymers include those described, but not limited to those described in column 3, line 46-column 4, line 8 of U.S. Pat. No. 5,354,556 which is incorporated by reference in its entirety.

The sustained release compositions provided herein may be formulated for parenteral administration, e.g., by intramuscular injections or implants for subcutaneous tissues and various body cavities and transdermal devices. In various embodiments, intramuscular injections are formulated as aqueous or oil suspensions. In an aqueous suspension, the sustained release effect is due to, in part, a reduction in solubility of the active compound upon complexation or a decrease in dissolution rate. A similar approach is taken with oil suspensions and solutions, wherein the release rate of an active compound is determined by partitioning of the active compound out of the oil into the surrounding aqueous medium. Only active compounds which are oil soluble and have the desired partition characteristics are suitable. Oils that may be used for intramuscular injection include, but are not limited to, sesame, olive, arachis, maize, almond, soybean, cottonseed and castor oil.

A highly developed form of drug delivery that imparts sustained release over periods of time ranging from days to years is to implant a drug-bearing polymeric device subcutaneously or in various body cavities. The polymer material used in an implant, which must be biocompatible and nontoxic, include but are not limited to hydrogels, silicones, polyethylenes, ethylene-vinyl acetate copolymers, or biodegradable polymers.

### E. Evaluation of the Activity of the Compounds

The activity of the compounds as modulators of protein trafficking may be measured in the assays described herein that evaluate the ability of a compound to rescue an impairment in protein trafficking. For example, the yeast mutant cell line ypt1^{ts} can be used to identify compounds that rescue cells from the lethal phenotype of a mutant YPT1 allele (see, e.g., Examples and Schmitt et al. (1988) Cell 53:635-47). The activity may be measured, for example, in a whole yeast cell assay using 384-well screening protocol and an optical density measurement.

Table A details human orthologs of the yeast genes YPT1 and SAR1. As detailed herein, a cell (e.g., a mammalian cell or a yeast cell) that exhibits reduced expression or activity of a protein required for protein trafficking (e.g., a protein of Table A) can be used to screen candidate agents for their ability to rescue the cell from a protein trafficking defect.

TABLE A
Human Counterparts of Yeast Genes YPT1 and SAR1

| Yeast | | DNA Accession | Protein Accession |
|---|---|---|---|
| Gene | Human Gene | Number | Number |
| Name | Name | (Human Gene) | (Human Gene) |
| YPT1 | Rab1a | NM_004161 | NP_004152.1 |
| | Rab1b | NM_030981 | NP_112243.1 |
| | Rab8b | NM_016530 | NP_057614.1 |
| | Rab8a | NM_005370 | NP_005361.2 |
| | Rab10 | NM_016131 | NP_057215.2 |
| | Rab13 | NM_002870 | NP_002861.1 |
| | Rab35 | NM_006861 | NP_006852.1 |
| | Rab11b | NM_004218 | NP_004209.1 |
| | Rab30 | NM_014488 | NP_055303.2 |
| | Rab11a | NM_004663 | NP_004654.1 |
| | Rab3a | NM_002866 | NP_002857.1 |
| | Rab3c | NM_138453 | NP_612462.1 |
| | Rab3d | NM_004283 | NP_004274.1 |
| | Rab3b | NM_002867 | NP_002858.2 |
| | Rab2 | NM_002865 | NP_002856.1 |
| | Rab43 | NM_198490 | NP_940892.1 |
| | Rab4a | NM_004578 | NP_004569.2 |
| | Rab2b | NM_032846 | NP_116235.2 |
| | Rab4b | NM_016154 | NP_057238.2 |
| | Rab25 | NM_020387 | NP_065120.1 |
| | Rab14 | NM_016322 | NP_057406.2 |
| | Rab37 | NM_001006638 | NP_001006639.1 |
| | Rab18 | NM_021252 | NP_067075.1 |
| | Rab5b | NM_002868 | NP_002859.1 |
| | Rab33a | NM_004794 | NP_004785.1 |
| | Rab26 | NM_014353 | NP_055168.2 |
| | Rab5a | NM_004162 | NP_004153.2 |
| | Rab19b | NM_001008749 | NP_001008749.1 |
| | Rab5c | NM_201434 | NP_958842.1 |
| | Rab33b | NM_031296 | NP_112586.1 |
| | Rab39b | NM_171998 | NP_741995.1 |
| | Rab39 | NM_017516 | NP_059986.1 |
| | Rab31 | NM_006868 | NP_006859.2 |
| | Rab15 | NM_198686 | NP_941959.1 |
| | Rab40c | NM_021168 | NP_066991.2 |
| | Rab27b | NM_004163 | NP_004154.2 |
| | Rab22a | NM_020673 | NP_065724.1 |
| | Rab6b | NM_016577 | NP_057661.2 |
| | Rab40b | NM_006822 | NP_006813.1 |
| | Rasef | NM_152573 | NP_689786.2 |
| | Rab21 | NM_014999 | NP_055814.1 |
| | Rab27a | NM_183236 | NP_899059.1 |
| | Loc286526 | NM_001031834 | NP_001027004.1 |
| | Rab40a | NM_080879 | NP_543155.2 |
| | Rab6a | NM_198896 | NP_942599.1 |
| | Rab17 | NM_022449 | NP_071894.1 |
| | Rab6c | NM_032144 | NP_115520.1 |
| | Rab7 | NM_004637 | NP_004628.4 |
| | Rab9a | NM_004251 | NP_004242.1 |
| | Rab7l1 | NM_003929 | NP_003920.1 |
| | Rab9b | NM_016370 | NP_057454.1 |
| | Rab34 | NM_031934 | NP_114140.2 |
| | Rab7b | NM_177403 | NP_796377.2 |
| | Rab41 | NM_001032726 | NP_001027898.1 |
| | Rab23 | NM_183227 | NP_899050.1 |
| | Rab32 | NM_006834 | NP_006825.1 |
| | Rab38 | NM_022337 | NP_071732 |
| | Rab36 | NM_004914 | NP_004905 |
| | Rab28 | NM_001017979 | NP_001017979 |
| | Rab20 | NM_017817 | NP_060287 |
| | Rab12 | NM_001025300 | NP_001020471 |
| SAR1 | Sar1a | NM_020150 | NP_064535 |
| | Sar1b | NM_001033503 | NP_001028675 |
| SEC23 | Sec23a | NM_006364.2 | NP_006355.2 |
| | Sec23b | NM_006363.4 | NP_006354 |

In addition, efficacy of a compound can be evaluated before (first in time), concomitantly or subsequently to the above-mentioned test modalities by monitoring, e.g., (i) modulation (e.g., an improvement) of the stability of a trafficking defective protein, (ii) modulation (e.g., an improvement) of proper, physiological trafficking of the trafficking defective protein, or (iii) modulation (e.g., a restoration) in one or more functions of a trafficking defective protein. For example, in some cases, proteins (e.g., protein mutants such as ΔF508 CFTR) are prematurely degraded. Thus, the efficacy of a given compound to modulate protein trafficking can be determined by monitoring the stability of a protein in the presence as compared to the absence of the compound. For example, cells expressing a trafficking defective protein (e.g., expressing endogenously or expressing an exogenous transgene encoding a trafficking defective protein such as ΔF508 CFTR) can be cultured in the presence or absence of a compound for at least 1 hour (e.g., at least 2 hours, at least 4 hours, at least 6 hours, at least 8 hours, at least 12 hours, at least 16 hours, at least 24 hours, at least 36 hours, or at least 48 hours). Cell lysates can be prepared from the different populations of cells, suspended in Laemmli buffer (with or without reducing agent) and subjected to sodium dodecyl sulfate-polyacrylamide gel electrophoresis (SDS-PAGE). Using antibodies that specifically recognize the trafficking defective protein (e.g., CFTR), the amount of the protein in the presence as compared to in the absence of a compound can be determined by western or dot-blotting techniques. An increase in the amount of a trafficking defective protein in the presence of a compound as compared to in the absence of the compound indicates that the compound modulates (e.g., stabilizes) a trafficking defective protein (Vij et al. (2006) J. Biol. Chem. 281(25):17369-17378). Where a modified state (e.g., glycosylation or phosphorylation) of a protein is indicative of increased stability, a change in the modified state of a protein can also be used to determine if a compound stabilizes the trafficking defective protein. For example, the amount of glycosylated CFTR (e.g., ΔF508 CFTR) can be assessed in the presence as compared to the absence of a compound. An increase in the glycosylated form of the protein is an indicated that the compound has stabilized CFTR (e.g., ΔF508 CFTR).

It is understood that routine adaptation of this assay can be used to monitor any trafficking defective protein. Furthermore, steady-state levels (e.g., protein turnover or the degradation rate) of a protein can also be monitored in the presence and absence of a compound (e.g., see Van Goor et al. (2006) Am. J. Physiol. Lung. Cell Mol. Physiol. 290:L1117-L1130).

Another method of determining modulation of a trafficking defective protein is an in situ staining method. For example, where a protein (e.g., ΔF508 CFTR or G601S-hERG) is prematurely degraded before reaching the cell surface, the efficacy of a compound to modulate the trafficking defective protein can be determined as a change (e.g., an increase) in the amount of surface expression of the protein. Thus, an increase in the amount protein expression at the cell surface in the presence of a compound as compared to the surface expression in the absence of a compound indicates that compound modulates (e.g., stabilizes) the trafficking defective protein. Immunostaining methods are well known to those of skill in the art and include embodiments where the cells are still viable (e.g., confocal microscopy of live cells such as mammalian cells) or staining of fixed cells (e.g., immunohistochemistry). The cells can be attached to a solid support (e.g., a tissue culture plate or poly-lysine coated glass slide) or can be in solution (e.g., for fluorescence assisted cell sorting (FACS) analysis). A primary antibody specific for a trafficking defective protein are applied (e.g., administered, delivered, contacted) to cells. The primary antibody itself can be labeled with a detectable label (e.g., a different colored fluorophore (e.g., rhodamine, texas red, FITC, Green fluorescent protein, Cy3, Cy5). Alternatively, a secondary agent, such as a secondary antibody, can be detectably labeled and the primary antibody unlabeled. The primary antibody can also be conjugated to a first member of a binding pair (e.g., biotin or streptavidin) and the second member of the binding pair detectably labeled. Use of an appropriate microscope (e.g., a confocal microscope) with the appropriate optical filters can identify the position of the labeled antibodies in a given cell. An increase in signal from the detectable label from the cell surface indicates that more protein is expressed on the cell surface. Of course, it is understood that this method can be applied to trafficking defective proteins that localize to other compartments (e.g., organelles such as nucleus, lysosome, ER, Golgi, or mitochondria) of the cell. It can be useful to use another antibody or dye to identify another control protein known to localize to the given compartment of interest. Typically, the second protein is labeled with a different detectable label than the trafficking defective protein of interest. The position of both labels is then determined by the preceding methods. When each of the positions of the two proteins are determined (i.e., the location of their respective detectable label within the cell as determined by antibody binding), if they are found to occupy the same space, the two proteins are said to co-localize and thus, the trafficking defective protein has localized to the proper cellular position (i.e., when two proteins co-localize in the absence of a compound but do not co-localize in the presence of a compound, this can indicate that the compound has inhibited the interaction between the two proteins). Examples of this method are described in, for example, Morello et al. (2000) J. Clin. Invest. 105(7):887-895 and Liu et al. (2003) Proc. Natl. Acad. Sci. USA 100(26):15824-15829. Optionally the cells can be fixed, for example, using paraformaldehyde or formaldehyde, and permeabilized using a detergent (e.g., Triton-X¹00).

The efficacy of a compound to modulate a trafficking defective protein can also be assessed by monitoring an increase in the activity of the trafficking defective protein. For example, the ΔF508 CFTR is a PKA-regulated chloride channel, and thus an increase in the stability of the CFTR protein can be determined by an increase in, e.g., membrane potential response to forskolin or induction of cAMP-mediated chloride efflux (see, e.g., Vij et al. (2006) J. Biol. Chem. 281(25):17369-17378 and Van Goor et al. (2006) Am. J. Physiol. Lung. Cell Mol. Physiol. 290:L1117-L1130). Alpha-galactosidase-A, the trafficking defective protein in Fabry's disease, is an enzyme that metabolizes certain lipids. Therefore, the efficacy of a compound to modulate alpha-galactosidase-A can be determined by assessing the cellular activity of alpha-galactosidase in the presence as compared to in the absence of a compound. An increase in activity in the presence of the compound as compared to in the absence of the compound indicates that compound modulates (e.g., stabilizes) the alpha-galactosidase-A protein. Methods of monitoring for alpha-galactosidase activities in cells can be found in, e.g., Ioannou et al. (1998) Biochem. J. 332:789-797. Methods for monitoring the in vitro and in vivo enzymatic activities of trafficking defective proteins causative of their respective disorder characterized by impaired protein traffickings, other than CFTR and alpha-galactosidase-A, are known in the art.

Protein trafficking (e.g., endoplasmic reticulum-mediated protein trafficking) can also be detected and measured using in vitro (cell-free) methods. Thus, the efficacy of a compound to modulate, e.g., a trafficking defective protein or various steps of protein trafficking (e.g., formation or docking of COPII vesicles) can be determined using such in vitro methods. Suitable in vitro methods for detecting or measuring endoplasmic-reticulum mediated protein trafficking are described in, e.g., Rexach et al. (1991) J. Cell Biol. 114(2):219-229; Segev (1991) Science 252(5012):1553-1556; Balch et al. (1984) Cell 39(2 Pt 1):405-416; Wattenberg (1991) J Electron Microsc Tech 17(2):150-164; Beckers et al. (1989) J. Cell Biol. 108(4):1245-1256; and Moreau et al. (1991) J. Biol. Chem. 266(7):4322-4328, the contents of each of which are incorporated herein by reference in their entirety. For example, transfer of a protein of interest from endoplasmic reticulum to Golgi can be detected or measured. First, a reporter protein is labeled in a cell, e.g., by metabolically labeling the protein using ³⁵S-methionine or by expressing a detectably-labeled form of the protein in a cell (a fusion protein comprising the protein of interest and green fluorescent protein). “Donor” membrane fractions containing endoplasmic reticulum can be obtained from the cells containing labeled protein. “Acceptor” membrane fractions containing Golgi apparatus can be prepared from cells not containing labeled protein. Transport of the labeled protein is accompanied by post-translational modification. Often the reporter protein is a glycoprotein whose carbohydrate chains are modified during ER to Golgi transport. Acceptor and donor fractions are mixed and incubated with required cofactors. Transport is monitored by the increase in the post-translationally modified form of the labeled protein. Methods for detecting the post-translationally modified labeled protein are described herein and can include western, dot blotting, lectin binding, and suspectability to glycosidases. When the detectable label is a fluorescent or luminescent label, a fluorimeter or luminometer can be utilized. When the detectable label is a radioactive label (see below), scintillation counter, X-ray film, or radiometer. It is understood that a protein need not be detectably labeled. A protein initially present in the Donor fraction (e.g., a protein specifically expressed in the Donor cell population), but not present in the Acceptor fraction can be distinguished using, e.g., western blotting techniques.

In vitro methods of detecting protein trafficking (e.g., endoplasmic reticulum-mediated protein trafficking) can also involve measuring vesicle budding, uncoating, tethering, or docking or fusion with the Golgi apparatus (see, e.g., Rexach et al., supra, and Bonifacino et al. (2004) Cell 116:153-166).

To determine if a compound modulates the in vitro transfer of a protein from endoplasmic reticulum to Golgi (e.g., any step of the transfer of a protein from endoplasmic reticulum to Golgi), a compound can be contacted to the Acceptor fraction, Donor fraction, or both before or during the incubation. The compound could be added to either Donor or Acceptor cell populations prior to preparing the membrane fractions. As described herein (see, e.g., Examples), compounds that inhibit the proteasome (e.g., proteosome expression or activity) can also be screened through the assays described herein (e.g., ypt1ts mutant assay) to determine if they rescue endoplasmic reticulum-mediated transport. In vitro and in vivo (cell-based) methods of detecting and/or measuring proteasome activity are known in the art and are described, for example, in Chuhan et al. (2006) Br. J. Cancer 95(8):961-965; Rubin et al. (1998) EMBO J. 17(17):4909-4919; Glickman et al. (1999) Mol. Biol. Rep. 26(1-2):21-8; and Grimes et al. (2005) Int. J. Oncol. 27(4):1047-1052. In vitro methods of determining whether a candidate compound inhibits the proteasome, e.g., proteasome activity, can include contacting isolated proteasome complexes with a candidate compound and measuring the activity of the isolated proteasomes contacted with the candidate compound. A decrease in the activity of a proteasome contacted with a compound as compared to proteasome activity in the absence of the compound indicates that the candidate compound inhibits proteasome activity in vitro. In vivo methods of determining whether a candidate compound inhibits the proteasome can include, e.g., contacting a cell with a candidate compound and measuring the activity of proteasomes in the cell. For example, measuring the turnover of proteins known to be degraded by the proteasome. A decrease in the activity of proteasomes in a cell contacted with a compound as compared to proteasome activity in a cell in the absence of the compound indicates that the candidate compound inhibits proteasome activity in vivo. Examples of proteosome inhibitors include, e.g., MG132, MG15, LLnL, ALLnL, bortezomib/PS-341/VELCADE®, NPI-0052, epoxomicin, and lactacystin (Myung et al. (2001) Med. Res. Reviews 21(4):245-273; Montagut et al. (2006) Clin Transl Oncol. 8(5):313-317; and Chuhan et al. (2006) Br. J. Cancer 95(8):961-965).

For example, modulators of α-synuclein toxicity may be measured in standard assays (see, e.g., U.S. patent application Ser. No. 10/826,157, filed Apr. 16, 2004; U.S. Patent Application Publication No. 2003/0073610; and the examples). The activity may be measured in a whole yeast cell assay using 384-well screening protocol and an optical density measurement. Expression of human α-synuclein in yeast inhibits growth in a copy-number dependent manner (see, e.g., Outeiro, et al. (2003) Science 302(5651):1772-5). Expression of one copy of α-syn::GFP has no effect on growth, while two copies result in complete inhibition. The cessation of growth is accompanied by a change in α-syn::GFP localization. In cells with one copy, α-syn::GFP associates with the plasma membrane in a highly selective manner. When expression is doubled, α-synuclein migrates to the cytoplasm where it forms large inclusions that are similar to Lewy bodies seen in diseased neurons.

The compounds provided herein can be screened in this assay for α-synuclein toxicity rescue. Briefly, the humanized strain is exposed to compounds in 384-well plates under conditions that induce α-synuclein expression. After incubation for 24 or 48 hours, or both, growth is measured. Compounds that inhibit toxicity will restore growth and are detected as an increase in turbidity (OD₆₀₀).

Additional assays can be used to screen compounds to assess their ability to modulate α-synuclein toxicity. These assays include, for example, screening for compounds that modulate α-synuclein induced toxicity in human neuroglioma cells (see, e.g., McLean et al. (2004) Biochem Biophys Res Commun. 321(3):665-69) or in worms or primary neurons (see, e.g., Cooper et al. (2006) Science 313(5785):324-8 and supplementary materials).

### F. Methods of Producing a Protein

The compounds described herein enhance endoplasmic reticulum-mediated transport and thus can be used in methods to enhance protein production in a cell. The protein produced by the methods can be a naturally occurring or a non-naturally occurring protein. The protein can be produced naturally by a cell (e.g., without any genetic manipulation of the cell), can be encoded by a heterologous nucleic acid introduced into a cell, or can be produced by a cell following the insertion or activation of sequences that regulate expression of a gene encoding the protein.

A “heterologous nucleic acid” refers to a nucleotide sequence that has been introduced into a cell by the use of recombinant techniques. Accordingly, a “heterologous nucleic acid” present in a given cell does not naturally occur in the cell (e.g., has no corresponding identical sequence in the genome of the cell) and/or is present in the cell at a location different than that where a corresponding identical sequence naturally exists (e.g., the nucleotide sequence is present in a different location in the genome of the cell or is present in the cell as a construct not integrated in the genome).

Any protein that is produced by a cell can be used in the methods described herein. For example, proteins such as cytokines, lymphokines, and/or growth factors can be produced. Examples of such proteins include, but are not limited to, Erythropoietin, Interleukin 1-Alpha, Interleukin 1-Beta, Interleukin-2, Interleukin-3, Interleukin-4, Interleukin-5, Interleukin-6, Interleukin-7, Interleukin-8, Interleukin-9, Interleukin-10, Interleukin-11, Interleukin-12, Interleukin-13, Interleukin-14, Interleukin-15, Lymphotactin, Lymphotoxin Alpha, Monocyte Chemoattractant Protein-1, Monocyte Chemoattractant Protein-2, Monocyte Chemoattractant Protein-3, Megapoietin, Oncostatin M, Steel Factor, Thrombopoietin, Vascular Endothelial Cell Growth Factor, Bone Morphogenetic Proteins, Interleukin-1 Receptor Antagonist, Granulocyte-Colony Stimulating Factor, Leukemia Inhibitory Factor, Granulocyte-Macrophage Colony-Stimulating Factor, Macrophage Colony-Stimulating Factor, Interferon Gamma, Interferon Beta, Fibroblast Growth Factor, Tumor Necrosis Factor Alpha, Tumor Necrosis Factor Beta, Transforming Growth Factor Alpha, Gonadotropin, Nerve Growth Factor, Platelet-Derived Growth Factor, Macrophage Inflammatory Protein 1 Alpha, Macrophage Inflammatory Protein 1 Beta, and Fas Ligand. Cells producing a non-naturally occurring, variant of any the above polypeptides can also be used in the methods described herein.

In addition to the proteins described above, the methods described herein can also be used to produce a fusion protein that contains all or a portion of a given protein fused to a sequence of amino acids that direct secretion of the fusion protein from a cell. In some cases, such fusion proteins can allow for the secretion of a polypeptide sequence that is not typically secreted from a cell. For example, all or a portion of a protein (e.g., a membrane associated protein such as a receptor or an intracellular protein) can be fused to a portion of an immunoglobulin molecule (e.g., to the hinge region and constant region CH2 and CH3 domains of a human IgG1 heavy chain).

The protein produced by the methods described herein can be an antibody or an antigen-binding fragment of an antibody. The antibody can be directed against an antigen, e.g., a protein antigen such as a soluble polypeptide or a cell surface receptor. For example, the antibody can be directed against a cell surface receptor involved in immune cell activation, a disease-associated antigen, or an antigen produced by a pathogen. The term “antibody” refers to an immunoglobulin molecule or an antigen-binding portion thereof. As used herein, the term “antibody” refers to a protein containing at least one, for example two, heavy chain variable regions (“VH”), and at least one, for example two, light chain variable regions (“VL”). The VH and VL regions can be further subdivided into regions of hypervariability, termed “complementarity determining regions” (“CDR”), interspersed with regions that are more conserved, termed “framework regions” (FR). The antibody can further include a heavy and light chain constant region, to thereby form a heavy and light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region contains three domains, CH1, CH2, and CH3. The light chain constant region contains one domain, CL. The variable region of the heavy and light chains contains a binding domain that interacts with an antigen.

The protein can be a fully human antibody (e.g., an antibody made in a mouse genetically engineered to produce an antibody from a human immunoglobulin sequence), a humanized antibody, or a non-human antibody, e.g., a rodent (mouse or rat), goat, or primate (e.g., monkey) antibody.

### G. Methods of Treating a Disorder Characterized by Impaired Protein Trafficking

GTP-bound Rab proteins such as Rab1, the homolog of yeast ypt1, are involved in the global regulation of vesicle transport. As detailed throughout the specification and in the Examples, compounds identified in the ypt^{ts} mutant rescue screening assay can be useful to stabilize trafficking defective proteins, e.g., by modulating the Rab-ypt1 pathway. Thus, the compounds disclosed herein (and pharmaceutical compositions comprising same) can be useful in methods to treat one or more symptoms of a variety of disorders characterized by impaired protein trafficking. As described in Example 4, compounds identified using the ypt1^{ts} mutant rescue screen are also capable of stabilizing ΔF508 CFTR. Thus the compounds described herein can be particularly useful in treating or preventing one or more symptoms of cystic fibrosis.

Types of disorders characterized by impaired protein trafficking that could be treated through the administration of one or more compounds (or pharmaceutical compositions of the same) described herein can include, e.g., hereditary emphysema, hereditary hemochromatosis, oculocutaneous albinism, protein C deficiency, type I hereditary angioedema, congenital sucrase-isomaltase deficiency, Crigler-Najjar type II, Laron syndrome, hereditary Myeloperoxidase, primary hypothyroidism, congenital long QT syndrome, thyroxine binding globulin deficiency, familial hypercholesterolemia, familial chylomicronemia, abeta-lipoproteinema, low plasma lipoprotein a levels, hereditary emphysema with liver injury, congenital hypothyroidism, osteogenesis imperfecta, hereditary hypofibrinogenemia, alpha-1antichymotrypsin deficiency, nephrogenic diabetes insipidus, neurohypophyseal diabetes, insipidus, Charcot-Marie-Tooth syndrome, Pelizaeus Merzbacher disease, von Willebrand disease type IIA, combined factors V and VIII deficiency, spondylo-epiphyseal dysplasia tarda, choroideremia, I cell disease, Batten disease, ataxia telangiectasias, acute lymphoblastic leukemia, acute myeloid leukemia, myeloid leukemia, ADPKD-autosomal dominant polycystic kidney disease, microvillus inclusion disease, tuberous sclerosis, oculocerebro-renal syndrome of Lowe, amyotrophic lateral sclerosis, myelodysplastic syndrome, Bare lymphocyte syndrome, Tangier disease, familial intrahepatic cholestasis, X-linked adreno-leukodystrophy, Scott syndrome, Hermansky-Pudlak syndrome types 1 and 2, Zellweger syndrome, rhizomelic chondrodysplasia puncta, autosomal recessive primary hyperoxaluria, Mohr Tranebjaerg syndrome, spinal and bullar muscular atrophy, primary ciliary diskenesia (Kartagener's syndrome), Miller Dieker syndrome, lissencephaly, motor neuron disease, Usher's syndrome, Wiskott-Aldrich syndrome, Optiz syndrome, Huntington's disease, hereditary pancreatitis, anti-phospholipid syndrome, overlap connective tissue disease, Sjögren's syndrome, stiff-man syndrome, Brugada syndrome, congenital nephritic syndrome of the Finnish type, Dubin-Johnson syndrome, X-linked hypophosphosphatemia, Pendred syndrome, persistent hyperinsulinemic hypoglycemia of infancy, hereditary spherocytosis, aceruloplasminemia, infantile neuronal ceroid lipofuscinosis, pseudoachondroplasia and multiple epiphyseal, Stargardt-like macular dystrophy, X-linked Charcot-Marie-Tooth disease, autosomal dominant retinitis pigmentosa, Wolcott-Rallison syndrome, Cushing's disease, limb-girdle muscular dystrophy, mucoploy-saccharidosis type IV, hereditary familial amyloidosis of Finish, Glycogen storage disease type IV (Andersen's disease), sarcoma, chronic myelomonocytic leukemia, cardiomyopathy, faciogenital dysplasia, Torsion disease, Huntington and spinocerebellar ataxias, hereditary hyperhomosyteinemia, polyneuropathy, lower motor neuron disease, pigmented retinitis, seronegative polyarthritis, interstitial pulmonary fibrosis, Raynaud's phenomenon, Wegner's granulomatosis, preoteinuria, CDG-Ia, CDG-Ib, CDG-Ic, CDG-Id, CDG-Ie, CDG-If, CDG-IIa, CDG-IIb, CDG-IIc, CDG-IId, Ehlers-Danlos syndrome, multiple exostoses, Griscelli syndrome (type 1 or type 2), or X-linked non-specific mental retardation. In addition, disorders characterized by impaired protein trafficking can also include lysosomal storage disorders such as, but not limited to, Fabry disease, Farber disease, Gaucher disease, GM₁-gangliosidosis, Tay-Sachs disease, Sandhoff disease, GM₂ activator disease, Krabbe disease, metachromatic leukodystrophy, Niemann-Pick disease (types A, B, and C), Hurler disease, Scheie disease, Hunter disease, Sanfilippo disease, Morquio disease, Maroteaux-Lamy disease, hyaluronidase deficiency, aspartylglucosaminuria, fucosidosis, mannosidosis, Schindler disease, sialidosis type 1, Pompe disease, Pycnodysostosis, ceroid lipofuscinosis, cholesterol ester storage disease, Wolman disease, Multiple sulfatase, galactosialidosis, mucolipidosis (types II, III, and IV), cystinosis, sialic acid storage disorder, chylomicron retention disease with Marinesco-Sjögren syndrome, Hermansky-Pudlak syndrome, Chediak-Higashi syndrome, Danon disease, or Geleophysic dysplasia.

Symptoms of a disorder characterized by impaired protein trafficking are numerous and diverse and can include one or more of, e.g., anemia, fatigue, bruising easily, low blood platelets, liver enlargement, spleen enlargement, skeletal weakening, lung impairment, infections (e.g., chest infections or pneumonias), kidney impairment, progressive brain damage, seizures, extra thick meconium, coughing, wheezing, excess saliva or mucous production, shortness of breath, abdominal pain, occluded bowel or gut, fertility problems, polyps in the nose, clubbing of the finger/toe nails and skin, pain in the hands or feet, angiokeratoma, decreased perspiration, corneal and lenticular opacities, cataracts, mitral valve prolapse and/or regurgitation, cardiomegaly, temperature intolerance, difficulty walking, difficulty swallowing, progressive vision loss, progressive hearing loss, hypotonia, macroglossia, areflexia, lower back pain, sleep apnea, orthopnea, somnolence, lordosis, or scoliosis. It is understood that due to the diverse nature of the trafficking defective proteins and the resulting disease phenotypes (e.g., a disorder characterized by impaired protein trafficking), a given disorders will generally present only symptoms characteristic to that particular disorder. For example, a patient with cystic fibrosis can present a particular subset of the above-mentioned symptoms such as, but not limited to, persistent coughing, excess saliva and mucus production, wheezing, coughing, shortness of breath, enlarged liver and/or spleen, polyps of the nose, diabetes, fertility problems, increased infections (e.g., respiratory infections such as pneumonias), or occluded gut or bowel.

Depending on the specific nature of the disorder, a patient can present these symptoms at any age. In many cases, symptoms can present in childhood or in early adulthood. For example, symptoms of cystic fibrosis often present at birth when a baby's gut becomes blocked by extra-thick muconium.

Following administration of one or more of the disclosed compounds (or pharmaceutical compositions) to a subject (e.g., a human patient), the efficacy of the treatment in ameliorating one or more symptoms of a disorder characterized by impaired protein trafficking can be assessed by comparing the number and/or severity of one or more symptoms presented by a patient before and after treatment. Alternatively, where administration of the compounds is used to prevent the occurrence of a disorder characterized by impaired protein trafficking, treatment efficacy can be assessed as a delay in presentation of, or a failure to present, one or more symptoms of a disorder characterized by impaired protein trafficking. The efficacy of a treatment (e.g., a compound or composition described herein) over time (e.g., a progressive improvement) in ameliorating one or more symptoms of a disorder characterized by impaired protein trafficking can be determined by assessing, e.g., the number or severity of one or more symptoms at multiple time points following treatment. For example, a subject (e.g., a patient) can have an initial assessment of the severity of his or her disorder (e.g., the number or severity of one or more symptoms of a disorder characterized by impaired protein trafficking), administered treatment, and then assessed subsequently to the treatment two or more times (e.g., at one week and one month; at one month at two months; at two weeks, one month, and six months; or six weeks, six months, and a year). Where one or more compounds or compositions are administered to a subject for a limited period of time (e.g., a predetermined duration) or number of administrations, the effect of treatment on ameliorating one or more symptoms of a disorder characterized by impaired protein trafficking can be assessed at various time points after the final treatment. For example, following the last administration of a dose of one or more compounds, the number or severity of a patient's symptoms can be assessed at 1 month (e.g., at 2 months, at 6 months, at one year, at two years, at 5 years or more) subsequent to the final treatment.

The efficacy of a treatment with one or more compounds (or compositions) described herein on one or more symptoms of a disorder characterized by impaired protein trafficking can be assessed as a monotherapy or as part of a multi-therapeutic regimen. For example, the compound(s) can be administered in conjunction with other clinically relevant treatments for disorder characterized by impaired protein traffickings including, but not limited to, physical or respiratory therapy, antibiotics, anti-asthma therapies, cortisteroids, vitamin supplements, pulmozyme treatments, CEREZYME®, CEREDASE®, MYOZYME®, insulin, FABRYZYME®, dialysis, transplants (e.g., liver or kidney), stool softeners or laxatives, anti-blot clotting agents (anti-coagulants), pain medications, and/or angioplasty. It is understood that due to the diverse activities of trafficking defective proteins and the diverse clinical manifestations of the associated disorders (e.g., Fabry's disease, cystic fibrosis, Gaucher's disease, Pompe disease, and the like) the “other clinically relevant treatments” can also include treatments beyond those above. For example, other or additional clinically relevant treatments for cystic fibrosis include, e.g., antibiotics, pulmozyme treatments, vitamin supplements, stool softeners or laxatives, insulin for cystic-fibrosis related diabetes, anti-asthma therapies, or corticosteroids.

A compound or pharmaceutical composition thereof described herein can be administered to a subject as a combination therapy with another treatment (another active ingredients), e.g., a treatment for a disorder characterized by impaired protein trafficking such as cystic fibrosis or a lysosomal storage disease. For example, the combination therapy can include administering to the subject (e.g., a human patient) one or more additional agents that provide a therapeutic benefit to the subject who has, or is at risk of developing, (or suspected of having) a disorder characterized by impaired protein trafficking such as cystic fibrosis. Thus, the compound or pharmaceutical composition and the one or more additional agents are administered at the same time. Alternatively, the compound can be administered first in time and the one or more additional agents administered second in time. The one or more additional agents can be administered first in time and the compound administered second in time. The compound can replace or augment a previously or currently administered therapy (also, see below). For example, upon treating with a compound of the invention, administration of the one or more additional agents can cease or diminish, e.g., be administered at lower levels. Administration of the previous therapy can also be maintained. In some instances, a previous therapy can be maintained until the level of the compound (e.g., the dosage or schedule) reaches a level sufficient to provide a therapeutic effect. The two therapies can be administered in combination.

It will be appreciated that in instances where a previous therapy is particularly toxic (e.g., a treatment for disorder characterized by impaired protein trafficking carrying significant side-effect profiles) or poorly tolerated by a subject (e.g., a patient), administration of the compound can be used to offset and/or lessen the amount of the previous therapy to a level sufficient to give the same or improved therapeutic benefit, but without the toxicity.

In some instances, when the subject is administered a compound or pharmaceutical composition of the invention, the first therapy is halted. The subject can be monitored for a first pre-selected result, e.g., an improvement in one or more symptoms of a disorder characterized by impaired protein trafficking such as any of those described herein (e.g., see above). In some cases, where the first pre-selected result is observed, treatment with the compound is decreased or halted. The subject can then be monitored for a second pre-selected result after treatment with the compound is halted, e.g., a worsening of a symptom of disorder characterized by impaired protein trafficking. When the second pre-selected result is observed, administration of the compound to the subject can be reinstated or increased, or administration of the first therapy reinstated, or the subject is administered both a compound and first therapy, or an increased amount of the compound and the first therapeutic regimen.

Methods of assessing the effect of a therapy (e.g., a compound or composition of the invention) are known in the art of medicine and include assessing the change (e.g., the improvement) in one or more symptoms of a disorder characterized by impaired protein trafficking such as any of those described herein (see above). In addition, while the invention is not limited by any particular theory or mechanism of action, because the compounds identified herein can function at the molecular level to correct the disorder characterized by impaired protein trafficking, assessing the effect of a therapy on patient having a disorder characterized by impaired protein trafficking can be done by assessing, e.g., (i) an improvement of the stability of a trafficking defective protein, (ii) improvement of proper, physiological trafficking of the trafficking defective protein, or (iii) a restoration in one or more functions of a trafficking defective protein (see above under “E. Evaluation of the Activity of the Compounds”).

In particular, efficacy of treatment (e.g., administration of one or more compounds or pharmaceutical compositions described herein) of cystic fibrosis can be monitored, e.g., by performing a “sweat test” before and after treatment. The sweat test is generally conducted by a physician or medical practitioner. A colorless, odorless chemical is placed on the skin, which causes it to sweat, and a device collects the sweat. A sweat test can take 30 minutes to 1 hour, depending on how long it takes to collect the subject's perspiration. Chloride levels in the subject's perspiration are measured (e.g., using a SWEAT-CHEK™ Sweat Conductivity Analyzer, Discovery Diagnostics, Ontario, Canada) and, for example, a relative score of <40 indicates normality, a score of 40-59 is an intermediate range, and a score of >60 indicates that the subject still has profound disease. Efficacy of a treatment of cystic fibrosis can also be determined using a nasal potential difference (NPD) test. The test is especially useful for subjects (e.g., patients) who have normal chloride levels as determined by sweat tests. The NPD test requires 2 electrodes, connected to a voltmeter such as the THOLY-MEDICAP® device), one placed on the nasal mucosa of the inferior turbinate and the other placed subcutaneously on the forearm. Generally, a reading less than −40 mV is considered abnormal. Thus, a patient who's NPD test readings improve to over −40 mV can be one considered to improve (see, for example, Domingo-Ribas et al. (2006) Arch Bronconeumol. 42:33-38).

### EXAMPLES

The following examples are provided for illustrative purposes only and are not intended to limit the scope of the invention.

### Example 1

### Compounds that Restore Growth of a ypt1^{ts} Mutant

The yeast mutant cell line ypt1^{ts} suppresses, in a temperature dependent fashion, the dominant-lethal phenotype of a mutant YPT1 allele (Schmitt et al. (1988) Cell 53:635-47). The yeast mutant cell line ypt1^{ts} contains an allele of YPT1 that has two point mutations: one that changes an asparagine at position 121 to a isoleucine (N121I) and another that changes an alanine at position 161 to a valine (A161V). The N121I mutation causes dominant lethality by itself, but lethality is suppressed by the second mutation, resulting in a recessive loss of function phenotype at the restrictive temperatures. ypt1^{ts} cells grow normally at temperatures up to 25° C., but are growth arrested at 37° C. (Id.). At the non-permissive temperature of 37° C., ypt1^{ts} mutants accumulate ER membranes, small vesicles, and unprocessed invertase and exhibit cytoskeletal defects and enhanced calcium uptake (Id). ypt1^{ts} mutant cells can be rescued from growth arrest by the provision of extracellular calcium (Id).

Compounds were screened to assess their ability to restore growth of ypt1^{ts} cells. The effect of the compounds was measured on ypt1^{ts} cells cultured at room temperature (permissive temperature), 37° C. (non-permissive temperature), and 35° C. (semi-permissive temperature). Certain compounds (and analogs thereof) that rescue α-synuclein toxicity were found to also rescue ypt1^{ts} toxicity.

To determine if the test compounds could rescue the ypt1^{ts} mutant phenotype, ypt1^{ts} cells were grown overnight in synthetic complete (SC) media supplemented with 2% glucose at room temperature. Log phase cells were diluted into SC 2% glucose media to an OD600 of 0.003. 100 μL of this culture was then aliquoted into each well of 96-well flat bottom microtiter plates. 1 μL of the test compounds dissolved in DMSO (at a concentration range from 5 mM-0.005 mM) or of DMSO alone was added to each well (50 μM-0.05 μM final concentration in 1% DMSO). Plates were mixed by vortexing and incubated at 35° C. and 37° C. Compound rescue of the ypt1^{ts} temperature sensitive defect was assessed by measuring the OD₆₀₀ (optical density at 600 nm; cell growth) of the cultures. Plates incubated at 35° C. were measured at 24 and 40 hours incubation time while plates grown at 37° C. were measured after 40 hours of incubation.

Assays monitoring the rescue of ypt1^{ts} mutants were performed using a vehicle, a positive control (calcium), and the compounds identified in Table II. Results in Table II corresponding to an MRC (Minimum Rescue Concentration) for ypt1^{ts} of greater than or equal to 50 micromolar are labeled +; of an MRC for ypt1^{ts} of 10-50 micromolar are labeled ++; and of an MRC for ypt1^{ts} of less than 10 micromolar are labeled +++. Multiple results for a particular compound are separated by a comma.

The finding that the above compounds can rescue the ypt1^{ts} protein trafficking defect indicates that the compounds can be used to treat or prevent a variety of disorders characterized by impaired protein trafficking, e.g., in mammals or in mammalian cells. While such a yeast assay can be effective for screening compounds to identify some compounds which also have activity in mammalian cells or in mammals, it is noted various compounds may have activity in mammalian cells or in mammals without displaying activity in such a yeast assay.

TABLE II

| ID | Ypt1-ts |
|---|---|
| 1 | + |
| 2 | + |
| 3 | + |
| 4 | + |
| 5 | +++ |
| 6 | ++ |
| 7 | + |
| 8 | + |
| 9 | + |
| 10 | + |
| 11 | + |
| 12 | + |
| 13 | + |
| 14 | + |
| 15 | + |
| 16 | + |
| 17 | + |
| 18 | + |
| 19 | +++ |
| 20 | ++ |
| 21 | + |
| 22 | ++ |
| 23 | ++ |
| 24 | ++ |
| 25 | +++ |
| 26 | +++, + |
| 27 | ++ |
| 28 | ++ |
| 29 | +++ |
| 30 | + |
| 31 | +++ |
| 32 | + |
| 33 | ++ |
| 34 | ++ |
| 35 | + |
| 36 | + |
| 37 | + |
| 38 | + |
| 39 | + |
| 40 | + |
| 41 | + |
| 42 | + |
| 43 | + |
| 44 | + |
| 45 | + |
| 46 | + |
| 47 | + |
| 48 | + |
| 49 | ++ |
| 50 | ++ |
| 51 | + |
| 52 | ++ |
| 53 | + |
| 54 | + |
| 55 | +, +++ |
| 56 | ++ |
| 57 | ++ |
| 58 | +++ |
| 59 | + |
| 60 | + |
| 61 | + |
| 62 | + |
| 63 | + |
| 64 | ++ |
| 65 | ++ |
| 66 | ++ |
| 67 | ++ |
| 68 | ++ |
| 69 | + |
| 70 | + |
| 71 | +++ |
| 72 | +++ |
| 73 | +++ |
| 74 | +++ |
| 75 | +++ |
| 76 | ++ |
| 77 | +++ |
| 78 | +++ |
| 79 | |
| 80 | + |
| 81 | + |
| 82 | + |
| 83 | + |
| 84 | ++, + |
| 85 | + |
| 86 | ++ |
| 87 | + |
| 88 | ++ |
| 89 | + |
| 90 | + |
| 91 | +++ |
| 92 | + |

### Example 2A

### Compounds can Modulate Activity of ΔF508 CFTR

Ussing Chamber Assay: Fischer rat thyroid (FRT) cells stably expressing ΔF508 CFTR (cystic fibrosis transmembrane conductance regulator) were cultured as previously described in Am J. Physiol. (1994) 266 L405-413. Monolayers of cells were grown on Snapwell inserts (Corning Inc.) at the air/liquid interface. The monolayers were treated with compound for 24 hours. The inserts are mounted in Ussing chambers (Harvard Apparatus) and short-circuit currents are measured using a voltage clamp apparatus (WPI, Inc.). A mucosal to serosal gradient in chloride concentration is imposed and the basolateral membrane is permeabilized using amphotericin. Short circuit currents are measured upon addition of the agonists forskolin, isobutylmethylxanthine, and genistein to maximally activate CFTR.

In this assay compound 25 (see Table I for structure) showed a short circuit current after the addition of all three agonists of ˜65 uA/cm². This is comparable to the cold correction controls, which demonstrated ˜70 uA/cm² in these experiments, and indicates that the compounds are capable of modulating the activity of ΔF508 CFTR.

### Example 2B

### Defects in ΔF508 CFTR Trafficking are Corrected by the Compounds

Fisher rat thyroid (FRT) cells stably expressing ΔF508 CFTR and a halide-sensitive variant of yellow fluorescent protein (YFP) were seeded into microtiter plates and allowed to grow for 24 hours at 37° C. and 5% CO₂. See Pedemonte et al., J. Clin. Invest. 115(9) 2564-2571 (2005), the entire teachings of which are incorporated herein by reference. Compounds in dimethylsulfoxide (DMSO) solution were pre-diluted into cell culture medium, the medium was removed from cells, and then fresh medium containing the compounds was applied. The cells were incubated for a further 24 hours at 37° C. and 5% CO₂.

Activity of CFTR was assayed by removing the medium, washing the cell monolayer with phosphate-buffered saline solution (PBS), and then applying PBS containing forskolin and genistein. After 30 min incubation at 37° C., the plates were placed in a fluorescence plate reader equipped with a reagent injector. After measuring an initial fluorescence value, iodide-containing buffer was injected and the decrease in fluorescence was followed at excitation and emission wavelengths of 485 and 530 nm, respectively.

Corrector activity was calculated as follows. Normalized endpoint fluorescence was calculated by dividing the endpoint fluorescence after iodide injection by the initial fluorescence reading and multiplication by 100. Corrector EC₅₀ values were calculated from the activity vs. concentration data using a 4-parameter log fit. The bottom of the curve was constrained to zero activity (DMSO control) while the slope, EC₅₀, and top of the curve were fitted to the data. The EC₅₀ was determined as the concentration that corresponds to the inflection point of the fitted curve. The corrector activity EC₅₀ values for the various compounds were measured and are shown in Table III, in the following ranges: less than two micromolar, indicated by ++++; 2-5 micromolar, indicated by +++, 5-10 micromolar, indicated by ++; and 15 micromolar or greater, indicated by +.

For certain compounds, activity was analyzed compared to the DMSO control but an EC₅₀ value was not obtained. Compounds which nevertheless displayed activity compared to the DMSO control are indicated by #, and compounds which did not display such activity are indicated by Compounds tested for activity compared to the DMSO control at 25 micromolar are labeled with a single # or *. Compounds tested for activity compared to the DMSO control at 25 and 2.5 micromolar, respectively, are labeled with two such symbols. For example, “#, #” means activity was observed at both 25 and 2.5 micromolar concentrations; “*, #” means no activity was observed at 25 micromolar but activity was observed at 2.5 micromolar; “#,*” means activity was observed at 25 but not 2.5 micromolar; “*, *” means no activity observed at either concentration. These results are also shown in Table III.

TABLE III

| ID | Corrector EC50 |
|---|---|
| 1 | + |
| 2 | ++ |
| 3 | + |
| 4 | + |
| 5 | + |
| 6 | + |
| 7 | #, # |
| 8 | + |
| 10 | + |
| 11 | #, # |
| 12 | #, # |
| 13 | #, # |
| 16 | + |
| 17 | #, # |
| 19 | *, # |
| 20 | *, * |
| 21 | *, # |
| 22 | #, # |
| 23 | #, # |
| 24 | +++ |
| 25 | +++ |
| 27 | +++ |
| 29 | ++ |
| 30 | + |
| 31 | ++ |
| 32 | + |
| 33 | ++ |
| 34 | +++ |
| 37 | #, # |
| 38 | + |
| 39 | + |
| 40 | + |
| 41 | + |
| 42 | #, # |
| 43 | + |
| 44 | + |
| 46 | + |
| 47 | + |
| 48 | #, # |
| 49 | #, * |
| 50 | #, # |
| 51 | *, # |
| 52 | #, # |
| 53 | ++ |
| 55 | *, # |
| 57 | #, # |
| 58 | #, # |
| 59 | #, # |
| 60 | *, # |
| 61 | #, # |
| 62 | #, # |
| 63 | #, # |
| 65 | #, # |
| 67 | + |
| 69 | #, # |
| 70 | *, # |
| 71 | #, # |
| 72 | *, # |
| 73 | #, # |
| 74 | + |
| 75 | #, # |
| 76 | *, # |
| 77 | + |
| 78 | #, # |
| 80 | + |
| 81 | #, # |
| 82 | #, # |
| 83 | #, # |
| 84 | *, # |
| 86 | +++ |
| 87 | *, # |
| 89 | #, # |
| 90 | + |
| 92 | #, # |
| 93 | #, # |
| 94 | ++ |
| 95 | + |
| 96 | #, # |
| 97 | +++ |
| 98 | #, # |
| 99 | + |
| 100 | #, # |
| 101 | + |
| 102 | #, # |
| 103 | #, # |
| 104 | + |
| 105 | #, # |
| 107 | #, # |
| 110 | #, # |
| 111 | #, # |
| 114 | #, # |
| 115 | #, # |
| 116 | #, # |
| 117 | + |
| 118 | + |
| 119 | *, # |
| 120 | #, # |
| 123 | #, * |
| 124 | #, # |
| 125 | + |
| 126 | *, # |
| 127 | #, # |
| 128 | *, # |
| 129 | ++ |
| 131 | + |
| 132 | + |
| 134 | *, # |
| 135 | #, # |
| 139 | *, # |
| 140 | #, # |
| 141 | #, # |
| 142 | *, # |
| 143 | *, # |
| 144 | #, # |
| 145 | #, # |
| 146 | #, # |
| 149 | #, # |
| 150 | #, # |
| 151 | #, # |
| 152 | #, # |
| 153 | #, # |
| 154 | #, * |
| 155 | #, # |
| 156 | *, # |
| 157 | #, # |
| 158 | #, # |
| 162 | #, # |
| 163 | #, # |
| 164 | + |
| 165 | ++ |
| 167 | + |
| 168 | + |
| 169 | ++ |
| 170 | ++ |
| 171 | + |
| 172 | + |
| 173 | + |
| 174 | + |
| 175 | + |
| 176 | + |
| 177 | + |
| 178 | + |
| 179 | + |
| 180 | ++ |
| 181 | + |
| 182 | ++ |
| 183 | + |
| 184 | ++ |
| 185 | + |
| 186 | + |
| 187 | ++ |
| 188 | ++ |
| 189 | + |
| 190 | ++ |
| 191 | + |
| 192 | +++ |
| 193 | + |
| 194 | + |
| 195 | ++ |
| 196 | ++ |
| 197 | ++ |
| 198 | + |
| 199 | #, # |
| 200 | + |
| 201 | ++ |
| 202 | ++ |
| 203 | + |
| 205 | + |
| 206 | + |
| 207 | + |
| 208 | + |
| 209 | + |
| 210 | ++ |
| 211 | +++ |
| 212 | + |
| 213 | ++ |
| 214 | + |
| 215 | + |
| 216 | + |
| 217 | + |
| 218 | ++ |
| 219 | +++ |
| 220 | + |
| 221 | + |
| 222 | + |
| 223 | + |
| 224 | + |
| 226 | + |
| 227 | + |
| 228 | + |
| 229 | + |
| 230 | + |
| 231 | + |
| 232 | + |
| 233 | + |
| 234 | + |
| 235 | + |
| 236 | + |
| 237 | + |
| 238 | + |
| 239 | + |
| 240 | ++ |
| 241 | + |
| 242 | ++++ |
| 243 | + |
| 244 | + |
| 245 | + |
| 246 | ++ |
| 247 | +++ |
| 248 | + |
| 249 | + |
| 250 | + |
| 251 | + |
| 252 | +++ |
| 253 | + |
| 254 | + |
| 255 | + |
| 256 | + |
| 257 | ++++ |
| 258 | + |
| 259 | + |
| 260 | + |
| 261 | ++++ |
| 262 | +++ |
| 263 | + |
| 264 | + |
| 265 | ++ |
| 266 | ++ |
| 267 | + |
| 268 | + |
| 269 | + |
| 270 | + |
| 271 | ++++ |
| 272 | ++++ |
| 273 | + |
| 274 | +++ |
| 275 | + |
| 276 | ++ |
| 277 | + |
| 278 | + |
| 279 | + |
| 280 | + |
| 281 | + |
| 282 | + |
| 283 | + |
| 284 | + |
| 285 | + |
| 286 | + |
| 287 | + |
| 288 | + |
| 289 | ++ |
| 290 | ++++ |
| 291 | + |
| 292 | + |
| 293 | + |
| 294 | + |
| 295 | + |
| 296 | + |
| 297 | + |
| 298 | ++++ |
| 299 | ++++ |
| 300 | ++ |
| 301 | ++ |
| 302 | ++++ |
| 303 | + |
| 304 | ++ |
| 305 | + |
| 306 | + |
| 307 | + |
| 308 | ++ |
| 309 | +++ |
| 310 | ++++ |
| 311 | +++ |
| 312 | + |
| 313 | + |
| 314 | + |
| 315 | ++++ |
| 316 | ++ |
| 317 | + |
| 318 | + |
| 319 | + |
| 320 | + |
| 321 | ++++ |
| 322 | +++ |
| 323 | + |
| 324 | + |
| 325 | + |
| 326 | + |
| 327 | + |
| 328 | + |
| 329 | + |
| 330 | + |
| 331 | + |
| 332 | + |
| 333 | +++ |
| 334 | + |
| 335 | ++ |
| 336 | ++++ |
| 337 | ++++ |
| 338 | ++ |
| 339 | ++ |
| 340 | + |
| 341 | + |
| 342 | + |
| 343 | + |
| 344 | + |
| 345 | + |
| 346 | + |
| 347 | + |
| 348 | + |
| 349 | +++ |
| 350 | ++++ |
| 351 | +++ |
| 352 | ++++ |
| 353 | + |
| 354 | + |
| 355 | +++ |
| 356 | ++++ |
| 357 | + |
| 358 | + |
| 359 | + |
| 360 | + |
| 361 | + |
| 362 | + |
| 363 | + |
| 364 | ++++ |
| 365 | +++ |
| 366 | + |
| 367 | ++ |
| 368 | ++ |
| 369 | +++ |
| 370 | + |
| 371 | ++++ |
| 372 | +++ |
| 373 | ++ |
| 374 | ++++ |
| 375 | ++++ |
| 376 | +++ |
| 377 | + |
| 378 | + |
| 379 | + |
| 380 | + |
| 381 | + |
| 382 | + |
| 383 | + |
| 384 | + |
| 385 | + |
| 386 | + |
| 387 | + |
| 388 | + |
| 389 | + |
| 390 | + |
| 391 | +++ |
| 392 | + |
| 393 | ++ |
| 394 | + |
| 395 | ++ |
| 396 | ++ |
| 397 | + |
| 398 | + |
| 399 | + |
| 400 | + |
| 401 | +++ |
| 402 | + |
| 403 | + |
| 404 | + |
| 405 | + |
| 411 | + |
| 412 | + |
| 413 | + |
| 414 | + |
| 415 | + |
| 416 | + |
| 417 | + |
| 418 | + |
| 419 | + |
| 420 | + |
| 421 | +++ |
| 422 | +++ |
| 423 | ++++ |
| 424 | + |
| 425 | +++ |
| 426 | ++ |

### Example 2C

### ΔF508 CFTR Trafficking is Potentiated by the Compounds

Fisher rat thyroid (FRT) cells stably expressing ΔF508 CFTR and a halide-sensitive variant of yellow fluorescent protein (YFP) were seeded into microtiter plates and allowed to grow for 24 hours at 37° C. and 5% CO₂, as described above. The medium was replaced with fresh medium and the cells were incubated for a further 24 hours at 27° C. and 5% CO₂ to increase the level of mutant CFTR at the cell surface.

Compounds in DMSO solution were pre-diluted into phosphate-buffered saline solution (PBS) containing forskolin. Activity of CFTR was assayed by removing the medium, washing the cell monolayer with PBS, and then applying the compounds diluted in PBS containing forskolin. After 30 min of incubation at 37° C., the plates were placed in a fluorescence plate reader equipped with a reagent injector. After measuring an initial fluorescence value, iodide-containing buffer was injected and the decrease in fluorescence was followed at excitation and emission wavelengths of 485 and 530 nm, respectively.

Potentiator activity was calculated as follows. Normalized endpoint fluorescence was calculated by dividing the endpoint fluorescence after iodide injection by the initial fluorescence reading and multiplication by 100. Potentiator EC₅₀ values were calculated by the same method as corrector EC₅₀ values. Table IV shows the potentiator activity for the various compounds according to the same symbolic scheme described above for the corrector activities.

TABLE IV

| ID | Potentiator EC50 |
|---|---|
| 2 | ++++ |
| 4 | # |
| 7 | # |
| 8 | # |
| 10 | ++++ |
| 11 | # |
| 13 | # |
| 14 | # |
| 30 | # |
| 31 | + |
| 32 | # |
| 33 | ++++ |
| 34 | ++++ |
| 35 | # |
| 38 | + |
| 39 | + |
| 42 | # |
| 43 | + |
| 44 | + |
| 46 | + |
| 47 | + |
| 49 | # |
| 57 | # |
| 67 | +++ |
| 69 | * |
| 74 | # |
| 77 | # |
| 78 | # |
| 80 | + |
| 83 | # |
| 86 | ++ |
| 90 | ++++ |
| 92 | * |
| 93 | * |
| 94 | + |
| 95 | +++ |
| 97 | +++ |
| 98 | * |
| 99 | +++ |
| 101 | # |
| 104 | +++ |
| 118 | + |
| 131 | +++ |
| 132 | + |
| 139 | * |
| 156 | # |
| 157 | # |
| 158 | # |
| 165 | ++++ |
| 167 | ++++ |
| 168 | ++++ |
| 169 | ++++ |
| 170 | + |
| 172 | ++++ |
| 173 | + |
| 174 | ++++ |
| 175 | ++++ |
| 176 | ++++ |
| 177 | ++++ |
| 178 | +++ |
| 179 | ++++ |
| 180 | ++++ |
| 183 | + |
| 184 | ++++ |
| 185 | ++++ |
| 187 | + |
| 188 | + |
| 189 | ++++ |
| 190 | + |
| 191 | ++ |
| 193 | + |
| 194 | + |
| 195 | + |
| 196 | ++++ |
| 197 | ++++ |
| 198 | # (at 12 μm) |
| 200 | ++++ |
| 201 | ++ |
| 202 | + |
| 203 | # |
| 205 | ++++ |
| 206 | + |
| 207 | ++++ |
| 208 | ++++ |
| 209 | + |
| 210 | ++++ |
| 212 | + |
| 216 | ++++ |
| 217 | + |
| 218 | ++++ |
| 220 | + |
| 224 | + |
| 226 | ++++ |
| 227 | + |
| 228 | ++++ |
| 233 | + |
| 236 | ++++ |
| 237 | + |
| 238 | + |
| 240 | ++++ |
| 241 | + |
| 242 | +++ |
| 244 | ++ |
| 245 | ++ |
| 246 | + |
| 247 | +++ |
| 252 | ++ |
| 256 | ++++ |
| 257 | ++++ |
| 261 | ++++ |
| 262 | +++ |
| 263 | + |
| 264 | +++ |
| 265 | ++++ |
| 266 | ++++ |
| 267 | +++ |
| 268 | + |
| 269 | + |
| 270 | + |
| 271 | ++ |
| 272 | + |
| 273 | + |
| 274 | + |
| 275 | + |
| 276 | ++++ |
| 277 | + |
| 278 | ++++ |
| 279 | + |
| 280 | + |
| 281 | + |
| 282 | + |
| 283 | + |
| 284 | + |
| 285 | + |
| 286 | + |
| 287 | + |
| 288 | + |
| 289 | +++ |
| 290 | +++ |
| 291 | + |
| 292 | + |
| 293 | + |
| 294 | + |
| 295 | + |
| 296 | + |
| 297 | +++ |
| 298 | + |
| 299 | + |
| 300 | + |
| 301 | ++++ |
| 302 | + |
| 303 | + |
| 304 | + |
| 305 | + |
| 306 | + |
| 307 | + |
| 308 | + |
| 309 | + |
| 310 | + |
| 311 | ++ |
| 312 | ++ |
| 313 | +++ |
| 314 | + |
| 315 | +++ |
| 316 | +++ |
| 317 | + |
| 318 | + |
| 319 | + |
| 320 | +++ |
| 321 | ++++ |
| 322 | +++ |
| 323 | ++++ |
| 324 | + |
| 325 | + |
| 326 | + |
| 327 | ++++ |
| 328 | + |
| 329 | + |
| 330 | + |
| 331 | +++ |
| 332 | ++++ |
| 333 | ++++ |
| 334 | + |
| 335 | + |
| 336 | ++ |
| 341 | ++ |
| 342 | + |
| 343 | ++++ |
| 344 | + |
| 345 | +++ |
| 346 | + |
| 347 | + |
| 349 | +++ |
| 350 | + |
| 351 | ++++ |
| 352 | ++++ |
| 353 | ++ |
| 356 | + |
| 411 | + |
| 412 | + |
| 413 | + |

### Example 2D

### Certain Compounds Both Potentiate ΔF508 CFTR and Correct Defects in ΔF508 CFTR Trafficking

Fisher rat thyroid (FRT) cells stably expressing ΔF508 CFTR and a halide-sensitive variant of yellow fluorescent protein (YFP) were seeded into microtiter plates and allowed to grow for 24 hours at 37° C. and 5% CO₂, as described above. Compounds in DMSO solution were pre-diluted into cell culture medium, the medium was removed from cells, and fresh medium containing compounds was applied. The cells were incubated a further 24 hours at 37° C. and 5% CO₂.

Activity of CFTR was assayed after adding a small volume of a stock solution of forskolin in DMSO, and then mixing well. After 30 min incubation at 37° C., the plates were placed in a fluorescence plate reader equipped with a reagent injector. After measuring an initial fluorescence value, iodide-containing buffer was injected and the decrease in fluorescence was followed at excitation and emission wavelengths of 485 and 530 nm, respectively.

Dual corrector and potentiator activity was calculated as follows. Normalized endpoint fluorescence was calculated by dividing the endpoint fluorescence after iodide injection by the initial fluorescence reading and multiplication by 100. The activity of the negative control, DMSO, was assigned a value of 0%. Dual activity EC₅₀ values were calculated by the same method as corrector EC₅₀ values.

The following is a list of compounds that have been put through the assay (the numbers correspond to Table I). Some of the compounds showed low response at the highest concentration tested.
2
5
16
24
25
27
29
31
33
34
67
86
95
97
118
129
165
202
240
242
246
247
257
261
265
266
270
271
276
289
290
300
311
315
332
333
336
337
348
349
350
351
355
366
401

### Example 3

### Rescue of Cell Viability from α-Synuclein-Induced Cytotoxicity

Treatment of TS217 cells with 0.1 μg/mL tetracycline for three to six days induces expression of α-synuclein, which can be cytotoxic. To determine inhibition of α-synuclein-induced cytotoxicity, TS217 cells plated in 96 well tissue culture plates were cultured with 0.1 μg/mL tetracycline for 5 days in the presence of either compound 90 (see Table I for structure) (0.08 μM, 0.15 μM, and 0.3 μM) or DMSO as a control or in the presence of either forskolin (0.3 μM, 1 μM, 3 μM, and 10 μM) or DMSO as a control. After the five day treatment, cells were lysed and assayed for intracellular ATP concentration as a function of cell viability. The relative viability of the cells was assessed by measuring the cellular ATP level in cell lysates using a VIALIGHT® Plus Bioassay kit (Cambrex, Rockland, Me.). Relative cell viability was calculated as the ratio of induced cells to control cells (cells not treated with tetracycline), as an indication of α-synuclein-induced cytotoxicity. Relative cell viability decreased by over 50% from day 3 to day 6 in the absence of any other toxicity-inducing agents, indicating that expression of α-synuclein alone in these cells was capable of causing cell death. By contrast, cells treated with compound 90 at 3 μM concentration reduced the α-synuclein-induced cytotoxicity by 45% (P<0.02) as compared to the control wells lacking the compound. Thus, compound 90 rescues cell viability from α-synuclein-induced cytotoxicity.

### Example 4

### α-Synuclein (aS) Screening

Yeast Strains

Parental W303: MAT a/α ade2-1/ade2-1 his3-11,15/his3-11,15 leu2-3,112/leu2-3,112

trp1-1/trp1-1 ura3-1/ura3-1 can1-100/can1-100

Phenotype: Requires adenine, histidine, leucine, tryptophan, and uracil for growth. Resistant to canavanine.

Fx-109: MAT a/α ade2-1/ade2-1 his3-11,15/his3-11,15 leu2-3,112/leu2-3,112

trp1-1/trp1-1 GALp-aS-GFP::TRP1/GALp-aS-GFP::TRP1 ura3-1/ura3-1

GALp-aS-GFP::URA3/GALp-aS-GFP::URA3 can1-100/can1-100 pdr1::KanMX/pdr1::KanMX erg6::KanMX/erg6::KanMX

Phenotype: Unable to grow on galactose due to expression of aS. Requires histidine, leucine, and adenine for growth. Resistant to canavanine and kanamycin. Hypersensitive to drugs.

Media and Reagents

Based on the genotype of the strain to be tested, choose the appropriate supplementation for the synthetic media. Strains containing integrated constructs (eg, aS) can be grown in medium which maintains selection for the construct (see below). CSM (Qbiogene) is a commercially-available amino acid mix for growing Saccharomyces cerevisiae. It can be obtained lacking one or more amino acids as required. For the aS and control strains, media lacking tryptophan and uracil (-Trp-Ura) can be used (available from Qbiogene, Inc., Carlsbad, Calif.).

To make liquid synthetic medium, mix the components listed in Tables B, C, and D. After the components have dissolved, sterilize by filtration (Millipore Stericup Cat#SCGPU11RE) into a sterile bottle.

TABLE B
Synthetic Complete Medium

| | | | | Amount | | |
|---|---|---|---|---|---|---|
| Component | Vendor | Catalogue # | Size | per L | | Final Conc. |
| Yeast Nitrogen | Difco | 291920 | 2 kg | 6.7 | g | 0.67% (w/v) |
| Base without | | | | | | |
| amino acids | | | | | | |
| Carbon source: | See below | See below | See below | 20 | g | 2% (w/v) |
| one of glucose, | | | | | | |
| galactose, | | | | | | |
| raffinose | | | | | | |
| CSM: strain | Qbiogene | See below | See below | ~0.8 | g | |
| determines | | | | (according | | |
| type | | | | to mfr) | | |
| MilliQ Water | — | — | | 1 | L | — |

TABLE C
Carbon Sources

| | | | | | | |
|---|---|---|---|---|---|---|
| Glucose (also known | Fisher | D16-10 | 10 | kg | 20 g | 2% (w/v) |
| as dextrose) | | | | | | |
| Galactose | SIGMA | G-0750 | 1 | kg | 20 g | 2% (w/v) |
| Raffinose | Difco | 217410 | 100 | g | 20 g | 2% (w/v) |

TABLE D
CSM

| | | | | | |
|---|---|---|---|---|---|
| CSM-Trp-Ura | Qbiogene | 4520-522 | 100 g | 0.72 g | See Qbiogene |
| for aS and | | | | | web page |
| control strain | | | | | |
| CSM for the | Qbiogene | 4500-022 | 100 g | 0.79 g | See Qbiogene |
| parental strain | | | | | web page |

384-Well Screening Protocol Using Optical Density

Day 1

Innoculate an appropriate volume of SRaffinose-Trp-Ura medium with Fx-109 strain.

Incubate with shaking at 30° C. overnight until cells reach log or mid-log phase (OD₆₀₀ 0.5-1.0; 0.1 OD600 corresponds to ˜1.75×10 E6 cells).

Day 2

Spin down cells at room temperature, remove medium, and resuspend in an equivalent volume of SGalactose-Trp-Ura medium. Measure the OD₆₀₀ and dilute cells to 0.001. Robotically transfer 30 μl of cell suspension (MicroFill, Biotek) to each well of a 384-well plate (NUNC 242757).

Add 100 nl drug in DMSO (Cybio) to each well (final conc. 17 μg/ml drug and 0.333% DMSO)

For the positive controls add glucose to final concentrations of 0.1% and 1%.

Incubate plates at 30° C. without shaking in a humidified chamber for 24 and/or 48 hours.

Day 3 (24 Hours Later) and/or Day 4 (48 Hours Later)

Read OD₆₅₀ (Envision, Perkin Elmer) and also visually inspect wells for growth of yeast culture.

### Example 5

### ypt1^{ts} Mutant Active Compounds Can Stabilize ΔF508 CTFR

The compounds can be tested for their ability to stabilize ΔF508 CTFR. CFBE cells, a cell line generated by transformation of cystic fibrosis tracheo-bronchial cells (ΔF508 CTFR homozygous) with SV40 (Bruscia et al. (2002) Gene Ther. 9(11):683-685), can be cultured with 10 μM of the selected compounds, or 10 μM VRT-325 for 16 hours at 37° C. (VRT-325 is described in, e.g., Van Goor et al. (2006) Am. J. Physiol. Lung Cell Mol. Physiol. 290:L1117-L1130). A population of cells can also be cultured with the dimethyl sulfoxide (DMSO) solvent as a control.

Following incubation, cells were lysed, solubilized in Laemmli buffer, and subjected to SDS-PAGE. CFTR protein were visualized by western blotting using an antibody specific for CFTR. Culturing CFBE cells with compound 25 (see Table I for structure) increased the amount of cellular ΔF508 CFTR protein. This compound also increased the amount of the glycosylated form of ΔF508 CFTR indicates increased trafficking of this protein through the Golgi apparatus. The effects of compound 25 on stabilizing ΔF508 CFTR is comparable or better than the effects of the known CFTR stabilizer VRT-325.

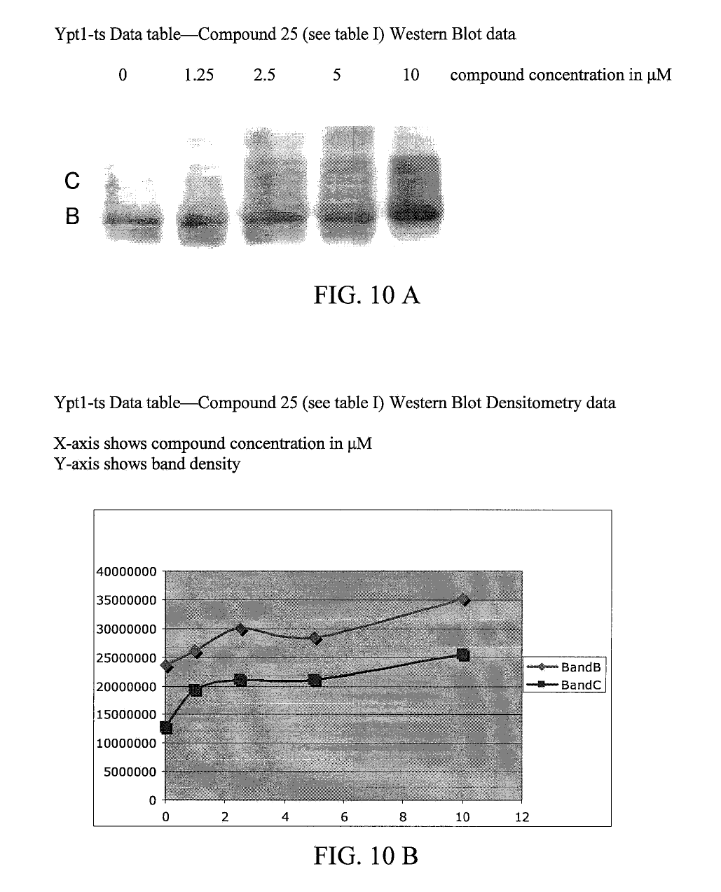

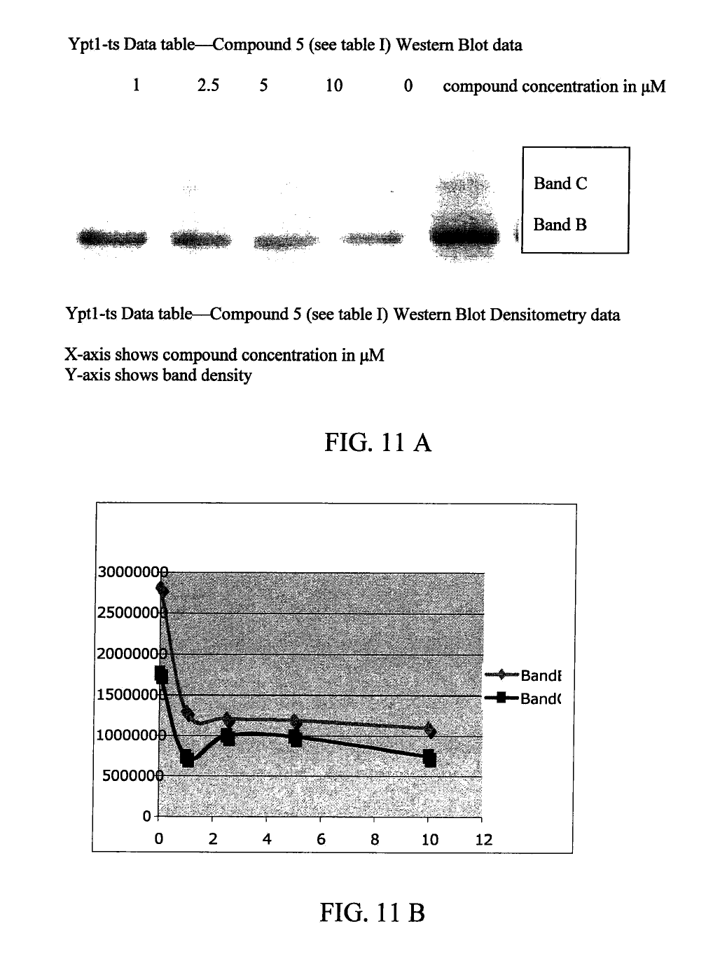

The effect of different concentrations of compound 25 on ΔF508 CFTR was tested in a dose response experiment. CFBE cells were grown at 37° C. for 16 hours in the presence of 0, 1.25, 2.5, 5, or 10 μM of compound 25. Following incubation, lysates were prepared from the treated cells, the lysates were solubilized in Laemmli buffer, and were then subjected to SDS-PAGE. The relative amounts of glysosylated and unglycosylated ΔF508 CFTR protein were visualized by western blotting (FIG. 10A), and the band intensities wer quantitated by scanning and densitometry (FIG. 10B). As compared to the amount of protein in the absence of compound 25, the concentrations tested (1.25-10 μM) showed increased glycosylated (Band B) and unglycosylated (Band C) ΔF508 CFTR proteins. Compound 5 (Table I) was also tested. CFBE cells were grown at 37° C. for 16 hours in the presence of 0, 1, 2.5, 5, or 10 μM of compound 5. Following incubation, lysates were prepared from the treated cells, the lysates were solubilized in Laemmli buffer, and were then subjected to SDS-PAGE. The relative amounts of glysosylated and unglycosylated ΔF508 CFTR protein were visualized by western blotting (FIG. 11A), and the band intensities wer quantitated by scanning and densitometry (FIG. 11B). As compared to the amount of protein in the absence of compound 5, the concentrations tested (1-10 μM) showed increased glycosylated (Band B) and unglycosylated (Band C) ΔF508 CFTR proteins.

These data indicate that compounds identified in the ypt1^{ts} mutant rescue screening assay such as compound 25 can stabilize ΔF508 CFTR protein and thus are useful in treating cystic fibrosis.

### Example 6

### Compounds can Restore Growth of a sar1^{ts} Mutant

The sar1^{ts} mutant yeast strain (ATCC, Manassas, Va.) carries a temperature sensitive mutant allele of the SAR1 gene, which can permit the strain to grow at 25° C., but undergo growth arrest at 35° C. or higher. Inactivation of the mutant Sar1^{ts} protein at 35° C. can prevent the formation of transport vesicles at the ER, causing a block in ER to golgi trafficking (Saito et al. (1998) J. Biochem. (Tokyo) 124(4):816-823).

To identify compounds that rescue the sar1^{ts} mutant phenotype, the mutant strain can be first grown at 25° C. in rich media overnight. The strain can be diluted to an OD₆₀₀ of 0.004 in SC media with 2% glucose, and mixed with various dilutions of test compounds (0.05 to 50 μM) in media with 2% glucose. The cells can be incubated at 25° C. or 35° C. for 72 hours. Rescue of the sar1^{ts} mutant phenotype can be scored as an increase in the OD₆₀₀ (concentration of the yeast cells) cultured in the presence of a test compound as compared to cells cultured in the absence of the test compound.

Control compounds can be used, such as cycloheximide and hygromycin, which can rescue the sar1^{ts} mutant phenotype. Compounds which increase in the OD₆₀₀ concentration are active compounds.

### Example 7

### Compounds can Restore Growth of a sec23^{ts} Mutant

The sec23-2^{ts} mutant yeast strain carries a temperature sensitive mutant allele of the SEC23 gene, which can permit the strain to grow normally at 25° C., but can undergo growth arrest at 30° C. or higher. Inactivation of the Sec23 temperature-sensitive mutant protein at the restrictive temperature can prevent the formation of transport vesicles at the ER resulting in a block in ER to golgi trafficking (see, e.g., Hicke et al. (1989) EMBO J. 8(6):1677-1684 and Castillo-Flores et al. (2005) J. Biol. Chem. 280(40):34033-34041).

To identify compounds that rescue the sec23^{ts} mutant phenotype, the mutant strain can be first grown at 25° C. in rich media overnight. The strain can be diluted to an OD₆₀₀ of 0.004 in SC media with 2% glucose, and mixed with various dilutions of test compounds (0.05 to 50 μM) in media with 2% glucose. The cells can be incubated at 25° C. or 30° C. for 24 hours. Rescue of the sec23^{ts} mutant phenotype can be scored as an increase in the OD₆₀₀ of cells cultured in the presence of the a compound as compared to cells cultured in the absence of the test compound. Compounds which increase in the OD₆₀₀ concentration are active compounds.

### Synthetic Examples

Compounds described herein were prepared using the schemes and processes described above and further exemplified as set forth below.

### Example 8

### 3-(4-Chloro-phenyl)-1-cyclopropylmethyl-1H-pyrazolo[3,4-d]pyrimidin-4-ylamine (Compound No. 29)

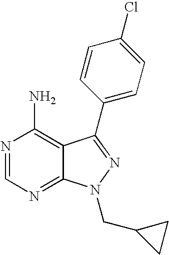

Step A:

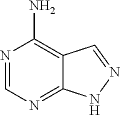

A mixture of commercially available 5-amino-1H-pyrazole-4-carbonitrile (16.22 g, 0.15 mol) and formamide (84.6 ml) was heated at 180° C. for 4 hr under a nitrogen atmosphere. The solution was cooled to ambient temperature and the crystals were separated, washed with water and dried to afford 1H-pyrazolo[3,4-d]pyrimidin-4-ylamine product (18.6 g, 0.13 mol).

Step B:

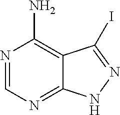

A mixture of 1H-pyrazolo[3,4-d]pyrimidin-4-ylamine (11.75 g, 0.09 mol) (Step A) and N-iodosuccinimide (25.45 g, 0.11 mol) in dimethylformamide (300 ml) was stirred at 50° C. for 24 hr. A second batch of N-iodosuccinimide (3.92 g, 0.02 mol) was added and the solution stirred for additional 24 hr. Upon standing at room temperature, a precipitate was formed which was separated by filtration and washed with dimethylformamide and ethanol to afford 10.05 g of 3-iodo-1H-pyrazolo[3,4-d]pyrimidin-4-ylamine. The filtrate was concentrated in vacuo to about one half of the original volume and 500 ml of water was added. The precipitated product was separated by filtration and washed with ethanol to afford a second batch of the product (10.53 g, combined yield 20.58 g, 0.08 mol); LC/MS, API-ES, Pos, (M+H)⁺, 262.1.

Step C:

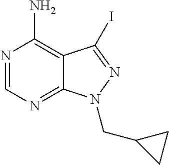

3-Iodo-1H-pyrazolo[3,4-d]pyrimidin-4-ylamine (1.0 g, 3.83 mmol) (Step B), cyclopropyl-methanol (0.83 g, 11.51 mmol) and triphenylphosphine (2.01 g, 7.66 mmol) were dissolved in anhydrous tetrahydrofuran (50 ml) and stirred at 0° C. Diethylazodicarboxylate (1.33 g, 7.63 mmol) was slowly added and the solution stirred at 0° C. for 15 min. Solution was allowed to warm to room temperature and stirred for 1 hr. Solvent was evaporated in vacuo and product adsorbed on silica gel. Flash chromatography on silica gel (eluent, hexane:ethyl acetate, 50:50 to 20:80) followed by trituration with acetonitrile afforded 1-cyclopropylmethyl-3-iodo-1H-pyrazolo[3,4-d]pyrimidin-4-ylamine (0.77 g, 2.44 mmol); LC/MS, API-ES, Pos, (M+H)⁺, 316.1.

Step D:

1-Cyclopropylmethyl-3-iodo-1H-pyrazolo[3,4-d]pyrimidin-4-ylamine (0.12 g, 0.38 mmol) (Step C), 4-chlorophenylboronic acid (0.65 g, 0.42 mmol), tetrakistriphenylphosphine palladium (0.03 g, 0.02 mmol) and sodium carbonate (0.09 g, 0.85 mmol) were mixed in 1,2-dimethoxyethane (10 ml) and water (5 ml) and the solution refluxed under argon for 6 hr. Water was added and the product was extracted with ethyl acetate (2×25 ml). Evaporation of the solvent followed by flash chromatography on silica gel (eluent, hexane:ethyl acetate, 50:50 to 10:90) afforded the title compound (0.04 g, 0.13 mmol); LC/MS, API-ES, Pos, (M+H)⁺, 300.1.

### Example 9

### 1-tert-Butyl-3-(4-fluoro-phenyl)-1H-pyrazolo[3,4-d]pyrimidin-4-ylamine (Compound No. 9)

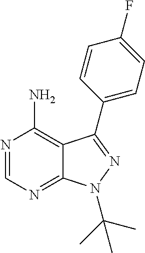

Step A:

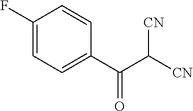

To a stirred solution of malononitrile (2.08 g, 31.5 mmol) in 50 ml of anhydrous tetrahydrofuran at 0° C. was slowly added sodium hydride (60%, 2.52 g, 63 mmol) in portions and solution stirred for 10 min. A solution of 4-fluorobenzoyl chloride (5.0 g, 31.5 mmol) in tetrahydrofuran (25 ml) was slowly added via an addition funnel and solution stirred at ambient temperature for 1 hr. Dilute hydrochloric acid (1 mol/L, 100 ml) was added and the product extracted with ethyl acetate. The organic layer was washed with water, brine, and evaporated to afford a residue which was triturated with hexane to afford 2-(4-fluoro-benzoyl)-malononitrile (4.98 g, 26.5 mmol); LC/MS, API-ES, Neg, (M−H)⁻, 187.0.

Step B:

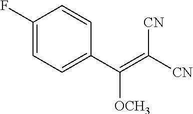

2-(4-Fluoro-benzoyl)-malononitrile (4.98 g, 26.47 mmol) (Step A) was dissolved in a mixture of anhydrous acetonitrile (100 ml) and methanol (10 ml) and trimethylsilyl diazomethane (2M solution in diethyl ether, 19.9 ml, 39.8 mmol) was added. Solution was stirred at 0° C. under a nitrogen atmosphere and N,N-diisopropylethylamine (6.84 g, 52.9 mmol) was slowly added. The solution was stirred at ambient temperature for 18 hr and solvent evaporated in vacuo. The residue was adsorbed on silica gel and purified by chromatography (eluent, hexane:ethyl acetate, 80:20 to 70:30) to afford 2-[(4-fluoro-phenyl)-methoxy-methylene]-malononitrile (2.83 g, 13.9 mmol) as an oil; LC/MS, API-ES, Pos, (M+H)⁺, 203.0.

Step C:

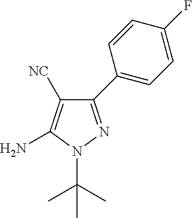

2-[(4-Fluoro-phenyl)-methoxy-methylene]-malononitrile (2.80 g, 13.85 mmol) (Step B) was dissolved in anhydrous ethanol (75 ml) and t-butylhydrazine hydrochloride (1.73 g, 13.88 mmol) was added followed by triethylamine (1.54 g, 15.27 mmol). The solution was refluxed for 2 hr and solvent evaporated. The product was purified by flash column chromatography on silica gel (eluent, hexane:ethyl acetate, 80:20 to 30:70) to afford 5-amino-1-tert-butyl-3-(4-fluoro-phenyl)-1H-pyrazole-4-carbonitrile (3.02 g, 11.7 mmol); LC/MS, API-ES, Pos, (M+H)⁺, 259.1.

Step D:

5-Amino-1-tert-butyl-3-(4-fluoro-phenyl)-1H-pyrazole-4-carbonitrile (0.82 g, 3.16 mmol) was mixed with formamide (5 ml) and the mixture heated at 180° C. under a nitrogen atmosphere for 3 hr. Upon cooling, the product separated as crystalline material which was separated by filtration, washed with water and dried to afford the title compound (0.73 g, 2.56 mmol); LC/MS, API-ES, Pos, (M+H)⁺, 286.1.

### Example 10

### 3-Benzo[b]thiophen-2-yl-1-tert-butyl-1H-pyrazolo[3,4-d]pyrimidin-4-ylamine (Compound No. 16)

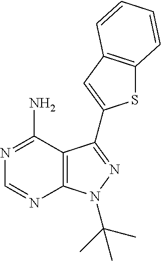

Step A:

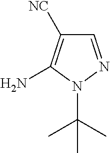

A mixture of t-butylhydrazine hydrochloride (4.67 g, 53 mmol) and triethylamine (5.35 g, 53 mmol) in anhydrous ethanol (250 ml) was stirred and ethoxymethylene malononitrile (6.47 g, 53 mmol) was slowly added in portions. The mixture was heated at reflux for 3 hr. The solvent was removed in vacuo and the product was crystallized from ethyl acetate-hexane followed by ether to afford 5-amino-1-tert-butyl-1H-pyrazole-4-carbonitrile as light pale brown crystals (5.6 g, 34.1 mmol); LC/MS, API-ES, Neg, (M−H)⁻, 163.0.

Step B:

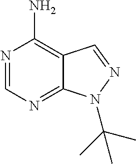

A mixture of 5-amino-1-tert-butyl-1H-pyrazole-4-carbonitrile (5.5 g, 33.5 mmol) (Step A) and formamide (68 ml) was heated at 185° C. for 3 hr under nitrogen atmosphere. The mixture was added to water and extracted with ethyl acetate. The organic layer was washed with saturated sodium bicarbonate solution followed by aqueous wash and brine. The organic layer was dried (anhydrous sodium sulfate) and the solvent was removed in vacuo to afford a residue which was crystallized from small amount of ether to afford 1-tert-butyl-1H-pyrazolo[3,4-d]pyrimidin-4-ylamine (Compound No. 12; 3.91 g, 20.4 mmol); LC/MS, API-ES, Pos, (M+H)⁺, 192.1.

Step C:

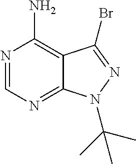

1-tert-Butyl-1H-pyrazolo[3,4-d]pyrimidin-4-ylamine (1.6 g, 8.37 mmol) (Step B) was suspended in water (30 ml) and bromine (2.68 g, 16.7 mmol) was added. The mixture was stirred at ambient temperature for 1 hr followed by stirring at 100° C. for 1 hr. After cooling, the precipitated product was separated by filtration. The residue was stirred in 50 ml of 5% aqueous sodium hydrogen sulfite solution for 0.5 hr and the solution was treated with 10 ml of saturated aqueous sodium bicarbonate. The precipitate was separated by filtration, washed with water and dried to afford 3-bromo-1-tert-butyl-1H-pyrazolo[3,4-d]pyrimidin-4-ylamine (Compound No. 107; 1.46 g, 5.40 mmol); LC/MS, API-ES, Pos, (M+H)⁺, 270.0 and 272.0.

Step D:

3-Bromo-1-tert-butyl-1H-pyrazolo[3,4-d]pyrimidin-4-ylamine (351 mg, 1.3 mmol) (Step C), benzo[b]thiophen-2-ylboronic acid (255 mg, 1.43 mmol), tetrakistriphenylphosphine palladium (90 mg, 0.07 mmol) and sodium carbonate (330 mg, 3.11 mmol) were mixed in 1,2-dimethoxyethane (20 ml) and water (10 ml) and the solution refluxed under argon for 6 hr. Water was added and the product was extracted with ethyl acetate (2×25 ml). Evaporation of the solvent followed by flash chromatography on silica gel (eluent, hexane:ethyl acetate, 80:20 to 65:35) afforded the title compound as an off-white powder (136 mg, 0.42 mmol); LC/MS, API-ES, Pos, (M+H)⁺, 324.1.

### Example 11

### 1-Ethyl-3-p-tolyl-1H-indol-4-ylamine (Compound No. 43)

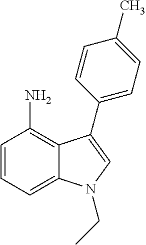

Step A:

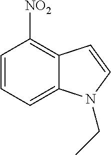

To a stirred solution of 4-nitroindole (2.5 g, 15.4 mmol) in 50 ml acetone at 0° C. was added 4.32 g (76.9 mmol) powdered potassium hydroxide and the solution stirred for 5 min. Ethyl iodide (4.8 g, 30.8 mmol) was added and the solution stirred vigorously for 15 min at ambient temperature. Toluene (300 ml) was added and the insoluble material was removed by filtration. The solution was washed with 5% aqueous citric acid followed by water, dried (anhydrous sodium sulfate) and solvent removed in vacuo. Residue was triturated with hexane-ethyl acetate (7:3) to afford 1-ethyl-4-nitro-1H-indole (2.6 g, 13.6 mmol); LC/MS, API-ES, Pos, (M+H)⁺, 191.1.

Step B:

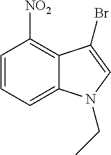

A solution of 1-ethyl-4-nitro-1H-indole (2.93 g, 15.4 mmol) (Step A) in anhydrous tetrahydrofuran (100 ml) was stirred at −78° C. N-bromosuccinimide (3.56 g, 20.0 mmol) was slowly added and the solution stirred at this temperature for 2 hr. Silica gel (8.0 g) was added and the solution evaporated in vacuo to afford a slurry that was flash chromatographed on silica gel (eluent, hexane:ethyl acetate, 90:10 to 80:20). 3-Bromo-1-ethyl-4-nitro-1H-indole was isolated as a pale yellow solid (2.48 g, 9.22 mmol); LC/MS, API-ES, Pos, (M+H)⁺, 269.0 and 271.0.

Step C:

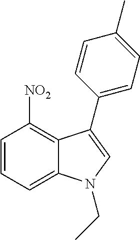

3-Bromo-1-ethyl-4-nitro-1H-indole (349.8 mg, 1.3 mmol) (Step B), 4-methylphenylboronic acid (194.4 mg, 1.43 mmol), tetrakistriphenylphosphine palladium (90.1 mg, 0.08 mmol) and sodium carbonate (330.7 mg, 3.12 mmol) were mixed in 1,2-dimethoxyethane (20 ml) and water (10 ml) and the solution refluxed under argon for 6 hr. Water was added and the product was extracted with ethyl acetate (3×25 ml). Evaporation of the solvent followed by flash chromatography on silica gel (eluent, hexane:ethyl acetate, 90:10 to 80:20) afforded 1-ethyl-4-nitro-3-p-tolyl-1H-indole (220 mg, 0.79 mmol); LC/MS, API-ES, Pos, (M+H)⁺, 281.1.

Step D:

1-Ethyl-4-nitro-3-p-tolyl-1H-indole (220 mg, 0.78 mmol) (Step C) was dissolved in a mixture of methanol and ethyl acetate (3:1, 50 ml) and 10 Pd/C (22 mg) was added. Hydrogen gas was bubbled gently through the solution for 2 hr. The catalyst was removed by filtration and the solvent evaporated. The product was purified by flash chromatography on silica gel (eluent, hexane:ethyl acetate, 90:10 to 80:20) to afforded the title compound (65 mg, 0.26 mmol) as a colorless oil; LC/MS, API-ES, Pos, (M+H)⁺, 251.2.

### Example 12

### 1-Ethyl-3-p-tolyl-1H-indazol-4-ylamine (Compound No. 44)

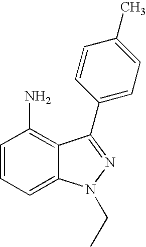

Step A:

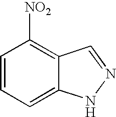

A solution of 2-methyl-3-nitro-phenylamine (5.5 g, 36.15 mmol) in glacial acetic acid (250 ml) was stirred at 0° C. Sodium nitrite (2.5 g, 36.15 mmol) dissolved in water (6 ml) was added to the stirred solution all at once and the stirring continued for 15 min. Yellow precipitate was removed by filtration and discarded and the solution stirred at ambient temperature for 4 hr. Solvent was removed in vacuo and water (20 ml) was added. The precipitate was separated by filtration and dried to afford the crude product. Chromatographic purification on silica gel (eluent, hexane:ethyl acetate, 70:30 to 50:50) afforded 4-nitro-1H-indazole (4.0 g, 24.52 mmol).

Step B:

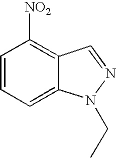

Sodium hydride (60%, 0.40 g, 10 mmol) was suspended in anhydrous dimethylformamide (8 ml) and stirred at −10° C. 4-Nitro-1H-indazole (1.0 g, 6.13 mmol) (Step A) dissolved in dimethylformamide (8 ml) was slowly added and the solution stirred for 20 min at this temperature. Ethyl iodide (1.05 g, 6.73 mmol) was added drop-wise and the solution stirred at ambient temperature for 2 hr. The solution was then poured on to ice-water and product extracted with methylene chloride. TLC and LC-MS analysis indicated the presence of two isomeric products that were separated by column chromatography on silica gel (eluent, hexane:ethyl acetate, 80:20 to 60:40) to afford 1-ethyl-4-nitro-1H-indazole (0.43 g, 2.24 mmol), LC/MS, API-ES, Pos, (M+H)⁺, 192.1, and the isomeric 2-ethyl-4-nitro-2H-indazole (0.48 g, 2.51 mmol); LC/MS, API-ES, Pos, (M+H)⁺, 192.1.

Step C:

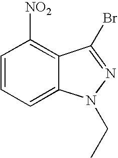

1-Ethyl-4-nitro-1H-indazole (0.43 g, 2.26 mmol) (Step B) was dissolved in glacial acetic acid (15 ml) and bromine (0.47 g, 2.94 mmol) was added. The solution was stirred at 80° C. for 30 min and a second batch of bromine (0.11 g, 0.68 mmol) was added and the solution stirred for an additional 30 min. Solution was added to a saturated aqueous solution of sodium bicarbonate and the product extracted with dichloromethane. Organic layer was washed with water and dried (anhydrous magnesium sulfate) and solvent evaporated in vacuo to afford a crude product. 3-Bromo-1-ethyl-4-nitro-1H-indazole was purified by flash column chromatography on silica gel (eluent, hexane:ethyl acetate, 80:20 to 70:30) (0.59 g, 2.18 mmol); LC/MS, API-ES, Pos, (M+H)⁺, 270.0 and 272.0.

Step D:

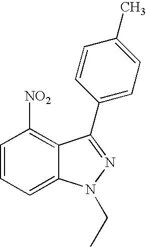

3-Bromo-1-ethyl-4-nitro-1H-indazole (0.59 g, 2.18 mmol) (Step C), 4-methylphenylboronic acid (0.36 g, 2.65 mmol), tetrakistriphenylphosphine palladium (0.15 g, 0.13 mmol) and sodium carbonate (0.55 g, 5.19 mmol) were mixed in 1,2-dimethoxyethane (20 ml) and water (10 ml) and the solution refluxed under argon for 8 hr. Water was added and the product was extracted with ethyl acetate (3×25 ml). Evaporation of the solvent followed by flash chromatography on silica gel (eluent, hexane:ethyl acetate, 90:10 to 80:20) afforded 1-ethyl-4-nitro-3-p-tolyl-1H-indazole (0.50 g, 1.77 mmol); LC/MS, API-ES, Pos, (M+H)⁺, 282.1.

Step E:

1-Ethyl-4-nitro-3-p-tolyl-1H-indazole (0.50 g, 1.77 mmol) (Step D) was dissolved in a mixture of methanol (80 ml) and ethyl acetate (20 ml) and 10% Pd/C (50 mg) was added. Hydrogen gas was gently bubbled through the solution with stirring at ambient temperature for 2 hr. The catalyst was removed by filtration over celite and the filtrate was evaporated in vacuo. Purification by flash chromatography on silica gel (eluent, hexane:ethyl acetate, 90:10 to 85:15) afforded the title compound (0.33 g, 1.31 mmol); LC/MS, API-ES, Pos, (M+H)⁺, 252.1.

### Example 13

### 1-tert-Butyl-3-(4-fluoro-phenyl)-1H-pyrazolo[3,4-d]pyrimidine-4,6-diamine (Compound No. 116)

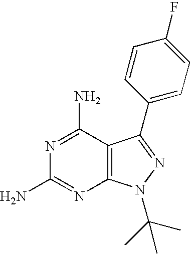

A mixture of 5-amino-1-tert-butyl-3-(4-fluoro-phenyl)-1H-pyrazole-4-carbonitrile (1.0 g, 3.87 mmol), guanidine carbonate (1.22 g, 6.77 mmol) and triethylamine (5 ml) was heated in a sealed tube at 205° C. for 2.5 hr. Water was added and the product extracted with ethyl acetate (4×30 ml). The organic layer was washed with water and brine, dried (anhydrous sodium sulfate) and evaporated. A fraction of the crude product (¼) was subjected to preparative reverse phase HPLC and the desired peak was pooled (water-acetonitrile gradient, 0.05% trifluoroacetic acid, 70:30 to 10:90, 20 min, linear gradient; flow, 15 ml/min; column, Phenomenex Luna 5μ C18, 100×21.2 mm; UV 254 and 218 nm). Evaporation of the solvent followed by crystallization from ether afforded the title compound (55 mg, 0.18 mmol); LC/MS, API-ES, Pos, (M+H)⁺, 301.1.

### Example 14

### [1-tert-Butyl-3-(4-fluoro-phenyl)-1H-pyrazolo[3,4-d]pyrimidin-4-yl]-methyl-amine (Compound No. 38) and [1-tert-Butyl-3-(4-fluoro-phenyl)-1H-pyrazolo[3,4-d]pyrimidin-4-yl]-dimethyl-amine (Compound No. 39)

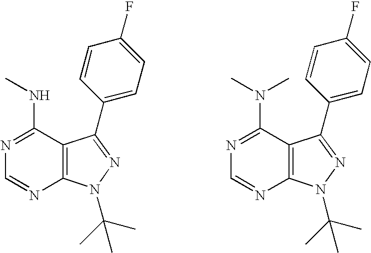

Sodium hydride (60%, 22 mg, 0.55 mmol) was suspended in anhydrous dimethylformamide (5 ml) and stirred at 0° C. 1-Tert-butyl-3-(4-fluoro-phenyl)-1H-pyrazolo[3,4-d]pyrimidin-4-ylamine (142.6 mg, 0.5 mmol) dissolved in 1 ml dimethylformamide was added and the solution stirred for 10 min. Methyl iodide (354.9 mg, 2.5 mmol) was added and the solution stirred at ambient temperature over night. Water was added and the product extracted with ethyl acetate. Organic layer was washed with water and brine, dried (anhydrous sodium sulfate) and evaporated to afford a product mixture. Flash chromatography on silica gel (eluent, hexane:ethyl acetate, 90:10 to 70:30) afforded the title compounds [1-tert-butyl-3-(4-fluoro-phenyl)-1H-pyrazolo[3,4-d]pyrimidin-4-yl]-dimethyl-amine (66.5 mg, 0.21 mmol); LC/MS, API-ES, Pos, (M+H)⁺, 314.1 and [1-tert-butyl-3-(4-fluoro-phenyl)-1H-pyrazolo[3,4-d]pyrimidin-4-yl]-methyl-amine (41.5 mg, 0.14 mmol); LC/MS, API-ES, Pos, (M+H)⁺, 300.1.

### Example 15

### N-[1-tert-Butyl-3-(4-fluoro-phenyl)-1H-pyrazolo[3,4-d]pyrimidin-4-yl]-acetamide (Compound No. 118) and N-Acetyl-N-[1-tert-butyl-3-(4-fluoro-phenyl)-1H-pyrazolo[3,4-d]pyrimidin-4-yl]-acetamide (Compound No. 117)

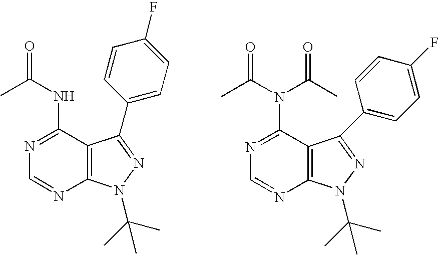

1-Tert-butyl-3-(4-fluoro-phenyl)-1H-pyrazolo[3,4-d]pyrimidin-4-ylamine (142.6 mg, 0.5 mmol) was dissolved in 2 ml of anhydrous pyridine and solution stirred at 0° C. Acetyl chloride (196.3 mg, 2.5 mmol) was added drop-wise and the solution stirred at ambient temperature over night. Water was added and the product extracted with ethyl acetate. Organic layer was washed with water and brine, dried (anhydrous sodium sulfate) and evaporated to afford a product mixture. Flash chromatography on silica gel (eluent, hexane:ethyl acetate, 90:10 to 70:30) afforded the title compounds N-acetyl-N-[1-tert-butyl-3-(4-fluoro-phenyl)-1H-pyrazolo[3,4-d]pyrimidin-4-yl]-acetamide (45.0 mg, 0.12 mmol); LC/MS, API-ES, Pos, (M+H)⁺370.1, and N-[1-tert-butyl-3-(4-fluoro-phenyl)-1H-pyrazolo[3,4-d]pyrimidin-4-yl]-acetamide (12.7 mg, 0.04 mmol); LC/MS, API-ES, Pos, (M+H)⁺328.1.

### Example 16

### N-[1-tert-Butyl-3-(4-fluoro-phenyl)-1H-pyrazolo[3,4-d]pyrimidin-4-yl]-benzamide (Compound No. 40)

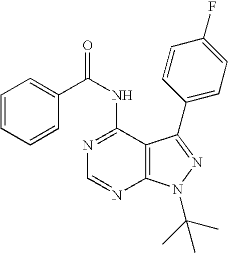

1-Tert-butyl-3-(4-fluoro-phenyl)-1H-pyrazolo[3,4-d]pyrimidin-4-ylamine (142.6 mg, 0.5 mmol) was dissolved in 2 ml of anhydrous pyridine and solution stirred at 0° C. Benzoyl chloride (351.4 mg, 2.5 mmol) was added drop-wise and the solution stirred at ambient temperature over night. Water was added and the product extracted with ethyl acetate. Organic layer was washed with water and brine, dried (anhydrous sodium sulfate) and evaporated to afford a product mixture. The residue was stirred in acetonitrile and the precipitate was separated by filtration. Flash chromatography on silica gel (eluent, hexane:ethyl acetate, 90:10 to 70:30) afforded the title compound (75.0 mg, 0.19 mmol); LC/MS, API-ES, Pos, (M+H)⁺390.1.

### Example 17

### 1-tert-Butyl-3-(4-chloro-phenyl)-1H-pyrazolo[3,4-d]pyrimidin-4-ylamine hydrochloride (Compound No. 407)

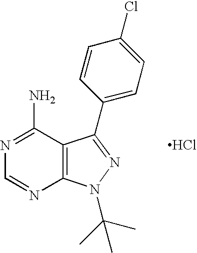

1-Tert-butyl-3-(4-chloro-phenyl)-1H-pyrazolo[3,4-d]pyrimidin-4-ylamine (100 mg, 0.33 mmol) was dissolved in 3 ml of anhydrous chloroform and ethereal HCl (1M solution, 0.4 ml, 0.4 mmol) was added. The solution was allowed to stand at ambient temperature for 1 hr. Upon partial evaporation of the solvent, a precipitate was formed that was separated by decantation and the residue washed with small amount of ether and dioxane to afford the title compound (80 mg, 0.24 mmol); LC/MS, API-ES, Pos, (M+H)⁺, parent ion for free base, 302.1.

### Example 18

### 4-(4-Amino-1-tert-butyl-1H-pyrazolo[3,4-d]pyrimidin-3-yl)-benzoic acid ethyl ester (Compound No. 123)

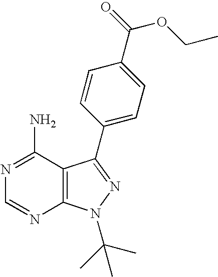

3-Bromo-1-tert-butyl-1H-pyrazolo[3,4-d]pyrimidin-4-ylamine (351 mg, 1.3 mmol), ethyl 4-(4,4,5,5-tetramethyl-1,3,2-dioxaborolan-2-yl)benzoate (395 mg, 1.43 mmol), tetrakistriphenylphosphine palladium (90 mg, 0.07 mmol) and sodium carbonate (330 mg, 3.11 mmol) were mixed in 1,2-dimethoxyethane (20 ml) and water (10 ml) and the solution refluxed under argon for 6 hr. Water was added and the product was extracted with ethyl acetate (3×25 ml). Evaporation of the solvent followed by flash chromatography on silica gel (eluent, hexane:ethyl acetate, 80:20 to 60:40) afforded the title compound that was crystallized form methanol (80 mg, 0.24 mmol); LC/MS, API-ES, Pos, (M+H)⁺, 340.1.

### Example 19

### 4-Amino-1-tert-butyl-3-p-tolyl-1H-pyrazolo[3,4-b]pyridine-5-carboxylic acid (Compound No. 45)

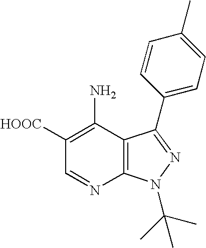

Step A:

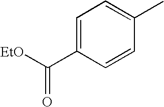

Thionyl chloride (22.3 ml, 0.3 mol) was added to 4-methyl-benzoic acid (27.1 g, 0.2 mol) in ethanol (200 ml) and the solution stirred overnight. The solvent was evaporated to give 4-methyl-benzoic acid ethyl ester (30 g, 0.18 mol) as a viscous liquid.

Step B:

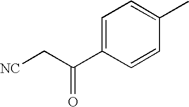

To a stirred solution of acetonitrile (48 ml, 0.92 mol) and toluene (100 ml), sodium hydride (22 g, 0.92 mol) was added in parts. After stirring at 50° C. for 2 hr, 4-methyl-benzoic acid ethyl ester (30 g, 0.18 mol) (Step A) in toluene (100 ml) was added and refluxed for 4 hr. The solvents were then evaporated under vacuum. The residue was quenched with ice (200 ml) and extracted with ethyl acetate. The organic layer was washed with brine, dried over anhydrous sodium sulfate, concentrated, and purified by column chromatography to give 3-oxo-3-p-tolyl-propionitrile (22 g, 0.14 mol).

Step C:

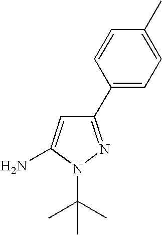

3-Oxo-3-p-tolyl-propionitrile (22 g, 0.14 mol) (Step B) was dissolved in isopropanol (500 ml), triethylamine (40 ml, 0.28 mol) was added, and the mixture was stirred for 5 min, then t-butyl hydrazine hydrochloride was added, and the mixture was refluxed for 5 hr under nitrogen. The reaction was cooled to room temperature and the solvent was removed in vacuo. The residue was dissolved in ethyl acetate, washed with water, brine, and dried over anhydrous sodium sulfate. The organic layer was filtered, concentrated under vacuum, loaded on a silica gel column and purified to give 2-tert-butyl-5-p-tolyl-2H-pyrazol-3-ylamine (24 g, 0.11 mol).

Step D:

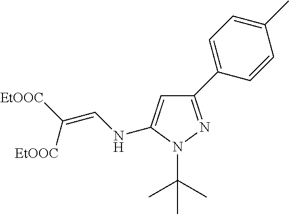

2-tert-Butyl-5-p-tolyl-2H-pyrazol-3-ylamine (10 g, 0.044 mol) (Step C) was stirred with diethyl(ethoxymethylene)malonate (9.5 g, 0.044 mol) at 120° C. for 4 hr. The mixture was dissolved in dichloromethane, adsorbed on silica gel and purified by column chromatography to give 2-[(2-tert-butyl-5-p-tolyl-2H-pyrazol-3-ylamino)-methylene]-malonic acid diethyl ester (10 g, 0.03 mol).

Step E:

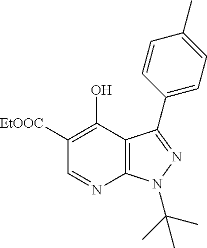

2-[(2-tert-Butyl-5-p-tolyl-2H-pyrazol-3-ylamino)-methylene]-malonic acid diethyl ester (5 g, 12.5 mmol) (Step D) was stirred in diphenyl ether (75 ml) at 190° C. for 48 hr. The resultant solution was cooled to room temperature, poured slowly on to a silica gel column and eluted with petroleum ether to give 1-tert-butyl-4-hydroxy-3-p-tolyl-1H-pyrazolo[3,4-b]pyridine-5-carboxylic acid ethyl ester (1.1 g, 3.11 mmol).

Step F:

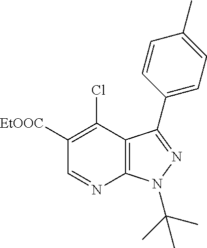

1-tert-Butyl-4-hydroxy-3-p-tolyl-1H-pyrazolo[3,4-b]pyridine-5-carboxylic acid ethyl ester (1.1 g, 3.1 mmol) (Step E) was refluxed in POCl₃ for 4 hr. The mixture was concentrated under vacuum to remove POCl₃. The residue was diluted with water and extracted with ethyl acetate. The extracts were dried (anhydrous sodium sulfate), filtered and the filtrate was concentrated and purified by column chromatography to give 1-tert-butyl-4-chloro-3-p-tolyl-1H-pyrazolo[3,4-b]pyridine-5-carboxylic acid ethyl ester (0.8 g, 2.15 mmol).

Step G:

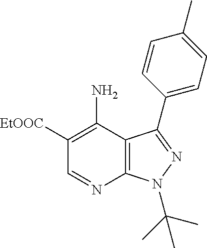

1-tert-Butyl-4-chloro-3-p-tolyl-1H-pyrazolo[3,4-b]pyridine-5-carboxylic acid ethyl ester (0.8 g, 2.2 mmol) was stirred in 25 ml of ethanol saturated with ammonia in a closed steel vessel at 110° C. for 12 hr. The cooled reaction mixture was concentrated and the residue was triturated with ether and filtered. The filtrate was dried (anhydrous sodium sulfate), filtered, concentrated, and purified by column chromatography to give 4-amino-1-tert-butyl-3-p-tolyl-1H-pyrazolo[3,4-b]pyridine-5-carboxylic acid ethyl ester (Compound No. 45; 0.5 g, 1.42 mmol).

Step H:

4-Amino-1-tert-butyl-3-p-tolyl-1H-pyrazolo[3,4-b]pyridine-5-carboxylic acid ethyl ester (0.5 g, 1.4 mmol) was stirred in ethanol (95%) and sodium hydroxide (0.24 g, 6.0 mmol) overnight at 50° C. The mixture was concentrated, the residue dissolved in water (600 ml), filtered and acidified with acetic acid. The precipitate formed was collected, washed with water and air dried to give 4-amino-1-tert-butyl-3-p-tolyl-1H-pyrazolo[3,4-b]pyridine-5-carboxylic acid (0.3 g, 0.92 mmol) as a white solid; LC/MS, APCI, Neg, (M−H)⁻, 323.3.

### Example 20

### 1-tert-Butyl-3-p-tolyl-1H-pyrazolo[3,4-b]pyridin-4-ylamine (Compound No. 69)

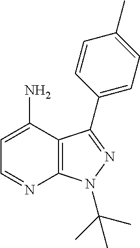

4-Amino-1-tert-butyl-3-p-tolyl-1H-pyrazolo[3,4-b]pyridine-5-carboxylic acid (0.1 g, 0.3 mmol) was heated at 180° C. under a nitrogen atmosphere for 48 hr. The resulting product was purified by column chromatography to give (20 mg, 0.07 mmol) of 1-tert-butyl-3-p-tolyl-1H-pyrazolo[3,4-b]pyridine-4-ylamine as a pale brown solid; LC/MS, APCI, Pos, (M+H)⁺, 281.5.

### Example 21

### 1-tert-Butyl-3-phenoxy-1H-pyrazolo[3,4-d]pyrimidin-4-ylamine (Compound No. 90)

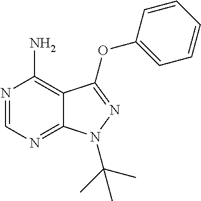

3-Bromo-1-tert-butyl-1H-pyrazolo[3,4-d]pyrimidin-4-ylamine (540 mg, 2 mmol) (Example 10, Step C) and phenol (753 mg, 8 mmol) were mixed with a few drops of 1-pentanol and heated at 120° C. for 5 min. Potassium carbonate (1.1 g, 8 mmol) and copper powder (51 mg, 0.8 mmol) were added and the mixture heated at 195° C. for 1 hr. Water was added and product extracted with methylene chloride. The organic layer was washed with water, dried (anhydrous sodium sulfate) and evaporated to afford a residue that was subjected to preparative reverse phase HPLC (water-acetonitrile gradient, 0.05% trifluoroacetic acid, 70:30 to 10:90, 20 min, linear gradient; flow, 15 ml/min; column, Phenomenex Luna 5μ C18, 100×21.2 mm; UV 254 and 218 nm). The desired peak was pooled and solvent evaporated in vacuo. Residue was dissolved in methylene chloride and solution washed with aqueous sodium bicarbonate followed by water and solvent evaporated to afford the title compound (50 mg, 0.18 mmol); LC/MS, API-ES, Pos, (M+H)⁺, 284.2.

### Example 22

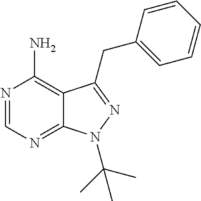

### 3-Benzyl-1-tert-butyl-1H-pyrazolo[3,4-d]pyrimidin-4-ylamine (Compound No. 11)

### Step A

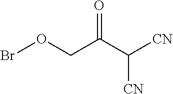

Malononitrile (8.95 g, 135.4 mmol) was dissolved in anhydrous tetrahydrofuran (400 ml) and the solution stirred under ice-water cooling. Sodium hydride (60% in mineral oil, 10.8 g, 270 mmol) was added in portions followed by drop-wise addition of benzyloxyacetyl chloride (25 g, 135.4 mmol in tetrahydrofuran, 50 ml). Solution was stirred at ambient temperature for 2 hr. 1M Hydrochloric acid (500 ml) was added and the solution extracted with ethyl acetate (3×250 ml). The organic layer was washed with water, dried (anhydrous sodium sulfate) and evaporated in vacuo to afford a residue that was triturated with hexane to afford 2-(2-benzyloxyacetyl)-malononitrile as an amorphous powder that was used as such for the next step; LC/MS, API-ES, Pos, (M+H)⁺, 215.2.

### Step B

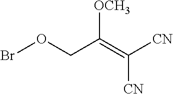

2-(2-Benzyloxyacetyl)-malononitrile from previous step (135.4 mmol), potassium carbonate (31.7 g, 230.2 mmol) and dimethyl sulfate (23.9 g, 189.5 mmol) in dioxane (500 ml) were stirred at 85° C. for 3 hr. The solution was filtered and solvent evaporated to afford a residue that was subjected to silica gel chromatography (eluent; hexane-ethyl acetate gradient) to afford 2-(2-benzyloxy-1-methoxy-ethylidene)-malononitrile (12.9 g, 56.5 mmol); LC/MS, API-ES, Pos, (M+H)⁺, 229.2.

### Step C

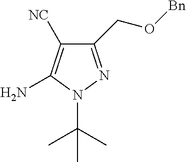

2-(2-Benzyloxy-1-methoxy-ethylidene)-malononitrile (12.9 g, 56.5 mmol), t-butylhydrazine hydrochloride (6.95 g, 55.7 mmol) and triethyl amine (7.3 g, 72.1 mmol) in anhydrous ethanol (300 ml) were heated under reflux for 2 hr. The insoluble material was removed by filtration and the solvent removed in vacuo to afford a residue which was subjected to flash silica gel chromatography. Elution with a gradient of hexane-ethyl acetate afforded 5-amino-3-benzyloxymethyl-1-tert-butyl-1H-pyrazole-4-carbonitrile (12.1 g, 42.5 mmol); LC/MS, API-ES, Pos, (M+H)⁺, 285.3.

### Step D

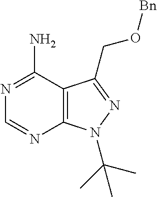

5-Amino-3-benzyloxymethyl-1-tert-butyl-1H-pyrazole-4-carbonitrile (12.1 g, 42.5 mmol) in formamide (170 ml) was heated at 185° C. for 2.5 hr. The solution was allowed to stand over night at ambient temperature and the deposited crystalline material was separated by filtration. The crystals were washed with formamide followed by water and air dried to afford 3-benzyloxymethyl-1-tert-butyl-1H-pyrazolo[3,4-d]pyrimidin-4-ylamine (Compound No. 81; 9.2 g, 29.5 mmol); LC-MS, API-ES, Pos, (M+H)⁺, 312.3.

### Step E

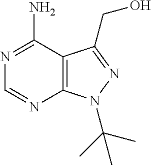

3-Benzyloxymethyl-1-tert-butyl-1H-pyrazolo[3,4-d]pyrimidin-4-ylamine (4.0 g, 12.9 mmol) was dissolved in anhydrous methylene chloride (130 ml) and stirred at −78° C. Boron trichloride (1 M solution in heptane, 51.8 ml, 51.8 mmol) was added drop-wise with stirring and the solution warmed to 0° C. and stirred at this temperature for 15 min. The solution was cooled to −78° C. and methanol (71 ml) was added. The solution was warmed to 0° C. and neutralized to pH 7 with ammonium hydroxide. Solution was filtered and the filtrate evaporated in vacuo to afford a residue which was crystallized from small amount of ether and hexane to afford (4-amino-1-tert-butyl-1H-pyrazolo[3,4-d]pyrimidin-3-yl)-methanol (Compound No. 148; 2.4 g, 10.8 mmol); LC-MS, API-ES, Pos, (M+H)⁺, 222.2.

### Step F

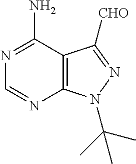

(4-Amino-1-tert-butyl-1H-pyrazolo[3,4-d]pyrimidin-3-yl)-methanol (2.93 g, 13.2 mmol) was dissolved in chloroform (150 ml) and manganese dioxide (11.5 g, 132.3 mmol) was added. The solution was stirred at ambient temperature for 20 hr and filtered through a plug of Celite. The filtrate was evaporated in vacuo and residue crystallized from acetonitrile to afford 4-amino-1-tert-butyl-1H-pyrazolo[3,4-d]pyrimidine-3-carbaldehyde (Compound No. 149; 2.3 g, 10.4 mmol); LC-MS, API-ES, Pos, (M+H)⁺, 220.2.

### Step G

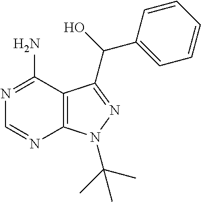

To a stirred solution of 4-amino-1-tert-butyl-1H-pyrazolo[3,4-d]pyrimidine-3-carbaldehyde (0.49 g, 2.2 mmol) in anhydrous tetrahydrofuran (14 ml) at 0° C. was added drop-wise phenylmagnesium bromide (1M in tetrahydrofuran, 2.45 ml, 2.45 mmol). The solution was stirred at 0° C. for 0.5 hr followed by stirring at ambient temperature for 1.5 hr. Saturated ammonium chloride solution (50 ml) was added and the product extracted with methylene chloride. The organic layer was washed with water, dried (anhydrous sodium sulfate) and solvent evaporated to afford a residue that was purified by flash chromatography on silica gel (eluent, hexane-ethyl acetate gradient) to afford (4-amino-1-tert-butyl-1H-pyrazolo[3,4-d]pyrimidin-3-yl)-phenyl-methanol (Compound No. 82; 0.19 g, 0.64 mmol); LC-MS, API-ES, Pos, (M+H)⁺, 298.3.

### Step H

(4-Amino-1-tert-butyl-1H-pyrazolo[3,4-d]pyrimidin-3-yl)-phenyl-methanol (20 mg, 0.06 mmol) was dissolved in trifluoroactic acid (1 ml) and the solution cooled in an ice bath. Triethylsilane (23 mg, 0.20 mmol) was added and the mixture was stirred over night. Solvent was evaporated, the residue dried in vacuo and triturated with hexane to afford the title compound as an off-white solid (15 mg, 0.05 mmol); LC-MS, API-ES, Pos, (M+H)⁺, 282.3.

### Example 23

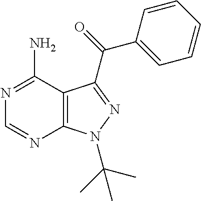

### (4-Amino-1-tert-butyl-1H-pyrazolo[3,4-d]pyrimidin-3-yl)-phenyl-methanone (Compound No. 83)

(4-Amino-1-tert-butyl-1H-pyrazolo[3,4-d]pyrimidin-3-yl)-phenyl-methanol (90 mg, 0.27 mmol) (Example 19, Step G) was dissolved in anhydrous methylene chloride (5 ml) and stirred at 0° C. Dess-Martin periodinane solution (0.3 M in dichloromethane, 1.85 ml, 0.55 mmol) was added and the solution stirred at this temperature for 1.5 hr. The reaction was quenched with aqueous sodium sulfite solution (1.3 M solution, 4 ml) followed by saturated sodium bicarbonate solution (4 ml) and stirred at 0° C. for 0.5 hr. The solution was extracted with dichloromethane, washed with water, dried (anhydrous sodium sulfate) and solvent removed in vacuo to afford a residue that was flash chromatographed on silica gel (eluent, hexane-ethyl acetate gradient) to afford the title compound (42 mg, 0.14 mmol); LC-MS, API-ES, Pos, (M+H)⁺, 296.3.

### Example 24

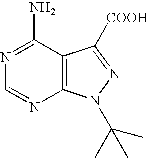

### 4-Amino-1-tert-butyl-1H-pyrazolo[3,4-d]pyrimidine-3-carboxylic acid (Compound No. 85)

4-Amino-1-tert-butyl-1H-pyrazolo[3,4-d]pyrimidine-3-carbaldehyde (150 mg, 0.68 mmol) was dissolved in acetone (5 ml) and a solution of potassium permanganate (216 mg, 1.36 mmol) in acetone-water (1:1, 2 ml) was added. The solution was stirred at ambient temperature over night. Acetic acid (3 ml) was added and the product extracted with methylene chloride. The organic layer was washed with water, dried (anhydrous sodium sulfate) and evaporated in vacuo to afford a residue that was crystallized from acetonitrile-methanol to afford the title compound (80 mg, 0.34 mmol); LC-MS, API-ES, Pos, (M+H)⁺, 236.2, API-ES, Neg, (M−H)⁻, 233.9.

### Example 25

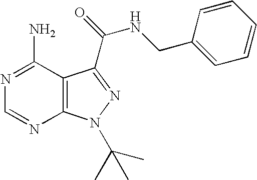

### 4-Amino-1-tert-butyl-1H-pyrazolo[3,4-d]pyrimidine-3-carboxylic acid benzylamide (Compound No. 160)

4-Amino-1-tert-butyl-1H-pyrazolo[3,4-d]pyrimidine-3-carboxylic acid (47 mg, 0.2 mmol) and benzyl amine (24 mg, 0.22 mmol) were dissolved in anhydrous dimethyl formamide (2 ml). Diisopropylethyl amine (77 mg, 0.6 mmol) and 2-(1H-benzotriazole-1-yl)-1,1,3,3-tetramethyluronium hexafluorophosphate (HBTU, 83 mg, 0.22 mmol) were added and the solution stirred at ambient temperature over night. The solution was filtered and subjected to preparative reverse phase HPLC (water-acetonitrile gradient, 0.05% formic acid, 80:20 to 10:90, 20 min, linear gradient; flow, 15 ml/min; column, Phenomenex Luna 5μ C18, 100×21.2 mm; UV 254 and 218 nm) to afford the title compound (31 mg, 0.1 mmol, crystallized from acetonitrile), LC-MS, API-ES, Pos, (M+H)⁺, 325.3.

### Example 26

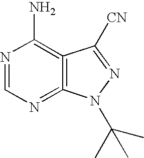

### 4-Amino-1-tert-butyl-1H-pyrazolo[3,4-d]pyrimidine-3-carbonitrile (Compound No. 159)

### Step A

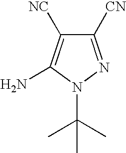

To the stirred suspension of finely ground tert-butylhydrazine hydrochloride (12.5 g, 0.1 mol) in ethanol (95%, 100 ml), NaOH solution (4.0 g, 0.1 mol) in ethanol (150 ml) was added under ice cooling. Finely ground tetracyanoethylene (12.8 g, 0.1 mol) was added, completing the transfer with ˜10 ml of ethanol. The mixture was stirred for 0.5 hr at ambient temperature, the white precipitate (consisting of the product and NaCl) was filtered from dark-red solution and washed with minimum amount of ethanol (5-10 ml). The precipitate was thoroughly washed with acetonitrile (5×50 ml) and filtered. The filtrate was concentrated under vacuum and recrystallized from ethyl ether to afford 5-amino-1-tert-butyl-1H-pyrazole-3,4-dicarbonitrile (8.63 g, 45.6 mmol) as a light-pink solid; LC-MS, API-ES, Neg, (M−H)⁻, 187.9.

### Step B

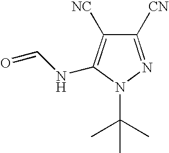

5-Amino-1-tert-butyl-1H-pyrazole-3,4-dicarbonitrile (6.56 g, 35 mmol) was refluxed in triethylorthoformate (60 ml, 350 mmol) for 3 days until reaction was complete (LCMS). The mixture was concentrated and dried under vacuum. The resulting yellow solid [N-(2-tert-butyl-4,5-dicyano-2H-pyrazol-3-yl)-formamide] was used in the next step without additional purification; LC-MS API-ES, Neg, (M−H)⁻, 215.9.

### Step C

N-(2-tert-Butyl-4,5-dicyano-2H-pyrazol-3-yl)-formamide (35 mmol, Step B) was dissolved in methanol (150 ml). Ammonia solution (7N in MeOH, 6.0 ml, 42 mmol) was added to the solution, and the mixture was stirred for 2 hr. The mixture was concentrated under vacuum and purified by silica gel chromatography (eluent dichloromethane-methanol, 100:0 to 80:20) to afford the title product as a yellow solid. Recrystallization from ethyl acetate afforded analytical sample as an off-white solid (3.67 g, 16.9 mmol); LC-MS, API-ES, Pos., (M+H)⁺, 217.2.

### Example 27

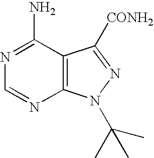

### 4-Amino-1-tert-butyl-1H-pyrazolo[3,4-d]pyrimidine-3-carboxylic acid amide (Compound No. 89)

4-Amino-1-tert-butyl-1H-pyrazolo[3,4-d]pyrimidine-3-carbonitrile (216 mg, 1 mmol) was mixed with concentrated aqueous ammonium hydroxide (28%, 4 ml) and hydrogen peroxide (30-35%, 1 ml). The mixture was stirred over night, filtered, and washed with water to afford the title compound (207 mg, 0.88 mmol) as an off-white solid; LC-MS, API-ES, Pos., (M+H)⁺, 235.2.

### Example 28

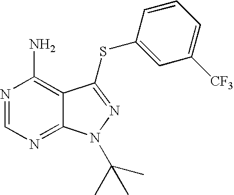

### 1-tert-Butyl-3-(3-trifluoromethyl-phenylsulfanyl)-1H-pyrazolo[3,4-d]pyrimidin-4-ylamine (Compound No. 244)

3-Bromo-1-tert-butyl-1H-pyrazolo[3,4-d]pyrimidin-4-ylamine (0.50 g, 1.85 mmol), CuI (0.0176 g, 0.093 mmol), potassium carbonate powder (0.511 g, 3.70 mmol), ethylene glycol (0.21 ml, 3.70 mmol), 2-propanol (1.86 ml) and 3-trifluoromethyl benzenethiol (0.33 g, 1.85 mmol) were placed in a microwave reactor tube with magnetic stirrer, degassed, and heated in a microwave reactor for 30 min at 130° C. The reaction mixture was added to saturated aqueous sodium thiosulfate and extracted with methylene chloride (3×15 ml). The organic layer was washed with water, brine, and dried over anhydrous sodium sulfate, filtered, concentrated, purified by flash silica gel column chromatography (eluent hexanes-EtOAc) followed by preparative RPHPLC (water-acetonitrile gradient, 0.05% formic acid) to obtain 1-tert-butyl-3-(3-trifluoromethyl-phenylsulfanyl)-1H-pyrazolo[3,4-d]pyrimidin-4-ylamine (0.30 g, 0.82 mmol); LC-MS, API-ES, Pos., (M+H)⁺, 340.1.

### Example 29

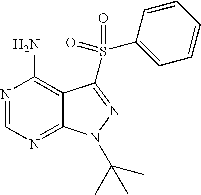

### 3-Benzenesulfonyl-1-tert-butyl-1H-pyrazolo[3,4-d]pyrimidin-4-ylamine (Compound No. 191)

### Step A

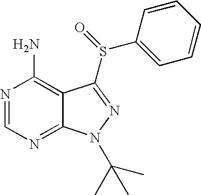

To the stirred solution of 1-tert-Butyl-3-phenylsulfanyl-1H-pyrazolo[3,4-d]pyrimidin-4-ylamine (380 mg, 1.27 mmol) in chloroform (30 ml), 3-chloroperoxybenzoic acid (70-75%, 1.56 g, 6.35 mmol) was added and the mixture was refluxed for 1 day until starting material in consumed (LCMS). The resulting mixture of oxides was washed with saturated aqueous NaHCO₃ (100 ml), the aqueous layer extracted with methylene chloride (3×20 ml), the extracts washed with brine (20 ml), dried over magnesium sulfate, concentrated under vacuum, and purified on silica gel (50 g, eluent methylene chloride-acetonitrile, 100:0 to 0:100) to afford two oxides: 3-benzenesulfonyl-1-tert-butyl-1H-pyrazolo[3,4-d]pyrimidin-4-ylamine (152 mg, 0.46 mmol) and 3-benzenesulfinyl-1-tert-butyl-1H-pyrazolo[3,4-d]pyrimidin-4-ylamine (Compound No. 173; 217 mg, 0.69 mmol) as a white solid; LC/MS, API-ES, Pos, (M+H)⁺, 316.1.

### Step B

3-Benzenesulfinyl-1-tert-butyl-1H-pyrazolo[3,4-d]pyrimidin-4-ylamine (370 mg, 1.17 mmol) was dissolved in acetonitrile (20 ml), 4-methylmorpholine N-oxide (0.56 g, 4.8 mmol) and osmium tetroxide solution (2.5% in butanol, 0.13 ml, 0.01 mmol) were added, and the mixture was heated in a microwave reactor at 70° C. for 90 min until reaction completion (LCMS). The mixture was concentrated under vacuum, diluted with methylene chloride (20 ml), partitioned with water (100 ml), and the aqueous layer extracted with methylene chloride (2×15 ml). The combined organic extracts were washed with brine (15 ml), dried over magnesium sulfate and concentrated under vacuum. The residue was purified by silica gel chromatography (eluent methylene chloride-acetonitrile, 100:0 to 0:100) to afford 3-benzenesulfonyl-1-tert-butyl-1H-pyrazolo[3,4-d]pyrimidin-4-ylamine (223 mg, 0.67 mmol) as an off-white crystals; LC/MS, API-ES, Pos, (M+H)⁺, 332.1.

### Example 30

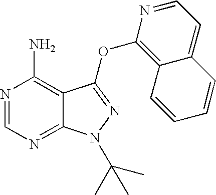

### 1-tert-Butyl-3-(isoquinolin-1-yloxy)-1H-pyrazolo[3,4-d]pyrimidin-4-ylamine (Compound No. 370)

### Step A

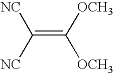

A mixture of tetracyanoethylene (12.8 g, 100 mmol) and urea (2.0 g, 33.3 mmol) in methanol (50 ml) was stirred at 50° C. for 0.5 hr. Ether (250 ml) was added and the solution was chilled to −78° C. The precipitate was separated by filtration while cold to afford 2-dimethoxymethylene-malononitrile (5.6 g, 40.5 mmol) as an off-white solid, LC/MS, API-ES, Pos, (M+H)⁺, 139.1. Processing of mother solution furnished additional 2.1 g, 15 mmol) of the product.

### Step B

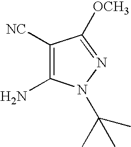

A mixture of 2-dimethoxymethylene-malononitrile (4.26 g, 30.9 mmol) (Step A), triethylamine (4.07 g, 40.2 mmol) and t-butylhydrazine hydrochloride (3.86 g, 30.9 mmol) in methanol (100 ml) was heated at reflux for 2 hr. Solution was filtered and solvent evaporated in vacuo to afford a residue which was subjected to flash column chromatography on silica gel (eluent; hexane:ethyl acetate, 90:10 to 10:90) to afford 5-amino-1-tert-butyl-3-methoxy-1H-pyrazole-4-carbonitrile (3.83 g, 193 mmol); LC/MS, API-ES, Pos, (M+H)⁺, 195.3.

### Step C

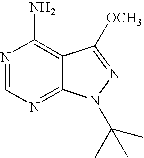

A mixture of 5-amino-1-tert-butyl-3-methoxy-1H-pyrazole-4-carbonitrile (3.8 g, 19.6 mmol) (Step B) and formamide (50 ml) was heated at 185° C. for 2 hr. The mixture was cooled to ambient temperature and the precipitated solid separated by filtration. The solid was washed with formamide followed by water and air dried to afford 1-tert-butyl-3-methoxy-1H-pyrazolo[3,4-d]pyrimidin-4-ylamine (Compound No. 312; 2.83 g, 12.8 mmol); LC/MS, API-ES, Pos, (M+H)⁺, 222.3.

### Step D

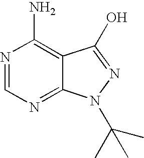

To a mixture of 1-tert-butyl-3-methoxy-1H-pyrazolo[3,4-d]pyrimidin-4-ylamine (2.83 g, 12.8 mmol) (Step C) and sodium iodide (3.83 g, 25.6 mmol) in acetonitrile (100 ml) was added trimethylsilyl chloride (2.78 g, 25.6 mmol) and the solution heated at reflux overnight under argon. LC-MS analysis indicated the presence of about 20% unreacted starting material. A second batch of the sodium iodide (0.58 g, 3.8 mmol) and trimethylsilyl chloride (0.42 g, 3.8 mmol) was added and the solution refluxed for additional 12 hr. The solution was filtered and solvent removed in vacuo to afford a residue. Crystallization from acetonitrile and water afforded 4-amino-1-tert-butyl-1H-pyrazolo[3,4-d]pyrimidin-3-ol (Compound No. 325; 2.2 g, 10.6 mmol); LC/MS, API-ES, Pos, (M+H)⁺, 208.3.

### Step E

A mixture of 4-amino-1-tert-butyl-1H-pyrazolo[3,4-d]pyrimidin-3-ol (Step D) (104 mg, 0.5 mmol), 1-chloroisoquinoline (98 mg, 0.6 mmol), and potassium carbonate (83 mg, 0.6 mmol) in anhydrous dimethylsulfoxide (2 ml) was stirred at 130° C. for 4 hr. Water (15 ml) was added and the product extracted with ethyl acetate (3×10 ml). Combined organic layer was washed with water, dried (anhydrous sodium sulfate) and evaporated in vacuo. The residue was subjected to preparative HPLC (water-acetonitrile gradient, 0.05% formic acid, 80:20 to 10:90, 20 min, linear gradient; flow, 15 ml/min; column, Phenomenex Luna 5μ C18, 100×21.2 mm; UV 254 and 218 nm) to afford 1-tert-butyl-3-(isoquinolin-1-yloxy)-1H-pyrazolo[3,4-d]pyrimidin-4-ylamine (23 mg, 0.07 mmol); LC/MS, API-ES, Pos, (M+H)⁺, 335.3.

### Example 31

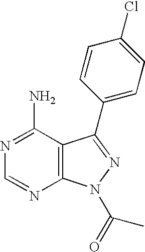

### 1-[4-Amino-3-(4-chloro-phenyl)-pyrazolo[3,4-d]pyrimidin-1-yl]-ethanone (Compound No. 181)

To a suspension of 3-(4-chlorophenyl)-1H-pyrazolo[3,4-d]pyrimidin-4-amine (100 mg, 0.41 mmol) in 5 ml dimethylformamide, pyridine (39 mg, 0.49 mmol) was added. Acetyl chloride (35 mg, 0.45 mmol) in 1 ml DMF was added dropwise. The reaction mixture was stirred at room temperature for 1 hr. Additional 1.2 equivalent of pyridine and 1.1 equivalent of acetyl chloride were added and the solution stirred for 1 hr. The process was repeated once and the mixture was poured into water and extracted with ethyl acetate. Organic layer was washed with water, dried (anhydrous sodium sulfate) and evaporated. The residue was crystallized from acetone to afford the title compound (40 mg, 0.14 mmol); LC/MS, API-ES, Pos, (M+H)⁺, 288.0, 290.0.

### Example 32

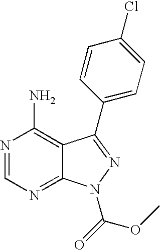

### 4-Amino-3-(4-chloro-phenyl)-pyrazolo[3,4-d]pyrimidine-1-carboxylic acid methyl ester (Compound No. 186)

To a suspension of 3-(4-chlorophenyl)-1H-pyrazolo[3,4-d]pyrimidin-4-amine (200 mg, 0.81 mmol) in 5 ml dimethylformamide, pyridine (77 mg, 0.98 mmol) was added. Methyl chloroformate (84 mg, 0.90 mmol) was added dropwise. Additional 1.2 equivalent of pyridine and 1.1 equivalent of methyl chloroformate were added followed by stirring for 1 hr. The process was repeated twice. The mixture was poured into water and extracted with ethyl acetate. The organic layer was washed with water, dried (anhydrous sodium sulfate) and evaporated. Trituration with ethyl acetate afforded the title compound (57 mg, 0.19 mmol); LC/MS, API-ES, Pos, (M+H)⁺, 304.0, 306.0.

### Example 33

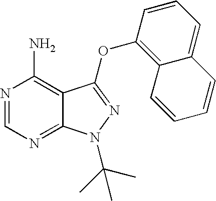

### 1-tert-Butyl-3-(naphthalen-1-yloxy)-1H-pyrazolo[3,4-d]pyrimidin-4-ylamine (Compound No. 198)

A mixture of 3-bromo-1-tert-butyl-1H-pyrazolo[3,4-d]pyrimidin-4-ylamine (500 mg, 1.85 mmol) (Example 10, Step C), 2,2,6,6-tetramethyl-heptane-3,5-dione (34.1 mg, 0.18 mmol), cuprous chloride (91.6 mg, 0.92 mmol) and cesium carbonate (1.21 g, 3.71 mmol) in N-methyl pyrrolidone (4 ml) was heated at 120° C. for 18 hr under an Argon atmosphere. Water was added and the product extracted with methylene chloride. Organic layer was washed with water, dried (anhydrous sodium sulfate) and evaporated in vacuo to afford a residue that was flash chromatographed on silica gel (eluent, hexane-ethyl acetate gradient). Fractions containing the desired material were combined and solvent evaporated. The residue was subjected to reverse phase preparative HPLC and the peaks were collected based on the UV absorption at 254 nm (water-acetonitrile gradient, 0.05% formic acid, 80:20 to 10:90, 20 min, linear gradient; flow, 15 ml/min; column, Phenomenex Luna 5μ C18, 100×21.2 mm; UV 254 and 218 nm). Fractions with the desired material were pooled and solvent evaporated in vacuo. Trituration with acetonitrile afforded the title compound (9 mg, 0.03 mmol), LC/MS, API-ES, Pos, (M+H)⁺, 334.2.

### Example 34

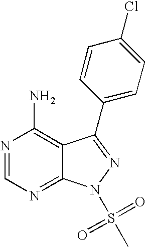

### 3-(4-Chloro-phenyl)-1-methanesulfonyl-1H-pyrazolo[3,4-d]pyrimidin-4-ylamine (Compound No. 203)

To a suspension of 3-(4-chlorophenyl)-1H-pyrazolo[3,4-d]pyrimidin-4-amine (200 mg, 0.81 mmol) in 5 ml methylene chloride, pyridine (77 mg, 0.98 mmol) was added followed by methanesulfonyl chloride (102 mg, 0.90 mmol). After 1.5 hr, additional 1.2 equivalent of pyridine was added and the reaction stirred overnight. 1.1 equivalent of methanesulfonyl chloride and 1.2 equivalent of pyridine were added and the reaction stirred for 4 hr at ambient temperature. The reaction was quenched with 5 ml of water and extracted with methylene chloride. The organic layer was washed with water, dried (anhydrous sodium sulfate) and evaporated to afford a residue that was subjected to column chromatography on silica gel (eluent; methylene chloride-methanol gradient) to afford the title compound (33 mg, 0.10 mmol); LC/MS, API-ES, Pos, (M+H)⁺, 324.0, 326.0.

### Example 35

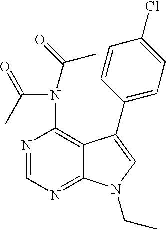

### N-Acetyl-N-[5-(4-chloro-phenyl)-7-ethyl-7H-pyrrolo[2,3-d]pyrimidin-4-yl]-acetamide (Compound No. 211)

To a stirred solution of 5-(4-Chloro-phenyl)-7-ethyl-7H-pyrrolo[2,3-d]pyrimidin-4-ylamine (42 mg, 0.15 mmol) in pyridine (2 ml) was added acetyl chloride (0.15 ml, 1.5 mmol) and the reaction mixture was stirred at 60° C. for 1 day until reaction completion (LCMS). The mixture was concentrated in vacuo and purified by chromatography on silica gel (10 g, eluent hexanes-ethyl acetate 100:0 to 0:100) to afford the title compound (36 mg, 0.10 mmol) as a white solid; LC/MS, API-ES, Pos, (M+H)⁺, 357.1, 359.1.

### Example 36

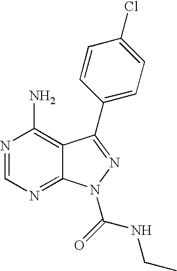

### 4-Amino-3-(4-chloro-phenyl)-pyrazolo[3,4-d]pyrimidine-1-carboxylic acid ethylamide (Compound No. 213)

### Step A

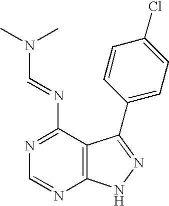

To a solution of 3-(4-chlorophenyl)-1H-pyrazolo[3,4-d]pyrimidin-4-amine (500 mg, 2.04 mmol) in dimethylformamide (7 ml), pyridine (193 mg, 2.44 mmol) and methanesulfonyl chloride (257 mg, 2.24 mmol) were added. After 1 hr of stirring at ambient temperature, additional pyridine (193 mg, 2.44 mmol) and methanesulfonyl chloride (257 mg, 2.24 mmol) were added and solution stirred for 2 hr. The reaction mixture was poured into water and basified with 20 ml of saturated aqueous sodium bicarbonate solution. The precipitated product was separated by filtration and washed with acetone to afford N′-[3-(4-chloro-phenyl)-1H-pyrazolo[3,4-d]pyrimidin-4-yl]-N,N-dimethyl-formamidine as a brown solid (397 mg, 1.32 mmol); LC/MS, API-ES, Pos, (M+H)⁺, 301.0, 303.1.

### Step B

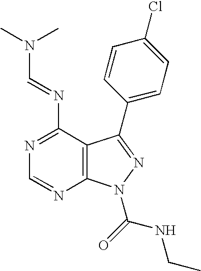

To a suspension of N′-(3-(4-chlorophenyl)-1H-pyrazolo[3,4-d]pyrimidin-4-yl)-N,N-dimethylformamidine (200 mg, 0.67 mmol) in 8 ml dioxane, ethyl isocyanate (0.05 ml, 0.67 mmol) was added. The reaction mixture was stirred at room temperature for 1 hr followed by heating at 60° C. for 2 hr. The mixture was stirred at 20° C. over night and an additional 1.2 equivalent of ethyl isocyanate was added followed by stirring at 40° C. for 8 hr. The mixture was evaporated in vacuo and the residue triturated with acetone to afford 3-(4-chlorophenyl)-4-(dimethylamino-methyleneamino)-pyrazolo[3,4-d]pyrimidine-1-carboxylic acid ethylamide (180 mg, 0.48 mmol).

### Step C

3-(4-Chloro-phenyl)-4-(dimethylamino-methyleneamino)-pyrazolo[3,4-d]pyrimidine-1-carboxylic acid ethylamide (150 mg, 0.40 mmol) was dissolved in 10 ml of 1 molar hydrochloric acid solution in acetonitrile. The mixture was stirred at 25° C. over night. Then the mixture was concentrated under reduced pressure and neutralized with saturated aqueous sodium carbonate solution (pH 8) and filtered. The solid was triturated with acetone to furnish the title compound (60 mg, 0.18 mmol); LC/MS, API-ES, Pos, (M+H)⁺, 316.7, 318.9.

### Example 37

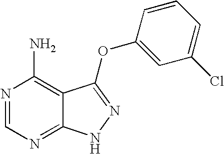

### 3-(3-Chloro-phenoxy)-1H-pyrazolo[3,4-d]pyrimidin-4-ylamine (Compound No. 220)

1-tert-Butyl-3-(3-chloro-phenoxy)-1H-pyrazolo[3,4-d]pyrimidin-4-ylamine (1 g, 3.3 mmol) (prepared from 3-chlorophenol and 3-bromo-1-tert-butyl-1H-pyrazolo[3,4-d]pyrimidin-4-ylamine according to a procedure similar to as described for Example 21) was added in portions to stirred concentrated sulfuric acid at 0° C. Solution was stirred at this temperature for 15 min followed by stirring at ambient temperature for 30 min. The solution was slowly poured on ice and the precipitate separated by filtration. The aqueous filtrate was neutralized with aqueous sodium carbonate and the precipitate separated by filtration. The combined precipitate was washed with water and air dried. Trituration with a small amount of methanol afforded the title compound (Compound No. 220; 0.61 g, 2.48 mmol); LC/MS, API-ES, Pos, (M+H)⁺, 262.0, 264.0.

### Example 38

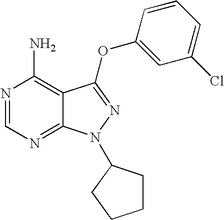

### 3-(3-Chloro-phenoxy)-1-cyclopentyl-1H-pyrazolo[3,4-d]pyrimidin-4-ylamine (Compound No. 221)

A mixture of 3-(3-chloro-phenoxy)-1H-pyrazolo[3,4-d]pyrimidin-4-ylamine (150 mg, 0.57 mmol), cyclopentyl bromide (169.9 mg, 1.14 mmol) and potassium carbonate (173.1 mg, 1.25 mmol) in anhydrous dimethylformamide (2 ml) was stirred at 70° C. for 4 hr. Water was added and the product extracted with methylene chloride. Organic layer was washed with water, dried (anhydrous sodium sulfate) and evaporated in vacuo. The residue was subjected to reverse phase preparative HPLC (water-acetonitrile gradient, 0.05% formic acid, 80:20 to 10:90, 20 min, linear gradient; flow, 15 ml/min; column, Phenomenex Luna 5μ C18, 100×21.2 mm; UV 254 and 218 nm). Fractions containing the desired material were combined and solvent evaporated in vacuo to afford the title compound (19 mg, 0.06 mmol); LC/MS, API-ES, Pos, (M+H)⁺, 330.1, 332.1.

### Example 39

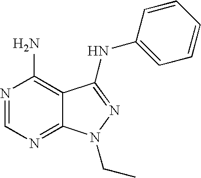

### 1-Ethyl-N³-phenyl-1H-pyrazolo[3,4-d]pyrimidine-3,4-diamine (Compound No. 223)

### Step A

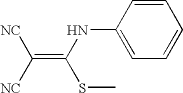

2-(Bis-methylsulfanyl-methylene)-malononitrile (3.40 g, 20 mmol) and aniline (1.82 ml, 20 mmol) were refluxed in ethanol (50 ml) for 1.5 h until reaction completion (LCMS). The mixture was allowed to cool down, the precipitate formed was filtered, washed with ethanol (3×7 ml), and dried on air to afford 2-(methylsulfanyl-phenylamino-methylene)-malononitrile (2.97 g, 13.8 mmol) as an off-white solid.

### Step B

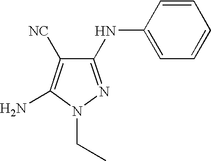

To the stirred suspension of 2-(methylsulfanyl-phenylamino-methylene)-malononitrile (215 mg, 1.0 mmol) and ethylhydrazine oxalate (150 mg, 1.0 mmol) in ethanol (20 ml), triethylamine (0.14 ml, 1.0 mmol) was added and the mixture was refluxed for 15 h, allowed to cool down, and concentrated in vacuo. LCMS analysis indicated the presence of two isomeric products that were separated by column chromatography on silica gel (50 g, eluent hexanes-ethyl acetate 100:0 to 0:100) to afford 5-amino-1-ethyl-3-phenylamino-1H-pyrazole-4-carbonitrile (93 mg, 0.41 mmol) as a white low-melting solid, LCMS, 3.42 min, API-ES, Pos, (M+H)⁺, 228.1, and the isomeric 3-amino-1-ethyl-5-phenylamino-1H-pyrazole-4-carbonitrile (39 mg, 0.17 mmol) as an off-white solid, LC/MS, 3.18 min.

### Step C

5-Amino-1-ethyl-3-phenylamino-1H-pyrazole-4-carbonitrile (92 mg, 0.40 mmol) was stirred in formamide (6 ml) at 190° C. for 6 h. The mixture was diluted with water (10 ml), filtered, washed with water (3×5 ml), dried, redissolved in THF (20 ml), treated with activated charcoal, filtered through celite, concentrated in vacuo, and purified by chromatography on silica gel (10 g, eluent, dichloromethane-methanol 100:0 to 80:30) to afford 1-ethyl-N³-phenyl-1H-pyrazolo[3,4-d]pyrimidine-3,4-diamine (41 mg, 0.16 mmol) as an off-white solid, LC/MS, API-ES, Pos, (M+H)⁺, 255.1.

### Example 40

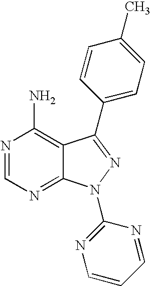

### 1-Pyrimidin-2-yl-3-p-tolyl-1H-pyrazolo[3,4-d]pyrimidin-4-ylamine (Compound No. 229)

A mixture of 3-p-tolyl-1H-pyrazolo[3,4-d]pyrimidin-4-ylamine (245.6 mg, 1 mmol), 2-chloropyrimidine (137.4 mg, 1.2 mmol) and potassium carbonate (165.6 mg, 1.2 mmol) in anhydrous dimethylsulfoxide (2 ml) was stirred at 110° C. for 2 hr under an Argon atmosphere. Water was added and the product extracted with methylene chloride. Organic layer was washed with water, dried (anhydrous sodium sulfate) and evaporated in vacuo. The residue was triturated with acetonitrile and subjected to reverse phase preparative chromatography (water-acetonitrile gradient, 0.05% formic acid, 80:20 to 10:90, 20 min, linear gradient; flow, 15 ml/min; column, Phenomenex Luna 5μ C18, 100×21.2 mm; UV 254 and 218 nm). The fractions containing the desired material were pooled and solvent evaporated in vacuo to afford the title compound (33 mg, 0.10 mmol); LC/MS, API-ES, Pos, (M+H)⁺, 324.0, 326.0.

### Example 41

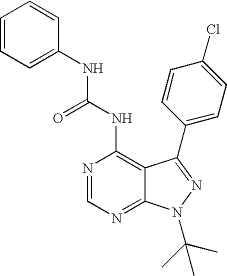

### 1-[1-tert-Butyl-3-(4-chloro-phenyl)-1H-pyrazolo[3,4-d]pyrimidin-4-yl]-3-phenyl-urea (Compound No. 230)

A mixture of 1-tert-butyl-3-(4-chlorophenyl)-1H-pyrazolo[3,4-d]pyrimidin-4-amine (100 mg, 0.33 mmol) and benzene isocyanate (39.5 mg, 0.33 mmol) in dioxane (2 ml) was stirred at room temperature overnight. The solvent was removed in vacuo and the residue triturated with acetone to afford the title compound (60 mg, 0.14 mmol); LC/MS, API-ES, Neg, (M−H)⁻, 419.3, 421.2.

### Example 42

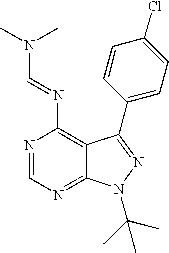

### N′-[1-tert-Butyl-3-(4-chloro-phenyl)-1H-pyrazolo[3,4-d]pyrimidin-4-yl]-N,N-dimethyl-formamidine (Compound No. 232)

1-tert-Butyl-3-(4-chlorophenyl)-1H-pyrazolo[3,4-d]pyrimidin-4-amine (150 mg, 0.50 mmol) was dissolved in 2 ml dimethylformamide dimethylacetal. The mixture was stirred at room temperature over night. The solvent was evaporated in vacuo and the product purified by column chromatography on silica gel (eluent; methylene chloride:methanol, 10:1) to afford the product as a sticky solid. The material was treated with hexane in an ultrasonic bath. The product separated as a precipitate which was removed by filtration to afford the title compound (84 mg, 0.24 mmol); LC/MS, API-ES, Pos, (M+H)⁺, 357.3, 359.3.

### Example 43

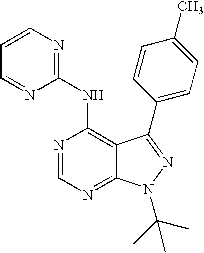

### (1-tert-Butyl-3-p-tolyl-1H-pyrazolo[3,4-d]pyrimidin-4-yl)-pyrimidin-2-yl-amine (Compound No. 234)

### Step A

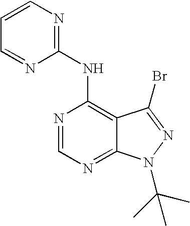

A mixture of 3-bromo-1-tert-butyl-1H-pyrazolo[3,4-d]pyrimidin-4-ylamine (1.35 g, 5.0 mmol) (Example 10, Step C), 2-chloropyrimidine (1.15 g, 10.0 mmol) and potassium carbonate (1.38 g, 10.0 mmol) in anhydrous dimethyl sulfoxide (10 ml) was heated with stirring at 120° C. for 12 hr under an Argon atmosphere. Water was added and the product extracted with methylene chloride. The organic layer was washed with water, dried (anhydrous sodium sulfate) and evaporated. The residue was stirred in acetonitrile at ambient temperature and the precipitated material was separated by filtration and air dried to afford (3-bromo-1-tert-butyl-1H-pyrazolo[3,4-d]pyrimidin-4-yl)-pyrimidin-2-yl-amine (0.69 g, 1.98 mmol). The compound was used as such for the next step.

### Step B

A mixture of (3-bromo-1-tert-butyl-1H-pyrazolo[3,4-d]pyrimidin-4-yl)-pyrimidin-2-yl-amine (348.2 mg, 1 mmol), 4-methylbenzeneboronic acid (149.6 mg, 1.1 mmol), sodium carbonate (254.4 mg, 2.4 mmol) and tetrakistriphenylphosphine palladium (69.3 mg, 0.06 mmol) in ethyleneglycol dimethylether (25 ml) and water (12.5 ml) was heated at reflux for 6 hr under an atmosphere of argon. Water was added and the product extracted with methylene chloride. The organic layer was washed with water, dried (anhydrous sodium sulfate) and evaporated in vacuo. The residue was subjected to preparative reverse phase HPLC (water-acetonitrile gradient, 0.05% formic acid, 80:20 to 10:90, 20 min, linear gradient; flow, 15 ml/min; column, Phenomenex Luna 5μ C18, 100×21.2 mm; UV 254 and 218 nm). The fractions containing the desired material were combined and evaporated in vacuo. Trituration with methanol followed by acetonitrile afforded the title compound (113 mg, 0.31 mmol); LC/MS, API-ES, Pos, (M+H)⁺, 360.1.

### Example 44

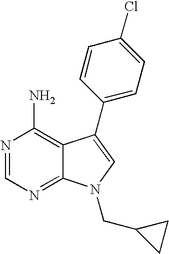

### 5-(4-Chloro-phenyl)-7-cyclopropylmethyl-7H-pyrrolo[2,3-d]pyrimidin-4-ylamine (Compound No. 242)

### Step A

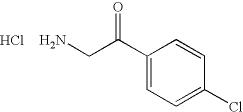

To a solution of hexamethylenetetramine (17.6 g, 125.5 mmol) in EtOH (500 ml) was added dropwise 2-bromo-4′-chloroacetophenone (26.6 g, 114.1 mmol) in chloroform (60 ml). Sodium iodide (17.1 g, 114.1 mmol) was added after 1 hr. The mixture stirred at room temperature for 24 hr. The precipitate was filtered and washed with cold ethanol. The material was diluted with ethanol (100 ml) and concentrated hydrochloric acid (36.5 ml, 433 mmol) and heated to 55° C. for 30 min. The mixture was cooled and allowed to evaporate for 24 hr. The solid was diluted with hexane, stirred for 15 min and filtered to furnish 2-amino-1-(4-chloro-phenyl)-ethanone hydrochloride (15.9 g, 77.2 mmol). The compound was used as such for the next step.

### Step B

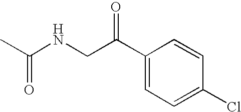

To acetic anhydride (30.5 ml, 323.2 mmol) cooled to 0° C. was added 2-amino-1-(4-chloro-phenyl)-ethanone hydrochloride (15.9 g, 77.2 mmol) followed by addition of sodium acetate (12.6 g, 154.3 mmol) in water (30 ml). The solution was kept at 0° C. for 30 min, warmed to room temperature, stirred for 4 hr, and diluted with 1N hydrochloric acid. The aqueous layer was extracted with methylene chloride. The organic layer was washed with brine, dried (MgSO₄) and concentrated in vacuo to afford N-[2-(4-chloro-phenyl)-2-oxo-ethyl]-acetamide (13.0 g, 61.4 mmol). The compound was used as such for the next step.

### Step C

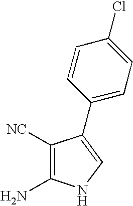

To a solution of N-[2-(4-chloro-phenyl)-2-oxo-ethyl]-acetamide (13.0 g, 61.4 mmol) in methanol (100 ml) was added malonitrile (5.5 g, 83.1 mmol). The solution was cooled to 0° C. and 50% aqueous potassium hydroxide was added until pH 10-12 was reached. The dark solution was stirred at 0° C. for 20 min and was then heated to 65° C. for 6 hr. The mixture was cooled to room temperature and poured over ice. The precipitate was filtered, washed with water, and dried in vacuo to afford 2-amino-4-(4-chloro-phenyl)-1H-pyrrole-3-carbonitrile (10.4 g, 47.8 mmol). The compound was used as such for the next step.

### Step D

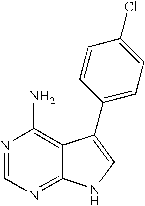

2-Amino-4-(4-chloro-phenyl)-1H-pyrrole-3-carbonitrile (3.6 g, 16.5 mmol) was diluted with formamide (25 ml, 628.3 mmol) and heated to 185° C. for 4 hr. The solution was cooled to room temperature, diluted with water, and extracted with ethyl acetate. The organic was concentrated. The resulting solid was triturated with water-acetone (4:1), filtered and triturated again with diethyl ether. The solid was dried in vacuo to afford 5-(4-chloro-phenyl)-7H-pyrrolo[2,3-d]pyrimidin-4-ylamine (2.9 g, 11.9 mmol). The compound was used as such for the next step.

### Step E

To 5-(4-chloro-phenyl)-7H-pyrrolo[2,3-d]pyrimidin-4-ylamine (190 mg, 0.78 mmol) in DMF (3 ml) was added sodium ethoxide (64 mg, 0.94 mmol). The mixture was stirred at room temperature for 15 min followed by addition of bromomethylcyclopropane (115 mg, 0.86 mmol). The solution was stirred for 16 hr. The crude material was purified via preparative reverse phase LC/MS (water-acetonitrile gradient, 0.05% formic acid, 95:5 to 5:95, 14 min, linear gradient; flow, 42 ml/min, column, Phenomenex Luna 5μ C18(2), 100×30 mm; UV 254 and 218 nm). The fractions containing the desired material were combined and evaporated in vacuo to give the title compound (16 mg, 53.5 mmol); LC/MS, API-ES, Pos, (M+H)⁺, 299.1.

### Example 45

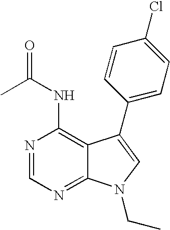

### N-[5-(4-Chloro-phenyl)-7-ethyl-7H-pyrrolo[2,3-d]pyrimidin-4-yl]-acetamide (Compound No. 246)

To a solution of 5-(4-chloro-phenyl)-7-ethyl-7H-pyrrolo[2,3-d]pyrimidin-4-ylamine (50 mg, 184 mmol) in acetic anhydride (1 ml) at 0° C. was added sodium acetate (30 mg, 368 mmol). The solution was warmed to room temperature and stirred for 4 hr. The crude material was purified via preparative reverse phase LC/MS (water-acetonitrile gradient, 0.05% formic acid, 95:5 to 5:95, 14 min, linear gradient; flow, 42 ml/min, column, Phenomenex Luna 5μ C18(2), 100×30 mm; UV 254 and 218 nm). The fractions containing the desired material were combined and evaporated in vacuo to give the title compound (18 mg, 57 mmol); LC/MS, API-ES, Pos, (M+H)⁺, 315.1.

### Example 46

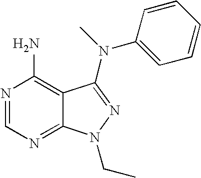

### 1-Ethyl-N³-methyl-N³-phenyl-1H-pyrazolo[3,4-d]pyrimidine-3,4-diamine (Compound No. 248)

To the stirred mixture of 1-ethyl-N³-phenyl-1H-pyrazolo[3,4-d]pyrimidine-3,4-diamine (100 mg, 0.4 mmol) and potassium carbonate (70 mg, 0.5 mmol) in anhydrous DMF (3 ml), was added methyl iodide (25 μl, 0.4 mmol) and the mixture was stirred for 1 day at 40° C. in a closed vial. Additional amount of methyl iodide (25 μl, 0.4 mmol) was introduced and the mixture was stirred for another 4 days at 40° C. The resulting mixture of products was filtered and purified by prep-HPLC on C18 reverse phase silica gel column (eluent, H₂O—CH₃CN—HCOOH, 95:5:0.05 to 5:95:0.05) followed by recrystallization from acetonitrile to afford the title compound (18 mg, 0.067 mmol) as a white powder; LC/MS, API-ES, Pos, (M+H)⁺, 269.1.

### Example 47

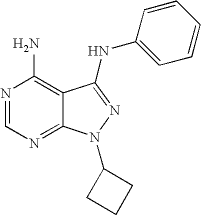

### 1-Cyclobutyl-N³-phenyl-1H-pyrazolo[3,4-d]pyrimidine-3,4-diamine (Compound No. 253)

### Step A

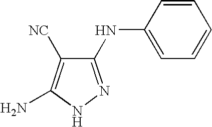

A solution of 2-(methylsulfanyl-phenylamino-methylene)-malononitrile (2.07 g, 9.6 mmol) (Example 39, Step A) and hydrazine-hydrate (0.45 ml, 10 mmol) in anhydrous ethanol (50 ml) was refluxed for 2 h under inert atmosphere (complete conversion by LCMS). The resulting mixture was concentrated in vacuo to afford pure 5-amino-3-phenylamino-1H-pyrazole-4-carbonitrile (1.88 g, 9.4 mmol) as an off-white solid.

### Step B

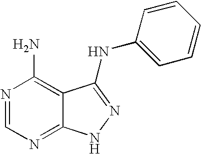

A mixture of 5-amino-3-phenylamino-1H-pyrazole-4-carbonitrile (1.88 g, 9.4 mmol) and formamide (30 ml) was stirred with a reflux condenser at 190° C. for 3 h (LCMS control). The reaction was allowed to cool down, the precipitate was filtered, washed with water (3×7 ml) and ether (2×5 ml), and dried in vacuo to afford pure N³-phenyl-1H-pyrazolo[3,4-d]pyrimidine-3,4-diamine (1.88 g, 8.3 mmol) as a light brown solid.

### Step C

To the stirred suspension of N³-phenyl-1H-pyrazolo[3,4-d]pyrimidine-3,4-diamine (226 mg, 1.0 mmol) and potassium carbonate (276 mg, 2.0 mmol) in anhydrous DMF (3 ml) under argon was added cyclobutyl bromide (200 μl, 2.0 mmol), and the resulting mixture was stirred in a sealed vial at 70° C. for 12 h, filtered, and purified by prep-HPLC on reverse phase C18 column (eluent H₂O—CH₃CN—HCOOH 95:5:0.05 to 5:95:0.05) followed by recrystallization from acetonitrile to afford the title 1-cyclobutyl-N³-phenyl-1H-pyrazolo[3,4-d]pyrimidine-3,4-diamine (133 mg, 0.48 mmol) as off-white solid; LC/MS, API-ES, Pos, (M+H)⁺, 281.1.

### Example 48

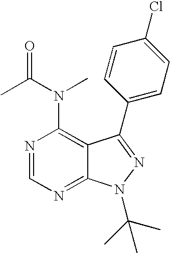

### N-[1-tert-Butyl-3-(4-chloro-phenyl)-1H-pyrazolo[3,4-d]pyrimidin-4-yl]-N-methyl-acetamide Compound No. 259)

### Step A

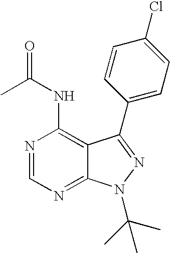

To the stirred suspension of 1-tert-butyl-3-(4-chloro-phenyl)-1H-pyrazolo[3,4-d]pyrimidin-4-ylamine (302 mg, 1.0 mmol) in pyridine (5 ml) was added acetic anhydride (0.30 ml, 3.2 mmol), the mixture was heated in a sealed vial at 70° C. for 1 h (LCMS control), concentrated in vacuo, and purified by chromatography on silica gel (20 g, eluent hexanes-ethyl acetate 100:0 to 50:50) followed by trituration with acetonitrile to afford N-[1-tert-Butyl-3-(4-chloro-phenyl)-1H-pyrazolo[3,4-d]pyrimidin-4-yl]-acetamide (Compound No. 2; 180 mg, 0.52 mmol) as an off-white solid, along with di-acylated by-product (Compound No. 97; 91 mg, 0.24 mmol).

### Step B

To the stirred suspension of N-[1-tert-Butyl-3-(4-chloro-phenyl)-1H-pyrazolo[3,4-d]pyrimidin-4-yl]-acetamide (68 mg, 0.2 mmol) and potassium carbonate (41 mg, 0.3 mmol) in anhydrous DMF (3 ml) was added methyl iodide (19 μl, 0.3 mmol), and the mixture was stirred overnight at ambient temperature, filtered, and purified by prep-HPLC on reverse phase C18 column (eluent H₂O—CH₃CN—HCOOH 95:5:0.05 to 5:95:0.05) to afford N-[1-tert-Butyl-3-(4-chloro-phenyl)-1H-pyrazolo[3,4-d]pyrimidin-4-yl]-N-methyl-acetamide (47 mg, 0.13 mmol) as a white solid; LC/MS, API-ES, Pos, (M+H)⁺, 358.1, 360.1.

### Example 49

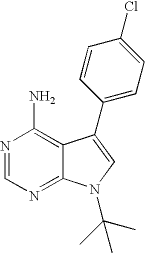

### 7-tert-Butyl-5-(4-chloro-phenyl)-7H-pyrrolo[2,3-d]pyrimidin-4-ylamine (Compound No. 261)

### Step A

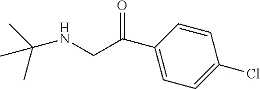

To a stirred solution of tert-butyl amine (6.8 g, 30 mmol) in toluene (20 mL) at 0° C. was added 4′-chloro-2-bromo-acetophenone (2.3 g, 20 mmol) in toluene (15 mL) dropwise over 5 min. The solution was stirred at 0° C. for 1.5 hr. Concentrated hydrochloric acid was added to pH 1-2 and the mixture was stirred at 0° C. for 15 min. The resulting precipitate was filtered and washed with diethyl ether. This material [2-tert-butylamino-1-(4-chloro-phenyl)-ethanone] was used for the next step as such.

### Step B

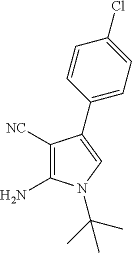

To a mixture of 2-tert-butylamino-1-(4-chloro-phenyl)-ethanone (0.98 g, 3.72 mmol), malonitrile (0.32 g, 4.84 mmol) in methanol (10 ml) at 0° C. was added 50% aqueous potassium hydroxide to pH 10-12. The dark colored solution was heated at 65° C. while maintaining pH for 4 hr. The material was poured over crushed ice. The precipitate was collected and washed with water to furnish 2-amino-1-tert-butyl-4-(4-chloro-phenyl)-1H-pyrrole-3-carbonitrile (0.35 g, 1.28 mmol).

### Step C

A stirred mixture of 2-amino-1-tert-butyl-4-(4-chloro-phenyl)-1H-pyrrole-3-carbonitrile (0.23 g, 0.86 mmol) and formamide (5 ml) was heated to 180° C. for 4 hr. The solution was cooled, diluted with water and extracted with methylene chloride. The organic layer was concentrated and purified via preparative reverse phase HPLC (water-acetonitrile gradient, 0.05% formic acid, 80:20 to 10:90, 20 min, linear gradient; flow, 15 ml/min; column, Phenomenex Luna 5μ C18, 100×21.2 mm; UV 254 and 218 nm). The fractions containing the desired material were combined and evaporated in vacuo to give the title compound (16 mg, 53 mmol); LC/MS, API-ES, Pos, (M+H)⁺, 301.1.

### Example 50

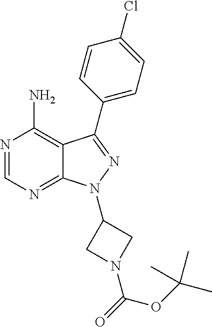

### 3-[4-Amino-3-(4-chloro-phenyl)-pyrazolo[3,4-d]pyrimidin-1-yl]-azetidine-1-carboxylic acid tert-butyl ester (Compound No. 267)

### Step A

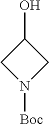

To a solution of azetidine-3-ol hydrochloride (1.00 g, 9.13 mmol) in ethanol (18 ml) at 0° C. was added triethyl amine (3.80 ml, 27.4 mmol) followed by di-t-butyl dicarbonate (2.18 g, 10.0 mmol). The mixture was stirred at room temperature for 30 min. The solution was concentrated under reduced pressure and the residue dissolved in ethyl acetate, washed with 10% citric acid followed by brine. The organic layer was dried (anhydrous sodium sulfate) and solvent removed in vacuo. The crude product was purified by flash chromatography on silica gel (eluent; hexane-ethyl acetate gradient) to afford 3-hydroxy-azetidine-1-carboxylic acid tert-butyl ester as a colorless oil (0.8 g, 4.62 mmol).

### Step B

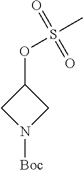

To a solution of tert-butyl 3-hydroxy-1-azetidinecarboxylate (770 mg, 4.45 mmol) in ethyl acetate (7 ml) was added triethyl amine (0.80 ml, 5.78 mmol) followed by methanesulfonyl chloride (0.41 ml, 5.34 mmol). The mixture was stirred at 0° C. for 1 hr. The solution was filtered and the residue washed with ethyl acetate. The organic filtrate was evaporated in vacuo to afford 3-methanesulfonyloxy-azetidine-1-carboxylic acid tert-butyl ester as a yellow oil (1 g, 3.98 mmol).

### Step C

A mixture of 3-(4-chlorophenyl)-1H-pyrazolo[3,4-d]pyrimidin-4-amine (684 mg, 2.79 mmol), 3-methanesulfonyloxy-azetidine-1-carboxylic acid tert-butyl ester (700 mg, 2.79 mmol) and cesium carbonate (1.18 g, 3.62 mmol) was heated in dimethylformamide (14 ml) at 90° C. for 24 hr under argon. The mixture was allowed to cool to room temperature, poured into ice water and extracted with 5% methanol in methylene chloride. The organic layer was dried (anhydrous sodium sulfate) and evaporated under reduced pressure. The product was purified by flash chromatography on silica gel (eluent; methylene chloride-methanol gradient) to afford the title compound (600 mg, 1.49 mmol); LC/MS, API-ES, Pos, (M+H)⁺, 400.9, 402.9.

### Example 51

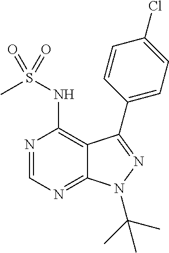

### N-[1-tert-Butyl-3-(4-chloro-phenyl)-1H-pyrazolo[3,4-d]pyrimidin-4-yl]-methanesulfonamide (Compound No. 274)

To a stirred solution of 1-tert-butyl-3-(4-chlorophenyl)-1H-pyrazolo[3,4-d]pyrimidin-4-amine (100 mg, 0.33 mmol) in dimethylformamide (1.5 ml) was added sodium hydride (60% in mineral oil, 40.0 mg, 0.85 mmol) at 0° C. After stirring for 20 min, methanesulfonylchloride (80.0 mg, 0.66 mmol) was slowly added and the mixture stirred at ambient temperature for one hr. The reaction was quenched by addition of cold water and extracted with ethyl acetate. The combined organic layer was washed with water followed by brine, dried (anhydrous sodium sulfate) and concentrated in vacuo. The residue was purified by column chromatography on silica gel (methylene chloride-methanol gradient) to afford the title compound (65 mg, 0.17 mmol); LC/MS, API-ES, Pos, (M+H)⁺, 380.0, 382.0.

### Example 52

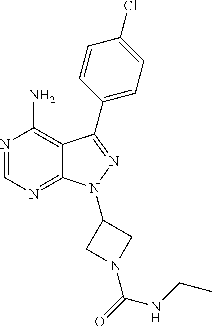

### 3-[4-Amino-3-(4-chloro-phenyl)-pyrazolo[3,4-d]pyrimidin-1-yl]azetidine-1-carboxylic acid ethylamide (Compound No. 275)

To a solution of 1-(azetidin-3-yl)-3-(4-chlorophenyl)-1H-pyrazolo[3,4-d]pyrimidin-4-amine dihydrochloride (200 mg, 0.54 mmol) in dioxane (5 ml) was added pyridine (0.14 ml, 1.71 mmol) and ethyl isocyanate (0.11 m, 1.34 mmol). The suspension was stirred at room temperature for 24 hr. Dimethylformamide (3 ml) was added and the mixture stirred at room temperature for additional 24 hr. Water was added and the product extracted with ethyl acetate, dried (anhydrous sodium sulfate) and solvent removed in vacuo. The crude product was purified by column chromatography on silica gel (eluent; methylene chloride:methanol, 25:1) to furnish the title compound (100 mg, 0.27 mmol); LC/MS, API-ES, Pos, (M+H)⁺, 372.1, 374.1.

### Example 53

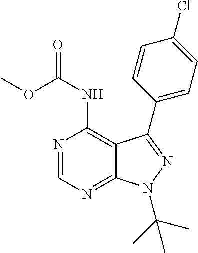

### [1-tert-Butyl-3-(4-chloro-phenyl)-1H-pyrazolo[3,4-d]pyrimidin-4-yl]-carbamic acid methyl ester (Compound No. 294)

Sodium hydride (60% in paraffin, 40.0 mg, 0.10 mmol) was added to a solution of 1-tert-butyl-3-(4-chlorophenyl)-1H-pyrazolo[3,4-d]pyrimidin-4-amine (200 mg, 0.66 mmol) in dimethylformamide (2 ml) followed by methyl chloroformate (0.06 ml, 0.80 mmol). The mixture was stirred at ambient temperature for 3 hr. Additional sodium hydride (20.0 mg, 0.50 mmol) and methyl chloroformate (0.03 ml, 0.40 mmol) were added and the mixture stirred for 3 hr. The mixture was diluted with water and the product extracted with ethyl acetate. The organic layer was dried over anhydrous sodium sulfate and solvent removed in vacuo. The residue was subjected to column chromatography (methylene chloride-methanol gradient) followed by preparative circular thin layer chromatography (Chromatotron) to afford the title compound (60 mg, 0.17 mmol); LC/MS, API-ES, Pos, (M+H)⁺, 360.1, 362.1.

### Example 54

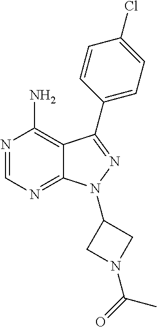

### 1-{3-[4-Amino-3-(4-chloro-phenyl)-pyrazolo[3,4-d]pyrimidin-1-yl]-azetidin-1-yl}-ethanone (Compound No. 295)

To a solution of 1-(azetidin-3-yl)-3-(4-chlorophenyl)-1H-pyrazolo[3,4-d]pyrimidin-4-amine dihydrochloride (200 mg, 0.54 mmol) in dimethylformamide (5 ml) was added pyridine (0.14 ml, 1.71 mmol) and acetyl chloride (0.11 ml, 1.60 mmol). The mixture was stirred at ambient temperature for 2.5 hr. The mixture was diluted with water and extracted with ethylacetate. The organic layer was washed with brine, dried (anhydrous sodium sulfate) and solvent removed in vacuo. Trituration with acetone afforded the title compound (64 mg, 0.19 mmol); LC/MS, API-ES, Neg, (M−H)⁻, 341.3, 343.4.

### Example 55

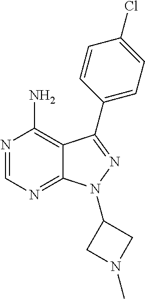

### 3-(4-Chloro-phenyl)-1-(1-methyl-azetidin-3-yl)-1H-pyrazolo[3,4-d]pyrimidin-4-ylamine (Compound No. 296)

A solution of 1-(azetidin-3-yl)-3-(4-chlorophenyl)-1H-pyrazolo[3,4-d]pyrimidin-4-amine dihydrochloride (150 mg, 0.40 mmol) in water was neutralized with aqueous sodium carbonate. The precipitate was separated by filtration and suspended in 5 ml of acetonitrile. Formaldehyde (0.16 ml, 1.61 mmol, 37% aqueous solution) and NaBH₃CN (40.0 mg, 0.64 mmol) were added to the suspension. The pH of the solution was maintained at 7 by addition of acetic acid and solution stirred for 2 hr. The mixture was concentrated in vacuo and the residue dissolved in ethyl acetate followed by washings with aqueous sodium carbonate solution. The organic layer was dried (anhydrous sodium sulfate) and solvent removed in vacuo. The residue was purified by column chromatography on silica gel (eluent; methylene chloride:methanol:ammonia, 100:1:0.5) to afford the title compound (37 mg, 0.12 mmol).

### Example 56

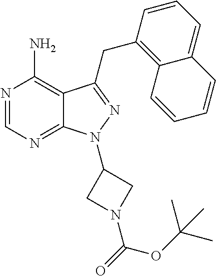

### 3-(4-Amino-3-naphthalen-1-ylmethyl-pyrazolo[3,4-d]pyrimidin-1-yl)-azetidine-1-carboxylic acid tert-butyl ester (Compound No. 297)

A mixture of 3-((naphthalen-1-yl)methyl)-1H-pyrazolo[3,4-d]pyrimidin-4-amine (5.00 g, 18.2 mmol), cesium carbonate (7.69 g, 23.6 mmol) and 1-(tert-butoxycarbonyl)azetidin-3-yl methanesulfonate (5.02 g, 20.0 mmol) in dimethylformamide (200 ml) was stirred at 85° C. over night. The mixture was added to water and extracted with ethyl acetate. The organic layers were washed with water, brine, dried (anhydrous sodium sulfate) and solvent removed in vacuo. The residue was purified by column chromatography on silica gel (eluent; methylene chloride:methanol, 50:1) to furnish the title compound (5.8 g, 13.5 mmol); LC/MS, API-ES, Pos, (M+H)⁺, 431.1.

### Example 57

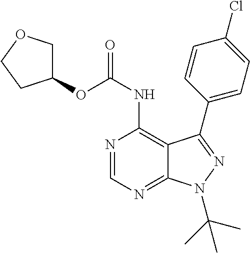

### [1-tert-Butyl-3-(4-chloro-phenyl)-1H-pyrazolo[3,4-d]pyrimidin-4-yl]-carbamic acid tetrahydro-furan-3-yl ester (Compound No. 301)

### Step A

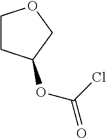

To a solution of (S)-tetrahydrofuran-3-ol (88.0 mg, 1.0 mmol) in 2 ml dichloromethane was added triphosgene (133 mg, 0.45 mmol). The solution was cooled to −40° C. and a solution of pyridine (105 mg, 1.33 mmol) in methylene chloride (2 ml) was slowly added over 5 min. The mixture was stirred at room temperature for 3.5 hr and the solvent was removed in vacuo to afford crude (S)-tetrahydrofuran-3-yl chloroformate.

### Step B

To a suspension of 1-tert-butyl-3-(4-chlorophenyl)-1H-pyrazolo[3,4-d]pyrimidin-4-amine (150 mg, 0.50 mmol) in 5 ml dimethylformamide was added sodium hydride (60% in mineral oil, 40 mg, 1.00 mmol). The mixture was stirred for 5 min at ambient temperature and (S)-tetrahydrofuran-3-yl chloroformate was added. After stirring over night, additional 3 equivalents of (S)-tetrahydrofuran-3-yl chloroformate and 4 equivalents of sodium hydride were added and the mixture stirred for 24 hr. Water was added and the product extracted with ethyl acetate, dried (anhydrous sodium sulfate) and the solvent was removed in vacuo. The residue was purified by column chromatography on silica gel (eluent; methylene chloride:methanol, 100:1) followed by preparative circular TLC (Chromatotron) to afford the title compound (20 mg, 0.05 mmol); LC/MS, API-ES, Neg, (M−H)⁻, 414.3, 416.1.

### Example 58

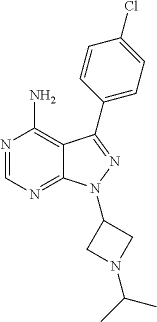

### 3-(4-Chloro-phenyl)-1-(1-isopropyl-azetidin-3-yl)-1H-pyrazolo[3,4-d]pyrimidin-4-ylamine (Compound No. 304)

A suspension of 1-(azetidin-3-yl)-3-(4-chlorophenyl)-1H-pyrazolo[3,4-d]pyrimidin-4-amine dihydrochloride (150 mg, 0.40 mmol) in 1,2-dichloroethane was basified (pH 8) by addition of aqueous sodium carbonate solution. The white solid was separated by filtration and suspended in 5 ml of 1,2-dichloroethane. Acetone (0.03 ml, 0.41 mmol), NaBH(OAc)₃ (119 mg, 0.56 mmol) and acetic acid (0.02 ml, 0.40 mmol) were added and the mixture stirred at ambient temperature for 24 hr. The mixture was diluted with aqueous sodium carbonate, extracted with methylene chloride, dried (anhydrous sodium sulfate) and evaporated. The residue was purified by column chromatography on silica gel (eluent; methylene chloride:methanol:ammonium hydroxide, 100:1:0.5) to afford the title compound (20 mg, 0.06 mmol); LC/MS, API-ES, Pos, (M+H)⁺, 343.3, 345.2.

### Example 59

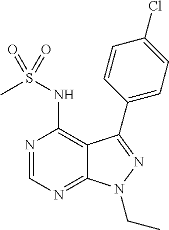

### N-[5-(4-Chloro-phenyl)-7-ethyl-7H-pyrrolo[2,3-d]pyrimidin-4-yl]-methanesulfonamide (Compound No. 305)

Sodium hydride (60% in mineral oil, 0.02 g, 0.73 mmol) was slowly added to a solution of 5-(4-chlorophenyl)-7-ethyl-7H-pyrrolo[2,3-d]pyrimidin-4-amine (0.10 g, 0.37 mmol) in 10 ml of anhydrous dimethylformamide. After stirring for 0.5 hr, methanesulfonyl chloride (0.08 g, 0.73 mmol) was added. The solution was stirred for two hr and additional two molar equivalent of sodium hydride followed by two molar equivalents of ethanesulfonyl chloride were added and solution stirred over night. Water was added and product extracted with ethyl acetate. The solvent was removed in vacuo and the residue purified by column chromatography on silica gel (hexane-acetone gradient) followed by trituration with hexane to afford the title compound (28 mg, 0.08 mmol); LC/MS, API-ES, Pos, (M+H)⁺, 351.0, 353.1.

### Example 60

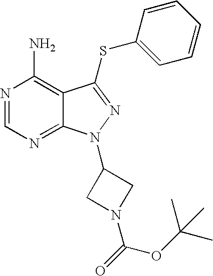

### 3-(4-Amino-3-phenylsulfanyl-pyrazolo[3,4-d]pyrimidin-1-yl)-azetidine-1-carboxylic acid tert-butyl ester (Compound No. 320)

A mixture of tert-butyl 3-(4-amino-3-iodo-1H-pyrazolo[3,4-d]pyrimidin-1-yl)azetidine-1-carboxylate (2.0 g, 4.8 mmol), benzenethiol (1.06 g, 9.60 mmol), K₂CO₃ (2.66 g, 19.2 mmol) and copper (0.12 g, 1.90 mmol) in 25 ml toluene was heated at reflux with stirring for 3 hr. The mixture was concentrated, diluted with water and extracted with ethyl acetate. The combined organic phase was washed with brine, dried (anhydrous sodium sulfate) and solvent removed in vacuo. The residue was purified by column chromatography on silica gel (methylene chloride-methanol gradient) to afford the title compound (1.2 g, 3.01 mmol); LC/MS, API-ES, Pos, (M+H)⁺, 399.0.

### Example 61

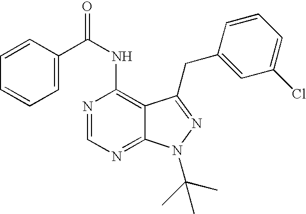

### N-[1-tert-Butyl-3-(3-chloro-phenoxy)-1H-pyrazolo[3,4-d]pyrimidin-4-yl]-benzamide (Compound No. 332)

1-tert-Butyl-3-(3-chloro-phenoxy)-1H-pyrazolo[3,4-d]pyrimidin-4-ylamine (200 mg, 0.63 mmol) was dissolved in anhydrous dimethylformamide (5 ml) and diisopropylethyl amine (244.2 mg, 1.89 mmol) and benzoic acid (184.6 mg, 1.51 mmol) were added. The solution was stirred and O-(7-azabenzotriazol-1-yl)-N,N,N′,N′-tetramethyluronium hexafluorophosphate (HATU) (622.8 mg, 1.63 mmol) was added and the solution stirred at ambient temperature for 24 hr. Water was added and the product extracted with methylene chloride. The organic layer was washed with water, dried (anhydrous sodium sulfate) and evaporated in vacuo to afford a residue that was subjected to reverse phase preparative HPLC (water-acetonitrile gradient, 0.05% formic acid, 80:20 to 10:90, 20 min, linear gradient; flow, 15 ml/min; column, Phenomenex Luna 5μ C18, 100×21.2 mm; UV 254 and 218 nm). The fractions containing the desired material were combined and evaporated in vacuo followed by crystallization from acetonitrile to afford the title compound (18 mg, 0.04 mmol); LC-MS, API-ES, Pos, (M+H)⁺, 422.1, 424.1.

### Example 62

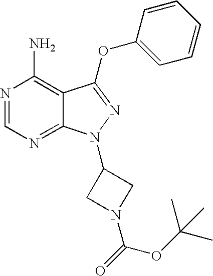

### 3-(4-Amino-3-phenoxy-pyrazolo[3,4-d]pyrimidin-1-yl)-azetidine-1-carboxylic acid tert-butyl ester (Compound No. 341)

tert-Butyl 3-(4-amino-3-iodo-1H-pyrazolo[3,4-d]pyrimidin-1-yl)azetidine-1-carboxylate (300 mg, 0.72 mmol), phenol (140 mg, 1.4 mmol), cesium carbonate (700 mg, 2.2 mmol), catalytic amount of CuI (14 mg, 0.072 mmol) and N,N-dimethylglycine hydrochloride (30 mg, 0.22 mmol) were added sequentially to dioxane (5 ml). The mixture was degassed under vacuum, flushed with argon and heated to 90° C. with stirring over night. The cooled mixture was filtered through a pad of Celite followed by washings with ethyl acetate. The combined filtrate was washed with brine, dried (anhydrous sodium sulfate) and solvent removed in vacuo. The residue was subjected to column chromatography on silica gel (hexane-ethyl acetate gradient) followed be reverse phase preparative HPLC to afford the title compound (18 mg, 0.05 mmol); LC/MS, API-ES, Pos, (M+H)⁺, 383.2.

### Example 63

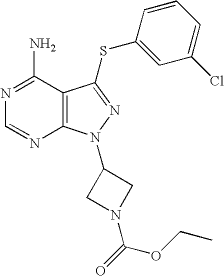

### 3-[4-Amino-3-(3-chloro-phenylsulfanyl)-pyrazolo[3,4-d]pyrimidin-1-yl]-azetidine-1-carboxylic acid ethyl ester (Compound No. 345)

To a solution of 3-(3-chlorophenylthio)-1-(azetidin-3-yl)-1H-pyrazolo[3,4-d]pyrimidin-4-amine (0.10 g, 0.30 mmol) in dimethylformamide (15 ml) was added pyridine (0.07 g, 0.90 mmol) and ethyl chloroformate (0.04 g, 0.36 mmol). The mixture was stirred at ambient temperature for 3 hr. The mixture was diluted with water and extracted with methylene chloride. The combined organic layer was washed with brine, dried (anhydrous sodium sulfate) and solvent removed in vacuo. The residue was purified by column chromatography on silica gel (methylene chloride-methanol gradient) to afford the title compound (69 mg, 0.17 mmol); LC/MS, API-ES, Neg, (M−H)⁻, 403.3, 405.1.

### Example 64

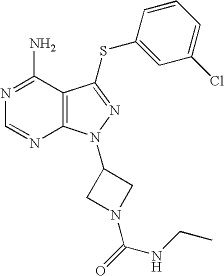

### 3-[4-Amino-3-(3-chloro-phenylsulfanyl)-pyrazolo[3,4-d]pyrimidin-1-yl]-azetidine-1-carboxylic acid ethylamide (Compound No. 346)

To a solution of 3-(3-chlorophenylthio)-1-(azetidin-3-yl)-1H-pyrazolo[3,4-d]pyrimidin-4-amine (0.10 g, 0.30 mmol) in dimethylformamide (15 ml) was added pyridine (0.07 g, 0.90 mmol) and ethyl isocyanate (0.03 g, 0.39 mmol). The mixture was stirred at ambient temperature for 4 hr. The mixture was diluted with water and extracted with methylene chloride. The combined organic layer was washed with brine, dried (anhydrous sodium sulfate) and solvent removed in vacuo. The residue was purified by column chromatography on silica gel (methylene chloride-methanol gradient) to afford title compound (68 mg, 0.16 mmol); LC/MS, API-ES, Pos, (M+H)⁺, 404.1, 406.1.

### Example 65

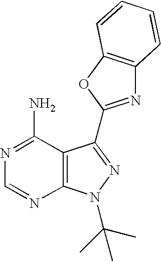

### 3-Benzooxazol-2-yl-1-tert-butyl-1H-pyrazolo[3,4-d]pyrimidin-4-ylamine (Compound No. 355)

### Step A

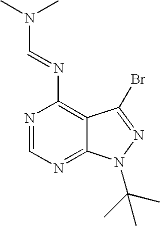

To a stirred solution of dimethylformamide dimethylacetal (291.2 mg, 2.22 mmol) in toluene (20 ml) was added 3-bromo-1-tert-butyl-1H-pyrazolo[3,4-d]pyrimidin-4-ylamine (500.0 mg, 1.85 mmol). The solution was heated at 110° C. for 5 hr. Solvent was removed in vacuo and the product purified by silica gel chromatography (hexane-ethyl acetate gradient) to afford N′-(3-bromo-1-tert-butyl-1H-pyrazolo[3,4-d]pyrimidin-4-yl)-N,N-dimethylformamidine (420.0 mg, 1.29 mmol); LC/MS, API-ES, Pos, (M+H)⁺, 325.1, 327.1.

### Step B

Triphenylphosphine (160 mg, 0.61 mmol), benzooxazole (185 mg, 1.54 mmol), cesium carbonate (1.0 g, 3.1 mmol) and N′-(3-bromo-1-tert-butyl-1H-pyrazolo[3,4-d]pyrimidin-4-yl)-N,N-dimethylformamidine (600 mg, 1.8 mmol) were added sequentially to 10 ml anhydrous and oxygen free dimethylacetamide. The mixture was flushed with argon and a catalytic amount of Pd(OAc)₂ (35 mg, 0.15 mmol) was added. The mixture was degassed under vacuum, flushed with argon and heated at 120° C. with stirring over night. The resulting mixture was cooled and diluted with methylene chloride and filtered through a pad of celite. The filtrate was washed with brine and water, and dried over anhydrous sodium sulfate followed by evaporation in vacuo. The residue was subjected to column chromatography on silica gel (hexane-ethyl acetate gradient) followed by preparative circular TLC (Chromatotron) (hexane-ethyl acetate gradient). Crystallization from ether and hexane afforded the title compound (0.2 g, 0.64 mmol); LC/MS, API-ES, Pos, (M+H)⁺, 309.1.

### Example 66

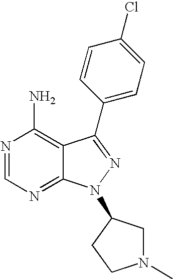

### 3-(4-Chloro-phenyl)-1-(1-methyl-pyrrolidin-3-yl)-1H-pyrazolo[3,4-d]pyrimidin-4-ylamine (Compound No. 356)

### Step A

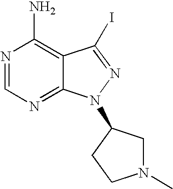

A suspension of 3-iodo-1H-pyrazolo[3,4-d]pyrimidin-4-ylamine (200 mg, 0.766 mmol), PPh₃ (400 mg, 1.5 mmol), (S)-1-methyl-3-pyrrolidinol (160 mg, 1.5 mmol) in anhydrous THF (10 mL) was cooled to 0° C. under argon and DEAD (270 mg, 1.5 mmol) was added to the mixture. After the addition, the reaction mixture was stirred for 3 hr at ambient temperature. TLC analysis of the mixture indicated the disappearance of the starting material. The resulting mixture was concentrated in vacuo to remove the solvent and the product purified by column chromatography on silica gel (methylene chloride-methanol gradient) to afford 3-iodo-1-(1-methyl-pyrrolidin-3-yl)-1H-pyrazolo[3,4-d]pyrimidin-4-ylamine as a light yellow solid (135 mg, 0.39 mmol).

### Step B

To a stirred solution of 3-iodo-1-(1-methyl-pyrrolidin-3-yl)-1H-pyrazolo[3,4-d]pyrimidin-4-ylamine (130 mg, 0.37 mmol) (step A) in DME (4 ml) and water (2 ml) was added sequentially 4-chlorophenylboronic acid (71 mg, 0.45 mmol) and Na₂CO₃ (80 mg, 0.76 mmol). The mixture was flushed with argon before catalytic amount of Pd(PPh₃)₄ (44 mg, 0.038 mmol) was added. The mixture was degassed and charged with argon three times. The mixture was heated at reflux for 3 hr. Water was added and the product extracted with ethyl acetate. The combined extracts were washed with brine, dried over anhydrous sodium sulfate, filtered and concentrated to give a residue that was subjected to column chromatography on silica gel (methylene chloride-methanol gradient) followed by preparative circular TLC (Chromatotron) (methylene chloride:methanol, 15:1) to afford the title compound (30 mg. 0.09 mmol); LC/MS, API-ES, Pos, (M+H)⁺, 329.2, 331.2.

### Example 67

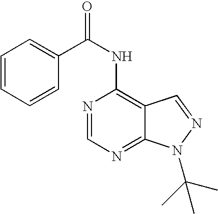

### N-(1-tert-Butyl-1H-pyrazolo[3,4-d]pyrimidin-4-yl)-benzamide (Compound No. 388)

To a solution of 1-tert-butyl-1H-pyrazolo[3,4-d]pyrimidin-4-amine (100 mg, 0.52 mmol) in pyridine (2 ml), was added benzoyl chloride (110 mg, 0.78 mmol). The reaction mixture was stirred at ambient temperature for 3 hr. Water was added and the product extracted with ethyl acetate. The organic layer was separated, dried (anhydrous sodium sulfate) and evaporated in vacuo. The product was purified by column chromatography on silica gel (hexane-ethyl acetate gradient) and recrystallization (hexane and acetone) to yield the title compound (100 mg, 0.34 mmol); LC/MS, API-ES, Pos, (M+H)⁺, 296.1.

### Example 68

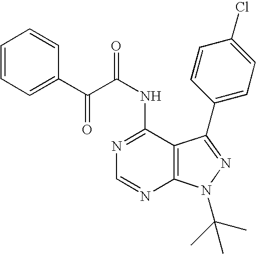

### N-[1-tert-Butyl-3-(4-chloro-phenyl)-1H-pyrazolo[3,4-d]pyrimidin-4-yl]-2-oxo-2-phenyl-acetamide (Compound No. 395)

To a dry, round bottom flask was added benzoylformic acid (99 mg, 0.66 mmol), HATU (252 mg, 0.66 mmol), and dimethylformamide (3 ml). This solution was stirred at 25° C. for 15 min, followed by addition of 1-tert-butyl-3-(4-chloro-phenyl)-1H-pyrazolo[3,4-d]pyrimidin-4-ylamine (100 mg, 0.33 mmol) and DIEA (220 μL, 1.33 mmol). The resulting mixture was stirred at 25° C. for 16 hr. The crude material was purified via reverse phase preparative HPLC-MS, concentrated, and triturated with acetonitrile to afford the title compound (17 mg, 0.04 mmol); LC/MS, API-ES, Pos, (M+H)⁺, 434.9, 436.9.

### Example 69

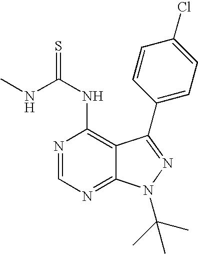

### 1-[1-tert-Butyl-3-(4-chloro-phenyl)-1H-pyrazolo[3,4-d]pyrimidin-4-yl]-3-methyl-thiourea (Compound No. 397)

1-tert-Butyl-3-(4-chloro-phenyl)-1H-pyrazolo[3,4-d]pyrimidin-4-ylamine (100 mg, 0.33 mmol) was dissolved in anhydrous dioxane (5 ml) and solution stirred at 0° C. Sodium hydride (60% in paraffin oil, 15.8 mg, 0.4 mmol) was added and the solution stirred for 5 min. Methyl isothiocyanante (28.9 mg, 0.39 mmol) was added and the solution stirred at ambient temperature for 30 min. Water was added and the product extracted with methylene chloride. The organic layer was washed with water, dried (anhydrous sodium sulfate) and evaporated in vacuo. The residue was subjected to reverse phase preparative HPLC (water-acetonitrile gradient, 0.05% formic acid, 80:20 to 10:90, 20 min, linear gradient; flow, 15 mL/min; column, Phenomenex Luna 5μ C18, 100×21.2 mm; UV 254 and 218 nm). The peek containing the desired material was pooled and solvent evaporated in vacuo to afford a residue that was crystallized from acetonitrile to afford the title compound (16 mg, 0.04 mmol); LC/MS, API-ES, Pos, (M+H)⁺, 375.0, 377.0.

### Example 70

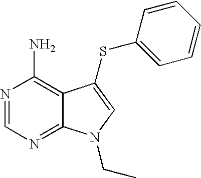

### 7-Ethyl-5-phenylsulfanyl-7H-pyrrolo[2,3-d]pyrimidin-4-ylamine (Compound No. 333)

A mixture of 7-ethyl-5-iodo-7H-pyrrolo[2,3-d]pyrimidin-4-amine (0.10 g, 0.35 mmol), benzenethiol (0.08 g, 0.69 mmol), copper (0.01 g, 0.14 mmol) and potassium carbonate (0.19 g, 1.39 mmol) in 10 ml toluene was stirred over night at 110° C. After filtration and concentration, the residue was purified by column chromatography on silica gel (hexane:acetone gradient, 10:1, 8:1, 6:1) to afford the title compound as a gray solid (35 mg, 0.13 mmol); LC/MS, API-ES, Pos, (M+H)⁺, 271.5.

### Example 71

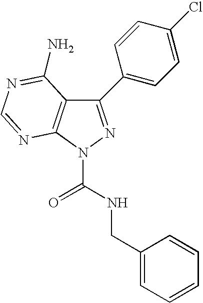

### 4-amino-N-benzyl-3-(4-chlorophenyl)-1H-pyrazolo[3,4-d]pyrimidine-1-carboxamide (Compound 213)

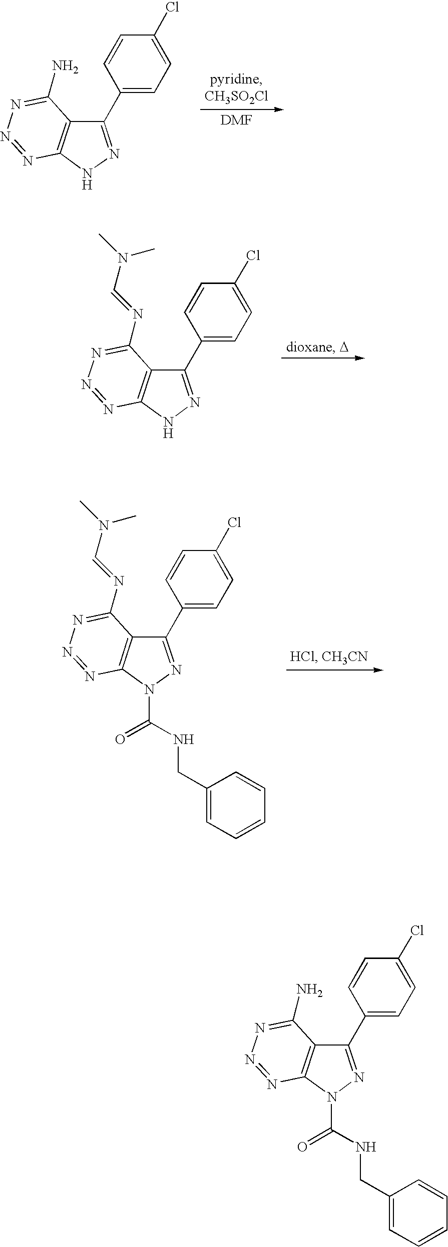

Step A:

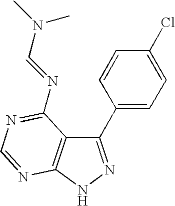

To a suspension of 3-(4-chlorophenyl)-1H-pyrazolo[3,4-d]pyrimidin-4-amine (500 mg, 2.04 mmol) in 7 ml DMF, pyridine (193 mg, 2.44 mmol), and methanesulfonyl chloride (257 mg, 2.24 mmol) were added. After 1 h, TLC (CH₂Cl₂:MeOH=15:1) shows a new spot (R_{f}=0.6) and starting material has not disappeared totally. Then additional 1.2 eq. pyridine and 1.1 eq. methanesulfonyl chloride were added every 1 h. After adding two times, the starting material disappeared totally. The reaction mixture was poured into water, but there was no precipitate. Then 20 ml sat. NaHCO₃ was added. The product was separated by filtration, washed with acetone to afford the title compound as a brown solid (397 mg, 64%). MS (ESI, pos.), m/z, 301.0 (MH⁺, 100%), 303.1 (MH⁺, 27%).

Step B:

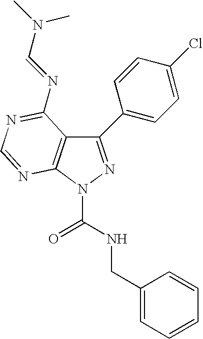

To a suspension of N′-(3-(4-chlorophenyl)-1H-pyrazolo[3,4-d]pyrimidin-4-yl)-N,N-dimethylformamidine (200 mg, 0.67 mmol) in 15 ml dioxane, benzylisocyanate (88.0 mg, 0.67 mmol) was added. The reaction mixture was heated at 50° C. for 1 h, TLC (CH₂Cl₂:MeOH=15:1) shows there were two new spots (R_{f}=0.4, weak; R_{f}=0.5, strong) and starting material has not disappeared totally. After 2 h, the reaction mixture was heated to 80° C. for 3 h. The reaction was stirred at room temperature overnight. TLC shows there was still small amount of starting material. Then additional 1 eq. benzylisocyanate was added and the reaction was heated at 80° C. for 2 h until the reaction was complete. The reaction mixture was cooled down. The precipitate was filtered off, washed with acetone to afford the title compound as a yellow solid (248 mg, 85%). MS (ESI, pos.), m/z, 433.9 (MH⁺, 100%), 435.8 (MH⁺, 37%), 866.4 (2 MH⁺, 41%), 868.4 (2MH⁺, 30%).

Step C:

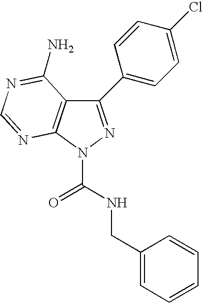

N-benzyl-3-(4-chlorophenyl)-4-((E)-formamido)-1H-pyrazolo[3,4-d]pyrimidine-1-carboxamide (60.0 mg, 0.14 mmol) was dissolved in 10 ml HCl/CH₃CN solution (1 M). The reaction mixture was stirred overnight at 25° C. TLC (CH₂Cl₂:MeOH=15:1) shows the reaction was complete. Then the reaction mixture was concentrated, neutralized with sat. NaHCO₃ to adjust to pH=7˜8. The precipitate was filtered off, and washed with acetone to afford the title compound as a white solid (40 mg, 76%). MS (ESI, pos.), m/z, 378.9 (MH⁺, 14%), 441.4 (MNa⁺+CH₃CN, 83%), 778.8 (2MNa⁺, 100%); ¹H NMR (C₅D₅N, 400 MHz, 080128 BL-13-007-2_{—}1H) δ=9.98 (t, J=6.0 Hz, 1H), 8.65 (s, 1H), 7.87 (d, J=8.8 Hz, 2H), 7.62 (d, J=7.2 Hz, 2H), 7.36-7.41 (m, 4H), 7.29 (dd, J=7.2, 7.6 Hz, 1H), 4.92 (d, J=6.0 Hz, 2H).

Since modifications will be apparent to those of skill in the art, it is intended that the invention be limited only by the scope of the appended claims.

1. A method of treating a subject for a disorder characterized by impaired protein trafficking, comprising administering to the subject an effective amount of a compound represented by the following structural formula: 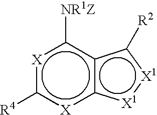
or pharmaceutically acceptable salts thereof, wherein:
m is 1 or 2;
each X is independently N, CH, or C(C₁-C₄ alkyl);
each X¹ is independently N, NR³, CH, or C(C₁-C₄ alkyl);
R¹ and Z are each independently R⁵, C(O)R⁵, COOR⁵, C(O)NR⁵R⁵, or S(O)ₘR⁵; or, NR¹Z, taken together, is N═CH—NR⁵R⁵
R² and R³ are each independently H, halo, pseudohalo, CN, SR⁵, R⁵, OR⁵, OC(O)R⁵, NR⁵R⁵, NR⁵R⁶, COOR⁵, NO₂, C(O)R⁵, C(O)C(O)R⁵, C(O)NR⁵R⁵, S(O)ₘR⁵, S(O)ₘNR⁵R⁵, NR⁵C(O)NR⁵R⁵, NR⁵C(O)C(O)R⁵, NR⁵C(O)R⁵, NR⁵(COOR⁵), NR⁵C(O)R⁸, NR⁵S(O)ₘNR⁵R⁵, NR⁵S(O)ₘR⁵, NR⁵S(O)ₘR⁸, NR⁵C(O)C(O)NR⁵R⁵, NR⁵C(O)C(O)NR⁵R⁶, P(O)R⁵R⁵, P(O)(NR⁵R⁵)₂, P(O)(NR⁵R⁶)₂, P(O)(NR⁶R⁶)₂, or P(O)(OR⁵)₂;
R⁴ is independently H, halo, pseudohalo, CN, SR⁵, OR⁵, OC(O)R⁵, NR⁵R⁵, NR⁵R⁶, COOR⁵, NO₂, C(O)R⁵, C(O)C(O)R⁵, C(O)NR⁵R⁵, S(O)ₘR⁵, S(O)ₘNR⁵R⁵, NR⁵C(O)NR⁵R⁵, NR⁵C(O)C(O)R⁵, NR⁵C(O)R⁵, NR⁵(COOR⁵), NR⁵C(O)R⁸, NR⁵S(O)ₘNR⁵R⁵, NR⁵S(O)ₘR⁵, NR⁵S(O)ₘR⁸, NR⁵C(O)C(O)NR⁵R⁵, or NR⁵C(O)C(O)NR⁵R⁶; or optionally substituted alkyl, aryl, aralkyl, heteroaryl, or heteroaralkyl; and
each R⁵, R⁶, and R⁸ is independently H or optionally substituted alkyl, alkenyl, alkynyl, cycloalkyl, cycloalkenyl, aryl, heteroaryl, or heterocyclyl,
wherein the disorder is not a synucleinopathy.

2. A method of increasing protein trafficking in a cell, comprising contacting the cell with an effective amount of a compound represented by the following structural formula: 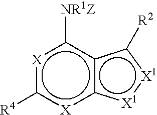
or pharmaceutically acceptable salts thereof, wherein:
m is 1 or 2;
each X is independently N, CH, or C(C₁-C₄ alkyl);
each X¹ is independently N, NR³, CH, or C(C₁-C₄ alkyl);
R¹ and Z are each independently R⁵, C(O)R⁵, COOR⁵, C(O)NR⁵R⁵, or S(O)ₘR⁵; or, NR¹Z, taken together, is N═CH—NR⁵R⁵
R² and R³ are each independently H, halo, pseudohalo, CN, SR⁵, R⁵, OR⁵, OC(O)R⁵, NR⁵R⁵, NR⁵R⁶, COOR⁵, NO₂, C(O)R⁵, C(O)C(O)R⁵, C(O)NR⁵R⁵, S(O)ₘR⁵, S(O)ₘNR⁵R⁵, NR⁵C(O)NR⁵R⁵, NR⁵C(O)C(O)R⁵, NR⁵C(O)R⁵, NR⁵(COOR⁵), NR⁵C(O)R⁸, NR⁵S(O)ₘNR⁵R⁵, NR⁵S(O)ₘR⁵, NR⁵S(O)ₘR⁸, NR⁵C(O)C(O)NR⁵R⁵, NR⁵C(O)C(O)NR⁵R⁶, P(O)R⁵R⁵, P(O)(NR⁵R⁵)₂, P(O)(NR⁵R⁶)₂, P(O)(NR⁶R⁶)₂, or P(O)(OR⁵)₂;
R⁴ is independently H, halo, pseudohalo, CN, SR⁵, OR⁵, OC(O)R⁵, NR⁵R⁵, NR⁵R⁶, COOR⁵, NO₂, C(O)R⁵, C(O)C(O)R⁵, C(O)NR⁵R⁵, S(O)ₘR⁵, S(O)ₘNR⁵R⁵, NR⁵C(O)NR⁵R⁵, NR⁵C(O)C(O)R⁵, NR⁵C(O)R⁵, NR⁵(COOR⁵), NR⁵C(O)R⁸, NR⁵S(O)ₘNR⁵R⁵, NR⁵S(O)ₘR⁵, NR⁵S(O)ₘR⁸, NR⁵C(O)C(O)NR⁵R⁵, or NR⁵C(O)C(O)NR⁵R⁶; or optionally substituted alkyl, aryl, aralkyl, heteroaryl, or heteroaralkyl; and
each R⁵, R⁶, and R⁸ is independently H or optionally substituted alkyl, alkenyl, alkynyl, cycloalkyl, cycloalkenyl, aryl, heteroaryl, or heterocyclyl,
wherein the cell is not characterized by impaired synuclein trafficking.

3. A method of treating a subject for a disorder characterized by impaired protein trafficking, comprising administering to the subject an effective amount of a compound represented by the following structural formula: 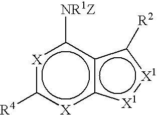
or pharmaceutically acceptable salts thereof, wherein:
m is 1 or 2;
each X is independently N or CH;
each X¹ is independently N, NR³ or CH;
R¹ and Z are each independently R⁵, C(O)R⁵, COOR⁵, C(O)NR⁵R⁵, or S(O)ₘR⁵;
R² and R³ are each independently H, halo, pseudohalo, CN, SR⁵, R⁵, OR⁵, OC(O)R⁵, NR⁵R⁵, NR⁵R⁶, COOR⁵, NO₂, C(O)R⁵, C(O)C(O)R⁵, C(O)NR⁵R⁵, S(O)ₘR⁵, S(O)ₘNR⁵R⁵, NR⁵C(O)NR⁵R⁵, NR⁵C(O)C(O)R⁵, NR⁵C(O)R⁵, NR⁵(COOR⁵), NR⁵C(O)R⁸, NR⁵S(O)ₘNR⁵R⁵, NR⁵S(O)ₘR⁵, NR⁵S(O)ₘR⁸, NR⁵C(O)C(O)NR⁵R⁵, NR⁵C(O)C(O)NR⁵R⁶, P(O)R⁵R⁵, P(O)(NR⁵R⁵)₂, P(O)(NR⁵R⁶)₂, P(O)(NR⁶R⁶)₂, or P(O)(OR⁵)₂;
R⁴ is independently H, halo, pseudohalo, CN, SR⁵, OR⁵, OC(O)R⁵, NR⁵R⁵, NR⁵R⁶, COOR⁵, NO₂, C(O)R⁵, C(O)C(O)R⁵, C(O)NR⁵R⁵, S(O)ₘR⁵, S(O)ₘNR⁵R⁵, NR⁵C(O)NR⁵R⁵, NR⁵C(O)C(O)R⁵, NR⁵C(O)R⁵, NR⁵(COOR⁵), NR⁵C(O)R⁸, NR⁵S(O)ₘNR⁵R⁵, NR⁵S(O)ₘR⁵, NR⁵S(O)ₘR⁸, NR⁵C(O)C(O)NR⁵R⁵, or NR⁵C(O)C(O)NR⁵R⁶; or optionally substituted alkyl, aryl, aralkyl, heteroaryl, or heteroaralkyl; and
each R⁵, R⁶, and R⁸ is independently H or optionally substituted alkyl, alkenyl, alkynyl, cycloalkyl, cycloalkenyl, aryl, heteroaryl, or heterocyclyl,
wherein the disorder is not a synucleinopathy.

4. A method of increasing protein trafficking in a cell, comprising contacting the cell with an effective amount of a compound represented by the following structural formula: 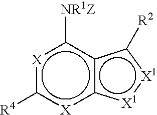
or pharmaceutically acceptable salts thereof, wherein:
m is 1 or 2;
each X is independently N or CH;
each X¹ is independently N, NR³ or CH;
R¹ and Z are each independently R⁵, C(O)R⁵, COOR⁵, C(O)NR⁵R⁵, or S(O)ₘR⁵;
R² and R³ are each independently H, halo, pseudohalo, CN, SR⁵, R⁵, OR⁵, OC(O)R⁵, NR⁵R⁵, NR⁵R⁶, COOR⁵, NO₂, C(O)R⁵, C(O)C(O)R⁵, C(O)NR⁵R⁵, S(O)ₘR⁵, S(O)ₘNR⁵R⁵, NR⁵C(O)NR⁵R⁵, NR⁵C(O)C(O)R⁵, NR⁵C(O)R⁵, NR⁵(COOR⁵), NR⁵C(O)R⁸, NR⁵S(O)ₘNR⁵R⁵, NR⁵S(O)ₘR⁵, NR⁵S(O)ₘR⁸, NR⁵C(O)C(O)NR⁵R⁵, NR⁵C(O)C(O)NR⁵R⁶, P(O)R⁵R⁵, P(O)(NR⁵R⁵)₂, P(O)(NR⁵R⁶)₂, P(O)(NR⁶R⁶)₂, or P(O)(OR⁵)₂;
R⁴ is independently H, halo, pseudohalo, CN, SR⁵, OR⁵, OC(O)R⁵, NR⁵R⁵, NR⁵R⁶, COOR⁵, NO₂, C(O)R⁵, C(O)C(O)R⁵, C(O)NR⁵R⁵, S(O)ₘR⁵, S(O)ₘNR⁵R⁵, NR⁵C(O)NR⁵R⁵, NR⁵C(O)C(O)R⁵, NR⁵C(O)R⁵, NR⁵(COOR⁵), NR⁵C(O)R⁸, NR⁵S(O)ₘNR⁵R⁵, NR⁵S(O)ₘR⁵, NR⁵S(O)ₘR⁸, NR⁵C(O)C(O)NR⁵R⁵, or NR⁵C(O)C(O)NR⁵R⁶; or optionally substituted alkyl, aryl, aralkyl, heteroaryl, or heteroaralkyl; and
each R⁵, R⁶, and R⁸ is independently H or optionally substituted alkyl, alkenyl, alkynyl, cycloalkyl, cycloalkenyl, aryl, heteroaryl, or heterocyclyl,
wherein the cell is not characterized by impaired synuclein trafficking.

5. The method of claim 1, wherein the compound is represented by the following structural formula:

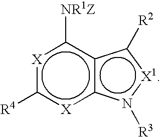

6. The method of claim 1, wherein the compound is represented by the following structural formula:

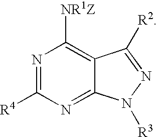

7. The method of claim 1, wherein R³ is selected from the group consisting of substituted or unsubstituted alkyl, cycloalkyl, aryl, and aralkyl.

8. The method of claim 1, wherein R² is hydrogen, halo, or optionally substituted aryl, heteroaryl, aralkyl, or aralkenyl.

9. The method of claim 1, wherein R¹ and Z are each independently selected from the group consisting of hydrogen, or substituted or unsubstituted alkyl, alkylcarbonyl, arylcarbonyl, aralkylcarbonyl, haloarylcarbonyl, arylsulfonyl, aralkylsulfonyl, and haloarylsulfonyl.

10. The method of claim 1, wherein R¹ is H and Z is H.

11. The method of claim 1, wherein R¹ is methyl and Z is H.

12. The method of claim 1, wherein R⁴ is H, alkyl, cycloalkyl, or alkylcycloalkyl.

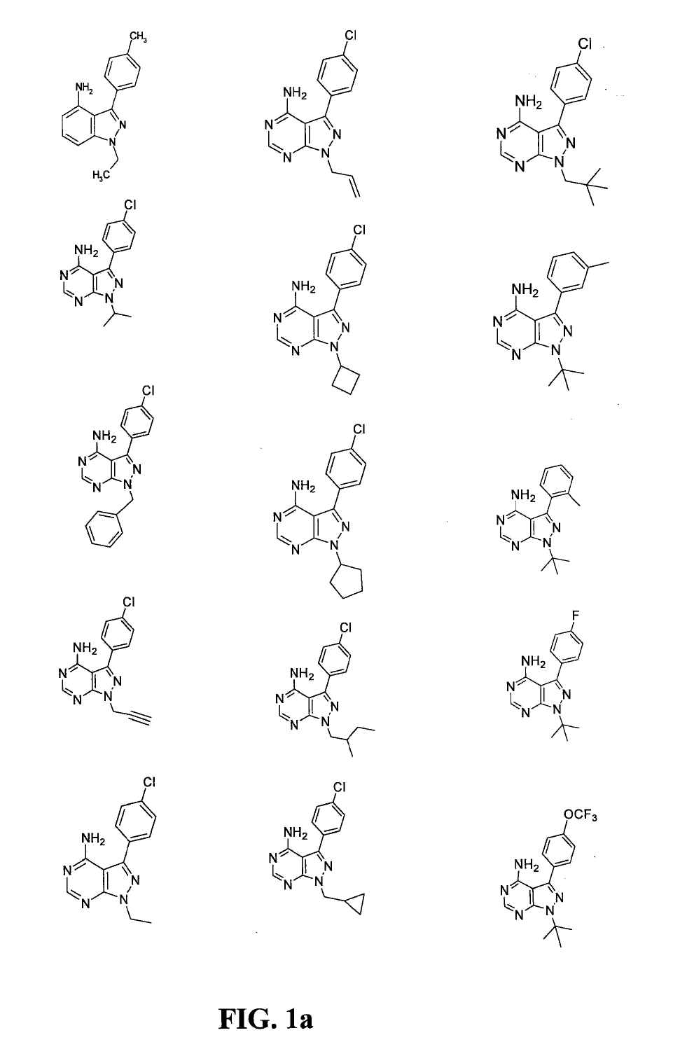

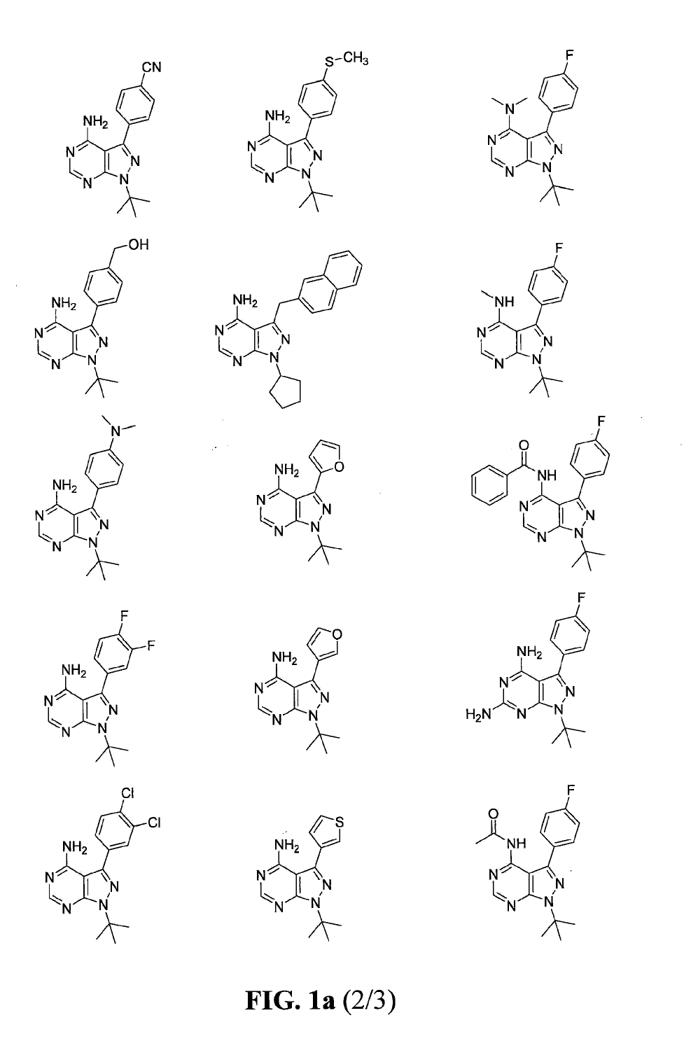

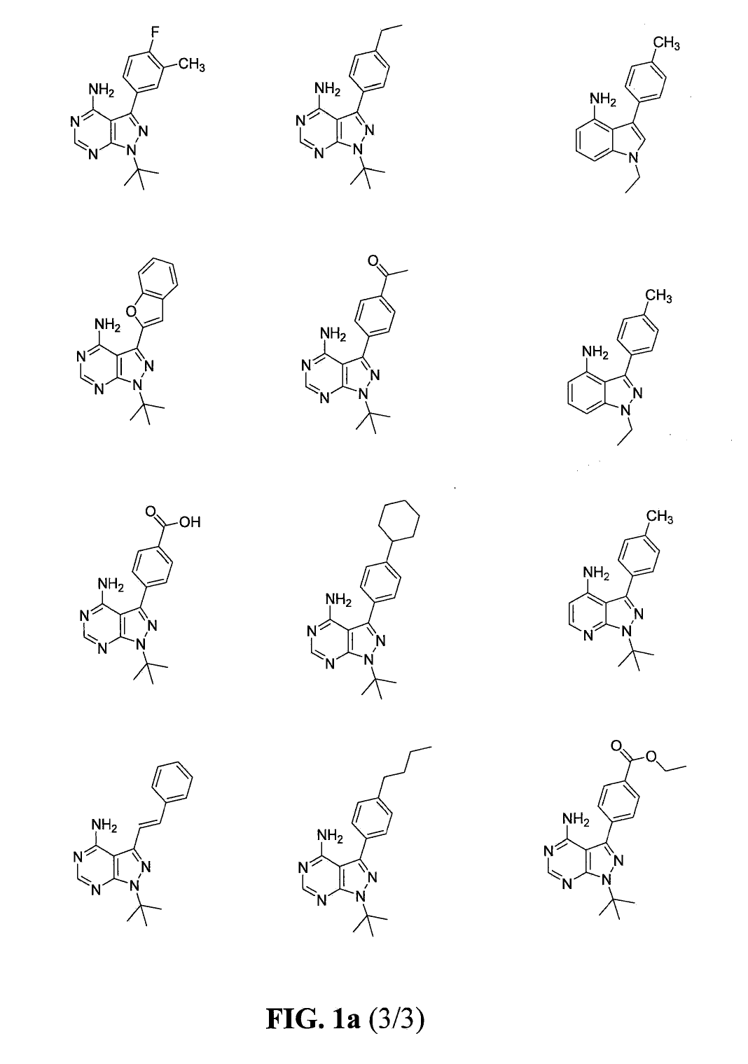

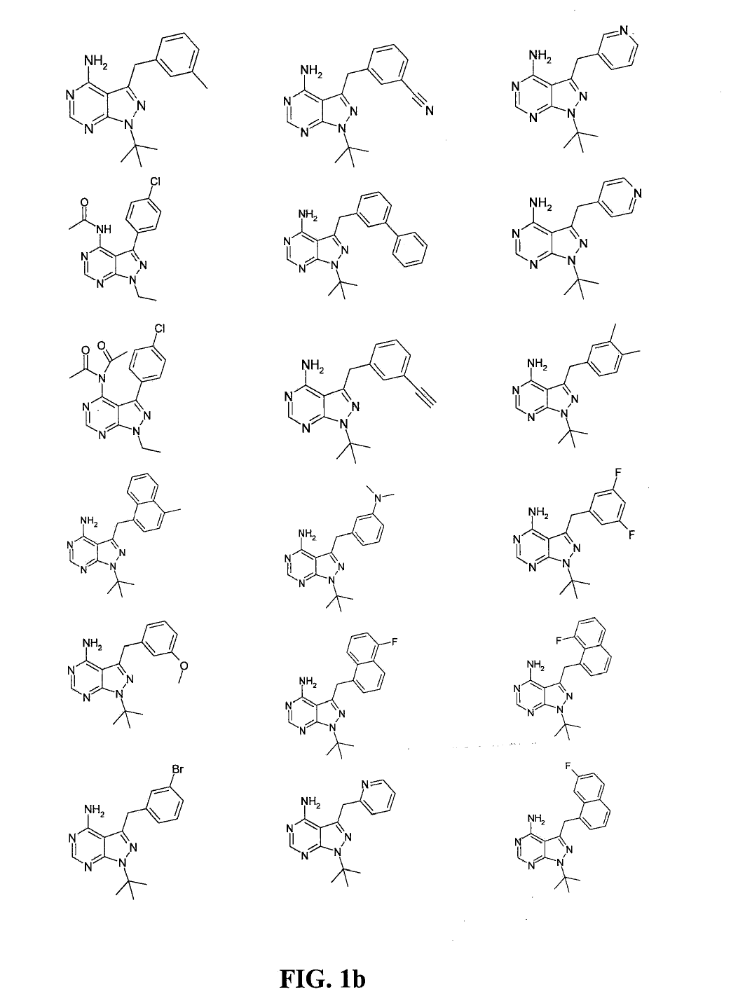

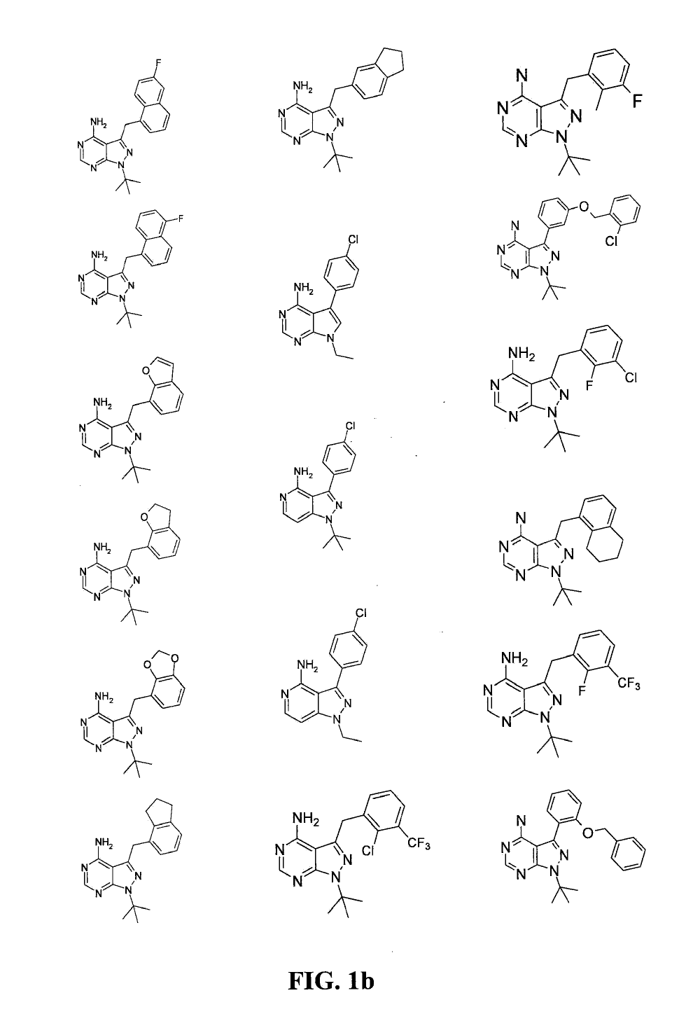

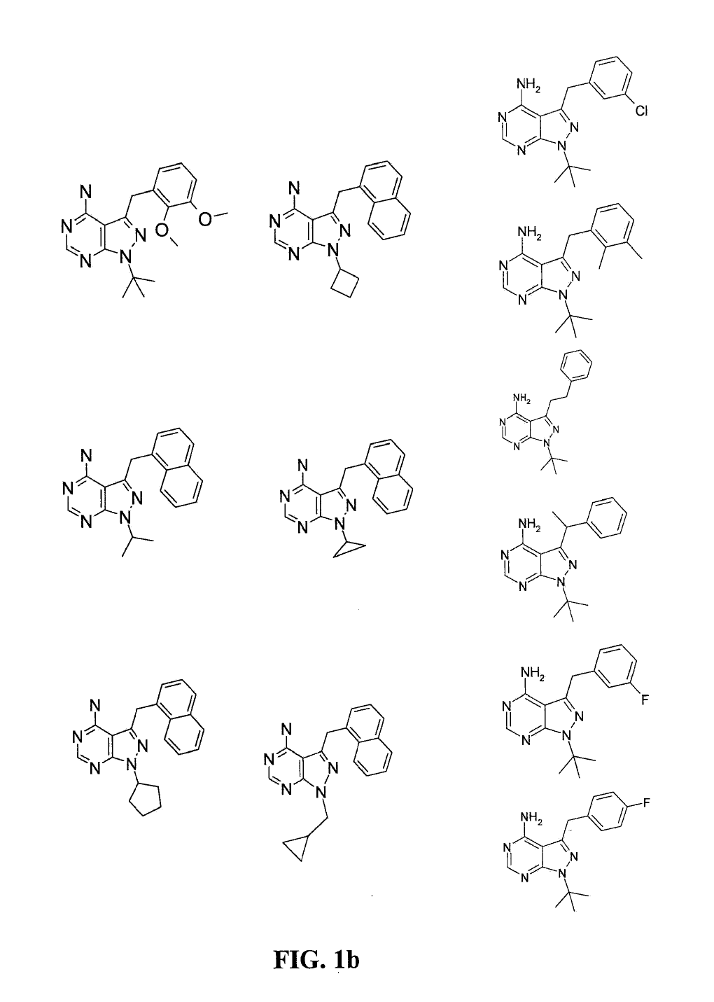

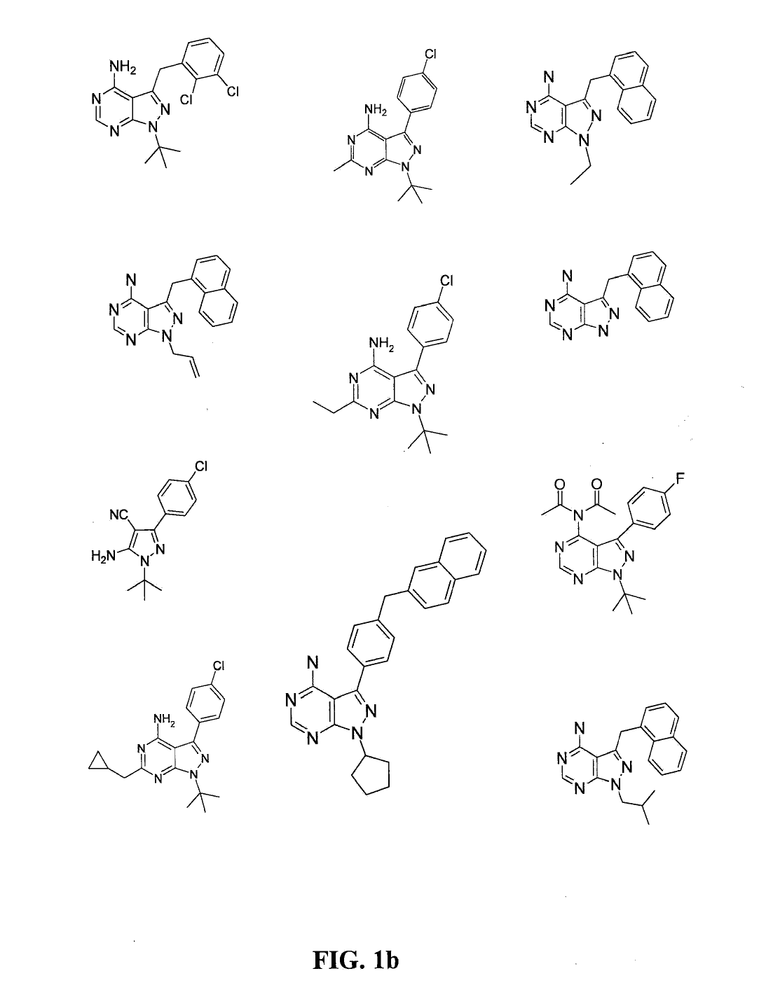

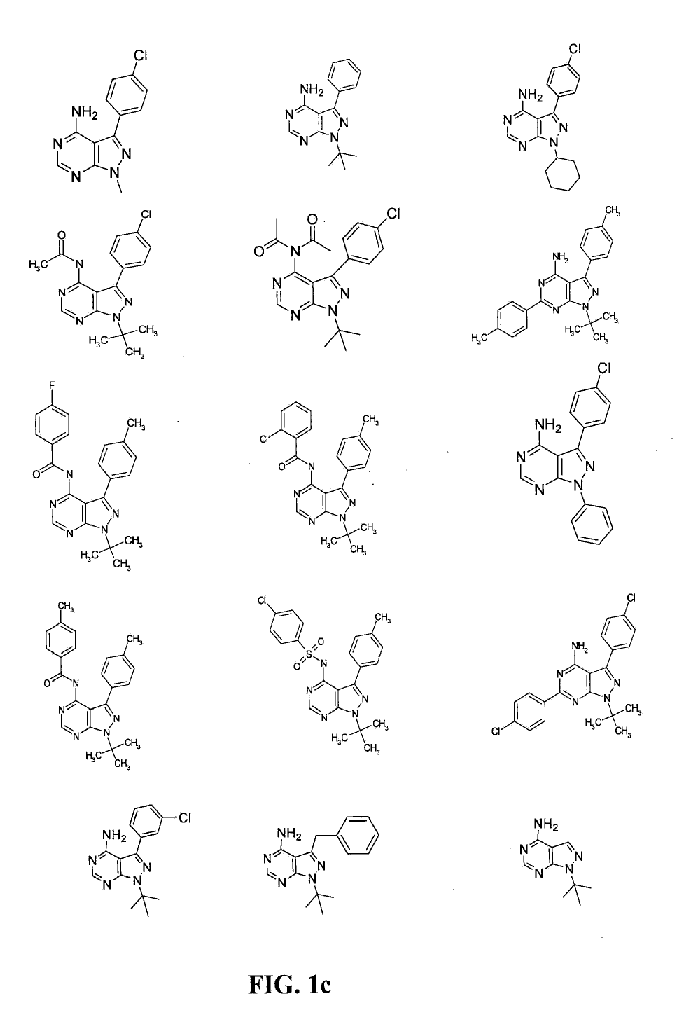

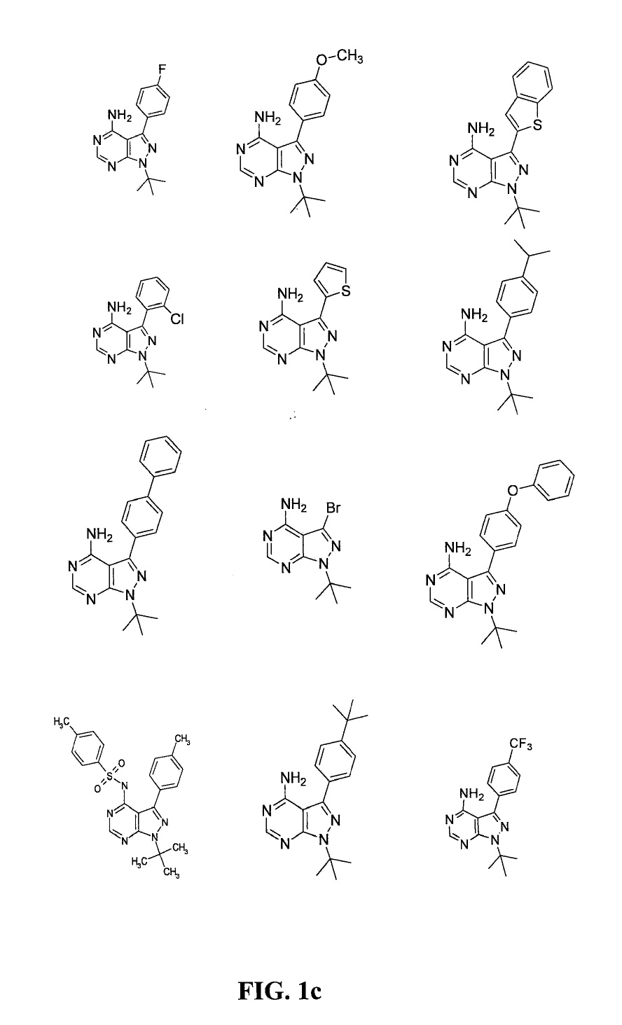

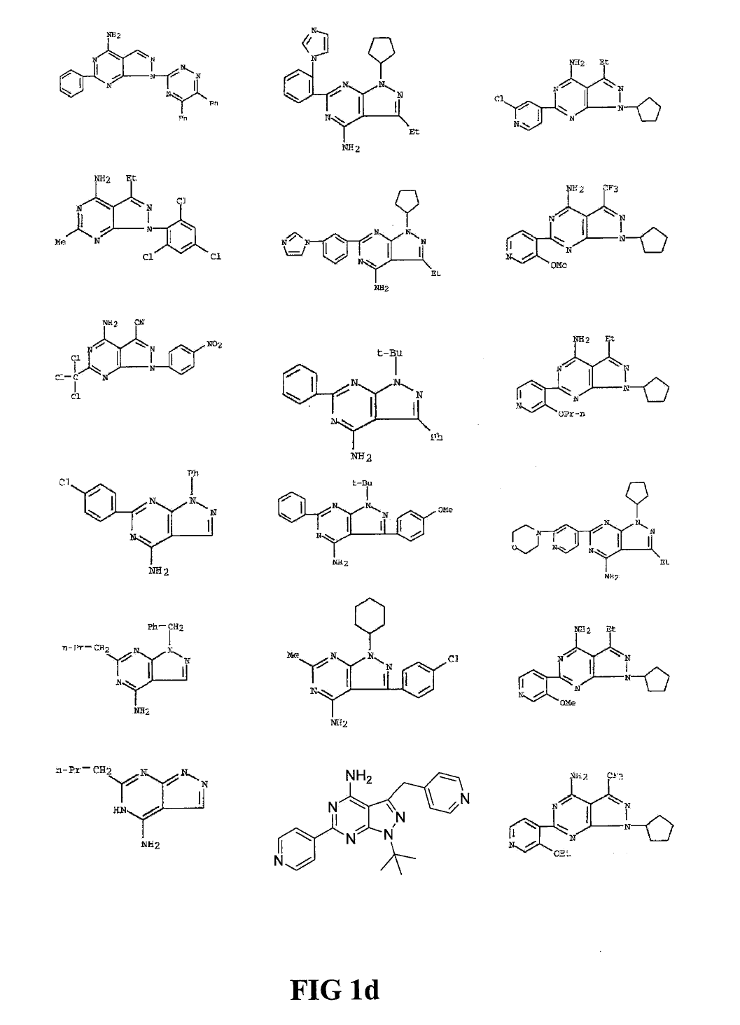

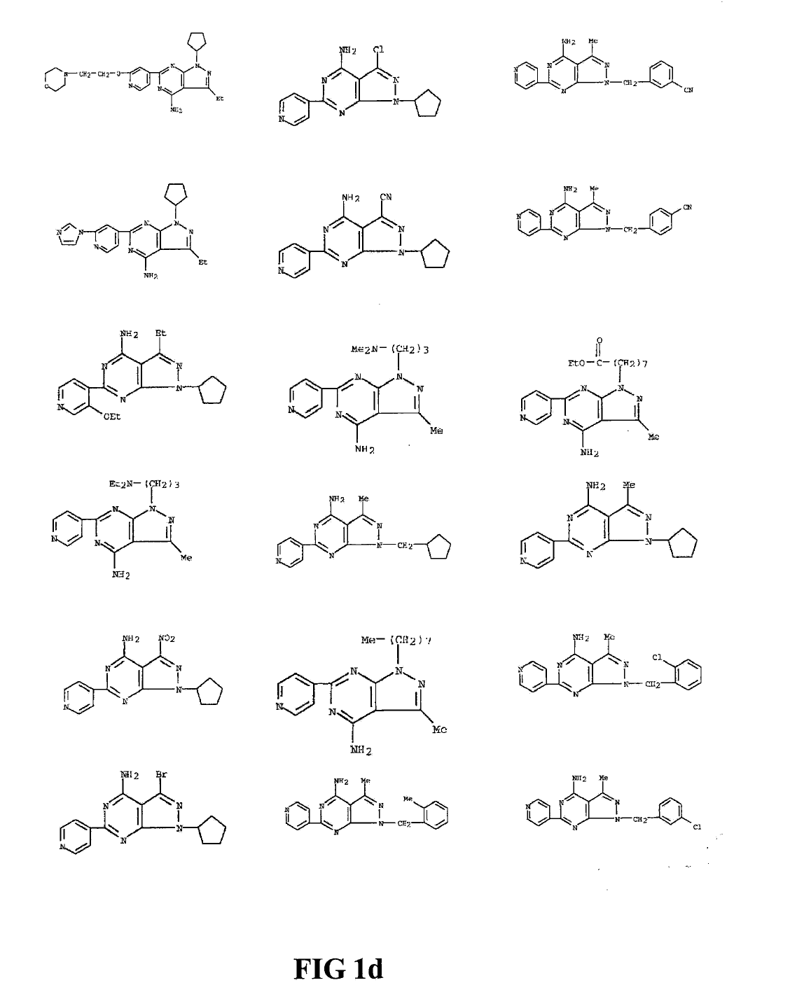

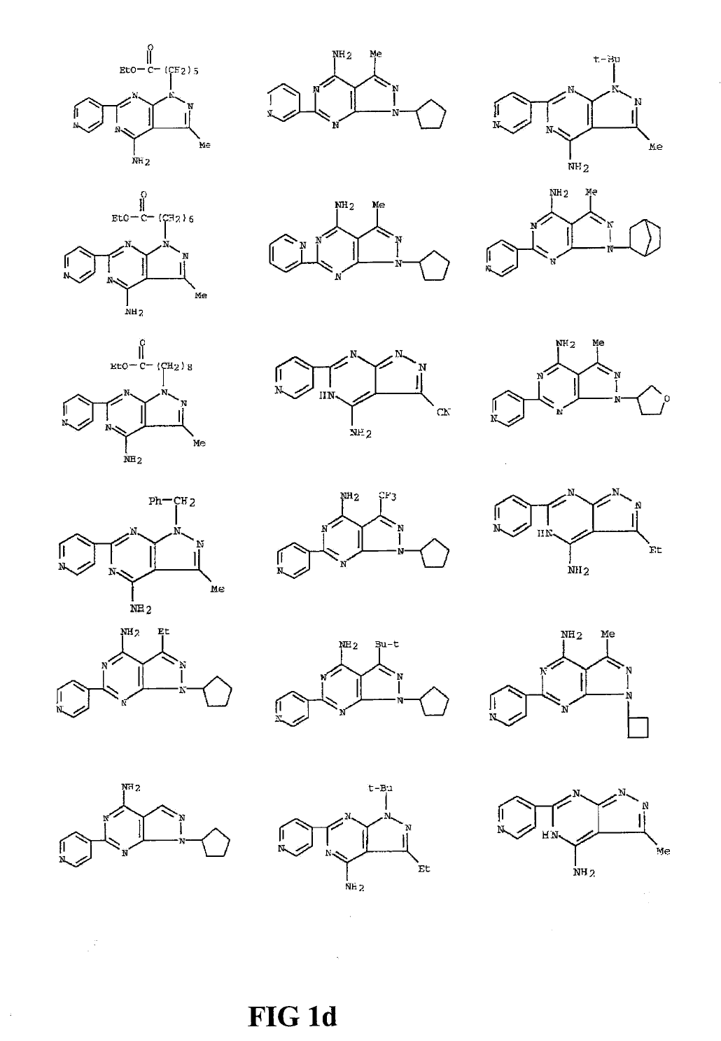

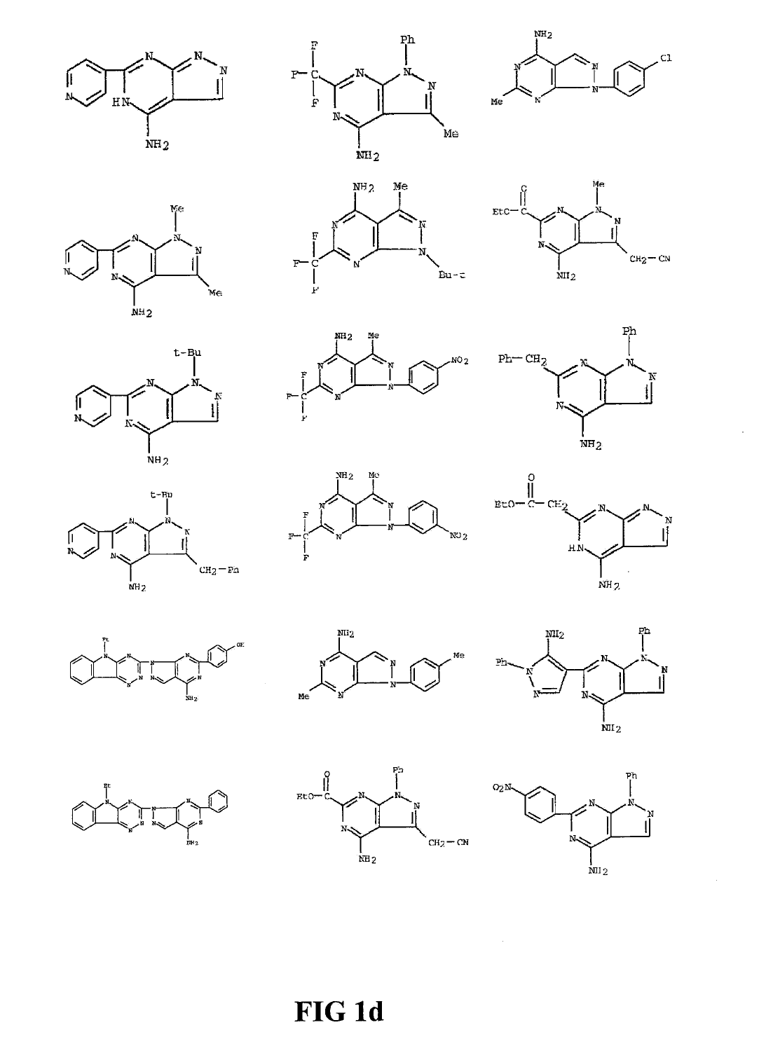

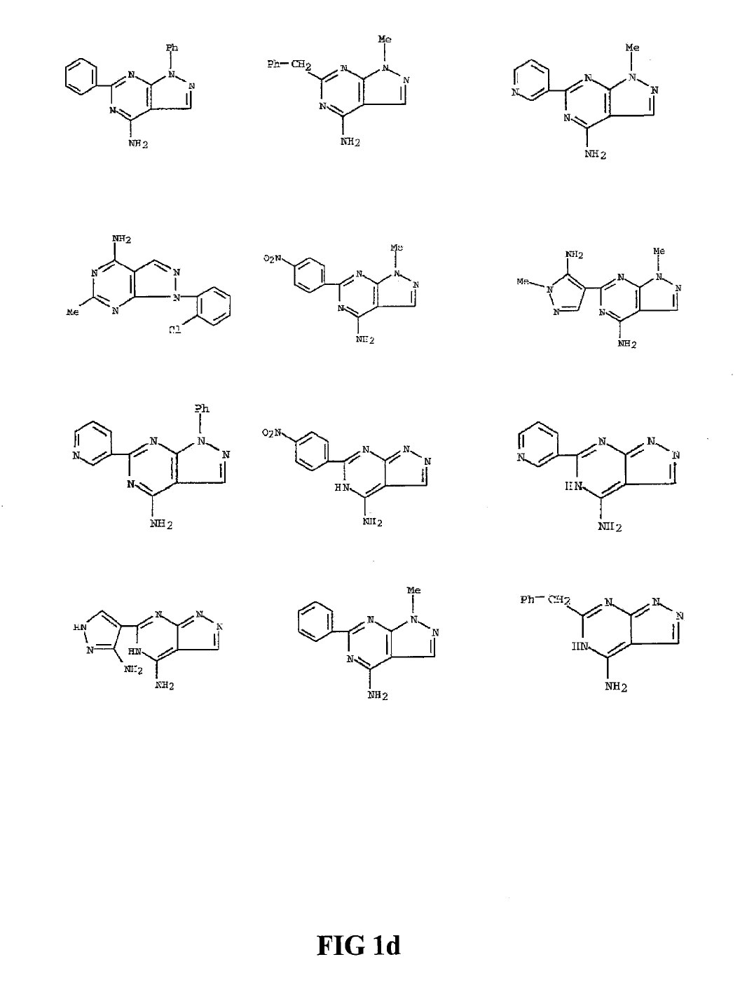

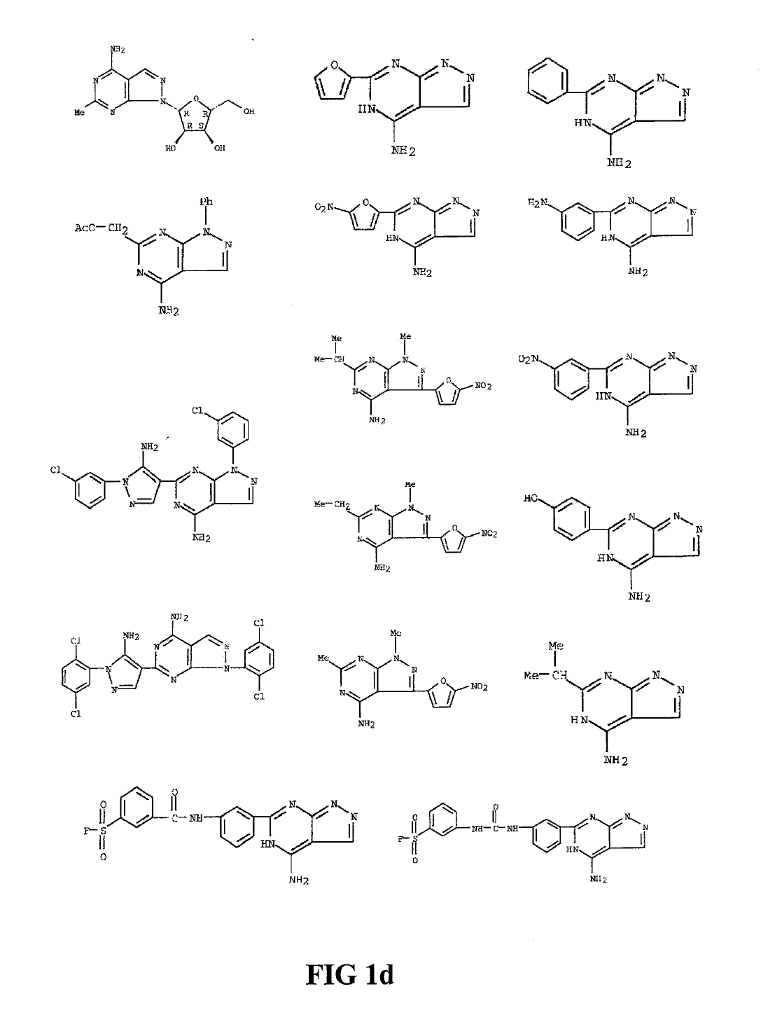

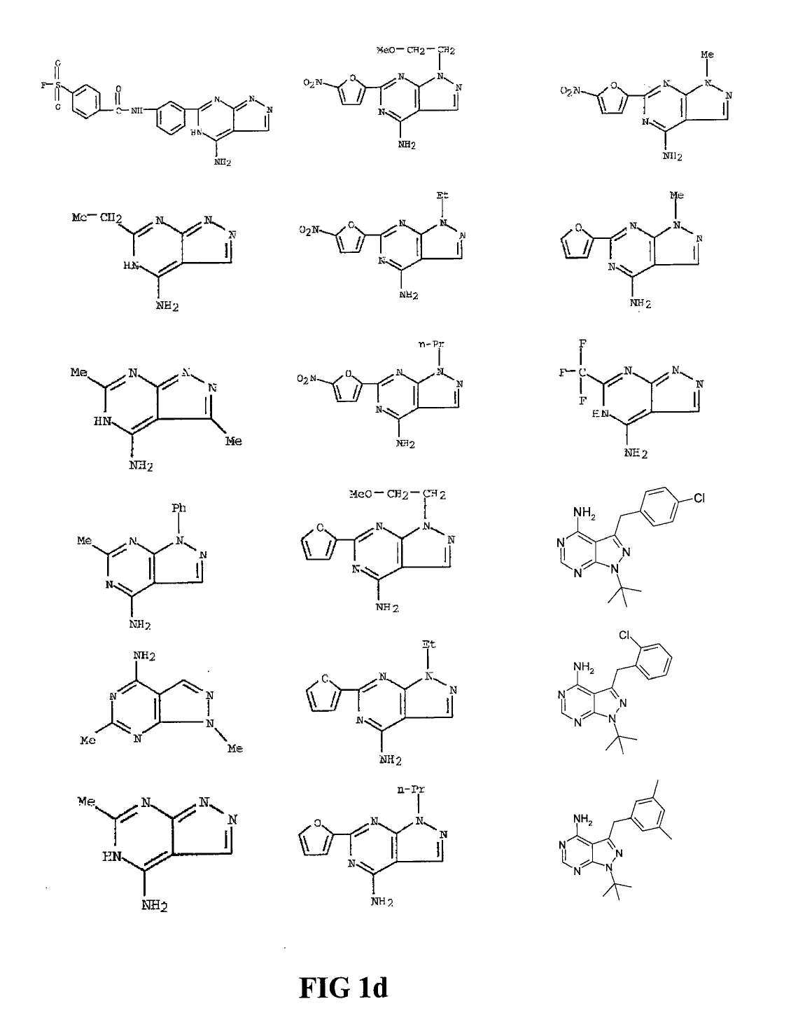

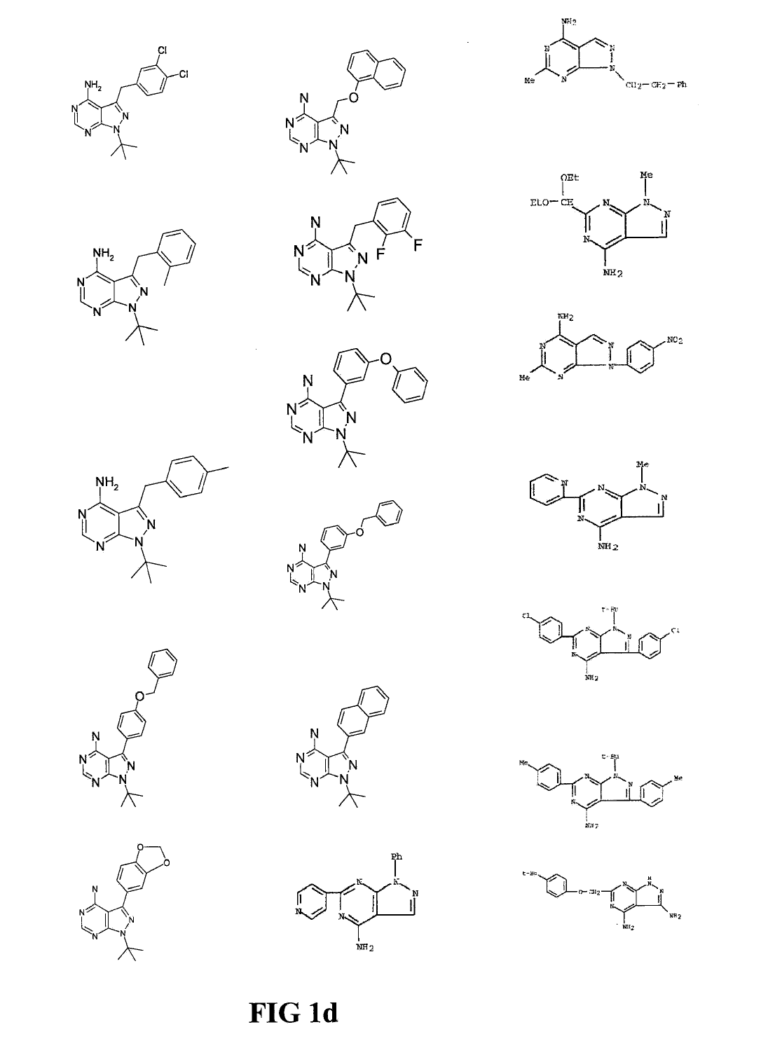

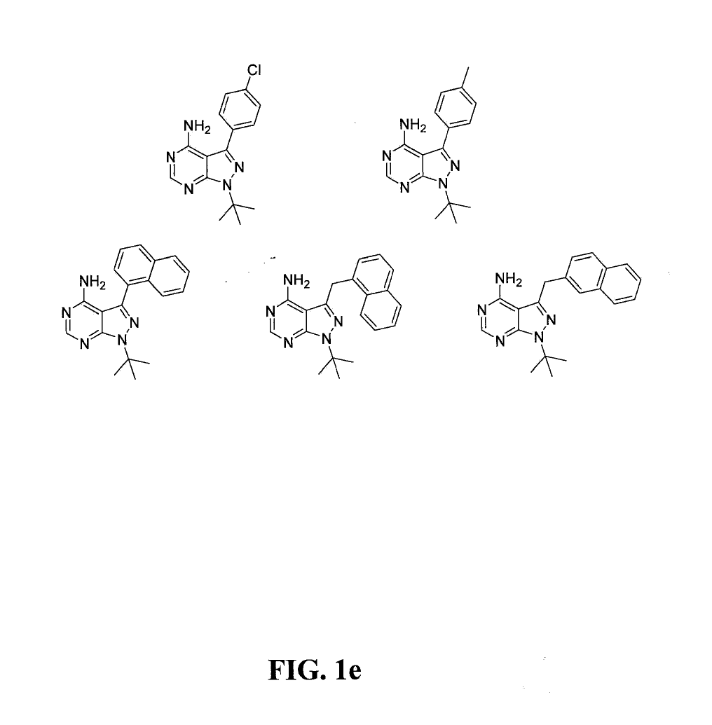

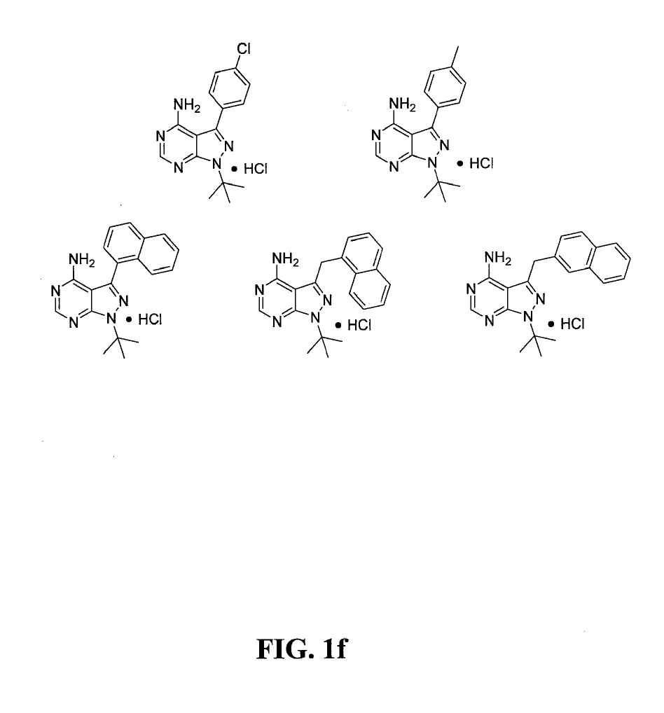

13. The method of claim 1, wherein the compound is selected from the compounds set forth in FIG. 1A, 1B, 1C, 1D, 1E, 1F, 2, 3A, 3B, 4A, 4B, 5A, 5B, 6, 7, 8A, 8B, 8C, 9A, 9B, 9C, or 9D.

14. The method of claim 1, wherein the compound is represented by one of the following structural formulae:

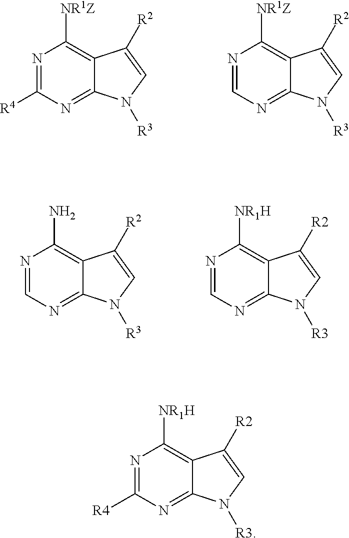

15. The method of claim 1, wherein the compound is represented by one of the following structural formulae:

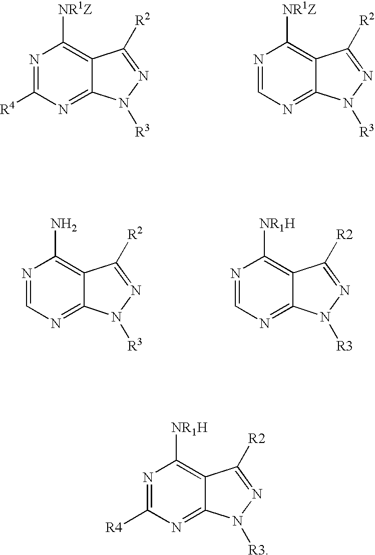

16. The method of claim 14, wherein R¹ is H.

17. The method of claim 14, wherein:
R² is H, halo, CN, NO₂, NH₂, or C₁-C₁₀ alkyl optionally substituted with 1-3 independent halo, SR⁵, OR⁵, OC(O)R⁵, NR⁵R⁵; COOR⁵, NO₂, CN, C(O)R⁵, OC(O)NR⁵R⁵, or C(O)NR⁵R⁵.

18. The method of claim 17, wherein R² is H, F, Cl, Br, CF₃, CCl₃, CN, NO₂, NH₂, or C₁-C₆ alkyl.

19. The method of claim 14, wherein R² is aryl, heteroaryl, aralkyl, or heteroaralkyl, each substituted with:
H, halo, SR⁵, OR⁵, OC(O)R⁵, NR⁵R⁵, COOR⁵, NO₂, CN, C(O)R⁵, OC(O)NR⁵R⁵, or C(O)NR⁵R⁵; or
aryl, C₁-C₁₀ alkyl, or C₂-C₁₀ alkenyl each optionally substituted with 1-3 independent aryl, halo, SR⁵, OR⁵, OC(O)R⁵, NR⁵R⁵, COOR⁵, NO₂, CN, C(O)R⁵, OC(O)NR⁵R⁵, or C(O)NR⁵R⁵.

20. The method of claim 19, wherein the optionally substituted aryl, heteroaryl, aralkyl, or heteroaralkyl groups in R² are selected from phenyl, napthyl, benzyl, phenylethylene, napthylmethylene, phenoxymethylene, napthyloxymethylene, pyridylmethylene, benzofurylmethylene, dihydrobenzofurylmethylene, benzodioxolmethylene, indanylmethylene, furyl, thienyl, pyridyl, benzothienyl, and benzofuryl.

21. The method of claim 19, wherein the optional substituents for the aryl, heteroaryl, aralkyl, or heteroaralkyl groups in R² are:
H, F, Cl, Br, OH, C₁-C₆ alkoxy, amino, C₁-C₆ alkylamino, COOH, COO—C₁-C₆ alkyl, NO₂, CN, or C(O)—C₁-C₆ alkyl; or
C₁-C₆ alkyl, C₂-C₆ alkenyl, or aryl optionally substituted with phenyl, F, Cl, Br, C₁-C₆ alkoxy, COOH, COO—C₁-C₆ alkyl, NO₂, or CN.

22. The method of claim 19, wherein R³ is:
H, C₃-C₁₀ cycloalkyl, or C₂-C₁₀ alkynyl; or
C₁-C₁₀ alkyl or C₂-C₁₀ alkenyl each optionally substituted with 1-3 halo, CF₃, SR⁵, OR⁵, OC(O)R⁵, NR⁵R⁵, COOR⁵, NO₂, CN, C(O)R⁵, OC(O)NR⁵R⁵, or C(O)NR⁵R⁵.

23. The method of claim 19, wherein R³ is:
H, C₁-C₈ alkyl optionally substituted with 1-3 halo, OR⁵, NR⁵R⁵, COOR⁵, C(O)R⁵, C(O)NR⁵R⁵, C₂-C₆ alkenyl, or C₂-C₆ alkynyl; or
cyclopropyl, cyclopropylmethyl, cyclobutyl, cyclobutylmethyl, cyclopentyl, cyclopentylmethyl, cyclohexyl, or cyclohexylmethyl.

24. The method of claim 17, wherein R³ is aryl, heteroaryl, aralkyl, heteroaralkyl, heterocyclyl, or heterocyclyalkyl, each substituted with:
H, alkyl, halo, OR⁵, OC(O)R⁵, NR⁵R⁵, COOR⁵, NO₂, CN, C(O)R⁵, OC(O)NR⁵R⁵, or C(O)NR⁵R⁵; or
optionally substituted aryl, heteroaryl, or heterocyclyl.

25. The method of claim 24, wherein the aryl, heteroaryl, aralkyl, heteroaralkyl, heterocyclyl, or heterocyclyalkyl groups represented by R³ are selected from benzyl, pyridyl, pyridylmethylene, furyl, thienyl, tetrahydrofuryl, or tetrahydrothienyl.

26. The method of claim 25, wherein substituents for the aryl, heteroaryl, aralkyl, heteroaralkyl, heterocyclyl, or heterocyclyalkyl groups represented by R³ are:
H, F, Cl, Br, SR⁵, OR⁵, NR⁵R⁵, COOR⁵, NO₂, CN, C(O)R⁵; or
C₁-C₆ alkyl, C₂-C₆ alkenyl, or aryl optionally substituted with phenyl, F, Cl, Br, SR⁵, OR⁵, COOR⁵, NO₂, or CN.

27. The method of claim 1, wherein R⁴ is independently aryl; heteroaryl; C₁-C₁₀ alkyl or C₂-C₁₀ alkenyl, each optionally substituted with 1-3 independent aryl, or heteroaryl; C₂-C₁₀ alkynyl; halo; haloalkyl; CF₃; SR⁵; OR⁵; OC(O)R⁵; NR⁵R⁵; NR⁵R⁶; COOR⁵; NO₂; CN; C(O)R⁵; C(O)C(O)R⁵; C(O)NR⁵R⁵; S(O)ₘR⁵; S(O)ₘNR⁵R⁵; NR⁵C(O)NR⁵R⁵; NR⁵C(O)C(O)R⁵; NR⁵C(O)R⁵; NR⁵(COOR⁵); NR⁵C(O)R⁸; NR⁵S(O)ₘNR⁵R⁵; NR⁵S(O)ₘR⁵; NR⁵S(O)ₘR⁸; NR⁵C(O)C(O)NR⁵R⁵; or NR⁵C(O)C(O)NR⁵R⁶.

28. The method of claim 27, wherein R⁴ is
H, OR⁵, OC(O)R⁵, NR⁵R⁵, COOR⁵, NO₂, CN, C(O)R⁵, C(O)C(O)R⁵, or C(O)NR⁵R⁵; or
C₁-C₁₀ alkyl optionally substituted with 1-3 halo, OR⁵, OC(O)R⁵, NR⁵R⁵; COOR⁵, NO₂, CN, C(O)R⁵, OC(O)NR⁵R⁵, or C(O)NR⁵R⁵.

29. The method of claim 27, wherein R⁴ is
H, CF₃, CCl₃, amino, C₁-C₆ alkoxy, COOH, COO—C₁-C₆ alkyl, OC(O)—C₁-C₆ alkyl, phenoxy, or alkylphenoxy; or
C₁-C₆ alkyl optionally substituted with amino, COOH, COO—C₁-C₆ alkyl or OC(O)—C₁-C₆ alkyl, or 1 or 2 C₁-C₆ alkoxy.

30. The method of claim 27, wherein R⁴ is an optionally substituted aryl, aralkyl, heteroaryl, or heteroaralkyl, wherein the optional substituents in R⁴ are halo, CF₃, SR⁵, OR⁵, OC(O)R⁵, NR⁵R⁵, COOR⁵, NO₂, CN, C(O)R⁵, OC(O)NR⁵R⁵, C(O)NR⁵R⁵, N(R⁵)C(O)R⁵, N(R⁵)(COOR⁵), or S(O)ₘNR⁵R⁵.

31. The method of claim 30, wherein the aryl, aralkyl, heteroaryl, and heteroaralkyl groups represented by R⁴ are selected from phenyl, benzyl, pyridyl, pyridylmethylene, furyl, furylmethylene, thienyl, thienylmethylene, pyrazolyl, and pyrazolylmethylene.

32. The method of claim 30, wherein the optional substituents for the aryl, aralkyl, heteroaryl, or heteroaralkyl groups represented by R⁴ are:
F, Cl, OH, amino, NO₂, C₁-C₆ alkoxy, C₁-C₆ alkyl, phenoxy, or alkylphenoxy; or
phenyl, imidazolyl, or morpholino optionally substituted with F, Cl, amino, NO₂, C₁-C₆ alkoxy, or C₁-C₆ alkyl.

33. The method of claim 1, wherein the compound is selected from the compounds identified in Table I.

34. A compound represented by the following structural formula: 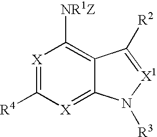
or a pharmaceutically acceptable salt thereof, wherein:
m is 1 or 2;
each X and X¹ is independently N, CH, or C(C₁-C₄ alkyl);
R¹ and Z are each independently H, R⁵, C(O)R⁶, COOR⁵, C(O)NR⁶R⁶, or S(O)ₘR⁵; or, NR¹Z, taken together, is N═CH—NR⁵R⁵
R² is SR⁹, OR⁵, OC(O)R⁵, NR⁵R⁵, NR⁵R⁶, COOR⁵, C(O)R⁵, C(O)H, C(O)C(O)R⁵, C(O)NR⁵R⁵, C(O)NR⁵R⁶, C(O)NR⁶R⁶, S(O)ₘR⁹, S(O)ₘNR⁵R⁵, S(O)ₘNR⁵R⁶, NR⁵C(O)NR⁵R⁵, NR⁶C(O)NR⁶R⁶, NR⁵C(O)C(O)R⁵, NR⁵C(O)C(O)R⁵, NR⁵C(O)R⁵, NR⁶C(O)R⁵, NR⁵(COOR⁵), NR⁶(COOR⁵), NR⁵C(O)R⁸, NR⁶C(O)R⁸, NR⁵S(O)ₘNR⁵R⁵, NR⁶S(O)ₘNR⁶R⁶, NR⁵S(O)ₘR⁵, NR⁶S(O)ₘR⁵, NR⁵S(O)ₘR⁸, NR⁶S(O)ₘR⁸, NR⁵C(O)C(O)NR⁵R⁵, NR⁶C(O)C(O)NR⁵R⁶, or NR⁵C(O)C(O)NR⁵R⁶;
R³ is R¹⁰, COOR⁵, C(O)R⁵, C(O)C(O)R⁵, C(O)NR⁵R⁵, C(O)NR⁵R⁶, C(O)NR⁶R⁶, S(O)ₘR⁵, S(O)ₘNR⁵R⁵, S(O)ₘNR⁵R⁶, P(O)R⁵R⁵, P(O)(NR⁵R⁵)₂, P(O)(NR⁵R⁶)₂, P(O)(NR⁶R⁶)₂, or P(O)(OR⁵)₂;
R⁴ is H, halo, pseudohalo, CN, SR⁵, OR⁵, OC(O)R⁵, NR⁵R⁵, NR⁵R⁶, COOR⁵, NO₂, C(O)R⁵, C(O)C(O)R⁵, C(O)NR⁵R⁵, C(O)NR⁵R⁶, C(O)NR⁶R⁶, S(O)ₘR⁵, S(O)ₘNR⁵R⁵, S(O)ₘNR⁵R⁶, NR⁵C(O)NR⁵R⁵, NR⁶C(O)NR⁶R⁶, NR⁵C(O)C(O)R⁵, NR⁵C(O)C(O)R⁵, NR⁵C(O)R⁵, NR⁶C(O)R⁵, NR⁵(COOR⁵), NR⁶(COOR⁵), NR⁵C(O)R⁸, NR⁶C(O)R⁸, NR⁵S(O)ₘNR⁵R⁵, NR⁶S(O)ₘNR⁶R⁶, NR⁵S(O)ₘR⁵, NR⁶S(O)ₘR⁵, NR⁵S(O)ₘR⁸, NR⁶S(O)ₘR⁸, NR⁵C(O)C(O)NR⁵R⁵, NR⁶C(O)C(O)NR⁵R⁶, or NR⁵C(O)C(O)NR⁵R⁶, NR⁶C(O)C(O)NR⁵R⁶, or NR⁵C(O)C(O)NR⁵R⁶, or optionally substituted alkyl, aryl, araalkyl, heteroaryl, or heteroaralkyl; and
each R⁵ is independently optionally substituted alkyl, alkenyl, alkynyl, cycloalkyl, cycloalkenyl, aryl, heteroaryl, or heterocyclyl,
each R⁶ and R⁸ is independently H or optionally substituted alkyl, alkenyl, alkynyl, cycloalkyl, cycloalkenyl, aryl, heteroaryl, or heterocyclyl,
each R⁹ is independently optionally substituted alkyl containing 2 or more carbons, alkenyl, alkynyl, cycloalkyl, cycloalkenyl, aryl, heteroaryl, or heterocyclyl,
each R¹⁰ is independently optionally substituted alkyl, alkenyl, alkynyl, cycloalkyl, cycloalkenyl, aryl, heteroaryl, or heterocyclyl, excluding optionally substituted dihydrofur-2-yl and tetrahydrofur-2-yl;
wherein:
when R² is C(O)R⁵ then R³ is not methyl, 2-propyl, cyclopentyl, or 4-piperidyl;
when each X and X¹ is N and R³ is CH₃, R⁴ is not N(CH₃)₂ or S-alkyl;
when Z, R¹ and R⁴ are H;
each X and X¹ is N;
R² is CO substituted with methyl, phenyl, 4-bromophenyl, 4-chlorophenyl, 4-chlorophenyl, naphth-2-yl, (3-methyl-5-phenyl)thiazol-2-yl, 4-(piperidin-1-ylsulfonyl)phenyl, thien-2-yl, or benzothiazol-2-yl, then R³ is not phenyl, 4-chlorophenyl, or 4-methylphenyl;
R² is CONH₂, then R³ is not methyl, phenyl, or CH₂OCH₂CH₂OH;
R² is alkoxy, then R³ is not tert-butyl;
each X is N; X¹ is CH;
R² is benzoyl substituted at the meta position with: NH₂, NHSO₂-(chloro-substituted phenyl), NHSO₂-thien-2-yl, NHCONH-(halo or methyl substituted phenyl), NHCONH-(methybenzyl), NHCONH-cyclohexyl, or NHCO-(chloro phenyl); then R³ is not CH₂-cyclopropyl;
R³ is CH₂O-benzyl, CH₂O-alkyl, alkyl or alkenyl optionally substituted with hydroxyl, alkoxy, hydroxyalkyl or hydroxyalkyloxy; or optionally substituted aralkyl; then R² is not CONH₂;
R² is S-phenyl substituted with NH₂, NC(O)O-t-butyl, NC(O)NH-(2-fluorophenyl), NS(O)₂-(mono or di-fluorophenyl) then R³ is not cyclopentyl;
each X and X¹ is CH;
R³ is 2-(morpholin-1-yl)ethylene; then R² is not CO-tetramethylcyclopropane;
R³ is methyl, then R² is not COH or carboxyl
each X is N, X¹ is N or CH, and R³ is 4-(4-methyl-piperizin-1-yl)cyclohexyl, 4-(N-morpholinyl)cyclohexyl or phenyl, R² is not CONH-(optionally substituted phenyl) or N (optionally substituted phenyl) C(O)(phenyl or alkylphenyl);
when each X and X¹ is N, R⁴ is H or phenyl, Z is H or optionally substituted phenyl, R¹ is H, and R² is NH-(pyridyl or optionally substituted phenyl), R³ is not methyl, hydroxyalkyl, benzyl or 6-p-tolylpyridazin-3-yl.

35. The compound of claim 34, wherein the compound is represented by the following structural formula:

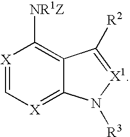

36. The compound of claim 34, wherein the compound is represented by the following structural formula:

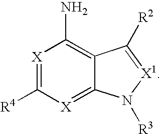

37. The compound of claim 34, wherein the compound is represented by the following structural formula:

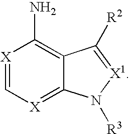

38. The compound of claim 34, wherein the compound is represented by the following structural formula:

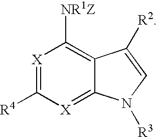

39. The compound of claim 34, wherein the compound is represented by the following structural formula:

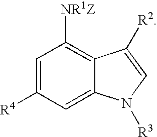

40. The compound of claim 34, wherein the compound is represented by one of the following structural formulae:

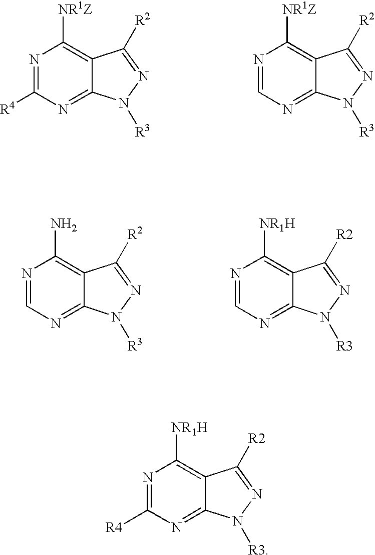

41. The compound of claim 34, wherein the compound is represented by one of the following structural formulae:

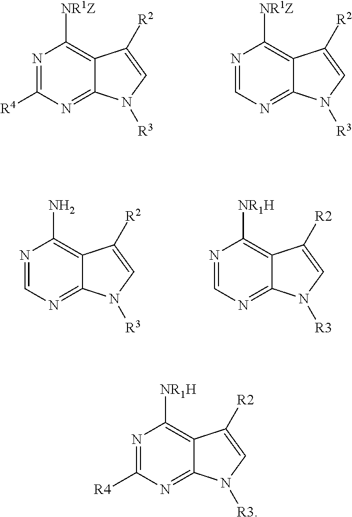

42. The compound of claim 34, wherein R² is SR⁹, OR⁵, OC(O)R⁵, NR⁵R⁵, NR⁵R⁶, COOR⁵, C(O)H, C(O)C(O)R⁵, C(O)NR⁵R⁵, C(O)NR⁵R⁶, S(O)ₘR⁹, S(O)ₘNR⁵R⁵, S(O)ₘNR⁵R⁶, NR⁵C(O)NR⁵R⁵, NR⁶C(O)NR⁶R⁶, NR⁵C(O)C(O)R⁵, NR⁵C(O)C(O)R⁵, NR⁵C(O)R⁵, NR⁶C(O)R⁵, NR⁵(COOR⁵), NR⁶(COOR⁵), NR⁵C(O)R⁸, NR⁶C(O)R⁸, NR⁵S(O)ₘNR⁵R⁵, NR⁶S(O)ₘNR⁶R⁶, NR⁵S(O)ₘR⁵, NR⁶S(O)ₘR⁵, NR⁵S(O)ₘR⁸, NR⁶S(O)ₘR⁸, NR⁵C(O)C(O)NR⁵R⁵, NR⁶C(O)C(O)NR⁵R⁶, or NR⁵C(O)C(O)NR⁵R⁶.

43. The compound of claim 34, wherein R² is SR⁹, OR⁵, OC(O)R⁵, NR⁵R⁵, NR⁵R⁶, COOR⁵, C(O)H, C(O)C(O)R⁵, S(O)ₘR⁹, S(O)ₘNR⁵R⁵, S(O)ₘNR⁵R⁶, NR⁵C(O)NR⁵R⁵, NR⁶C(O)NR⁶R⁶, NR⁵C(O)C(O)R⁵, NR⁵C(O)C(O)R⁵, NR⁵C(O)R⁵, NR⁶C(O)R⁵, NR⁵(COOR⁵), NR⁶(COOR⁵), NR⁵C(O)R⁸, NR⁶C(O)R⁸, NR⁵S(O)ₘNR⁵R⁵, NR⁶S(O)ₘNR⁶R⁶, NR⁵S(O)ₘR⁵, NR⁶S(O)ₘR⁵, NR⁵S(O)ₘR⁸, NR⁶S(O)ₘR⁸, NR⁵C(O)C(O)NR⁵R⁵, NR⁶C(O)C(O)NR⁵R⁶, or NR⁵C(O)C(O)NR⁵R⁶.

44. The compound of claim 34, wherein R² is independently is NR⁵R⁵, NR⁵R⁶, NR⁵C(O)NR⁵R⁵, NR⁶C(O)NR⁶R⁶, NR⁵C(O)C(O)R⁵, NR⁵C(O)C(O)R⁵, NR⁵C(O)R⁵, NR⁶C(O)R⁵, NR⁵(COOR⁵), NR⁶(COOR⁵), NR⁵C(O)R⁸, NR⁶C(O)R⁸, NR⁵S(O)ₘNR⁵R⁵, NR⁶S(O)ₘNR⁶R⁶, NR⁵S(O)ₘR⁵, NR⁶S(O)ₘR⁵, NR⁵S(O)ₘR⁸, NR⁶S(O)ₘR⁸, NR⁵C(O)C(O)NR⁵R⁵, NR⁶C(O)C(O)NR⁵R⁶, or NR⁵C(O)C(O)NR⁵R⁶.

45. The compound of claim 34, wherein R² is OR⁵.

46. The compound of claim 45, wherein R⁵ is optionally substituted aryl or heteroaryl.

47. The compound of claim 45, wherein R⁵ is optionally substituted alkyl, cycloalkyl or heteroalkyl.

48. The compound of claim 34, wherein R² is SR⁹.

49. The compound of claim 48, wherein R⁹ is optionally substituted aryl or heteroaryl.

50. The compound of claim 49, wherein R⁹ is optionally substituted cycloalkyl, heteroalkyl, or alkyl with 2 or more carbons.

51. The compound of claim 34, wherein R² is NR⁵R⁵ or NR⁵R⁶.

52. The compound of claim 51, wherein R⁵ is optionally substituted aryl or heteroaryl.

53. The compound of claim 51, wherein R⁵ is optionally substituted alkyl, cycloalkyl, or heteroalkyl.

54. The compound of claim 34, wherein R² is S(O)ₘR⁹, S(O)ₘNR⁵R⁵, or S(O)ₘNR⁵R⁶.

55. The compound of claim 54, wherein R⁵ is optionally substituted aryl or heteroaryl.

56. The compound of claim 54, wherein R⁵ is optionally substituted alkyl, cycloalkyl, or heteroalkyl.

57. The compound of claim 54, wherein R⁹ is optionally substituted aryl or heteroaryl.

58. The compound of claim 54, wherein R⁹ is optionally substituted cycloalkyl, heteroalkyl, or alkyl with 2 or more carbons.

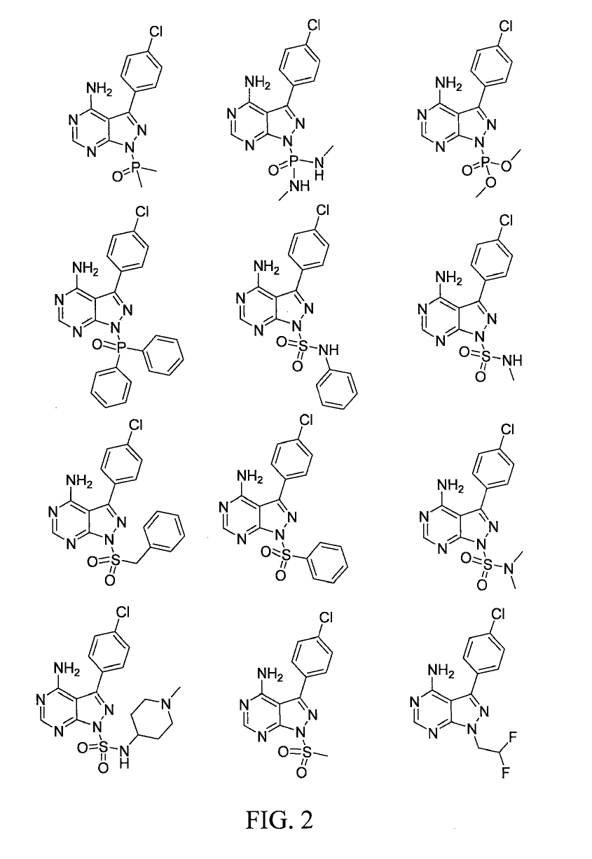

59. A compound as set forth in any one of FIG. 2, 3A, 4A, 5A, 5B, 6, or 7.

60. A method of treating a disorder characterized by impaired protein trafficking, comprising administering to a subject or contacting a cell with a compound of claim 34.

61. The method of claim 1, wherein the disorder is a lysosomal storage disorder.

62. The method of claim 61, wherein the lysosomal storage disorder is Fabry disease, Farber disease, Gaucher disease, GM₁-gangliosidosis, Tay-Sachs disease, Sandhoff disease, GM₂ activator disease, Krabbe disease, metachromatic leukodystrophy, Niemann-Pick disease (types A, B, and C), Hurler disease, Scheie disease, Hunter disease, Sanfilippo disease, Morquio disease, Maroteaux-Lamy disease, hyaluronidase deficiency, aspartylglucosaminuria, fucosidosis, mannosidosis, Schindler disease, sialidosis type 1, Pompe disease, Pycnodysostosis, ceroid lipofuscinosis, cholesterol ester storage disease, Wolman disease, Multiple sulfatase, galactosialidosis, mucolipidosis (types II, III, and IV), cystinosis, sialic acid storage disorder, chylomicron retention disease with Marinesco-Sjögren syndrome, Hermansky-Pudlak syndrome, Chediak-Higashi syndrome, Danon disease, or Geleophysic dysplasia.

63. The method of claim 1, wherein the disorder is characterized by an impaired delivery of cargo to a cellular compartment.

64. The method of claim 1, wherein the disorder is characterized by a Rab27a mutation or a deficiency of Rab27a.

65. The method of claim 64, wherein the disorder is Griscelli syndrome.

66. The method of claim 1, wherein the disorder is cystic fibrosis.

67. The method of claim 1, wherein the disorder is cystic fibrosis characterized by impaired protein trafficking.

68. The method of claim 1, wherein the disorder is cystic fibrosis characterized by impaired cystic fibrosis transmembrane conductance regulator (CFTR) activity.

69. The method of claim 1, wherein the disorder is cystic fibrosis characterized by impaired protein trafficking and by impaired cystic fibrosis transmembrane conductance regulator (CFTR) activity.

70. The method of claim 1, wherein the disorder is diabetes.

71. The method of 70, wherein the diabetes is diabetes mellitus.

72. The method of claim 1, wherein the disorder is hereditary emphysema (α-1-antitrypsin deficiency), hereditary hemochromatosis, oculocutaneous albinism, protein C deficiency, type I hereditary angioedema, congenital sucrase-isomaltase deficiency, Crigler-Najjar type II, Laron syndrome, hereditary Myeloperoxidase, primary hypothyroidism, congenital long QT syndrome, thyroxine binding globulin deficiency, familial hypercholesterolemia, familial chylomicronemia, abeta-lipoproteinema, low plasma lipoprotein a levels, hereditary emphysema with liver injury, congenital hypothyroidism, osteogenesis imperfecta, hereditary hypofibrinogenemia, α-1-antichymotrypsin deficiency, nephrogenic diabetes insipidus, neurohypophyseal diabetes, insipidus, Charcot-Marie-Tooth syndrome, Pelizaeus Merzbacher disease, von Willebrand disease type IIA, combined factors V and VIII deficiency, spondylo-epiphyseal dysplasia tarda, choroideremia, I cell disease, Batten disease, ataxia telangiectasias, acute lymphoblastic leukemia, acute myeloid leukemia, myeloid leukemia, ADPKD-autosomal dominant polycystic kidney disease, microvillus inclusion disease, tuberous sclerosis, oculocerebro-renal syndrome of Lowe, amyotrophic lateral sclerosis, myelodysplastic syndrome, Bare lymphocyte syndrome, Tangier disease, familial intrahepatic cholestasis, X-linked adreno-leukodystrophy, Scott syndrome, Hermansky-Pudlak syndrome types 1 and 2, Zellweger syndrome, rhizomelic chondrodysplasia puncta, autosomal recessive primary hyperoxaluria, Mohr Tranebjaerg syndrome, spinal and bullar muscular atrophy, primary ciliary diskenesia (Kartagener's syndrome), Miller Dieker syndrome, lissencephaly, motor neuron disease, Usher's syndrome, Wiskott-Aldrich syndrome, Optiz syndrome, Huntington's disease, hereditary pancreatitis, anti-phospholipid syndrome, overlap connective tissue disease, Sjögren's syndrome, stiff-man syndrome, Brugada syndrome, Finnish congenital nephritic syndrome, Dubin-Johnson syndrome, X-linked hypophosphosphatemia, Pendred syndrome, persistent hyperinsulinemic hypoglycemia of infancy, hereditary spherocytosis, aceruloplasminemia, infantile neuronal ceroid lipofuscinosis, pseudoachondroplasia and multiple epiphyseal, Stargardt-like macular dystrophy, X-linked Charcot-Marie-Tooth disease, autosomal dominant retinitis pigmentosa, Wolcott-Rallison syndrome, Cushing's disease, limb-girdle muscular dystrophy, mucoploy-saccharidosis type IV, Finnish hereditary familial amyloidosis, glycogen storage disease type IV, sarcoma, chronic myelomonocytic leukemia, cardiomyopathy, faciogenital dysplasia, Torsion disease, Huntington and spinocerebellar ataxias, hereditary hyperhomosyteinemia, polyneuropathy, lower motor neuron disease, pigmented retinitis, seronegative polyarthritis, interstitial pulmonary fibrosis, Raynaud's phenomenon, Wegner's granulomatosis, preoteinuria, CDG-Ia, CDG-Ib, CDG-Ic, CDG-Id, CDG-Ie, CDG-If, CDG-IIa, CDG-IIb, CDG-IIc, CDG-IId, Ehlers-Danlos syndrome, multiple exostoses, Griscelli syndrome (type 1 or type 2), or X-linked non-specific mental retardation.

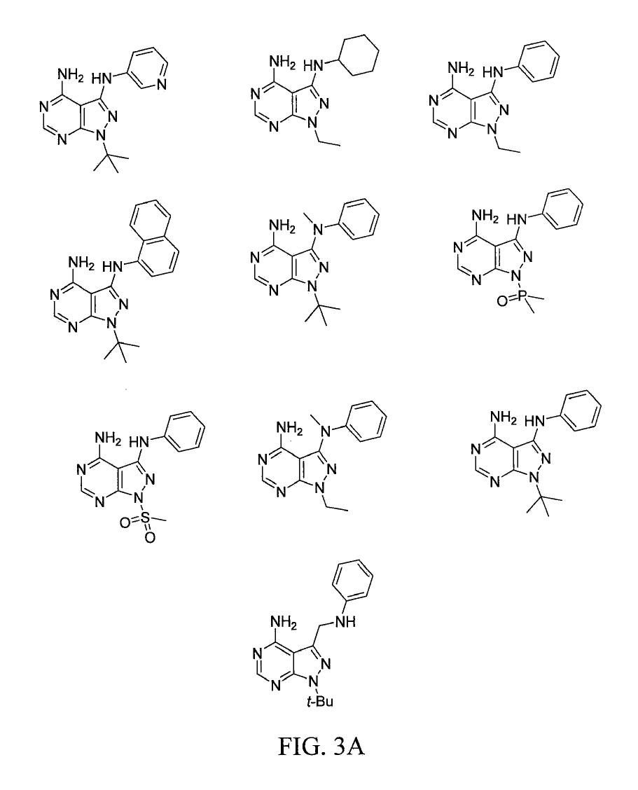

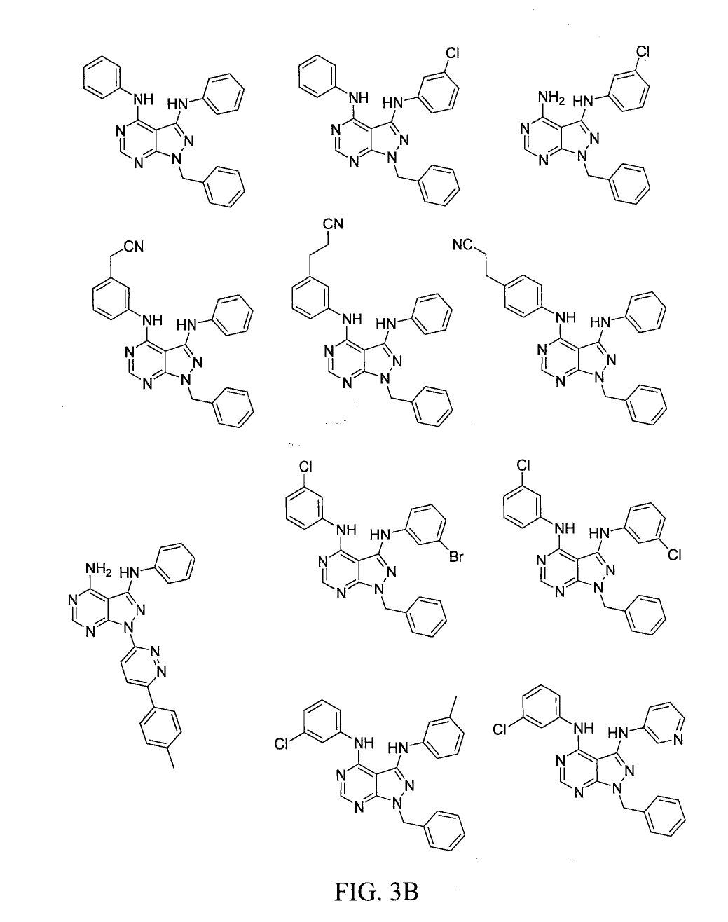

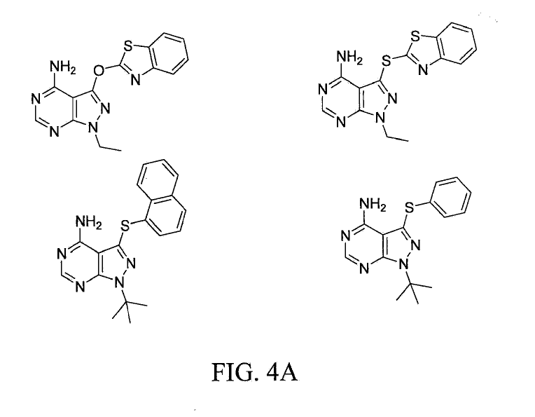

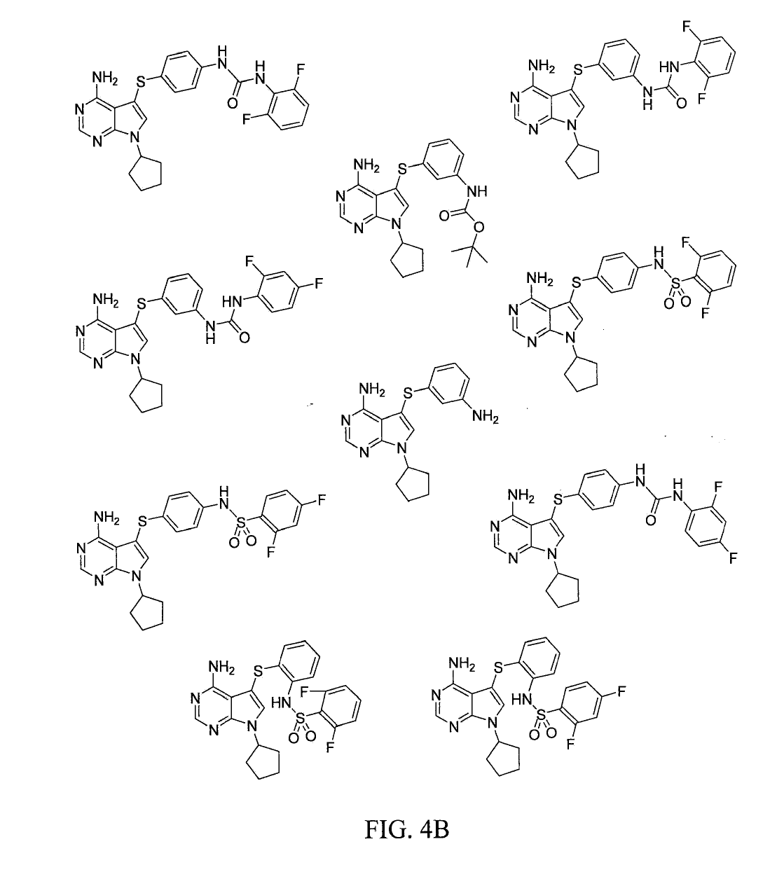

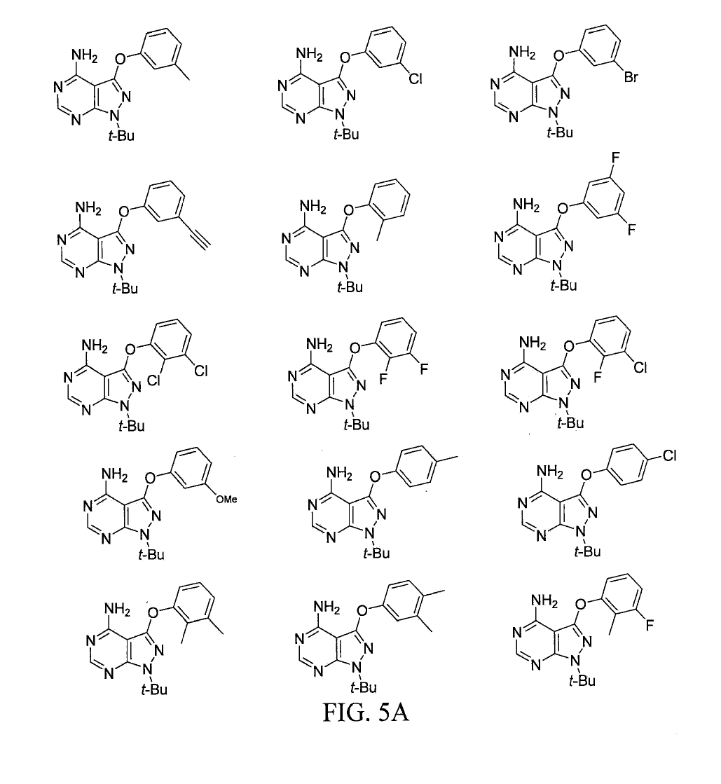

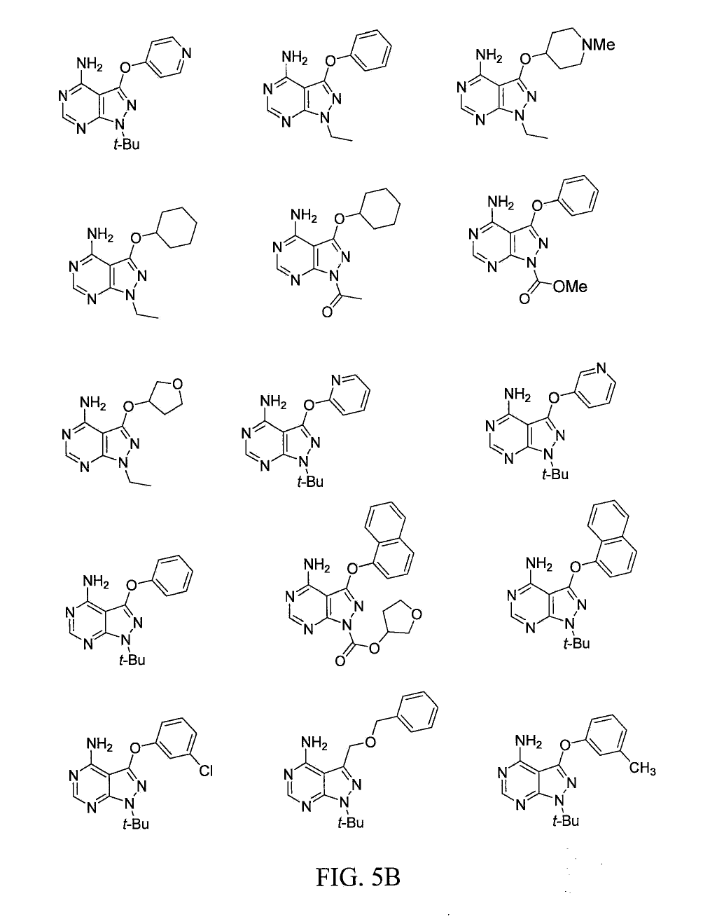

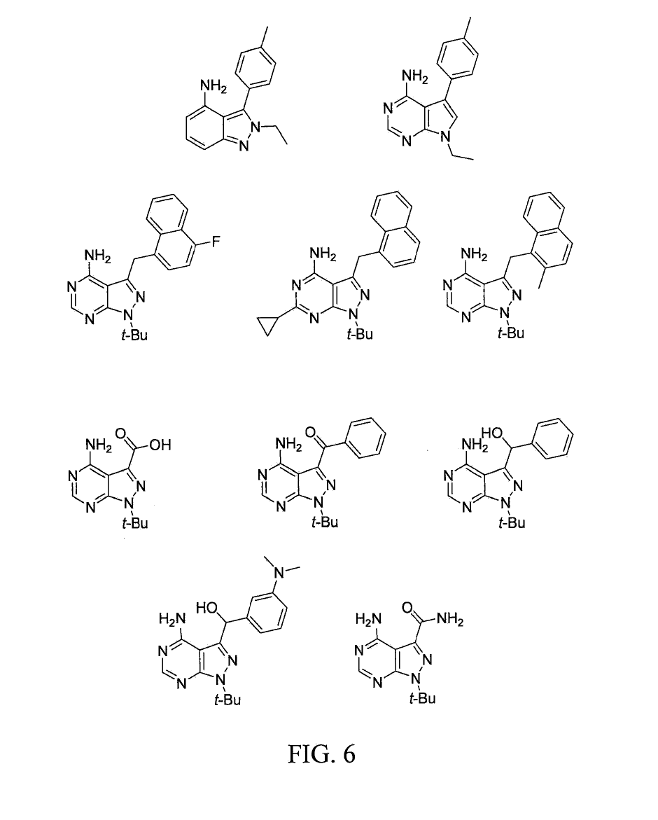

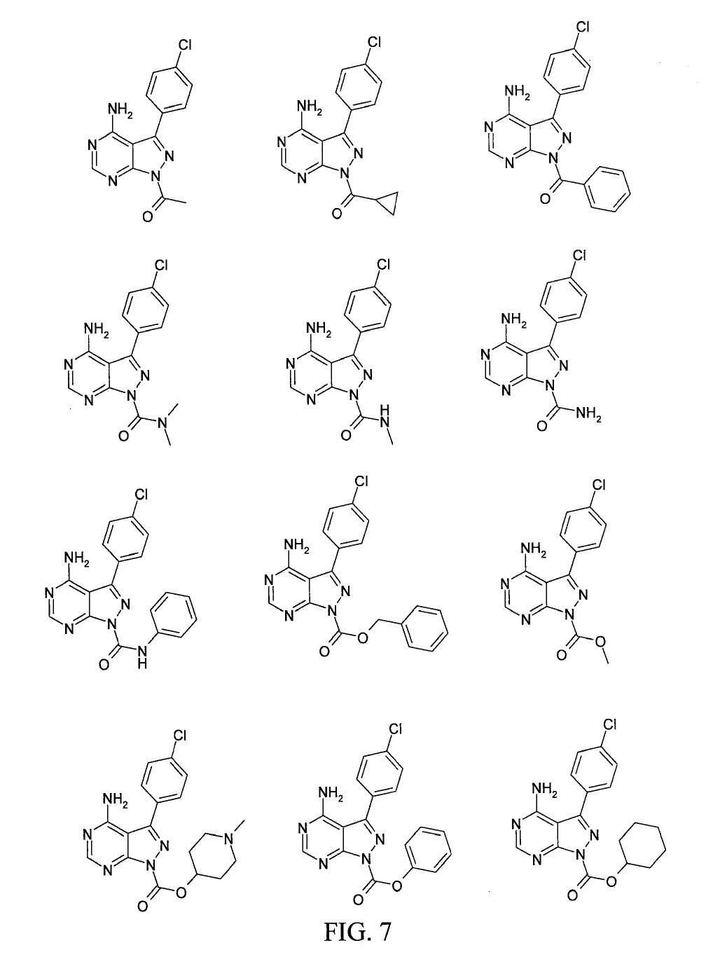

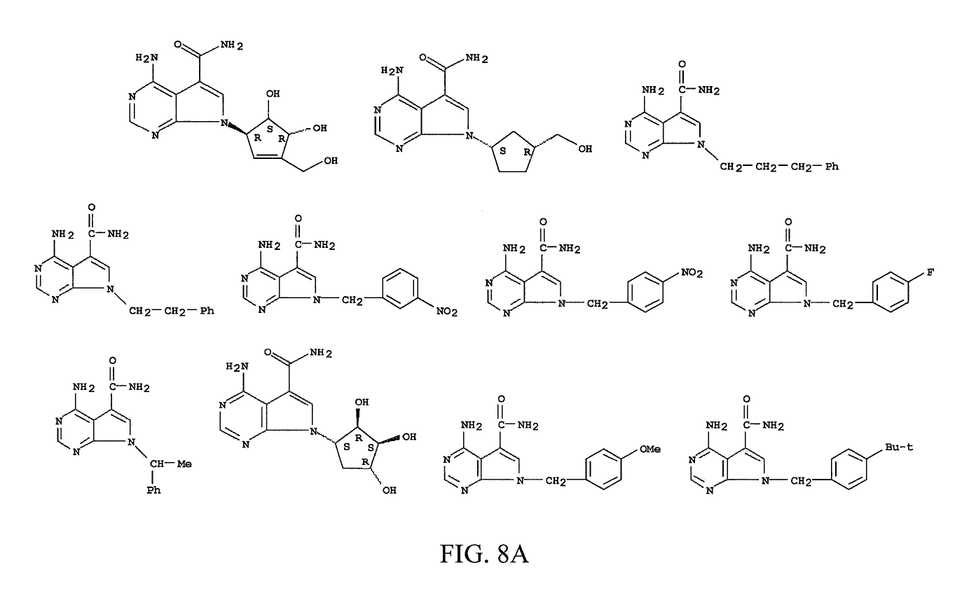

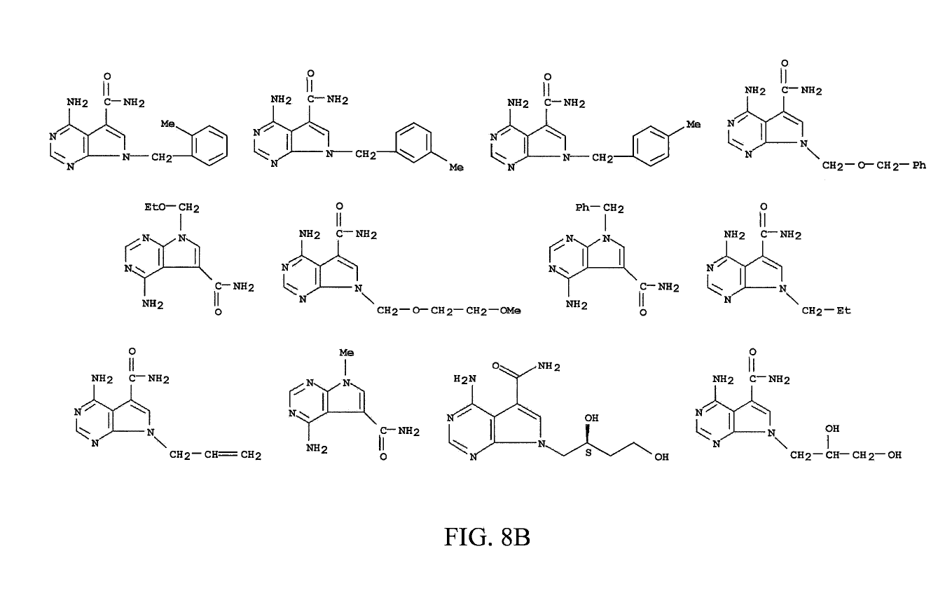

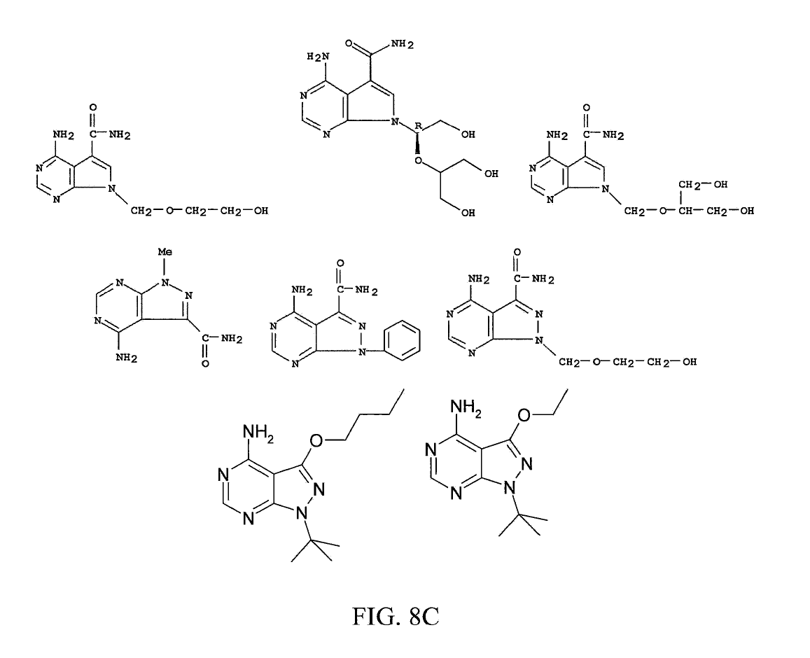

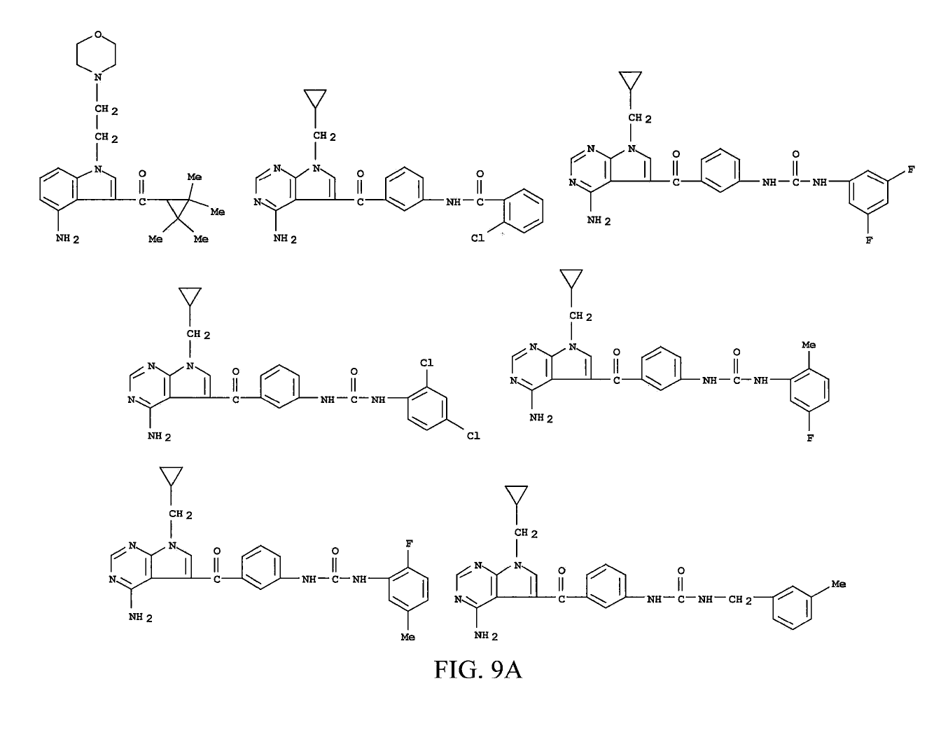

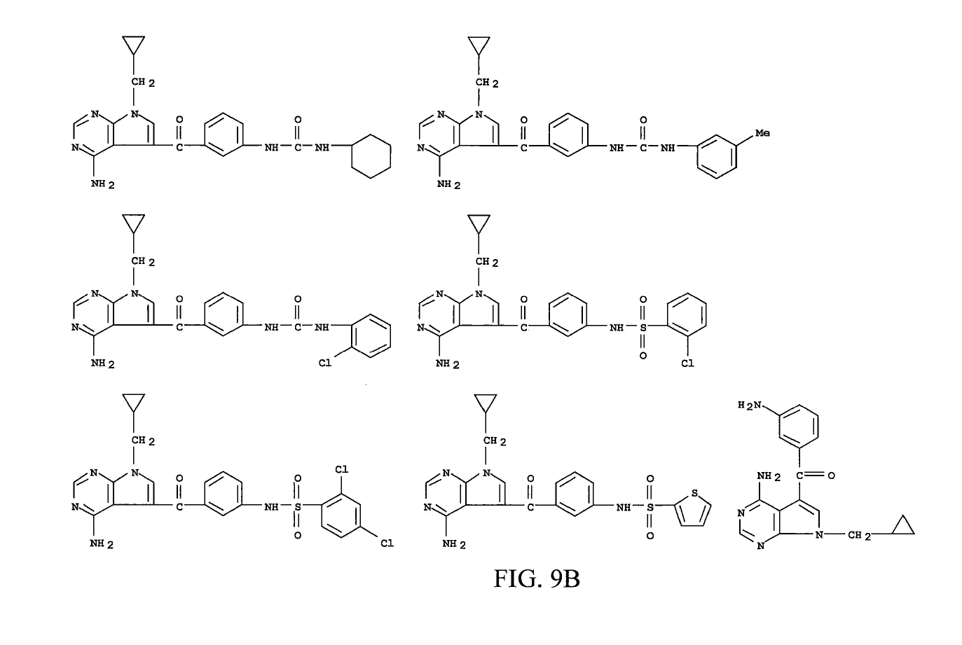

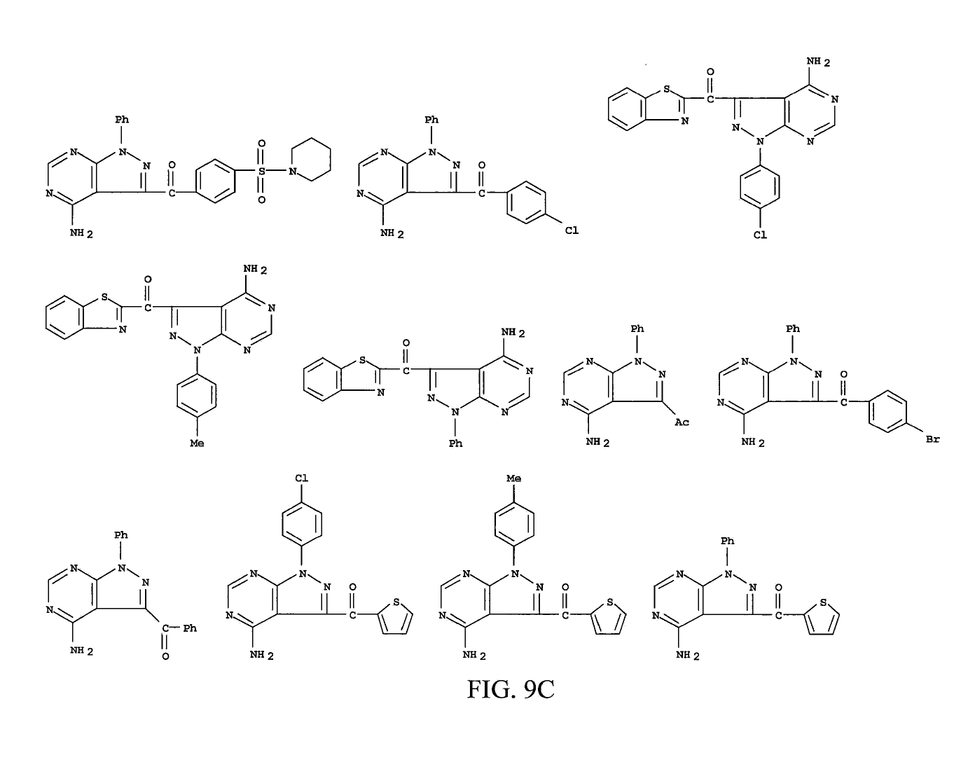

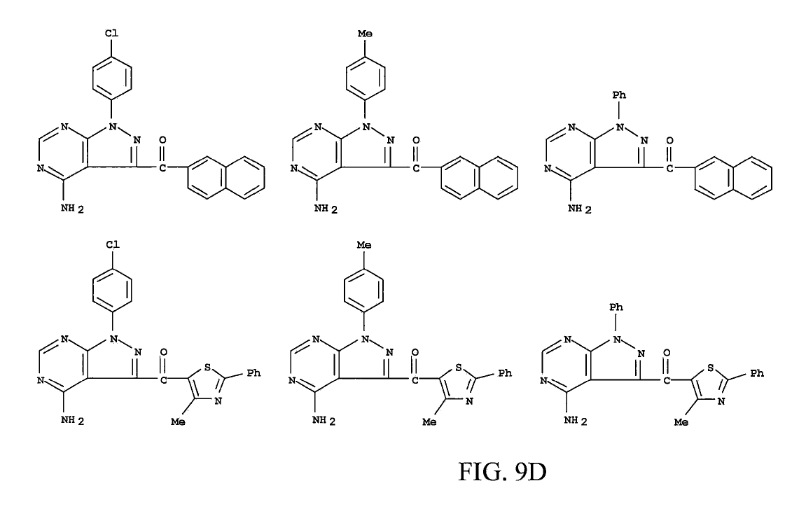

73. A method of treating a disorder characterized by impaired protein trafficking, comprising administering to a subject or contacting a cell with a compound represented in any of FIG. 3B, 4B, 8A, 8B, 8C, 9A, 9B, 9C, or 9D or pharmaceutically acceptable salts or derivatives thereof.

74. The method of claim 73, wherein the disorder is a synucleinopathy.

75. The method of claim 74, wherein the synucleinopathy is Parkinson's disease, familial Parkinson's disease, Lewy body disease, the Lewy body variant of Alzheimer's disease, dementia with Lewy bodies, multiple system atrophy, or the Parkinsonism-dementia complex of Guam.

76. The method of claim 1, wherein the subject is a human.

77. (canceled)

78. (canceled)

79. A method of producing a protein, the method comprising:
culturing a cell in the presence of a compound described in claim 1; and
purifying a protein produced by the cell,
wherein the culturing of the cell in the presence of the compound results in enhanced production of the purified protein as compared to culture of the cell in the absence of the compound.

80. The method of claim 79, wherein the protein is a recombinant protein encoded by a heterologous nucleic acid.

81. The method of claim 79 wherein the protein is a secreted protein.

82. (canceled)

83. (canceled)

84. (canceled)

85. (canceled)

86. A compound represented by the following structural formula: 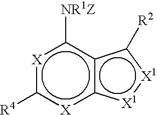
or pharmaceutically acceptable salts thereof, wherein:
m is 1 or 2;
each X is independently N, CH, or C(C₁-C₄ alkyl);
each X¹ is independently N, NR³, CH, or C(C₁-C₄ alkyl);
R¹ and Z are each independently R⁵, C(O)R⁵, COOR⁵, C(O)NR⁵R⁵, or S(O)ₘR⁵; or, NR¹Z, taken together, is N═CH—NR⁵R⁵
R² is N₃-substituted alkyl, alkenyl, alkynyl, cycloalkyl, cycloalkenyl, aryl, heteroaryl, or heterocyclyl, which may be further optionally substituted;
R³ is independently H, halo, pseudohalo, CN, SR⁵, R⁵, OR⁵, OC(O)R⁵, NR⁵R⁵, NR⁵R⁶, COOR⁵, NO₂, C(O)R⁵, C(O)C(O)R⁵, C(O)NR⁵R⁵, S(O)ₘR⁵, S(O)ₘNR⁵R⁵, NR⁵C(O)NR⁵R⁵, NR⁵C(O)C(O)R⁵, NR⁵C(O)R⁵, NR⁵(COOR⁵), NR⁵C(O)R⁸, NR⁵S(O)ₘNR⁵R⁵, NR⁵S(O)ₘR⁵, NR⁵S(O)ₘR⁸, NR⁵C(O)C(O)NR⁵R⁵, NR⁵C(O)C(O)NR⁵R⁶, P(O)R⁵R⁵, P(O)(NR⁵R⁵)₂, P(O)(NR⁵R⁶)₂, P(O)(NR⁶R⁶)₂, or P(O)(OR⁵)₂;
R⁴ is independently H, halo, pseudohalo, CN, SR⁵, OR⁵, OC(O)R⁵, NR⁵R⁵, NR⁵R⁶, COOR⁵, NO₂, C(O)R⁵, C(O)C(O)R⁵, C(O)NR⁵R⁵, S(O)ₘR⁵, S(O)ₘNR⁵R⁵, NR⁵C(O)NR⁵R⁵, NR⁵C(O)C(O)R⁵, NR⁵C(O)R⁵, NR⁵(COOR⁵), NR⁵C(O)R⁸, NR⁵S(O)ₘNR⁵R⁵, NR⁵S(O)ₘR⁵, NR⁵S(O)ₘR⁸, NR⁵C(O)C(O)NR⁵R⁵, or NR⁵C(O)C(O)NR⁵R⁶; or optionally substituted alkyl, aryl, aralkyl, heteroaryl, or heteroaralkyl; and
each R⁵, R⁶, and R⁸ is independently H or optionally substituted alkyl, alkenyl, alkynyl, cycloalkyl, cycloalkenyl, aryl, heteroaryl, or heterocyclyl.

87. A pharmaceutical composition, comprising the compound of claim 34 and a pharmaceutically acceptable carrier.
